(19) United States
(12) Patent Application Publication
(10) Pub. No.: US 2023/0391774 A1
(43) Pub. Date: Dec. 7, 2023

(54) Novel Compounds

(71) Applicant: AC Immune SA, Lausanne (CH)

(72) Inventors: Sreenivasachary Nampally, Ecublens (CH); Emanuele Gabellieri, Lausanne (CH); Veronique Dreyfus Dehlinger, Lausanne (CH)

(21) Appl. No.: 18/248,195

(22) PCT Filed: Oct. 11, 2021
(86) PCT No.: PCT/EP2021/078079

§ 371 Date: Apr. 6, 2023

Related U.S. Application Data
(60) Provisional application No. 63/092,105, filed on Oct. 15, 2020.

(51) Int. Cl. C07D 471/04; C07D 413/14; C07D 413/04; C07D 413/12; C07D 417/12; C07D 513/04; C07D 519/00; C07D 498/04; A61K 45/06; G01N 33/50

(52) U.S. Cl. CPC C07D 471/04; C07D 413/14; C07D 413/04; C07D 413/12; C07D 417/12; C07D 513/04; C07D 519/00; C07D 498/04; A61K 45/06; G01N 33/5058; G01N 33/5038

(57) ABSTRACT

The present invention relates to novel compounds that can be employed in the treatment, alleviation or prevention of a group of diseases, disorders and or abnormality associated with misfolding of Tau protein and/or pathological aggregation of Tau (Tubulin associated unit) protein including, but not limited to, Neurofibrillary Tangles (NFTs), such as Alzheimer's disease (AD). The present invention also relates to processes for the preparation of said compounds, pharmaceutical compositions comprising said compounds, methods using said compounds, combinations comprising said compounds, medicaments containing them, and their uses in diseases, disorders and/or abnormalities associated with misfolding of Tau protein and/or pathological aggregation of Tau (Tubulin associated unit) protein.

### FIELD OF THE INVENTION

The present invention relates to novel compounds that can be employed for the treatment, alleviation or prevention of diseases, disorders and/or abnormalities associated with misfolding of Tau protein and/or pathological aggregation of Tau (Tubulin associated unit) protein including, but not limited to, Neurofibrillary Tangles (NFTs), such as Alzheimer's disease (AD). The present invention also relates to processes for the preparation of said compounds, pharmaceutical compositions comprising said compounds, methods using said compounds, combinations comprising said compounds, medicaments containing them, and their uses in diseases, disorders and/or abnormalities associated with misfolding of Tau protein and/or pathological aggregation of Tau (Tubulin associated unit) protein.

### BACKGROUND OF THE INVENTION

Many aging diseases are based on or associated with extracellular or intracellular deposits of amyloid or amyloid-like proteins that contribute to the pathogenesis as well as to the progression of the diseases. The best characterized amyloid protein that forms extracellular aggregates is amyloid beta (Abeta, abeta or Aβ). Other examples of amyloid proteins that form extracellular aggregates are prion, ATTR (transthyretin) or ADan (ADanPP). Amyloid-like proteins that form mainly intracellular aggregates, include, but are not limited to Tau, alpha-synuclein, TAR DNA-binding protein 43 (TDP-43), and huntingtin (HTT). Diseases involving Tau aggregates are generally listed as tauopathies such as Alzheimer's disease (AD).

Amyloid or amyloid-like deposits result from misfolding of proteins followed by aggregation to give β-sheet assemblies in which multiple peptides or proteins are held together by inter-molecular hydrogen-bonds. While amyloid or amyloid-like proteins have different primary amino acid sequences, their deposits often contain many shared molecular constituents, in particular the presence of β-sheet quaternary structures. The association between amyloid deposits and diseases remains largely unclear. A diverse range of protein aggregates, including both those associated and not associated with disease pathologies, have been found to be toxic suggesting that the common molecular features of amyloid are implicated or responsible for disease on-set (Bucciantini et al., Nature, 2002, 416, 507-511). Various multimers of β-sheet aggregated peptides or proteins have also been associated with toxicity for different peptides or proteins ranging from dimers, through to soluble low molecular weight oligomers, protofibrils or insoluble fibrillar deposits.

Alzheimer's disease (AD) is a neurological disorder primarily thought to be caused by amyloid plaques, an extracellular accumulation of abnormal deposit of amyloid-beta (Abeta, abeta or Aβ) aggregates in the brain. The other major neuropathological hallmarks in AD are the intracellular neurofibrillary tangles (NFT) that originate by the aggregation of the hyperphosphorylated Tau protein, misfolded Tau or pathological Tau and its conformers. AD shares its etiopathology with many neurodegenerative tauopathies, in particular with specified types of frontotemporal dementia (FTD). The Tau protein is a freely soluble, “naturally unfolded” protein that binds avidly to microtubuli (MT) to promote their assembly and stability. MT are of major importance for the cytoskeletal integrity of neurons—and thereby for the proper formation and functioning of neuronal circuits, hence for learning and memory. The binding of Tau to MT is controlled by dynamic phosphorylation and de-phosphorylation, as demonstrated mainly in vitro and in non-neuronal cells. In AD brain, Tau pathology (tauopathy) develops later than amyloid pathology. However, it is still discussed controversially if Abeta protein is the causative agent in AD which constitutes the essence of the so-called amyloid cascade hypothesis (Hardy et al., Science 1992, 256, 184-185; Musiek et al., Nature Neurosciences 2015, 18(6), 800-806). The exact mechanisms that link amyloid to Tau pathology remain largely unknown, but are proposed to involve activation of neuronal signalling pathways that act on or by GSK3 and cdk5 as the major “Tau-kinases” (Muyllaert et al., Rev. Neurol. (Paris), 2006, 162, 903-907; Muyllaert et al., Genes Brain and Behav. 2008, Suppl 1, 57-66). Even if the tauopathy develops later than amyloid, it is not just an innocent side-effect but a major pathological executer in AD. In experimental mouse models the cognitive defects caused by amyloid pathology are nearly completely alleviated by the reduction in Tau protein (Roberson et al., Science, 2007, 316 (5825), 750-754) and similarly the severity of cognitive dysfunction and dementia in human AD patients correlates with the level of tau pathology not with amyloid beta pathology.

Diseases involving Tau aggregates are generally listed as tauopathies and they include, but are not limited to, Alzheimer's disease (AD), familial Alzheimer's disease (AD), primary age-related Tauopathy (PART), Creutzfeldt-Jacob disease, dementia pugilistica, Down's Syndrome, Gerstmann-Straussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17) also known as familiar FTLD-tau (MAPT), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease. (Williams et al., Intern. Med. J., 2006, 36, 652-660; Kovacs et al., J. Neuropathol. Exp. Neurol. 2008; 67(10): 963-975; Higuchi et al., Neuropsychopharmacology—5th Generation of Progress, 2002, Section 9, Chapter 94: 1339-1354; Hilton et al., Acta Neuropathol. 1995; 90(1):101-6; Iqbal et al., Biochimica et Biophysica Acta 1739 (2005), 198-210; McQuaid et al., Neuropathol. Appl. Neurobiol. 1994 April; 20(2):103-10; Vossel et al., Lancet Neurol. 2017; 16: 311-322; Stephan et al., Molecular Psychiatry (2012) 17, 1056-1076; Anderson et al., Brain (2008), 131, 1736-1748; Savica et al., JAMA Neurol. 2013; 70(7):859-866; Brown et al. Molecular Neurodegeneration 2014, 9:40; El Khoury et al., Front. Cell. Neurosci., 2014, Volume 8, Article 22: 1-18; Tanskanen et al., Ann. Med. 2008; 40(3):232-9; Gupta et al., Can J. Ophthalmol., vol. 43, No. 1, 2008: 53-60; Dickson et al., Int. J. Clin. Exp. Pathol. 2010; 3(1):1-23; Fernendez-Nogales et al., Nature Medicine, 20, 881-885 (2014); Bi et al., Nature Communications volume 8, Article number: 473 (2017); Murray et al., Biol. Psychiatry. 2014 Apr. 1; 75(7): 542-552).

Cummings et al. describe the latest clinical trials involving agents for the treatment of Alzheimer's disease (Cummings et al., Alzheimers Dement (N Y) 2019 Jul. 9; 5:272-293 and Cummings et al., Alzheimer's & Dementia: Translational Research & Clinical Interventions 3 (2017) 367-384). Among the approaches using small molecules, several Tau kinase inhibitors have been developed, despite being very challenging with respect to toxicity and specificity. Nevertheless, currently only one kinase inhibitor, Nilotinib, is tested in clinical trials. Lastly, among the Tau aggregation inhibitors only one, LMTX methylthioninium, also known as TRx0237 and LMTM, is currently in clinical trials (Cummings et al., 2017). Although in recent years, Tau-based treatments have become a point of increasing focus, there is no efficacious Tau modifying drug on the market. Therefore, there still is a need for identifying novel therapeutic agents that target the pathological Tau conformers that are known or presumed to cause tauopathies.

### SUMMARY OF THE INVENTION

The present invention provides compounds, or pharmaceutically acceptable salts thereof, pharmaceutical compositions thereof and combination thereof, that can be employed in the treatment, alleviation or prevention of a group of diseases, disorders and abnormalities associated with misfolding of Tau protein and/or pathological aggregation of Tau (Tubulin associated unit) protein including, but not limited to, Neurofibrillary Tangles (NFTs), such as Alzheimer's disease (AD). The invention further provides methods of treating, alleviation, or preventing diseases, disorders and abnormalities associated with misfolding of Tau protein and/or pathological aggregation of Tau (Tubulin associated unit) protein. Furthermore, there exists a need in the art for compounds which can be used as therapeutic agents for (a) decreasing Tau aggregates/NFTs, by recognizing aggregated Tau and disaggregating Tau, for example by changing the Tau aggregate molecular conformation, and/or (b) preventing the formation of Tau aggregates, and/or (c) interfering intracellularly with Tau aggregates. The present inventors have surprisingly found that these objects can be achieved by the compounds of the invention as described hereinafter.

The compounds of formula (I) of the invention display a high capability in decreasing Tau aggregates by, recognizing aggregated Tau and disaggregating Tau, for example by changing the Tau aggregate molecular conformation. Some compounds of the invention prevent the formation of Tau aggregates, and/or interfere intracellularly with Tau aggregates. While not wishing to be bound by theory, it is assumed that the compounds of the invention inhibit the Tau aggregation or disaggregate preformed Tau aggregates including when present intracellularly. Due to their unique design features, these compounds display properties such as appropriate lipophilicity, molecular weight, solubility, permeability and metabolic stability, which result in cell penetration, oral bioavailability, and brain uptake, adequate to be a successful medicament for the treatment, alleviation or prevention of tauopathies.

The present invention discloses novel compounds of the invention having capabilities to decrease Tau aggregates, recognize aggregated Tau and disaggregate Tau, for example by changing the Tau aggregate molecular conformation.

The present invention discloses some novel compounds having capabilities to prevent the formation of Tau aggregates, and/or to interfere intracellularly with Tau aggregates.

The present invention provides methods for the treatment of diseases, disorders and abnormalities associated with misfolding of Tau protein and/or pathological aggregation of Tau (Tubulin associated unit) protein including, but not limited to, Neurofibrillary Tangles (NFTs), such as Alzheimer's disease (AD), using compounds of the invention or a pharmaceutical composition thereof. The present invention further provides a pharmaceutical composition comprising a compound of the invention and a pharmaceutically acceptable carrier, diluent, adjuvant and/or excipient.

In particular, the present invention provides a compound of formula (I)

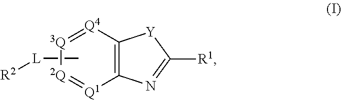

or a pharmaceutically acceptable salt thereof, wherein
Y is S or O;
R¹ is a mono or bicyclic heterocyclyl;
Q¹ and Q⁴ are different and independently selected from CH and N;
Q² and Q³ are different and independently selected from N, C, and C-L-R², wherein at least of Q² or Q³ is C-L-R²;
L is —NH(CO)—, C₂-C₄alkynyl, —NH—; or
L is a heteroaryl; or
L is a 5- to 8-membered saturated or unsaturated heterocyclyl optionally substituted with halo or C₁-C₄ alkyl; or
L is a bond
R² is selected from

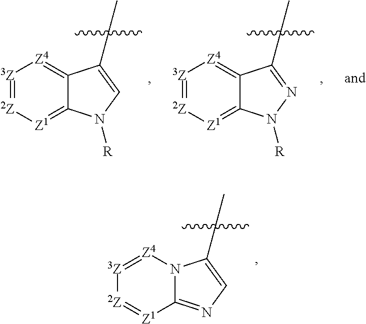

wherein
R is C₁-C₄ alkyl or H;
Z¹ is N, CH, C—F, and C—OCH₃;
Z^{1′} is N, CH, C—F, C—CH₃, and C—OCH₃;
Z² is N, CH, C—F, C—CH₃, and C—OCH₃;
Z³ or Z⁴ are independently selected from N, CH, C—F and C—CH₃; and
wherein when Z⁴ is N, at least one of Z¹, Z^{1′}, Z², Z³ is C—F.

In another aspect, the invention provides a pharmaceutical composition comprising a compound according to the definition of compound of formula (I), and optionally a pharmaceutically acceptable carrier, diluent, adjuvant and/or excipient.

In another aspect, the invention provides a compound of formula (I), or a combination, in particular a pharmaceutical composition, as disclosed herein, for use as a medicament.

In another aspect, the invention provides a compound of formula (I), as disclosed herein, or a pharmaceutical composition, for use in the treatment, alleviation or prevention of a disease, disorder or abnormality associated with misfolding of Tau protein and/or pathological aggregation of Tau protein.

In another aspect, the invention provides a method of treating, alleviating or preventing a disease, disorder or abnormality associated with misfolding of Tau protein and/or pathological aggregation of Tau protein comprising administering a compound of formula (I), as disclosed herein, or a pharmaceutical composition.

In another aspect, the invention provides a method of decreasing Tau aggregation, the method comprising administering a compound of formula (I), or a pharmaceutical composition, as defined herein, to a subject in need thereof.

In another aspect, the invention provides a method of preventing the formation of Tau aggregates and/or of inhibiting Tau aggregation, the method comprising administering a compound of formula (I), or a pharmaceutical composition, as defined herein, to a subject in need thereof.

In another aspect, the invention provides a method of interfering intracellularly with Tau aggregates, the method comprising administering an effective amount of a compound of formula (I), or a pharmaceutical composition, as defined herein, to a subject in need thereof.

In another aspect, the invention provides a combination comprising a therapeutically effective amount of a compound of formula (I), as defined herein, or a pharmaceutical composition, and one or more therapeutic agents.

In another aspect, the invention provides a mixture comprising a compound of formula (I), as disclosed herein, and one or more therapeutic agent different from the compound of formula (I), and optionally a pharmaceutically acceptable carrier, diluent, adjuvant and/or excipient.

Another aspect of the invention also relates to the use of a compound of formula (I), as an analytical reference or an in vitro screening tool.

The following clauses are also part of the invention:
A1. A compound of formula

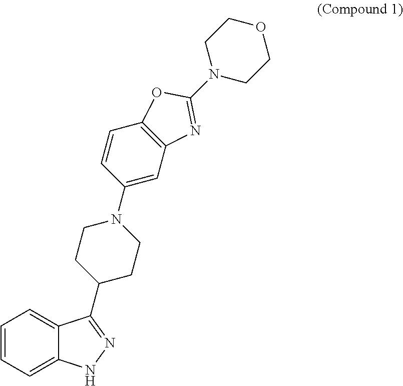 [SMILES: c1ccc2c(C3CCN(c4ccc5oc(N6CCOCC6)nc5c4)CC3)n[nH]c2c1]

or any tautomers, pharmaceutically acceptable salts, hydrates or solvates thereof.
A2. A pharmaceutical composition comprising the compound according to clause A1 and optionally a pharmaceutically acceptable carrier, diluent, adjuvant and/or excipient.
A3. The compound according to clause A1 for use as a medicament.
A4. The compound according to clause A1 for the manufacture of a medicament for decreasing Tau aggregation.
A5. The compound according to clause A1 for the manufacture of a medicament for treating, alleviating or preventing of a disorder or an abnormality associated with misfolding of Tau protein and/or pathological aggregation of Tau (Tubulin associated unit) protein.
A6. The compound according to clause A1 for use in the treatment, alleviation or prevention of a disorder or an abnormality associated with misfolding of Tau protein and/or pathological aggregation of Tau (Tubulin associated unit) protein.
A7. A method for treating, alleviating or preventing of a disorder or an abnormality associated with misfolding of Tau protein and/or pathological aggregation of Tau (Tubulin associated unit) protein comprising the step of administering a therapeutically effective amount of Compound 1 according to clause A1 to a patient in need thereof.
A8. The compound according to clause A5 or A6 and the method according to clause A7 wherein the disorder or the abnormality associated with misfolding of Tau protein and/or pathological aggregation of Tau (Tubulin associated unit) protein is selected from Alzheimer's disease (AD), familial AD, Primary Age-Related Tauopathy (PART), Creutzfeldt-Jacob disease, dementia pugilistica, Down's Syndrome, Gerstmann-Straussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17) also known familiar FTLD-tau (MAPT), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, subacute sclerosing panencephalitis (SSPE), Senile dementia of the neurofibrillary tangle type, Parkinson's disease, HIV-related dementia, adult onset diabetes, senile cardiac amyloidosis, glaucoma, ischemic stroke, psychosis in AD, Lafora disease and Huntington's disease.
A9. The compound according to clause A5 or A6 and the method according to clause A7 wherein the disorder or the abnormality associated with misfolding of Tau protein and/or pathological aggregation of Tau (Tubulin associated unit) protein is Alzheimer's disease (AD).
A10. The compound according to clause A5 or A6 and the method according to clause A7 wherein the disorder or the abnormality associated with misfolding of Tau protein and/or pathological aggregation of Tau (Tubulin associated unit) protein is progressive supranuclear palsy (PSP).
A11. The compound according to clause A5 or A6 and the method according to clause A7 wherein the disorder or the abnormality associated with misfolding of Tau protein and/or pathological aggregation of Tau (Tubulin associated unit) protein is frontotemporal dementia with Parkinsonism linked to chromosome 17 (FTDP-17) also known familiar FTLD-tau (MAPT).
A12. A mixture comprising a compound according to clause A1 and at least one further biologically active compound different from the compound according to claim 1, and optionally a pharmaceutically acceptable carrier, diluent, adjuvant and/or excipient.
A13. The mixture according to clause A12, wherein the further biologically active compound is a compound used in the treatment of amyloidosis.
A14. The mixture according to any one of clause A12 or A13, wherein the compound and/or the further biologically active compound is/are present in a therapeutically effective amount.
A15. Use of the compound according to clause A1 as an analytical reference or an in vitro screening tool.
A16. A method for producing Compound 1 comprising the step of deprotecting Compound 2 wherein Compound 2 is

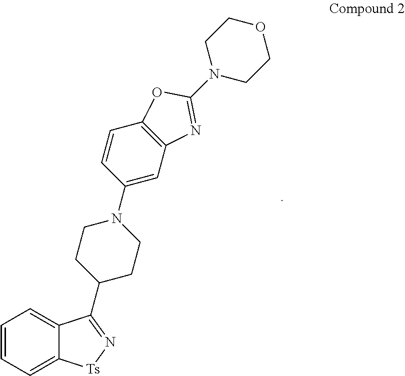

A17. The method according to clause A16 wherein the deprotection occurs in presence of strong base.

### BRIEF DESCRIPTION OF THE DRAWINGS

FIG. 1: Reduction of intracellular misfolded Tau with Compound 1 (Cpd1) at 20 nM.

FIG. 2: showed decreased of aggregated Tau with Compound 1 (Example 1) at 100 mg/kg in Cx-TBH.

### DEFINITIONS

Within the meaning of the present invention the following definitions apply, unless specified otherwise and when appropriate, terms used in the singular will also include the plural and vice versa.

It must be noted that as used herein and in the appended claims, the singular forms “a”, “an” and “the” include plural referents unless the context clearly dictates otherwise. Thus, for example, reference to “the compound” includes reference to one or more compounds; and so forth.

The term “C₁-C₄ alkyl” refers to a straight or branched hydrocarbon chain radical consisting solely of carbon and hydrogen atoms, containing no unsaturation, having from one to four carbon atoms, and which is attached to the rest of the molecule by a single bond. Examples of C₁-C₄ alkyl include, but are not limited to, methyl, ethyl, n-propyl, 1-methylethyl (iso-propyl), or n-butyl; preferably methyl.

The term “heterocyclyl” or “heterocyclic” refers to a stable 5- or 8-membered non-aromatic saturated, or unsaturated monocyclic ring, bicyclic or polycyclic ring radical which comprises 1, 2, or 3, heteroatoms individually selected from nitrogen, oxygen and sulfur, preferably the heteroatom is selected from nitrogen and oxygen. The heterocyclyl radical may be bonded via a carbon atom or heteroatom. Examples of heterocyclyl include, but are not limited to, azetidinyl, oxetanyl, pyrrolinyl, tetrahydrofuryl, tetrahydrothienyl, tetrahydropyranyl, perhydroazepinyl, pyrrolidyl, pyrrolidinyl, 6-oxa-3-azabicyclo[3.1.1]heptyl, tetrahydropyridinyl, piperidyl, piperazinyl, morpholinyl, 2-oxa-6-azaspiro[3.3]heptyl, or octahydrocyclopenta[c]pyrrolyl; preferably pyrrolidyl, pyrrolidinyl, 6-oxa-3-azabicyclo[3.1.1]heptyl, tetrahydropyridinyl, piperidyl, piperazinyl, morpholinyl, 2-oxa-6-azaspiro[3.3]heptyl, or octahydrocyclopenta[c]pyrrolyl.

The term “heteroaryl” refers to a 5- or 6-membered aromatic monocyclic ring radical which comprises 1, 2, 3 or 4 heteroatoms individually selected from nitrogen, oxygen and sulfur. The heteroaryl radical may be bonded via a carbon atom or heteroatom. Examples of heteroaryl include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, or pyridyl; preferably pyrazolyl.

The term “Halogen” or “halo” refers to bromo, chloro, fluoro or iodo. Preferably, “halo” is fluoro.

Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

The term “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of the invention and, which typically are not biologically or otherwise undesirable. In many cases, the compounds of the present invention are capable of forming acid and/or base salts by virtue of the presence of amino and/or carboxyl groups or groups similar thereto.

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric acid and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 23^{rd} ed., Mack Publishing Company, Easton, PA, 2020, the disclosure of which is hereby incorporated by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The patients or subjects in the present invention are typically animals, particularly mammals, more particularly humans.

“Tau” as used herein refers to a highly soluble microtubule binding protein mostly found in neurons and includes the major 6 isoforms, cleaved or truncated forms, and other modified forms such as arising from phosphorylation, glycosylation, glycation, prolyl isomerization, nitration, acetylation, polyamination, ubiquitination, sumoylation and oxidation.

“Aggregated Tau” refers to aggregated monomers of Tau peptides or proteins which are folded into the oligomeric or polymeric structures.

“Neurofibrillary Tangles” (NFTs) as used herein refer to insoluble aggregates of the hyperphosphorylated Tau protein containing paired helical filaments (PHF) and straight filaments. Their presence is a hallmark of AD and other diseases known as tauopathies.

“Therapeutically effective amount” means an amount of compound of the invention that is sufficient, when administered to a subject suffering from a disease, disorder, and/or abnormality to treat, reduce the incidence and/or severity of, and/or delay onset of, one or more symptoms of this disease, disorder, and/or this abnormality.

The term “subject” refers to primates (e.g., humans, male or female), dogs, rabbits, guinea pigs, pigs, rats and mice. Preferably, the subject is a human or an animal. More preferably, the subject is a human.

As defined herein, a subject is “in need of” a treatment if such subject would benefit biologically, medically or in quality of life from such treatment.

The term “pharmaceutical combination” or “combination” refers to a product that results from the mixing or combining of more than one therapeutic agent and includes both fixed combination into one dosage unit form, and non-fixed combination of the therapeutic agents, or a kit of parts for the combined administration, or a combined administration where a compound of the present invention and a combination partner (e.g. another drug as explained below, also referred to as “therapeutic agent”) may be administered independently at the same time or separately within time intervals, especially where these time intervals allow that the combination partners show a cooperative, e.g. synergistic effect. The single components may be packaged in a kit or separately. One or both of the components (e.g. powders or liquids) may be reconstituted or diluted to a desired dose prior to administration. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The definitions and preferred definitions given in the “Definition”-section apply to all of the embodiments described below unless stated otherwise.

### DETAILED DESCRIPTION OF THE INVENTION

The present invention is directed to a novel class of compounds that are useful in the treatment, alleviation or prevention of a group of diseases, disorders and/or abnormalities associated with misfolding of Tau protein and/or pathological aggregation of Tau (Tubulin associated unit) protein including, but not limited to, Neurofibrillary Tangles (NFTs), such as Alzheimer's disease (AD).

Various embodiments of the invention are described herein, it will be recognized that features specified in each embodiment may be combined with other specified features to provide further embodiments of the present invention.

Within certain aspects provided herein, the invention provides a compound of formula (I)

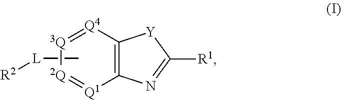

or a pharmaceutically acceptable salt thereof, wherein
Y is S or O;
R¹ is a mono or bicyclic heterocyclyl;
Q¹ and Q⁴ are different and independently selected from CH and N;
Q² and Q³ are different and independently selected from N, C, and C-L-R², wherein at least one of Q² or Q³ is C-L-R²
L is —NH(CO)—, C₂-C₄alkynyl, —NH—; or
L is a heteroaryl; or
L is a 5- to 8-membered saturated or unsaturated heterocyclyl optionally substituted with halo or C₁₋₄ alkyl; or
L is a bond
R² is selected from

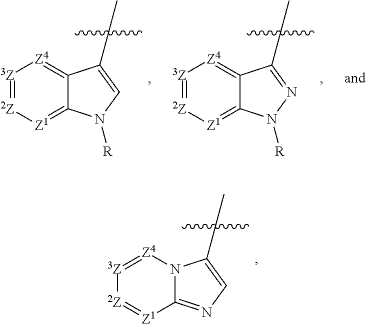

wherein
R is C₁-C₄ alkyl or H;
Z¹ is N, CH, C—F, or C—OCH₃;
Z^{1′} is N, CH, C—F, C—CH₃, or C—OCH₃;
Z² is N, CH, C—F, C—CH₃, or C—OCH₃;
Z³ or Z⁴ are independently selected from N, CH, C—F and C—CH₃; and
wherein when Z⁴ is N, at least one of Z¹, Z^{1′}, Z², Z³ is C—F.

Unless specified otherwise, the term “compounds of the invention” refers to compounds of formula (I) and subformulae thereof, pharmaceutically acceptable salts, hydrates, and solvates thereof, as well as all stereoisomers (including diastereoisomers and enantiomers), rotamers, tautomers, and isotopically compounds (including deuterium substitutions), as well as inherently formed moieties.

In another embodiment, the invention provides a compound of formula (I), having a formula (II):

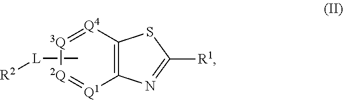

or a pharmaceutically acceptable salt thereof, wherein R¹, R², L, Q¹, Q², Q³ and Q⁴ are as defined herein above.

In yet another embodiment, the invention provides a compound of formula (I), having a formula (III):

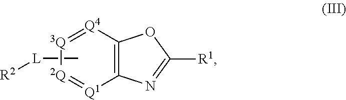

or a pharmaceutically acceptable salt thereof, wherein R¹, R², L, Q¹, Q², Q³ and Q⁴ are as defined herein above.

In another embodiment, the invention provides for a compound of formula (I), wherein R¹ is a mono or bicyclic heterocyclyl selected from the following:

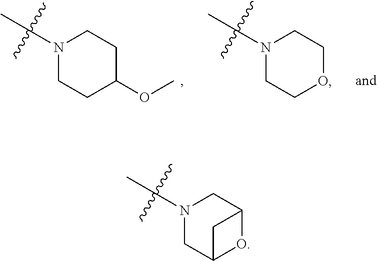

Preferably, R¹ is:

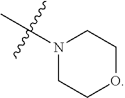 [SMILES: *N1CCOCC1]

In another embodiment, the invention provides for a compound of formula (I), wherein only one of Q¹, Q², Q³ and Q⁴ is N.

In yet another embodiment, the invention provides for a compound of formula (I), wherein Q¹, Q², Q³ and Q⁴ are all C, and wherein at least one of Q² or Q³ is C-L-R². Preferably, only one of Q² or Q³ is C-L-R².

In one embodiment, the invention provides for a compound of formula (I), wherein Q¹, Q², Q³ and Q⁴ are all C, as follows:

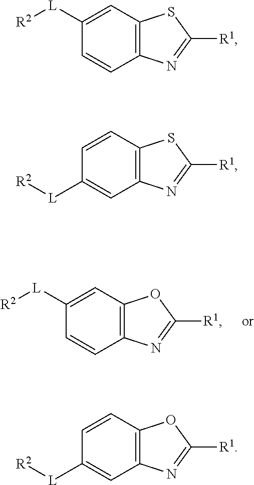

and wherein R¹, R², and L are as defined herein.

In a preferred embodiment, the invention provides for a compound of formula (I), in particular a compound of formula (II), wherein Q¹, Q², Q³ and Q⁴ are all C, as follows:

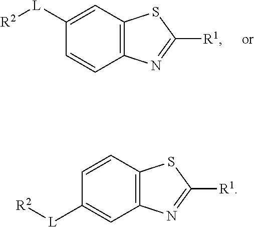

In a more preferred embodiment, the invention provides for a compound of formula (I), in particular a compound of formula (III), wherein Q¹, Q², Q³ and Q⁴ are all C, as follows:

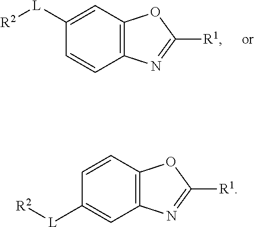

In one embodiment, the invention provides for a compound of formula (I), wherein one of Q¹, Q², Q³ and Q⁴ is N, as follows:

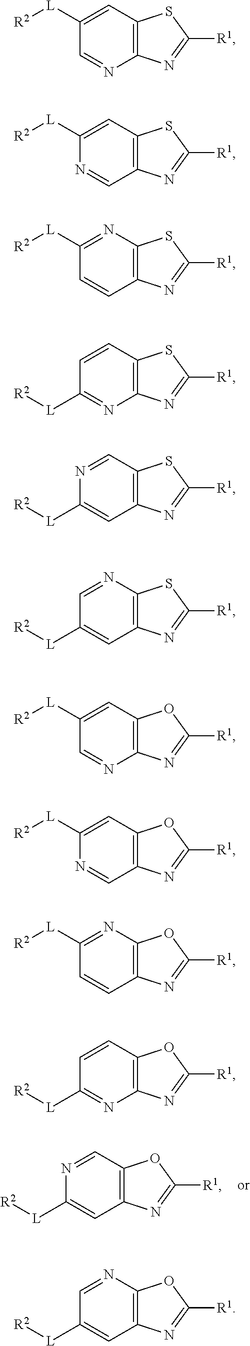

and wherein R¹, R², and L are as defined herein.

In a preferred embodiment, the invention provides for a compound of formula (I), in particular a compound of formula (II), wherein one of Q¹, Q², Q³ and Q⁴ is N, as follows:

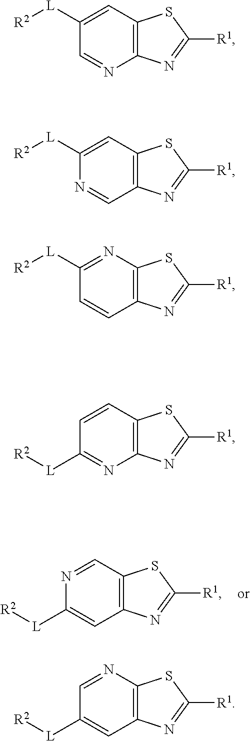

In a more preferred embodiment, the invention provides for a compound of formula (I), in particular a compound of formula (III), wherein one of Q¹, Q², Q³ and Q⁴ is N, as follows:

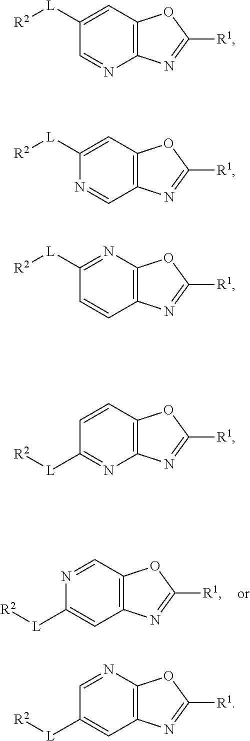

In yet one embodiment, the invention provides for a compound of formula (I), wherein R² is selected from

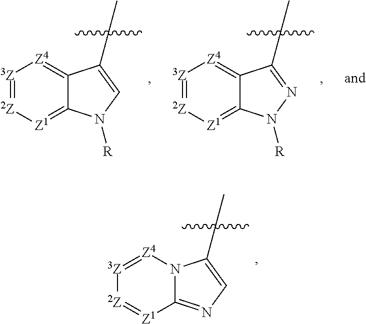

wherein R, Z¹, Z^{1′}, Z², Z³, and Z⁴ are as defined herein, and wherein no more than one of Z¹, Z^{1′}, Z², Z³, and Z⁴ is N.

In another embodiment, the invention provides for a compound of formula (I), wherein R² is selected from the following:

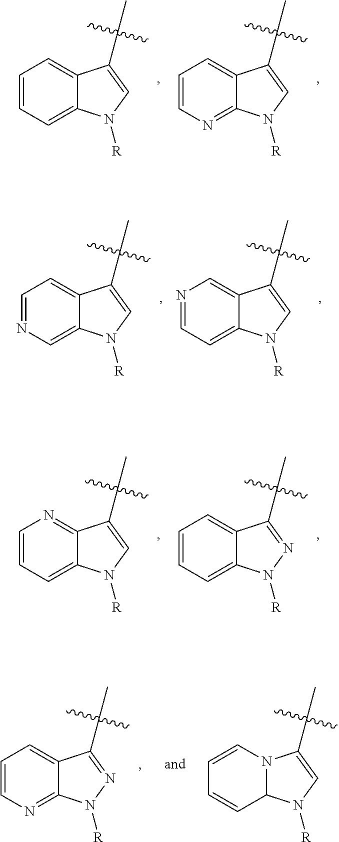

wherein R is C₁-C₄ alkyl or H; and R² is optionally substituted with 1 to 2 substituents independently selected from F, CH₃ and OCH₃.

Preferably, R is C₁-C₂ alkyl or H. More preferably R is methyl or H.

In another embodiment, the invention provides for a compound of formula (I), wherein when Q¹ is N, R² comprises two nitrogen atoms.

In one embodiment, the invention provides for a compound of formula (I), wherein L is preferably selected from the following: —NH(CO)—, C₂-C₄alkynyl, —NH—, heteroaryl, or 5- to 8-membered saturated or unsaturated heterocyclyl optionally substituted with halo or C₁-C₄ alkyl;

In yet another embodiment, the invention provides for a compound of formula (I), or a pharmaceutically acceptable salt thereof, wherein L is *—NH(CO)—, *—(CO)NH—, *—C₂-C₄alkynyl-, or *—NH—; wherein * is the position of bonding to R². Preferably, L is *—NH(CO)—, *—(CO)NH—, *—C₂ alkynyl-, or *—NH—. More preferably L is *—NH(CO)—, or *—(CO)NH—.

In yet another embodiment, the invention provides for a compound of formula (I), wherein L is a heteroaryl. Preferably L is a 5-membered aromatic monocyclic ring comprising 1, 2, 3 or 4 heteroatoms. More preferably, L is

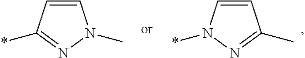

wherein * is the position of bonding to R².

In yet another embodiment, the invention provides for a compound of formula (I), wherein L is a 5- to 8-membered saturated or unsaturated heterocyclyl optionally substituted with halo or C₁-C₄ alkyl. Preferably, L is selected from

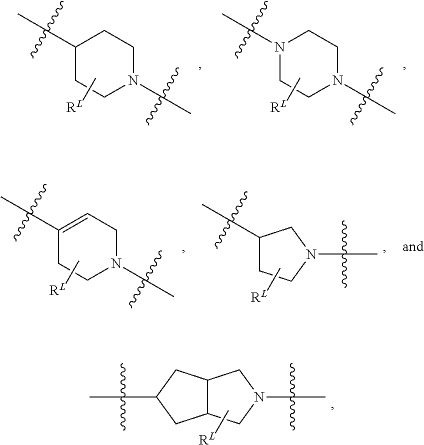

wherein R^{L} is H, C₁-C₄ alkyl, or halo.

More preferably, L is selected from

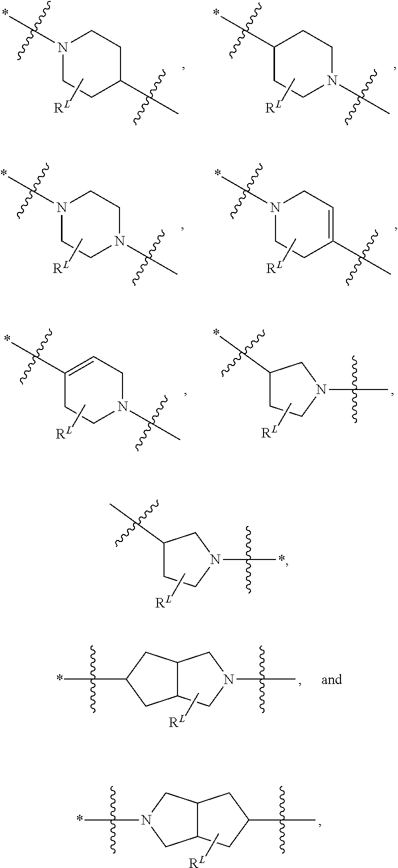

wherein R^{L} is H, C₁-C₄ alkyl, or halo and wherein * is the position of bonding to R².

Even more preferably, L is selected from

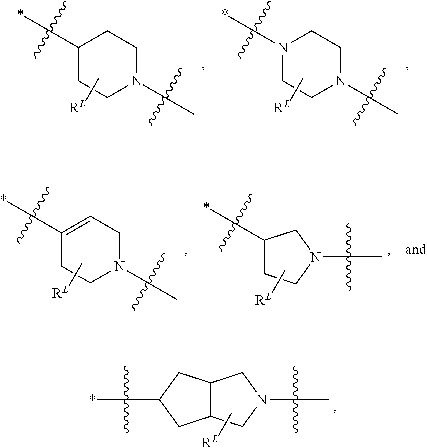

wherein R^{L} is H, C₁-C₄ alkyl, or halo and wherein * is the position of bonding to R².

Preferably, R^{L} is H, CH₃, or F. More preferably, R^{L} is H or F. Even more preferably, R^{L} is H.

In a preferred embodiment, L is selected from

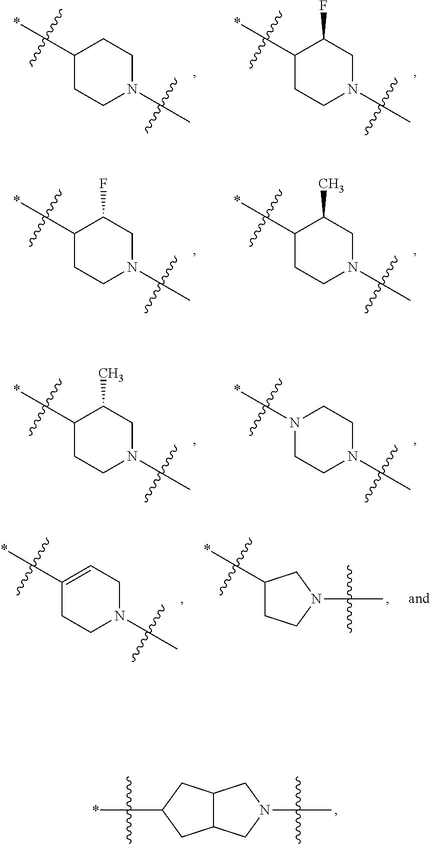

In a more preferred embodiment, L is selected from

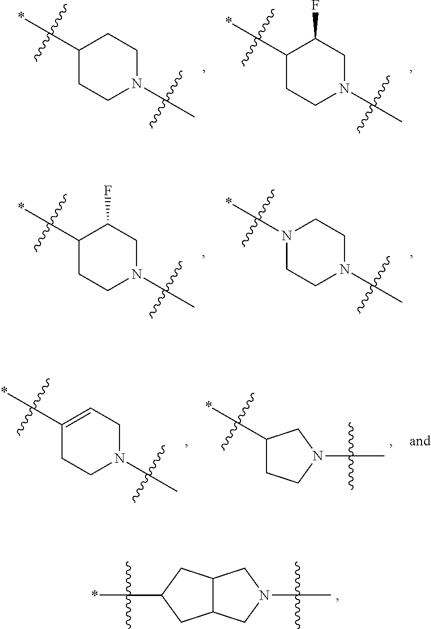

In an even more preferred embodiment, L is selected from

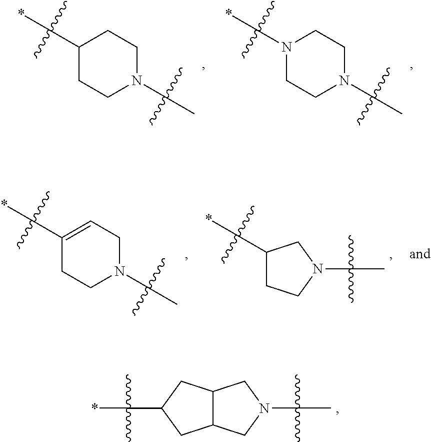

In one embodiment, the present invention provides for a compound of formula (I), wherein the compound is selected from:
5-(4-(1H-indazol-3-yl)piperidin-1-yl)-2-morpholinobenzo[d]oxazole;
5-(5-fluoro-1-methyl-1H-pyrrolo[2,3-b]pyridin-3-yl)-2-morpholinobenzo[d]oxazole; 5-(1H-indazol-3-yl)-2-morpholinobenzo[d]oxazole;
5-((1H-indazol-3-yl)ethynyl)-2-morpholinobenzo[d]oxazole;
5-((1H-indol-3-yl)ethynyl)-2-morpholinobenzo[d]oxazole;
N-(1H-indol-3-yl)-2-morpholinobenzo[d]oxazole-5-carboxamide;
5-(4-(1H-indazol-3-yl)-1H-pyrazol-1-yl)-2-morpholinobenzo[d]oxazole;
5-(4-(1H-indol-3-yl)-1H-pyrazol-1-yl)-2-morpholinobenzo[d]oxazole;
5-(3-(1H-indazol-3-yl)-1H-pyrazol-1-yl)-2-morpholinobenzo[d]oxazole;
5-(3-(1H-indol-3-yl)-1H-pyrazol-1-yl)-2-morpholinobenzo[d]oxazole;
N-(2-morpholinobenzo[d]oxazol-5-yl)-1H-indole-3-carboxamide;
N-(2-morpholinobenzo[d]oxazol-5-yl)-1H-pyrrolo[2,3-b]pyridine-3-carboxamide;
N-(2-morpholinobenzo[d]oxazol-6-yl)-1H-indole-3-carboxamide;
N-(2-morpholinobenzo[d]oxazol-6-yl)-1H-pyrrolo[2,3-b]pyridine-3-carboxamide;
N-(2-morpholinobenzo[d]oxazol-6-yl)-1H-indazole-3-carboxamide;
5-fluoro-N-(2-morpholinobenzo[d]thiazol-6-yl)-1H-indole-3-carboxamide;
N-(2-morpholinobenzo[d]oxazol-5-yl)-1H-indazole-3-carboxamide;
5-(4-(1H-indol-3-yl)piperidin-1-yl)-2-morpholinobenzo[d]oxazole;
5-(4-(1H-pyrrolo[2,3-b]pyridin-3-yl)piperidin-1-yl)-2-morpholinobenzo[d]oxazole;
5-(4-(1-methyl-1H-pyrrolo[2,3-b]pyridin-3-yl)piperidin-1-yl)-2-morpholinobenzo[d]oxazole;
5-(4-(5-fluoro-1H-pyrrolo[2,3-b]pyridin-3-yl)piperidin-1-yl)-2-morpholinobenzo[d]oxazole;
5-(4-(5-fluoro-1-methyl-1H-pyrrolo[2,3-b]pyridin-3-yl)piperidin-1-yl)-2-morpholinobenzo[d]oxazole;
4-(6-(4-(1H-indol-3-yl)piperidin-1-yl)thiazolo[4,5-c]pyridin-2-yl)morpholine;
5-(4-(imidazo[1,2-a]pyridin-3-yl)piperidin-1-yl)-2-morpholinobenzo[d]oxazole;
4-(6-(4-(5-fluoro-1-methyl-1H-pyrrolo[2,3-b]pyridin-3-yl)piperidin-1-yl)thiazolo[4,5-b]pyridin-2-yl)morpholine;
4-(6-(4-(5-fluoro-1-methyl-1H-pyrrolo[2,3-b]pyridin-3-yl)piperidin-1-yl)thiazolo[4,5-c]pyridin-2-yl)morpholine;
4-(6-(4-(1H-indol-3-yl)piperidin-1-yl)thiazolo[4,5-b]pyridin-2-yl)morpholine;
4-(6-(4-(imidazo[1,2-a]pyridin-3-yl)piperidin-1-yl)benzo[d]thiazol-2-yl)morpholine;
4-(6-(4-(6-fluoro-1H-pyrrolo[3,2-b]pyridin-3-yl)piperidin-1-yl)benzo[d]thiazol-2-yl)morpholine;
5-(4-(1-methyl-1H-pyrazolo[3,4-b]pyridin-3-yl)piperidin-1-yl)-2-morpholinobenzo[d]oxazole;
5-(4-(5-fluoro-1-methyl-1H-pyrrolo[2,3-b]pyridin-3-yl)-3,6-dihydropyridin-1(2H)-yl)-2-morpholinobenzo[d]oxazole;
5-(3-(5-fluoro-1-methyl-1H-pyrrolo[2,3-b]pyridin-3-yl)pyrrolidin-1-yl)-2-morpholinobenzo[d]oxazole;
5-(3-(5-fluoro-1-methyl-1H-pyrrolo[2,3-b]pyridin-3-yl)pyrrolidin-1-yl)-2-morpholinobenzo[d]oxazole;
4-(6-(4-(1H-indazol-3-yl)piperidin-1-yl)thiazolo[4,5-c]pyridin-2-yl)morpholine;
N-(1H-indazol-3-yl)-2-morpholinobenzo[d]oxazol-5-amine;
5-(3-(1H-indazol-3-yl)pyrrolidin-1-yl)-2-morpholinobenzo[d]oxazole;
5-(3-(1H-indazol-3-yl)pyrrolidin-1-yl)-2-morpholinobenzo[d]oxazole;
5-(3-(1H-indol-3-yl)pyrrolidin-1-yl)-2-morpholinobenzo[d]oxazole;
5-(3-(1H-indol-3-yl)pyrrolidin-1-yl)-2-morpholinobenzo[d]oxazole;
4-(6-(4-(1H-indazol-3-yl)piperidin-1-yl)thiazolo[4,5-b]pyridin-2-yl)morpholine;
4-(6-(4-(1H-indazol-3-yl)piperidin-1-yl)thiazolo[5,4-b]pyridin-2-yl)morpholine;
5-(4-(1H-indazol-3-yl)piperidin-1-yl)-2-morpholinooxazolo[4,5-b]pyridine;
4-(6-(4-(1H-indazol-3-yl)piperidin-1-yl)benzo[d]thiazol-2-yl)morpholine;
6-(4-(1H-indazol-3-yl)piperidin-1-yl)-2-morpholinooxazolo[5,4-c]pyridine;
5-((5-fluoro-1-methyl-1H-pyrrolo[2,3-b]pyridin-3-yl)ethynyl)-2-morpholinobenzo[d]oxazole;
6-(4-(1H-indazol-3-yl)piperidin-1-yl)-2-morpholinooxazolo[4,5-b]pyridine;
6-(4-(1H-indol-3-yl)piperidin-1-yl)-2-morpholinooxazolo[5,4-c]pyridine;
6-(4-(1H-indazol-3-yl)piperidin-1-yl)-2-morpholinooxazolo[5,4-b]pyridine;
5-(4-(1H-indazol-3-yl)-3,6-dihydropyridin-1(2H)-yl)-2-morpholinobenzo[d]oxazole;
5-(4-(5-fluoro-1-methyl-1H-indol-3-yl)piperidin-1-yl)-2-morpholinobenzo[d]oxazole;
6-(4-(1H-indol-3-yl)piperidin-1-yl)-2-morpholinobenzo[d]oxazole;
5-(4-(6-fluoro-1H-pyrrolo[3,2-b]pyridin-3-yl)piperidin-1-yl)-2-morpholinobenzo[d]oxazole;
5-(4-(1H-pyrrolo[2,3-c]pyridin-3-yl)piperidin-1-yl)-2-morpholinobenzo[d]oxazole;
5-(4-(1H-pyrrolo[3,2-c]pyridin-3-yl)piperidin-1-yl)-2-morpholinobenzo[d]oxazole;
5-(4-(1H-indol-3-yl)piperazin-1-yl)-2-morpholinobenzo[d]oxazole;
4-(5-(4-(5-fluoro-1-methyl-1H-pyrrolo[2,3-b]pyridin-3-yl)piperidin-1-yl)thiazolo[5,4-b]pyridin-2-yl)morpholine;
6-(4-(1H-indol-3-yl)piperidin-1-yl)-2-morpholinooxazolo[4,5-c]pyridine;
6-(4-(imidazo[1,2-a]pyridin-3-yl)piperidin-1-yl)-2-morpholinobenzo[d]oxazole;
5-(5-(5-fluoro-1-methyl-1H-pyrrolo[2,3-b]pyridin-3-yl)hexahydrocyclopenta[c]pyrrol-2(1H)-yl)-2-morpholinobenzo[d]oxazole;
5-(4-(5-fluoro-1H-indazol-3-yl)piperidin-1-yl)-2-morpholinobenzo[d]oxazole;
4-(5-(4-(1H-indazol-3-yl)piperidin-1-yl)thiazolo[4,5-b]pyridin-2-yl)morpholine;
4-(5-(4-(1H-indazol-3-yl)piperidin-1-yl)benzo[d]thiazol-2-yl)morpholine;
5-(4-(1H-indazol-3-yl)piperidin-1-yl)-2-morpholinooxazolo[5,4-b]pyridine;
4-(6-(4-(1H-indol-3-yl)piperidin-1-yl)thiazolo[5,4-b]pyridin-2-yl)morpholine;
4-(6-(4-(1H-indazol-3-yl)piperidin-1-yl)thiazolo[5,4-c]pyridin-2-yl)morpholine;
4-(6-(4-(1H-indol-3-yl)piperidin-1-yl)thiazolo[5,4-c]pyridin-2-yl)morpholine;
5-(4-(1-methyl-1H-indol-3-yl)piperidin-1-yl)-2-morpholinobenzo[d]oxazole;
5-(4-(5-fluoro-1H-indol-3-yl)piperidin-1-yl)-2-morpholinobenzo[d]oxazole;
6-(4-(5-fluoro-1-methyl-1H-pyrrolo[2,3-b]pyridin-3-yl)piperidin-1-yl)-2-morpholinobenzo[d]oxazole;
4-(5-(4-(1H-indol-3-yl)piperidin-1-yl)thiazolo[5,4-b]pyridin-2-yl)Morpholine;
6-(4-(1H-indazol-3-yl)piperidin-1-yl)-2-morpholinooxazolo[4,5-c]pyridine;
5-(4-(4-fluoro-1H-indazol-3-yl)piperidin-1-yl)-2-morpholinobenzo[d]oxazole;
5-(4-(6-fluoro-1H-indazol-3-yl)piperidin-1-yl)-2-morpholinobenzo[d]oxazole;
5-(4-(7-fluoro-1H-indazol-3-yl)piperidin-1-yl)-2-morpholinobenzo[d]oxazole;
5-(4-(5-methyl-1H-indazol-3-yl)piperidin-1-yl)-2-morpholinobenzo[d]oxazole;
5-(4-(6-methyl-1H-indazol-3-yl)piperidin-1-yl)-2-morpholinobenzo[d]oxazole;
5-(4-(7-methyl-1H-indazol-3-yl)piperidin-1-yl)-2-morpholinobenzo[d]oxazole;
5-(4-(1H-indazol-3-yl)piperazin-1-yl)-2-morpholinobenzo[d]oxazole;
5-(4-(1H-indazol-3-yl)piperidin-1-yl)-2-(4-methoxypiperidin-1-yl)benzo[d]oxazole;
4-(5-(3-(5-fluoro-1-methyl-1H-pyrrolo[2,3-b]pyridin-3-yl)pyrrolidin-1-yl)benzo[d]thiazol-2-yl)morpholine;
4-(6-(3-(5-fluoro-1-methyl-1H-pyrrolo[2,3-b]pyridin-3-yl)pyrrolidin-1-yl)benzo[d]thiazol-2-yl)morpholine;
4-(6-(3-(5-fluoro-1-methyl-1H-pyrrolo[2,3-b]pyridin-3-yl)pyrrolidin-1-yl)benzo[d]thiazol-2-yl)morpholine;
6-(3-(5-fluoro-1-methyl-1H-pyrrolo[2,3-b]pyridin-3-yl)pyrrolidin-1-yl)-2-morpholinooxazolo[5,4-b]pyridine;
6-(3-(5-fluoro-1-methyl-1H-pyrrolo[2,3-b]pyridin-3-yl)pyrrolidin-1-yl)-2-morpholinooxazolo[5,4-b]pyridine;
6-(3-(5-fluoro-1-methyl-1H-pyrrolo[2,3-b]pyridin-3-yl)pyrrolidin-1-yl)-2-morpholinooxazolo[5,4-c]pyridine;
6-(3-(5-fluoro-1-methyl-1H-pyrrolo[2,3-b]pyridin-3-yl)pyrrolidin-1-yl)-2-morpholinooxazolo[5,4-c]pyridine;
6-(3-(5-fluoro-1-methyl-1H-pyrrolo[2,3-b]pyridin-3-yl)pyrrolidin-1-yl)-2-morpholinobenzo[d]oxazole;
6-(3-(5-fluoro-1-methyl-1H-pyrrolo[2,3-b]pyridin-3-yl)pyrrolidin-1-yl)-2-morpholinobenzo[d]oxazole;
5-(3-(5-fluoro-1-methyl-1H-pyrrolo[2,3-b]pyridin-3-yl)pyrrolidin-1-yl)-2-morpholinooxazolo[4,5-b]pyridine;
5-(3-(5-fluoro-1-methyl-1H-pyrrolo[2,3-b]pyridin-3-yl)pyrrolidin-1-yl)-2-morpholinooxazolo[4,5-b]pyridine;
5-(4-(7-methoxy-1H-indazol-3-yl)piperidin-1-yl)-2-morpholinobenzo[d]oxazole;
5-(4-(4-methyl-1H-indazol-3-yl)piperidin-1-yl)-2-morpholinobenzo[d]oxazole;
5-(3-fluoro-4-(1H-indazol-3-yl)piperidin-1-yl)-2-morpholinobenzo[d]oxazole;
4-(5-(3-(5-fluoro-1-methyl-1H-pyrrolo[2,3-b]pyridin-3-yl)pyrrolidin-1-yl)benzo[d]thiazol-2-yl)morpholine;
5-(3-(5-fluoro-1-methyl-1H-pyrrolo[2,3-b]pyridin-3-yl)pyrrolidin-1-yl)-2-(4-methoxypiperidin-1-yl)benzo[d]oxazole;
5-(3-(5-fluoro-1-methyl-1H-pyrrolo[2,3-b]pyridin-3-yl)pyrrolidin-1-yl)-2-(4-methoxypiperidin-1-yl)benzo[d]oxazole;
3-(5-(3-(5-fluoro-1-methyl-1H-pyrrolo[2,3-b]pyridin-3-yl)pyrrolidin-1-yl)benzo[d]oxazol-2-yl)-6-oxa-3-azabicyclo[3.1.1]heptane;
3-(5-(3-(5-fluoro-1-methyl-1H-pyrrolo[2,3-b]pyridin-3-yl)pyrrolidin-1-yl)benzo[d]oxazol-2-yl)-6-oxa-3-azabicyclo[3.1.1]heptane;
6-(4-(1H-indol-3-yl)piperidin-1-yl)-2-morpholinooxazolo[4,5-b]pyridine;
5-(4-(1H-indol-3-yl)piperidin-1-yl)-2-morpholinooxazolo[5,4-b]pyridine;
5-(3-(5-fluoro-1-methyl-1H-pyrrolo[2,3-b]pyridin-3-yl)pyrrolidin-1-yl)-2-morpholinobenzo[d]oxazole;
6-(4-(6-fluoro-1H-pyrrolo[3,2-b]pyridin-3-yl)piperidin-1-yl)-2-morpholinobenzo[d]oxazole;
5-(4-(6-methoxy-1H-pyrrolo[2,3-b]pyridin-3-yl)piperidin-1-yl)-2-morpholinobenzo[d]oxazole;
5-(4-(6-fluoro-1-methyl-1H-pyrrolo[3,2-b]pyridin-3-yl)piperidin-1-yl)-2-morpholinobenzo[d]oxazole;
4-(5-(4-(1H-indazol-3-yl)piperidin-1-yl)thiazolo[5,4-b]pyridin-2-yl)morpholine;
5-(4-(1H-indol-3-yl)-3,6-dihydropyridin-1(2H)-yl)-2-morpholinobenzo[d]oxazole;
4-(5-(4-(1H-indol-3-yl)piperidin-1-yl)thiazolo[5,4-b]pyridin-2-yl)morpholine hydrochloride;
4-(6-(4-(5-fluoro-1-methyl-1H-pyrrolo[2,3-b]pyridin-3-yl)piperidin-1-yl)thiazolo[4,5-b]pyridin-2-yl)morpholine hydrochloride;
4-(6-(4-(5-fluoro-1-methyl-1H-pyrrolo[2,3-b]pyridin-3-yl)piperidin-1-yl)thiazolo[4,5-c]pyridin-2-yl)morpholine hydrochloride;
6-(4-(5-fluoro-1-methyl-1H-pyrrolo[2,3-b]pyridin-3-yl)piperidin-1-yl)-2-morpholinobenzo[d]oxazole hydrochloride;
5-(5-(5-fluoro-1-methyl-1H-pyrrolo[2,3-b]pyridin-3-yl)hexahydrocyclopenta[c]pyrrol-2(1H)-yl)-2-morpholinobenzo[d]oxazole hydrochloride;

or any tautomers, pharmaceutically acceptable salts, hydrates or solvates thereof.

In one aspect of the invention is provided a compound as following

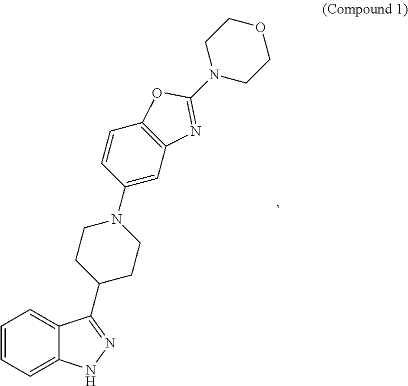 [SMILES: c1ccc2c(C3CCN(c4ccc5oc(N6CCOCC6)nc5c4)CC3)n[nH]c2c1]

or any tautomers, pharmaceutically acceptable salts, hydrates or solvates thereof. The compound of the invention is the compound of Example 1 (named Compound 1) thereafter.

In one aspect of the invention is provided a compound as following

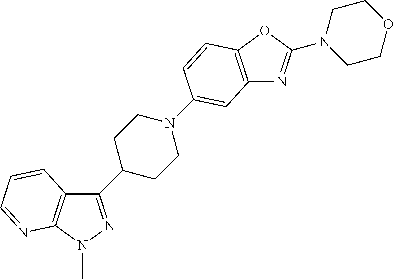 [SMILES: Cn1nc(C2CCN(c3ccc4oc(N5CCOCC5)nc4c3)CC2)c2cccnc21]

### Example 31

or any tautomers, pharmaceutically acceptable salts, hydrates or solvates thereof. The compound of the invention is named Example 31 thereafter.

In one aspect of the invention is provided a compound as following

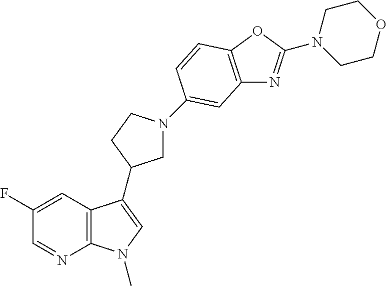 [SMILES: Cn1cc(C2CCN(c3ccc4oc(N5CCOCC5)nc4c3)C2)c2cc(F)cnc21]

### Example 33 and Example 34

or any tautomers, pharmaceutically acceptable salts, hydrates or solvates thereof. The compounds of the invention are named Example 33 and Example 34 thereafter.

In one aspect of the invention is provided a compound as following

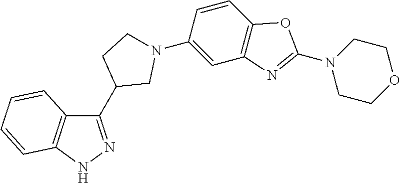 [SMILES: c1ccc2c(C3CCN(c4ccc5oc(N6CCOCC6)nc5c4)C3)n[nH]c2c1]

### Example 37 and Example 38

or any tautomers, pharmaceutically acceptable salts, hydrates or solvates thereof. The compounds of the invention are named Example 37 and Example 38 thereafter.

In one aspect of the invention is provided a compound as following

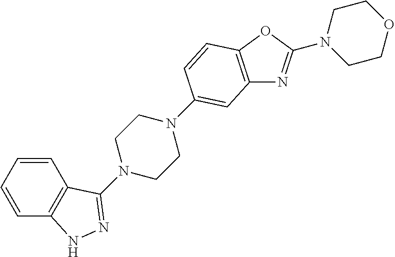 [SMILES: c1ccc2c(N3CCN(c4ccc5oc(N6CCOCC6)nc5c4)CC3)n[nH]c2c1]

### Example 79

or any tautomers, pharmaceutically acceptable salts, hydrates or solvates thereof. The compound of the invention is named Example 79 thereafter.

Preferred compounds are also illustrated in the examples.

In one embodiment, the invention provides a pharmaceutical composition comprising a compound of formula (I), and optionally a pharmaceutically acceptable carrier, diluent, adjuvant and/or excipient.

In an embodiment of the invention is provided a pharmaceutical composition comprising the Compound 1 (Example 1), and optionally a pharmaceutically acceptable carrier, diluent, adjuvant and/or excipient.

In another embodiment of the invention is provided a pharmaceutical composition comprising the compound from Example 31, and optionally a pharmaceutically acceptable carrier, diluent, adjuvant and/or excipient.

In another embodiment of the invention is provided a pharmaceutical composition comprising the compounds from Example 33 and Example 34, and optionally a pharmaceutically acceptable carrier, diluent, adjuvant and/or excipient.

In another embodiment of the invention is provided a pharmaceutical composition comprising the compounds from Example 37 and Example 38, and optionally a pharmaceutically acceptable carrier, diluent, adjuvant and/or excipient.

In yet another embodiment of the invention is provided a pharmaceutical composition comprising the compound from Example 79, and optionally a pharmaceutically acceptable carrier, diluent, adjuvant and/or excipient.

In one embodiment, the pharmaceutical composition, as disclosed herein above, comprises additionally a pharmaceutically acceptable carrier, diluent, adjuvant and/or excipient.

While it is possible for the compounds of the present invention to be administered alone, it is preferable to formulate them into a pharmaceutical composition in accordance with standard pharmaceutical practice. Thus, the invention also provides a pharmaceutical composition which comprises a therapeutically effective amount of a compound of formula (I), and optionally at least one pharmaceutically acceptable carrier, diluent, adjuvant and/or excipient.

The invention also provides a pharmaceutical composition which comprises a therapeutically effective amount of Compound 1 (Example 1) and optionally at least one pharmaceutically acceptable carrier, diluent, adjuvant and/or excipient.

In another example, the invention also provides a pharmaceutical composition which comprises a therapeutically effective amount of compound from Example 31 and optionally at least one pharmaceutically acceptable carrier, diluent, adjuvant and/or excipient.

In another example, the invention also provides a pharmaceutical composition which comprises a therapeutically effective amount of compound from Example 33 and Example 34 and optionally at least one pharmaceutically acceptable carrier, diluent, adjuvant and/or excipient.

In another example, the invention also provides a pharmaceutical composition which comprises a therapeutically effective amount of compound from Example 37 and Example 38 and optionally at least one pharmaceutically acceptable carrier, diluent, adjuvant and/or excipient.

In yet another example, the invention also provides a pharmaceutical composition which comprises a therapeutically effective amount of compound from Example 79 and optionally at least one pharmaceutically acceptable carrier, diluent, adjuvant and/or excipient.

Pharmaceutically acceptable excipients are well known in the pharmaceutical art, and are described, for example, in Remington's Pharmaceutical Sciences, 15^{th} Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

The pharmaceutical compositions of the invention can be produced in a manner known per se to the skilled person as described, for example, in Remington's Pharmaceutical Sciences, 15^{th} Ed., Mack Publishing Co., New Jersey (1975).

Pharmaceutically useful excipients that may be used in the formulation of the pharmaceutical composition of the present invention may comprise, for example, carriers, vehicles, diluents, solvents such as monohydric alcohols such as ethanol, isopropanol and polyhydric alcohols such as glycols and edible oils such as soybean oil, coconut oil, olive oil, safflower oil, cottonseed oil, sesame oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone, low melting waxes, and ion exchange resins.

The routes for administration (delivery) of the compounds of the invention include, but are not limited to, one or more of: oral (e. g. as a tablet, capsule, or as an ingestible solution), topical, mucosal (e. g. as a nasal spray or aerosol for inhalation), nasal, parenteral (e. g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, D-α-tocopheryl polyethylene glycol succinate (TPGS), disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

If the compounds of the present invention are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e. g. using a mixture of ethanol and the propellant as the solvent, which may additionally contain a lubricant, e. g. sorbitan trioleate. Capsules and cartridges (made, for example, from gelatin) for use in an inhaler or insufflator may be formulated to contain a powder mix of the compound and a suitable powder base such as lactose or starch.

Alternatively, the compounds of the present invention can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH was adjusted, sterile saline, or, preferably, as solutions in isotonic, pH was adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the invention may also be used in combination with other therapeutic agents. When a compound of the invention is used in combination with a second therapeutic agent active against the same disease, the dose of each compound may differ from that when the compound is used alone.

Thus, the invention relates to a combination comprising a therapeutically effective amount of a compound of formula (I), and one or more therapeutic agents. The one or more therapeutic agents can be selected, for example, from the group consisting of compounds against oxidative stress; anti-amyloid drug; anti-apoptotic compounds; metal chelators; inhibitors of DNA repair such as pirenzepine and metabolites; 3-amino-1-propanesulfonic acid (3 APS); 1,3-propanedisulfonate (1,3PDS); alpha-secretase activators; beta- and gamma-secretase inhibitors including BACE1; Tau proteins; neurotransmitters; beta-sheet breakers; attractants for amyloid beta clearing/depleting cellular components; inhibitors of N-terminal truncated amyloid beta including pyroglutamated amyloid beta 3-42; anti-inflammatory molecules; cholinesterase inhibitors (ChEIs) such as tacrine, rivastigmine, donepezil, and/or galantamine; M1 agonists; amyloid-beta or Tau modifying drugs; nutritive supplements; neurological drugs; corticosteroids, antibiotics, antiviral agents.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the second therapeutic agent may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manner as are known for such compounds in the art.

In one embodiment, the invention provides a method for treating, alleviating or preventing a disease, disorder or an abnormality associated with misfolding of Tau protein and/or pathological aggregation of Tau (Tubulin associated unit) protein comprising the step of administering a compound of formula (1), as defined above, or a pharmaceutical composition thereof, said pharmaceutical composition comprising a compound of formula (I).

For example, in an embodiment of the invention is provided a method for treating, alleviating or preventing a disease, disorder or an abnormality associated with misfolding of Tau protein and/or pathological aggregation of Tau (Tubulin associated unit) protein comprising the step of administering Compound 1 (Example 1) as defined above or a pharmaceutical composition thereof comprising said compound.

In another example, the invention provides a method for treating, alleviating or preventing a disease, disorder or an abnormality associated with misfolding of Tau protein and/or pathological aggregation of Tau (Tubulin associated unit) protein comprising the step of administering a compound as defined in Example 31 as defined above or a pharmaceutical composition thereof comprising said compound.

In another example, the invention also provides a method for treating, alleviating or preventing a disease, disorder or an abnormality associated with misfolding of Tau protein and/or pathological aggregation of Tau (Tubulin associated unit) protein comprising the step of administering a compound as defined in Example 33 and Example 34 as defined above or a pharmaceutical composition thereof comprising said compound.

In another example, the invention provides a method for treating, alleviating or preventing a disease, disorder or an abnormality associated with misfolding of Tau protein and/or pathological aggregation of Tau (Tubulin associated unit) protein comprising the step of administering a compound as defined in Example 37 and Example 38 as defined above or a pharmaceutical composition thereof comprising said compound.

In yet another example, the invention provides a method for treating, alleviating or preventing a disease, disorder or an abnormality associated with misfolding of Tau protein and/or pathological aggregation of Tau (Tubulin associated unit) protein comprising the step of administering a compound as defined in Example 79 as defined above or a pharmaceutical composition thereof comprising said compound.

In one embodiment, the invention relates to a method for treating, alleviating or preventing of a disease, disorder or an abnormality associated with misfolding of Tau protein and/or pathological aggregation of Tau (Tubulin associated unit) protein comprising the step of administering a therapeutically effective amount of a compound of formula (I), defined above, or a pharmaceutical composition comprising a compound of formula (I), to a patient in need thereof.

For example, the invention relates to a method for treating, alleviating or preventing of a disease, disorder or an abnormality associated with misfolding of Tau protein and/or pathological aggregation of Tau (Tubulin associated unit) protein comprising the step of administering a therapeutically effective amount of Compound 1 (Example 1), defined above, or a pharmaceutical composition thereof comprising said compound, to a patient in need thereof.

In another example, the invention relates to a method for treating, alleviating or preventing of a disease, disorder or an abnormality associated with misfolding of Tau protein and/or pathological aggregation of Tau (Tubulin associated unit) protein comprising the step of administering a therapeutically effective amount of a compound as defined in Example 31 defined above or a pharmaceutical composition thereof comprising said compound, to a patient in need thereof.

In another example, the invention relates to a method for treating, alleviating or preventing of a disease, disorder or an abnormality associated with misfolding of Tau protein and/or pathological aggregation of Tau (Tubulin associated unit) protein comprising the step of administering a therapeutically effective amount of a compound as defined in Example 33 and Example 34 defined above or a pharmaceutical composition thereof comprising said compound, to a patient in need thereof.

In another example, the invention relates to a method for treating, alleviating or preventing of a disease, disorder or an abnormality associated with misfolding of Tau protein and/or pathological aggregation of Tau (Tubulin associated unit) protein comprising the step of administering a therapeutically effective amount of a compound as defined in Example 37 and Example 38 defined above or a pharmaceutical composition thereof comprising said compound, to a patient in need thereof.

In yet another example, the invention relates to a method for treating, alleviating or preventing of a disease, disorder or an abnormality associated with misfolding of Tau protein and/or pathological aggregation of Tau (Tubulin associated unit) protein comprising the step of administering a therapeutically effective amount of a compound as defined in Example 79 defined above or a pharmaceutical composition thereof comprising said compound, to a patient in need thereof.

In one embodiment, the invention relates to a compound of formula (I), or a pharmaceutical composition comprising a compound of formula (I), for use as a medicament. In particular the invention relates to a compound of formula (I), for use as a medicament for treating, alleviating or preventing of a disease, disorder or an abnormality associated with misfolding of Tau protein and/or pathological aggregation of Tau (Tubulin associated unit) protein. In another embodiment, the invention relates to a compound of formula (I), for use in the treatment, alleviation, or prevention of a disease, disorder, or abnormality associated with misfolding of Tau protein and/or pathological aggregation of Tau Protein. In yet another embodiment, the invention relates to a pharmaceutical combination, as defined herein, for use in the treatment, alleviation, or prevention of a disease, disorder, or abnormality associated with misfolding of Tau protein and/or pathological aggregation of Tau Protein. In yet another embodiment, the invention relates to a pharmaceutical composition, comprising a compound of formula (I), as defined herein, for use in the treatment, alleviation, or prevention of a disease, disorder, or abnormality associated with misfolding of Tau protein and/or pathological aggregation of Tau Protein.

For example, the invention relates to Compound 1 (Example 1), as defined above, for use as a medicament. The invention also relates to a pharmaceutical combination comprising Compound 1 (Example 1) for use as a medicament.

In another example, the invention relates to a compound as defined in Example 31, as defined above, for use as a medicament. The invention also relates to a pharmaceutical combination comprising a compound as defined in Example 31 for use as a medicament.

In another example, the invention relates to a compound as defined in Example 33 and Example 34, as defined above, for use as a medicament. The invention also relates to a pharmaceutical combination comprising a compound as defined in Example 33 and Example 34 for use as a medicament.

In another example, the invention relates to a compound as defined in Example 37 and Example 38, as defined above, for use as a medicament. The invention also relates to a pharmaceutical combination comprising a compound as defined in Example 37 and Example 38 for use as a medicament.

In yet another example, the invention relates to a compound as defined in Example 79, as defined above, for use as a medicament. The invention also relates to a pharmaceutical combination comprising a compound as defined in Example 79 for use as a medicament.

In another embodiment, the invention relates to a compound of formula (I), for use in the treatment, alleviation or prevention of a disease, disorder or an abnormality associated with misfolding of Tau protein and/or pathological aggregation of Tau (Tubulin associated unit) protein. In yet another embodiment, the invention relates to a pharmaceutical composition comprising a compound of formula (I), for use in the treatment, alleviation or prevention of a disease, disorder or an abnormality associated with misfolding of Tau protein and/or pathological aggregation of Tau (Tubulin associated unit) protein.

For example, the invention relates to Compound 1 (Example 1), as defined above, for use in the treatment, alleviation or prevention of a disease, disorder or an abnormality associated with misfolding of Tau protein and/or pathological aggregation of Tau (Tubulin associated unit) protein.

In another example, the invention relates to a compound as defined in Example 31, as defined above, for use in the treatment, alleviation or prevention of a disease, disorder or an abnormality associated with misfolding of Tau protein and/or pathological aggregation of Tau (Tubulin associated unit) protein.

In another example, the invention relates to a compound as defined in Example 33 and Example 34, as defined above, for use in the treatment, alleviation or prevention of a disease, disorder or an abnormality associated with misfolding of Tau protein and/or pathological aggregation of Tau (Tubulin associated unit) protein.

In another example, the invention relates to a compound as defined in Example 37 and Example 38, as defined above, for use in the treatment, alleviation or prevention of a disease, disorder or an abnormality associated with misfolding of Tau protein and/or pathological aggregation of Tau (Tubulin associated unit) protein.

In yet another example, the invention relates to a compound as defined in Example 79, as defined above, for use in the treatment, alleviation or prevention of a disease, disorder or an abnormality associated with misfolding of Tau protein and/or pathological aggregation of Tau (Tubulin associated unit) protein.

In one embodiment, the invention relates to the use of a compound of formula (I), for the manufacture of a medicament for treating, preventing, or alleviating a disease, disorder or abnormality associated with misfolding of Tau protein and/or pathological aggregation of Tau (Tubulin associated unit) protein.

For example, the invention relates to the use of Compound 1 (Example 1), as defined above, for the manufacture of a medicament for treating, preventing or alleviating a disease, disorder or abnormality associated with misfolding of Tau protein and/or pathological aggregation of Tau (Tubulin associated unit) protein.

In another example, the invention relates to the use of a compound as defined in Example 31, as defined above, for the manufacture of a medicament for treating, preventing or alleviating a disease, disorder or abnormality associated with misfolding of Tau protein and/or pathological aggregation of Tau (Tubulin associated unit) protein.

In another example, the invention relates to the use of a compound as defined in Example 33 and Example 34, as defined above, for the manufacture of a medicament for treating, preventing or alleviating a disease, disorder or abnormality associated with misfolding of Tau protein and/or pathological aggregation of Tau (Tubulin associated unit) protein.

In another example, the invention relates to the use of a compound as defined in Example 37 and Example 38, as defined above, for the manufacture of a medicament for treating, preventing or alleviating a disease, disorder or abnormality associated with misfolding of Tau protein and/or pathological aggregation of Tau (Tubulin associated unit) protein.

In yet another example, the invention relates to the use of a compound as defined in Example 79, for the manufacture of a medicament for treating, preventing or alleviating a disease, disorder or abnormality associated with misfolding of Tau protein and/or pathological aggregation of Tau (Tubulin associated unit) protein.

In one embodiment, the invention relates to (i) a method for treating, alleviating or preventing of a disease, disorder or an abnormality associated with misfolding of Tau protein and/or pathological aggregation of Tau (Tubulin associated unit) protein; (ii) use in the treatment, alleviation or prevention of a disease, disorder or an abnormality associated with misfolding of Tau protein and/or pathological aggregation of Tau (Tubulin associated unit) protein; (iii) use for the manufacture of a medicament for treating, preventing or alleviating a disease, disorder or abnormality associated with misfolding of Tau protein and/or pathological aggregation of Tau (Tubulin associated unit) protein; (iv) a method of decreasing Tau aggregation; (v) a method of preventing the formation of Tau aggregates and/or of inhibiting Tau aggregation; or (vi) a method of interfering intracellularly with Tau aggregates; wherein the disease, disorder or an abnormality associated with misfolding of Tau protein and/or pathological aggregation of Tau (Tubulin associated unit) protein is selected from Alzheimer's disease (AD), familial Alzheimer's disease (AD), Primary Age-Related Tauopathy (PART), Creutzfeldt-Jacob disease, dementia pugilistica, Down's Syndrome, Gerstmann-Straussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17) also known familiar FTLD-Tau (MAPT), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, subacute sclerosing panencephalitis (SSPE), Senile dementia of the neurofibrillary tangle type, Parkinson's disease, HIV-related dementia, adult onset diabetes, senile cardiac amyloidosis, glaucoma, ischemic stroke, psychosis in Alzheimer's disease (AD), Lafora disease and Huntington's disease.

Preferably, the disease, disorder or the abnormality is selected from Alzheimer's disease (AD), Creutzfeldt-Jacob disease, dementia pugilistica, amyotrophic lateral sclerosis (ALS), argyrophilic grain disease, corticobasal degeneration (CBD), frontotemporal dementia with Parkinsonism linked to chromosome 17 (FTDP-17) also known familiar FTLD-Tau (MAPT), Pick's disease (PiD), progressive supranuclear palsy (PSP), tangle predominant dementia, Parkinson dementia complex of Guam, Hallervorden-Spatz disease, chronic traumatic encephalopathy (CTE), traumatic brain injury (TBI), and other frontotemporal lobar degeneration.

More preferably, the disease, disorder or the abnormality is selected from Alzheimer's disease (AD), corticobasal degeneration (CBD), Pick's disease (PiD), frontotemporal dementia with Parkinsonism linked to chromosome 17 (FTDP-17) also known familiar FTLD-Tau (MAPT) and progressive supranuclear palsy (PSP).

In another embodiment, the invention relates to (i) a method for treating, alleviating or preventing the disease, disorder or the abnormality associated with misfolding of Tau protein and/or pathological aggregation of Tau (Tubulin associated unit) protein; (ii) use in the treatment, alleviation or prevention of the disease, disorder or the abnormality associated with misfolding of Tau protein and/or pathological aggregation of Tau (Tubulin associated unit) protein; (iii) use for the manufacture of a medicament for treating, preventing or alleviating a disease, disorder or abnormality associated with misfolding of Tau protein and/or pathological aggregation of Tau (Tubulin associated unit) protein; (iv) a method of decreasing Tau aggregation; (v) a method of preventing the formation of Tau aggregates and/or of inhibiting Tau aggregation; or (vi) a method of interfering intracellularly with Tau aggregates wherein the disease, disorder or the abnormality is Alzheimer's disease (AD).

In another embodiment, the invention relates to (i) a method for treating, alleviating or preventing the disease, disorder or the abnormality associated with misfolding of Tau protein and/or pathological aggregation of Tau (Tubulin associated unit) protein; (ii) use in the treatment, alleviation or prevention of the disease, disorder or the abnormality associated with misfolding of Tau protein and/or pathological aggregation of Tau (Tubulin associated unit) protein; (iii) use for the manufacture of a medicament for treating, preventing or alleviating a disease, disorder or abnormality associated with misfolding of Tau protein and/or pathological aggregation of Tau (Tubulin associated unit) protein; (iv) a method of decreasing Tau aggregation; (v) a method of preventing the formation of Tau aggregates and/or of inhibiting Tau aggregation; or (vi) a method of interfering intracellularly with Tau aggregates wherein the disease, disorder or the abnormality is progressive supranuclear palsy (PSP).

In another embodiment, the invention relates to (i) a method for treating, alleviating or preventing the disease, disorder or the abnormality associated with misfolding of Tau protein and/or pathological aggregation of Tau (Tubulin associated unit) protein; (ii) use in the treatment, alleviation or prevention of the disease, disorder or the abnormality associated with misfolding of Tau protein and/or pathological aggregation of Tau (Tubulin associated unit) protein; (iii) use for the manufacture of a medicament for treating, preventing or alleviating a disease, disorder or abnormality associated with misfolding of Tau protein and/or pathological aggregation of Tau (Tubulin associated unit) protein; (iv) a method of decreasing Tau aggregation; (v) a method of preventing the formation of Tau aggregates and/or of inhibiting Tau aggregation; or (vi) a method of interfering intracellularly with Tau aggregates wherein the disease, disorder or the abnormality is frontotemporal dementia with Parkinsonism linked to chromosome 17 (FTDP-17) also known familiar FTLD-Tau (MAPT).

In one embodiment, the compound of formula (I) displays high capability in decreasing Tau aggregates by (a) recognizing aggregated Tau and disaggregating Tau, for example by changing the Tau aggregate molecular conformation, and/or (b) preventing the formation of Tau aggregates, and/or (c) interfering intracellularly with Tau aggregates, and/or (d) reducing Tau misfolding and hyperphosphorylation in vivo and/or (f) reducing neuroinflammatory markers. In one embodiment, the invention relates to a compound of formula (I) which can also be employed to decrease protein aggregation, in particular Tau aggregation. The ability of a compound of formula (I) to decrease Tau aggregation can, for example, be determined using the ThT assay (Hudson et al., FEBS J., 2009, 5960-72). In another embodiment, the compound of formula (I), as defined herein, can be used for decreasing tau aggregation in a subject. In yet another embodiment, the compound of formula (I), as defined herein can be used for the manufacture of a medicament for decreasing Tau aggregation. In another embodiment, the invention relates to a method of decreasing Tau aggregation, the method comprising administering an effective amount of a compound of formula (I), as defined herein, to a subject in need thereof. In yet another embodiment, the invention relates to a method of decreasing Tau aggregation, the method comprising administering a compound of formula (I), or a pharmaceutical composition comprising a compound of formula (I), as defined herein, to a subject in need thereof.

For example, Compound 1 (Example 1) of the invention can also be employed to decrease protein aggregation, in particular Tau aggregation. The ability of a compound to decrease of Tau aggregation can, for example, be determined using the ThT assay (Hudson et al., FEBS J., 2009, 5960-72). Thus, Compound 1 (Example 1), as defined above, can be used for decreasing tau aggregation in subject. Thus, Compound 1 (Example 1), as defined above, can be used for the manufacture of a medicament for decreasing Tau aggregation. In a method of decreasing Tau aggregation, Compound 1 (Example 1) can be administered in an effective amount to a subject in need thereof.

In another example, a compound as defined in Example 31 can also be employed to decrease protein aggregation, in particular Tau aggregation. Thus, a compound as defined in Example 31, can be used for decreasing Tau aggregation in subject, and a compound as defined in Example 31, can also be used for the manufacture of a medicament for decreasing Tau aggregation. In a method of decreasing Tau aggregation, a compound as defined in Example 31 can be administered in an effective amount to a subject in need thereof.

In another example, a compound as defined in Example 33 and Example 34 can also be employed to decrease protein aggregation, in particular Tau aggregation. Thus, a compound as defined in Example 33 and Example 34, can be used for decreasing tau aggregation in subject, and a compound as defined in Example 33 and Example 34, can also be used for the manufacture of a medicament for decreasing Tau aggregation. In a method of decreasing Tau aggregation, a compound as defined in Example 33 and Example 34 can be administered in an effective amount to a subject in need thereof.

In another example, a compound as defined in Example 37 and Example 38 can also be employed to decrease protein aggregation, in particular Tau aggregation. Thus, a compound as defined in Example 37 and Example 38, can be used for decreasing tau aggregation in subject, and a compound as defined in Example 37 and Example 38, can also be used for the manufacture of a medicament for decreasing Tau aggregation. In a method of decreasing Tau aggregation, a compound as defined in Example 37 and Example 38 can be administered in an effective amount to a subject in need thereof.

In yet another example, a compound as defined in Example 79 of the invention can also be employed to decrease protein aggregation, in particular Tau aggregation. Thus, a compound as defined in Example 79, can be used for decreasing tau aggregation in subject, and a compound as defined in Example 79, can also be used for the manufacture of a medicament for decreasing Tau aggregation. In a method of decreasing tau aggregation, a compound as defined in Example 79 can be administered in an effective amount to a subject in need thereof.

In one embodiment, the invention provides a method for preventing the formation of Tau aggregates and/or inhibiting Tau aggregation, wherein said method comprises administering an effective amount of a compound of formula (I) to a subject in need thereof. In yet another embodiment, the invention provides a method for preventing the formation of Tau aggregates and/or inhibiting Tau aggregation, wherein said method comprises administering a compound of formula (I), or a pharmaceutical composition comprising a compound of formula (I), to a subject in need thereof.

For example, the invention provides a method for preventing the formation of Tau aggregates and/or inhibiting Tau aggregation, wherein the method comprises administering an effective amount of Compound 1 (Example 1) to a subject in need thereof.

For example, the invention also provides a method for preventing the formation of Tau aggregates and/or inhibiting Tau aggregation, wherein the method comprises administering an effective amount of a compound as defined in Example 31 to a subject in need thereof.

For example, the invention also provides a method for preventing the formation of Tau aggregates and/or inhibiting Tau aggregation, wherein the method comprises administering an effective amount of a compound as defined in Example 33 and Example 34 to a subject in need thereof.

For example, the invention also provides a method for preventing the formation of Tau aggregates and/or inhibiting Tau aggregation, wherein the method comprises administering an effective amount of a compound as defined in Example 37 and Example 38 to a subject in need thereof.

In yet another example, the invention also provides a method for preventing the formation of Tau aggregates and/or inhibiting Tau aggregation, wherein the method comprises administering an effective amount of a compound as defined in Example 79 to a subject in need thereof.

In another embodiment, the invention provides a method of interfering intracellularly with Tau aggregates, wherein the method comprises administering an effective amount of a compound of formula (I) to a subject in need thereof. In yet another embodiment, the invention provides a method of interfering intracellularly with Tau aggregates, wherein the method comprises administering a compound of formula (I), or a pharmaceutical composition comprising a compound of formula (I), to a subject in need thereof.

For example, the invention provides for a method of interfering intracellularly with Tau aggregates, wherein the method comprises administering an effective amount of Compound 1 (Example 1) to a subject in need thereof.

In another example, the invention provides for a method of interfering intracellularly with Tau aggregates, wherein the method comprises administering an effective amount of a compound as defined in Example 31 to a subject in need thereof.

In another example, the invention provides for a method of interfering intracellularly with Tau aggregates, wherein the method comprises administering an effective amount of a compound as defined in Example 33 and Example 34 to a subject in need thereof.

In another example, the invention provides for a method of interfering intracellularly with Tau aggregates, wherein the method comprises administering an effective amount of a compound as defined in Example 37 and Example 38 to a subject in need thereof.

In yet another example, the invention provides for a method of interfering intracellularly with Tau aggregates, wherein the method comprises administering an effective amount of a compound as defined in Example 79 to a subject in need thereof.

Preferably, the above-mentioned uses and methods are applicable to animal or human subjects. More preferably, the subject is a human.

In one embodiment, the invention provides a pharmaceutical combination or combination comprising a compound of formula (I), and one or more therapeutic agents.

In yet another embodiment, the invention provides a mixture comprising a compound of formula (I), and one or more therapeutic agent different from the compound of formula (I), and optionally a pharmaceutically acceptable carrier, diluent, adjuvant and/or excipient. Said therapeutic agent being a further biologically active compound different from the compound of formula (I).

In yet another embodiment, the invention provides for a method of treating, alleviating, or preventing a disease, disorder or abnormality associated with misfolding of Tau protein and/or pathological aggregation of Tau protein comprising administering a compound of formula (I), as defined herein, wherein the compound is administered optionally in the presence of one or more therapeutic agent.

For example, the invention provides for a mixture comprising Compound 1 (Example 1), as defined above, and at least one further biologically active compound selected from a therapeutic agent different from Compound 1 (Example 1). In another example, the invention provides a pharmaceutical combination or combination comprising a Compound 1 (Example 1), and one or more therapeutic agents.

In another example, the invention provides for a pharmaceutical combination, a combination, or a mixture comprising a compound as defined in Example 31, as defined above and at least one further biologically active compound selected from a therapeutic agent different from compound of Example 31.

In another example, the invention provides for a pharmaceutical combination, a combination, or a mixture comprising a compound as defined in Example 33 and Example 34, as defined above and at least one further biologically active compound selected from a therapeutic agent different from compound of Example 33 and Example 34.

In another example, the invention provides for a pharmaceutical combination, a combination, or a mixture comprising a compound as defined in Example 37 and Example 38, as defined above and at least one further biologically active compound selected from a therapeutic agent different from compound of Example 37 and Example 38.

In yet another example, the invention provides for a pharmaceutical combination, a combination, or a mixture comprising a compound as defined in Example 79, as defined above and at least one further biologically active compound selected from a therapeutic agent different from compound of Example 79.

The nature of the further biologically active compound will depend on the intended use of the mixture. The further biologically active substance or compound may exert its biological effect by the same or a similar mechanism as the compound of formula (I) according to the invention or by an unrelated mechanism of action or by a multiplicity of related and/or unrelated mechanisms of action.

In one embodiment, the further biologically active compound is a compound used in the treatment of amyloidosis.

In one embodiment, the further biologically active compound is selected from neurological drugs, neuroinflammation inhibitors, anti-amyloid beta antibodies, amyloid beta aggregation inhibitors (including small molecules), anti-amyloid beta protein precursor (APP) antibodies, amyloid beta protein precursor (APP) inhibitors (including small molecules), anti-Tau antibodies, Tau aggregation inhibitors (including small molecules), anti-alpha-synuclein antibodies anti-alpha-synuclein inhibitors (including small molecules) and beta- and gamma-secretases inhibitors.

In one embodiment, the further biologically active compound is selected from neutron-transmission enhancers, psychotherapeutic drugs, acetylcholinesterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists.

In one embodiment, the further biologically active compound is selected from “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with a compound according to the invention and, optionally, a pharmaceutically acceptable carrier and/or a diluent and/or an excipient.

In one embodiment, the further biologically active compound is selected from compounds described in WO 2004/058258 (see especially pages 16 and 17) including therapeutic drug targets (pages 36 to 39), alkanesulfonic acids and alkanolsulfuric acids (pages 39 to 51), cholinesterase inhibitors (pages 51 to 56), NMDA receptor antagonists (pages 56 to 58), estrogens (pages 58 to 59), non-steroidal anti-inflammatory drugs (pages 60 and 61), antioxidants (pages 61 and 62), peroxisome proliferators-activated receptor (PPAR) agonists (pages 63 to 67), cholesterol-lowering agents (pages 68 to 75), amyloid inhibitors (pages 75 to 77), amyloid formation inhibitors (pages 77 to 78), metal chelators (pages 78 and 79), anti-psychotics and anti-depressants (pages 80 to 82), nutritional supplements (pages 83 to 89) and compounds increasing the availability of biologically active substances in the brain (see pages 89 to 93) and prodrugs (pages 93 and 94), which document is incorporated herein by reference.

The further biologically active compound can be identified as a second therapeutic agent.

The compounds of formula (I) according to the invention can also be provided in the form of a mixture with at least one further biologically active compound and/or a pharmaceutically acceptable carrier, a diluent, an excipient and/or adjuvant. The compound and/or the further biologically active compound are preferably present in a therapeutically effective amount.

When a compound of formula (I) (e.g. Compound 1 from Example 1) of the invention is used in combination with a further biologically active compound or a second therapeutic agent active against the same disease, the dose of each compound may differ from that when the compound is used alone.

In yet another embodiment, the invention relates to a combination as disclosed herein, or a mixture, as defined herein above, wherein one or more therapeutic agents are selected from the group consisting of compounds against oxidative stress; anti-amyloid drug; anti-apoptotic compounds; metal chelators; inhibitors of DNA repair such as pirenzepine and metabolites; 3-amino-1-propanesulfonic acid (3APS); 1,3-propanedisulfonate (1,3PDS); alpha-secretase activators; beta- and gamma-secretase inhibitors including BACE1; Tau proteins; neurotransmitters; beta-sheet breakers; attractants for amyloid beta clearing/depleting cellular components; inhibitors of N-terminal truncated amyloid beta including pyroglutamated amyloid beta 3-42; anti-inflammatory molecules; cholinesterase inhibitors (ChEIs) such as tacrine, rivastigmine, donepezil, and/or galantamine; M1 agonists; amyloid-beta or Tau modifying drugs; nutritive supplements; neurological drugs; corticosteroids, antibiotics, antiviral agents.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the second therapeutic agent may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manner as are known for such compounds in the art.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

A proposed dose of the compounds according to the present invention for administration to a human (of approximately 70 kg body weight) is 0.1 mg to 1.5 g, preferably 1 mg to 500 mg of the active ingredient per unit dose. The unit dose may be administered, for example, 1 to 4 times per day. The dose will depend on the route of administration. It will be appreciated that it may be necessary to make routine variations to the dosage depending on the age and weight of the patient as well as the severity of the condition to be treated. The precise dose and route of administration will ultimately be at the discretion of the attendant physician or veterinarian.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like. The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In one embodiment, the compounds of the invention can be used as an analytical reference or an in vitro screening tool for characterization of tissue with Tau pathology and/or for screening compounds targeting Tau pathology on such tissue.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diastereoisomeric mixtures and optically pure forms. Optically active (R)- and (S)-stereoisomers may be prepared using chiral synthons or chiral reagents or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included. The invention is also meant to include any pseudo-asymmetric carbon atom, represented herein as (r)- and (s)-, and which are invariant on reflection in a mirror but are reversed by exchange of any two entities, (PAC 1996, 68, 2193, Basic terminology of stereochemistry IUPAC recommendations 1996).

According to the present invention a compound as defined herein can be in the form of one of the possible stereoisomers, rotamers, atropisomers, tautomers or mixtures thereof, for example, as substantially pure geometric (cis or trans) stereoisomers, diastereomers, optical isomers (antipodes), racemates, or mixtures thereof. Any resulting mixtures of stereoisomers can be separated on the basis of the physicochemical differences of the constituents, into the pure or substantially pure geometric or optical isomers, diastereomers, racemates, for example, by chromatography and/or fractional crystallization. Any resulting racemates of final products or intermediates can be resolved into the optical antipodes by known methods, e.g., by separation of the diastereomeric salts thereof, obtained with an optically active acid or base, and liberating the optically active acidic or basic compound. Racemic products can also be resolved by chiral chromatography (e.g., high performance liquid chromatography (HPLC)) using a chiral adsorbent.

Pharmaceutically acceptable acid addition salts can be formed with organic acids and inorganic acids. For example, organic acids from which salts can be derived include, sulfosalicylic acid, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, and the like. Inorganic acids from which salts can be derived include, for example, sulfuric acid, hydrochloric acid, hydrobromic acid, nitric acid, phosphoric acid, and the like.

In the same way, pharmaceutically acceptable base addition salts can be formed with organic and inorganic bases. For example, organic bases from which salts can be derived include, primary, secondary, and tertiary amines, substituted amines including naturally occurring substituted amines, cyclic amines, basic ion exchange resins, and the like (e.g. isopropylamine, benzathine, cholinate, diethanolamine, diethylamine, lysine, meglumine, piperazine, and tromethamine). Example of inorganic bases from which salts can be derived include, for example, ammonium salts and metals from columns I to XII of the periodic table. Said salts are derived from, for example, sodium, potassium, ammonium, calcium, magnesium, iron, silver, zinc, and copper.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and delectability. ¹⁸F-labeled compounds are particularly suitable for imaging applications such as PET. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

General Schemes

The compounds of the present invention may be prepared in accordance with the definition of a compound of formula (I), as defined herein, by the routes described in the following Schemes or Examples. All methods described herein can be performed in any suitable order unless otherwise indicated herein, or otherwise clearly contradicted by the context. The use of any and all examples, or exemplary language (e.g. “such as”) as used herein is intended to merely illustrate the invention and does not pose a limitation on the scope claimed. In the following general methods, Y, R¹, R², R, Q¹, Q², Q³, Q⁴, Z¹, Z^{1′}, Z², Z³, Z⁴, L, and R^{L} are as previously defined in the above embodiments or limited to the designations in the Schemes. Unless otherwise stated, starting materials are either commercially available or are prepared by known methods.

### General Synthetic Schemes for the Preparation of Building Blocks of this Invention

### 1.1. General Synthetic Schemes for the Preparation of the Preparative Examples

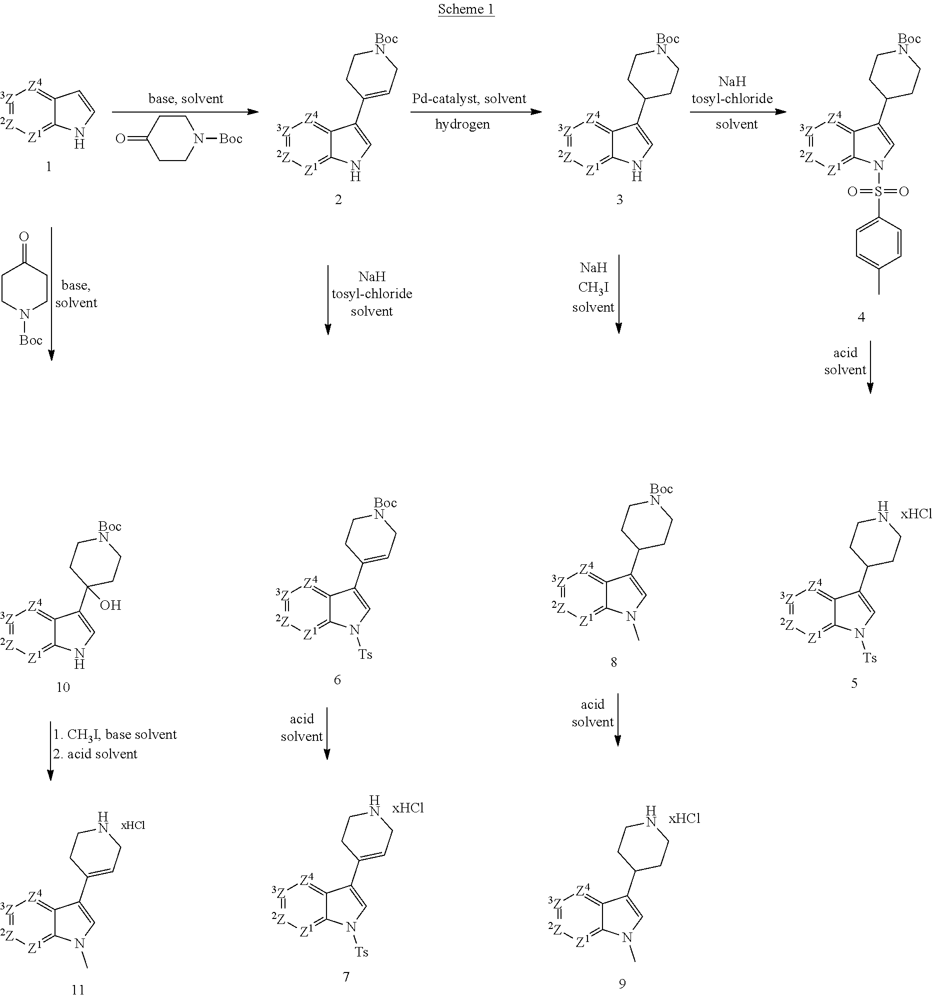

For example, commercially available indole-type derivative 1 with substituents Z¹, Z², Z³, Z⁴ as indicated in Scheme 1 (similar conditions can be used with different R² groups as defined herein) can be heated with commercially available 1-Boc-4-piperidone in the presence of a suitable base (e.g. potassium hydroxide, sodium methoxide, etc.) in a suitable solvent (e.g. MeOH, etc.) to afford product 2. The NH-moiety can be protected with a tosyl-protecting group employing sodium hydride and tosyl-chloride in a suitable solvent (e.g. THF, DMF) to afford compound 6. Cleavage of the Boc-protecting group by acid treatment (e.g. HCl, TFA) in a suitable solvent (e.g. CH₂Cl₂, dioxane) affords compound 7 as a HCl-salt. Alternatively, the double bond of product 2 can be reduced with hydrogen employing a suitable catalyst (e.g. Pd/C, Pd(OH)₂/C) in a suitable solvent (e.g. MeOH, EtOH) to afford 3. Tosyl-protection followed by Boc-cleavage affords 5 as HCl-salts. N-methylation of the NH-moiety of compound 3 employing sodium hydride and methyl iodide in a suitable solvent (e.g. THF, DMF) followed by acid treatment affords 9 as a HCl-salt.

Alternatively, the commercially available derivative 1 can be reacted at room temperature with commercially available 1-Boc-4-piperidone in the presence of a suitable base (e.g. potassium hydroxide) in a suitable solvent (e.g. EtOH) to afford product 10. N-methylation of the NH-moiety of 10 with sodium hydride and methyl iodide in a suitable solvent (e.g THF), followed by cleavage of the Boc-protecting group under acidic conditions (e.g. HCl, TFA) in a suitable solvent (e.g. CH₂Cl₂) gave 11 as a HCl-salt.

Alternatively, commercially available tert-butyl 5-oxohexahydrocyclopenta[c]pyrrole-2(1H)-carboxylate can be reacted with derivative 1 in the same manner as 1-Boc-4-piperidone, in the presence of a suitable base (e.g. potassium hydroxide, sodium methoxide) in a suitable solvent (e.g. MeOH) to afford the corresponding product.

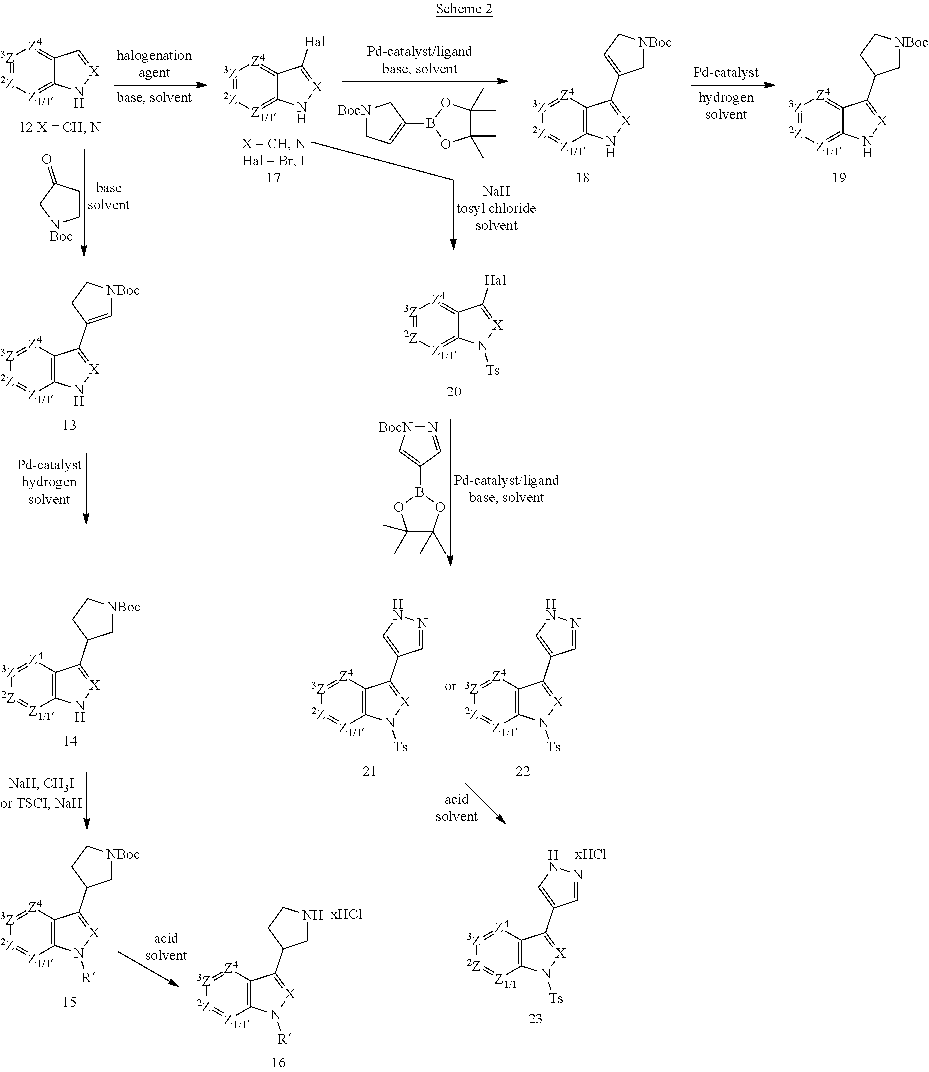

For example, commercially available derivative 12 as indicated in Scheme 2 can be reacted with commercially available N-Boc-3-pyrrolidinone in the presence of a suitable base (e.g. potassium hydroxide, sodium methoxide) in a suitable solvent (e.g. MeOH) to afford product 13. Compound 13 can be reduced with hydrogen employing a suitable catalyst (e.g. Pd/C) in a suitable solvent (e.g. MeOH) to afford 14. The NH-moiety of compound 14 can be reacted with sodium hydride and methyl iodide to afford 15 as an N-methyl derivative (R′ is Me) or can be reacted with tosyl chloride and sodium hydride in a suitable solvent to afford the tosyl protected compound 15 (R′ is Ts). Cleavage of the Boc-protecting group by acid treatment (e.g. HCl, TFA) in a suitable solvent (e.g. CH₂Cl₂, dioxane) affords 16 as a HCl-salt.

Alternatively, heterocycle 12, for example, can also be reacted with halogenation agents (e.g. iodine, N-bromo-succinimide) in the presence of a base (e.g. potassium hydroxide, sodium hydroxide etc.) in a suitable solvent (e.g. DMF, MeOH) to afford the halogenated derivatives 17 (Hal=Br, I) after purification. Derivative 17 can be reacted with commercially available tert-butyl 3-(4,4,5,5-tetramethyl-1,3,2-dioxaborolan-2-yl)-2,5-dihydro-1H-pyrrole-1-carboxylate in a Suzuki coupling employing a catalyst/ligand system (e.g. PdCl₂(dppf)₂×CH₂Cl₂, Pd[P(Ph)₃]₄), a base (e.g. Cs₂CO₃, Na₂CO₃, K₂CO₃), and a suitable solvent (e.g. dioxane/water) to afford the coupling product 18. The double bond of 18 can be reduced with hydrogen using a suitable catalyst (e.g. Pd/C) in a suitable solvent (e.g. MeOH, THF) to afford the derivative 19. The compound can be further reacted as disclosed above to afford the tosyl-protected compound. The reaction steps to afford compound 19 can also be performed with all R² derivatives as defined in claim 1.

The halogenated compound 17 (Hal=Br, or I) as indicated in Scheme 2 can be reacted with sodium hydride and tosyl-chloride in a suitable solvent (e.g. THF, DMF) to afford the tosyl-protected derivative 20. Alternatively, compound 12 with substituents Z¹, Z², Z³, Z⁴, X═CH can be halogenated with iodine in the presence of a base and treated in situ with tosyl-chloride in a suitable solvent (e.g DMF, etc.) to afford the tosyl-protected derivative 18 (with Hal is iodine) in one-step. The tosyl-protected derivative 20 can undergo a Suzuki coupling employing a catalyst/ligand system (e.g. PdCl₂(dppf)₂×CH₂Cl₂, Pd[P(Ph)₃]₄), a base (e.g. Cs₂CO₃, Na₂CO₃, K₂CO₃), and a suitable solvent (e.g. dioxane/water) to afford the palladium coupling product 21 or 22. Compound 21 (X═CH) can be isolated as a free base after purification, while compound 22 (X═N) is isolated with a boc moiety that can be cleaved under acidic conditions (e.g. HCl, TFA) in a suitable solvent (e.g. CH₂Cl₂, dioxane).

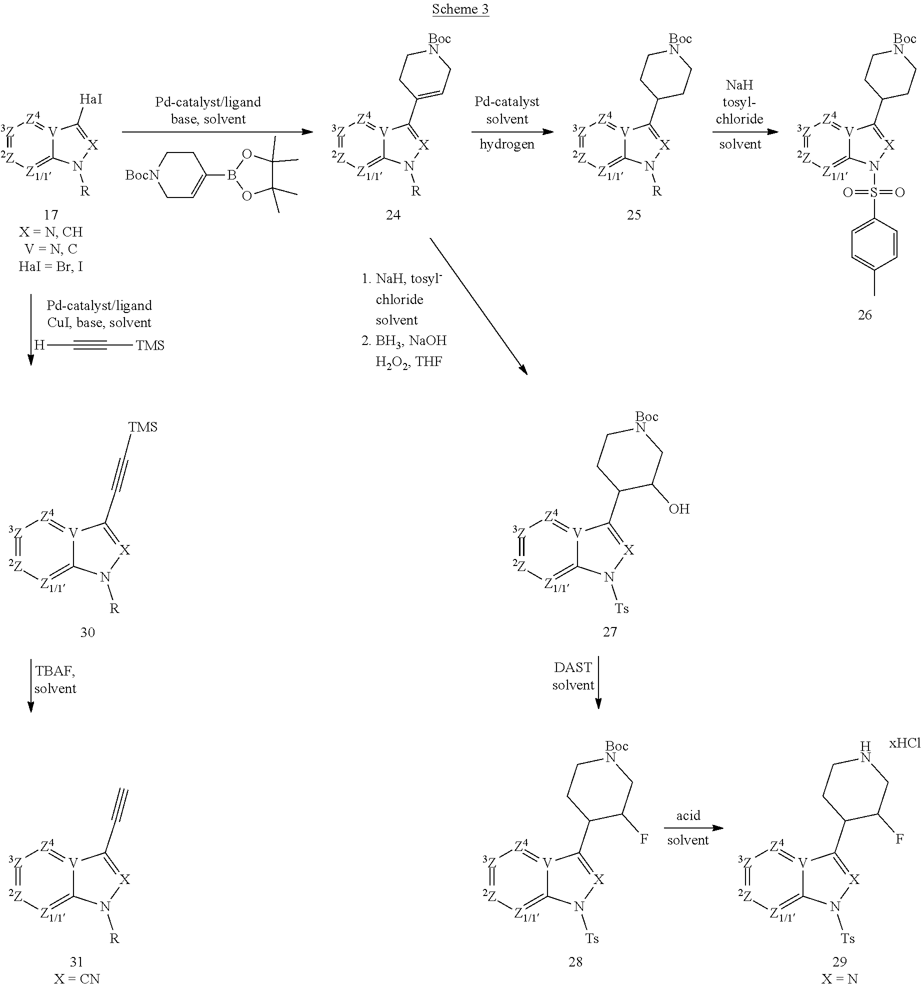

As an example, heterocycle 17 with substituents Z¹, Z^{1′}, Z², Z³, Z⁴, R, X, V and Hal as indicated in Scheme 3 can be reacted under Suzuki-coupling reactions with suitable commercially available boronic esters (e.g. tert-butyl 4-(4,4,5,5-tetramethyl-1,3,2-dioxaborolan-2-yl)-3,6-dihydropyridine-1(2H)-carboxylate, tert-butyl 3-(4,4,5,5-tetramethyl-1,3,2-dioxaborolan-2-yl)-2,5-dihydro-1H-pyrrole-1-carboxylate) in the presence of a suitable palladium catalyst/ligand system (e.g. Pd(dppf)Cl₂×CH₂Cl₂, Pd(PPh₃)₄), a suitable base (e.g. Na₂CO₃, K₂CO₃, Cs₂CO₃, etc.), and a suitable solvent (e.g. dioxane/water) to afford the coupling product 24 (e.g. X═N and V═C). Compound 24 (with R═H) can be reacted with sodium hydride and tosyl-chloride in a suitable solvent (e.g. THF, DMF, etc.), followed by a hydroboration-oxidation reaction using a borane complex (e.g. BH₃/THF) and hydrogen peroxide in the presence of a base (e.g. NaOH, etc.) in a suitable solvent (e.g. THF, etc.) to afford the syn-addition product 27. Employing diethylaminosulfur trifluoride in a suitable solvent (e.g. CH₂Cl₂) at low temperature (−60° C. or less) affords the corresponding fluoro-derivative 28. Cleavage of the Boc-protecting group under acidic conditions (e.g. HCl, TFA, etc.) using a suitable solvent (e.g. CH₂Cl₂, dioxane, etc.) affords 29 as a HCl-salt.

Compound 24 (with R═H or CH₃) can be reduced with hydrogen employing a suitable catalyst (e.g Pd/C, Pd(OH)₂/C, etc.) in a suitable solvent (e.g. MeOH, EtOH, etc.) to afford the fully saturated derivative 25, which can be further treated with acid (e.g. HCl, TFA, etc.) in a suitable solvent (e.g CH₂Cl₂, dioxane, etc.) to afford the compound as an HCl-salt. Compound 25 with R═H can be treated with sodium hydride and tosyl-chloride in a suitable solvent (e.g. THF, DMF, etc.) to afford the tosyl-protected compound 26. The Boc-protecting group can be cleaved by acid treatment (e.g. HCl, TFA, etc.) in a suitable solvent (e.g. CH₂Cl₂, dioxane, etc.) to afford the compound as an HCl salt (Similar to transformation of compound 4 to 5 in Scheme 1) Alternatively, compound 24 with R═H can be reacted with sodium hydride and tosyl-chloride in a suitable solvent (e.g. THF, DMF, etc.) and then acidic conditions (e.g. HCl, TFA, etc.) in a suitable solvent (e.g. CH₂Cl₂, dioxane, etc.) to cleave the Boc-protecting group and obtain the compound as an HCl-salt in a similar manner to Scheme 1 (transformation of compound 2 to 7).

Alternatively, derivative 17 (with Hal=1) can be reacted with trimethylsilyl-acetylene in a Sonogashira reaction (e.g. PdCl₂(PPh₃)₂, copper (I)-iodide, triethylamine) in a suitable solvent (e.g. THF) to afford the coupling product 30. Compound 31 can be obtained by cleavage of the silyl-protecting group with tetra-butylammonium fluoride in a suitable solvent (e.g. THF).

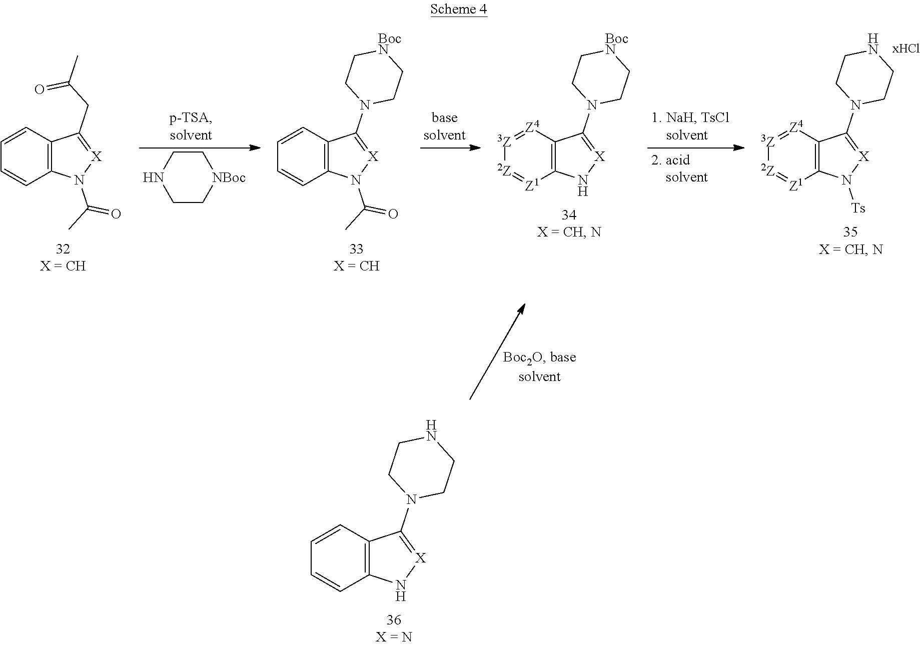

Commercially available 1-acetyl-1H-indol-3-yl acetate 32 was reacted with tert-butyl piperazine-1-carboxylate in the presence of p-toluenesulfonic acid in a suitable solvent (e.g. toluene) to afford product 33. The acetyl-protecting group (33) can be cleaved by reacting it with base (e.g. triethylamine) in a suitable solvent (e.g. MeOH) to afford compound 34 (X═CH). Then, protection of the NH-moiety (34) with the tosyl-protecting group using sodium hydride and tosyl-chloride in a suitable solvent (e.g. THF, DMF) followed by cleavage of the Boc-protecting group under acidic conditions (e.g. HCl, TFA) in a suitable solvent (e.g. CH₂Cl₂, dioxane) afforded 35 as a HCl-salts.

Alternatively, compound 34 (X═N) can be obtained by reacting commercially available 3-(piperazin-1-yl)-1H-indazole (36) with di-tert-butyl dicarbonate in a suitable solvent (e.g. CH₂Cl₂) in the presence of a base (e.g. triethylamine).

### 1.2. General Synthetic Schemes for the Preparation of the Core Structure

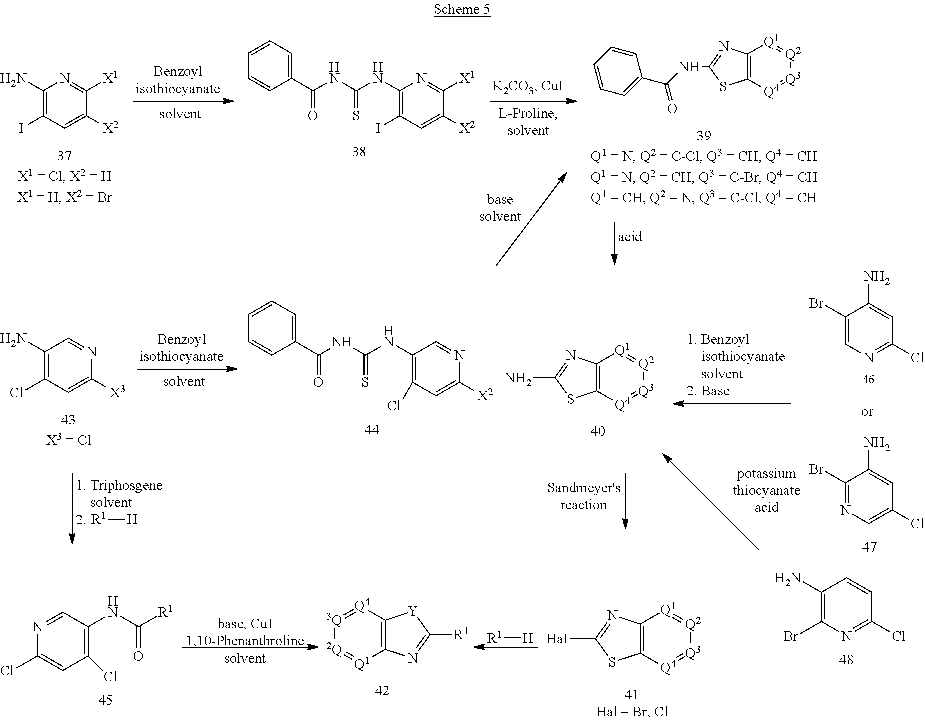

Commercially available pyridine derivative 37/43 can be reacted with benzoyl isothiocyanate in a suitable solvent (e.g. acetone) to afford 38/44. Copper mediated ring closure using a base (e.g. K₂CO₃), a catalyst (e.g. L-proline) in a suitable solvent (e.g. dioxane), followed by acid (e.g. H₂SO₄, 70% H₂SO₄) mediated cleavage of the benzoyl-moiety afforded 39. Alternatively, the ring closure of 44 can be achieved under basic conditions (e.g. NaOMe) in the presence of a suitable solvent (e.g. NMP). Compound 41 can be obtained by acid mediated (e.g. H₂SO₄, 70% H₂SO₄) cleavage of the benzoyl-moiety (40) followed by replacement of the amine by a halogen group using Sandmeyer reaction conditions (e.g. iso-amylnitrite, copper (II)-bromide or copper (II)-chloride; or NaNO₂, copper (I)-chloride) in a solvent (e.g. CH₃CN). Compound 42 (Y═N, wherein at least one of Q² or Q³ comprise a halogen group) can be obtained by reacting 41 with R¹—H (e.g. morpholine, 4-methoxypiperidine, 6-oxa-3-azabicyclo[3.1.1]heptane) either in neat conditions or in the presence of a suitable base (e.g. K₂CO₃, triethylamine) and solvent (e.g. CH₂Cl₂, CH₃CN).

Alternatively, compound 42 can be obtained by reacting the corresponding commercially available benzo[d]oxazole and benzo[d]thiazole derivatives with R¹—H (e.g. morpholine, 4-methoxypiperidine, 6-oxa-3-azabicyclo[3.1.1]heptane) either in neat conditions or in the presence of a suitable base (e.g. K₂CO₃, triethylamine) and solvent (e.g. CH₂Cl₂, CH₃CN).

Commercially available 4,6-dichloropyridin-3-amine 43 can alternatively be reacted with triphosgene in a suitable solvent followed by addition of R¹—H (e.g. morpholine) to obtain 45. Compound 42 can be obtained by reacting 45 with copper (I)-iodide, a base (e.g. Cs₂CO₃, etc.), 1,10-phenanthroline, and a suitable solvent (e.g. dioxane).

Alternatively, commercially available pyridine derivatives (46/47) can be reacted with benzoyl isothiocyanate in a solvent (e.g. acetone) followed by ring closure in the presence of a base (e.g. NaOH) in a suitable solvent (e.g. MeOH) to afford 40. Alternatively, commercially available pyridine 48 can be reacted with potassium thiocyanate in the presence of a suitable acid (e.g. HCl) to afford compound 40.

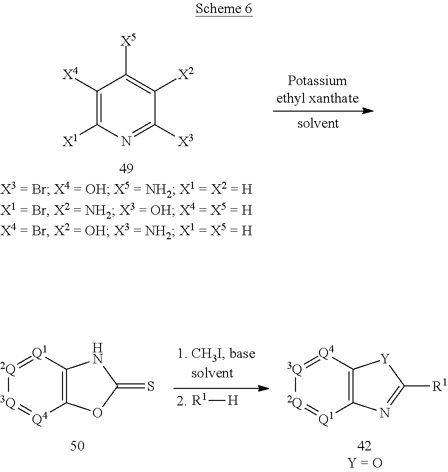

Compound 49 can be reacted with potassium ethyl xanthate in a suitable solvent (e.g. pyridine) to afford the cyclization product 50 containing a pyridine-thione moiety. Methylation of the S-atom using methyl iodide in the presence of a base (e.g. K₂CO₃) in a suitable solvent (e.g. ethyl acetate), followed by addition of R¹—H (e.g. morpholine, 4-methoxypiperidine, 6-oxa-3-azabicyclo[3.1.1]heptane) using appropriate reaction conditions (neat or triethylamine/CH₂Cl₂) yielded 42 (Y═N, wherein at least one of Q² or Q³ comprise a halogen group).

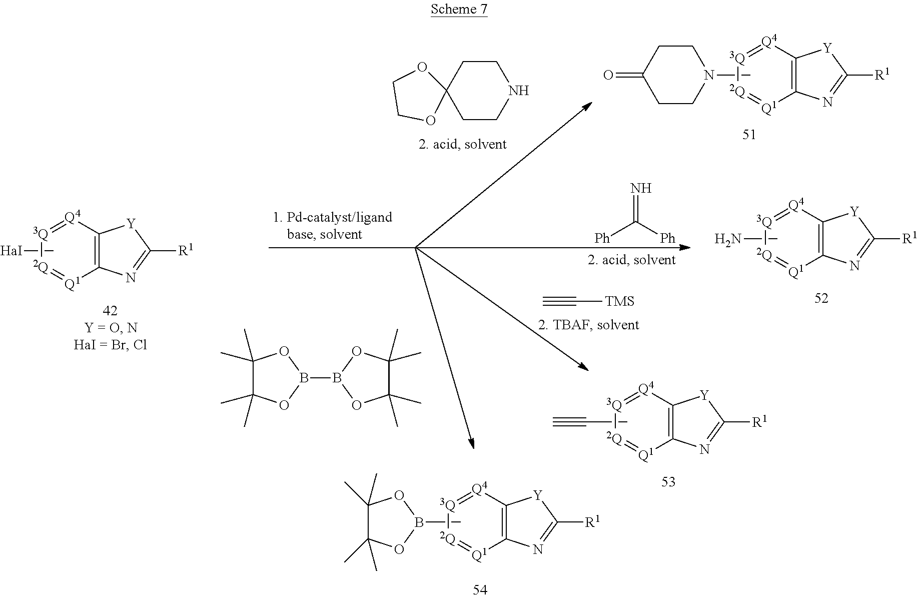

Compound 42 prepared as described in Scheme 5 and Scheme 6 can be reacted with the appropriate L group (as defined herein) using a palladium coupling reaction.

For example, 42 can be reacted with commercially available 1,4-dioxa-8-azaspiro[4.5]decane in the presence of a palladium catalyst/ligand system (e.g. Pd(OAc)₂/XPhos), a base (e.g. Cs₂CO₃), and a suitable solvent (e.g. dioxane), followed by aqueous cleavage of the acetal-moiety with acid (e.g. HCl) to obtain 51.

Alternatively, 42 can be reacted with diphenylmethanimine employing Buchwald-Hartwig cross coupling reaction conditions (e.g. Pd₂(dba)₃, Ruphos, NaOtBu, dioxane) followed by acid (e.g. 1.5N HCl) in a suitable solvent (e.g. THF) to afford 52.

In another example, 42 can be reacted with ethynyltrimethylsilane followed by addition of tetra-butylammonium fluoride in a suitable solvent (e.g. THF) to afford 53.

In yet another example, 54 can be prepared by reacting 42 with commercially available 4,4,4′,4′,5,5,5′,5′-octamethyl-2,2′-bi(1,3,2-dioxaborolane) under Suzuki conditions employing a palladium catalyst/ligand system (e.g. PdCl₂(dppf)×CH₂Cl₂), a base (e.g. KOAc), and a suitable solvent (e.g. dioxane).

### 1.3. General Synthetic Schemes for the Preparation of the Examples

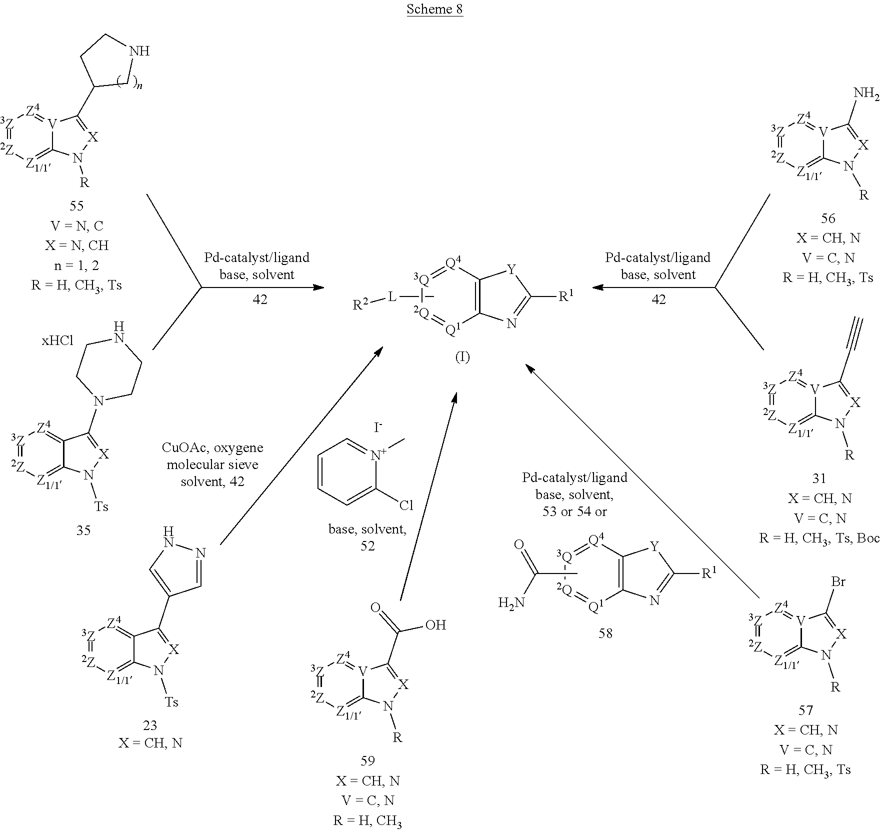

The compound of formula (I) can be obtained by reacting the appropriate Preparative Example (e.g. compounds 55, 35, 31, 56) with compound (42) using a palladium coupling (e.g. Buchwald-Hartwig cross coupling reaction, Sonogashira reaction, Suzuki reaction) as described in the examples of the present invention.

In another example, compound of formula (I) can be obtained by reacting 23 with 42 in the presence copper (II) acetate, molecular sieves, and pyridine while exposed to air, and then, cleaving the tosyl protecting group with a suitable base (e.g. NaOtBu) in the presence of a suitable solvent (e.g. dioxane/MeOH) In yet another example, compound of formula (I) can be obtained by reacting the appropriate 57 with, for example, compounds 58, 53, or 54 using a palladium coupling (e.g. Sonogashira coupling conditions)

Alternatively, 59 (e.g. commercially available 1H-indole-3-carboxylic acid and 1H-pyrrolo[2,3-b]pyridine-3-carboxylic acid) can be reacted with morpholinobenzo[d]oxazol-amines employing the Mukaiyama reagent conditions (2-chloro-N-methylpyridinium iodide), a suitable base (e.g. triethylamine) in a suitable solvent (e.g. CH₂Cl₂) to afford compound of formula (I).

In one example, the Compound 1 (Example 1) of the present invention can be synthesized as exemplified below This method is only given for illustrative purposes and should not to be construed as limiting. For example, the method for producing Compound 1 (Example 1) comprises the step of deprotecting Compound 2. Preferably, the deprotection of Compound 2 occurs in presence of strong base such as NaOtBu.

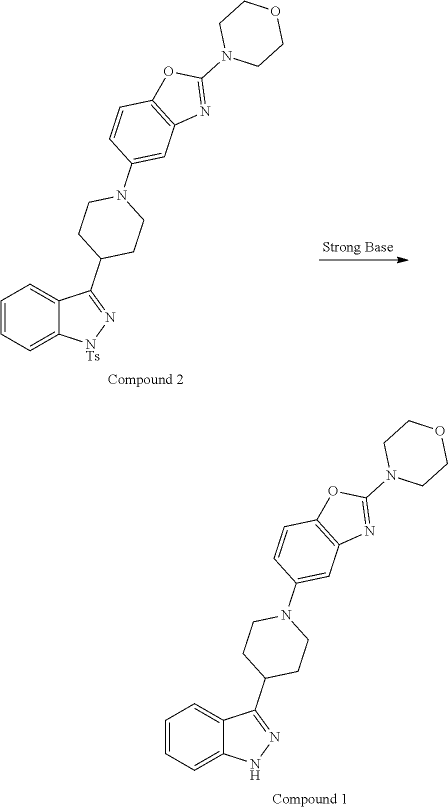 [SMILES: Cc1ccc(S(=O)(=O)n2nc(C3CCN(c4ccc5oc(N6CCOCC6)nc5c4)CC3)c3ccccc32)cc1 ; c1ccc2c(C3CCN(c4ccc5oc(N6CCOCC6)nc5c4)CC3)n[nH]c2c1]

### EXAMPLES

### Examplification of the Invention

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

Unless otherwise stated, all reagents and solvents were obtained from commercial sources and used without further purification. All starting materials, building blocks, reagents, acids, bases, dehydrating agents, solvents, and catalysts utilized to synthesis the compounds of the present invention are either commercially available or can be produced by organic synthesis methods known to one of ordinary skill in the art.

The chemical names were generated using ChemBioDraw Ultra v20.1 from CambridgeSoft.

Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

### Abbreviations

| | |
|---|---|
| ACN | acetonitrile |
| Boc | tert-Butyloxycarbonyl |
| BrettPhos Pd G3 | BrettPhos palladacycle 3rd generation |
| DAST | Diethylaminosulfur trifluoride |
| DCM | Dimethylformamide |
| DMF | Dichloromethane |
| EDTA | Ethylenediaminetetraacetic acid |
| EGTA | Ethylene glycol-bis(β-aminoethyl |
| | ether)-N,N,N′,N′-tetraacetic acid |
| EtOAc | Ethyl acetate |
| Eq. | Equivalent |
| HF-Pyridine | Hydrogen Fluoride Pyridine |
| HPLC | High performance liquid chromatography |
| LCMS | Liquid Chromatograph-Mass Spectrometry |
| NBS | N-Bromosuccinimide |
| NCS | N-Cholorosuccinimide |
| NMP | N-metyl-2-pyrrolidone |
| PMSF | Phenylmethylsulfonyl fluoride |
| rt or RT | Room temperature |
| TBAF | Tetra-n-butylammonium fluoride |
| TBDPSCl | Ctert-butyldiphenylsilyl chloride |
| TEA | Triethylamine |
| TFA | Trifluoroacetic acid |
| THF | tetrahydrofuran |
| TMS | Trimethylsilyl group |
| Ts or Tos | Tosyl |
| TMS-Cl | trimethylsilyl chloride |

Analytical Details

NMR: ¹H-NMR spectra were recorded on BrukerAV 300 and 400 MHz spectrometers in deuterated solvents. Chemical shifts (δ) are reported in parts per million and coupling constants (J values) in hertz. Spin multiplicities are indicated by the following symbols: s (singlet), d (doublet), t (triplet), q (quartet), m (multiplet), bs (broad singlet). Deuterated solvents are given in parentheses and have a chemical shifts of dimethyl sulfoxide (δ 2.50 ppm), methanol (δ 3.31 ppm), chloroform (δ 7.26 ppm), or other solvent as indicated in NMR spectral data.

MS: Mass spectra were obtained on an Agilent 1290 Infinity II spectrometer with a 6130 Chemstation and an Agilent 1200 Infinity II spectrometer with a 6130 Chemstation. GC-MS data were collected using an Agilent 7890B gas chromatograph and 5977B mass spectrometer. Infrared spectra were obtained on a PerkinElmer spectrometer. Chromatography was performed using silica gel (Fluka: Silica ge10l 60, 0.063-0.2 mm) and suitable solvents as indicated in specific examples.

Flash Column Chromatography System: Flash purification was conducted with a Biotage Isolera with HP-Sil or KP-NH SNAP cartridges (Biotage) and the solvent gradient indicated in specific examples.

Thin layer chromatography (TLC): TLC was carried out on silica gel plates with UV detection.

Chiral separation by Supercritical Fluid Chromatography (SFC): Chiral separation was conducted with a PIC LAB Hybrid 10-20 system equipped with double piston CO₂ pump and modified pump, autosampler, automatic backpressure regulator (ABPR), and PDA detector; operated with PIC analytical software, using suitable solvents as indicated in specific examples

### SYNTHESIS OF THE PREPARATIVE EXAMPLES

### Preparative Example 1: 3-(piperidin-4-yl)-1-tosyl-1H-indazole Hydrochloride

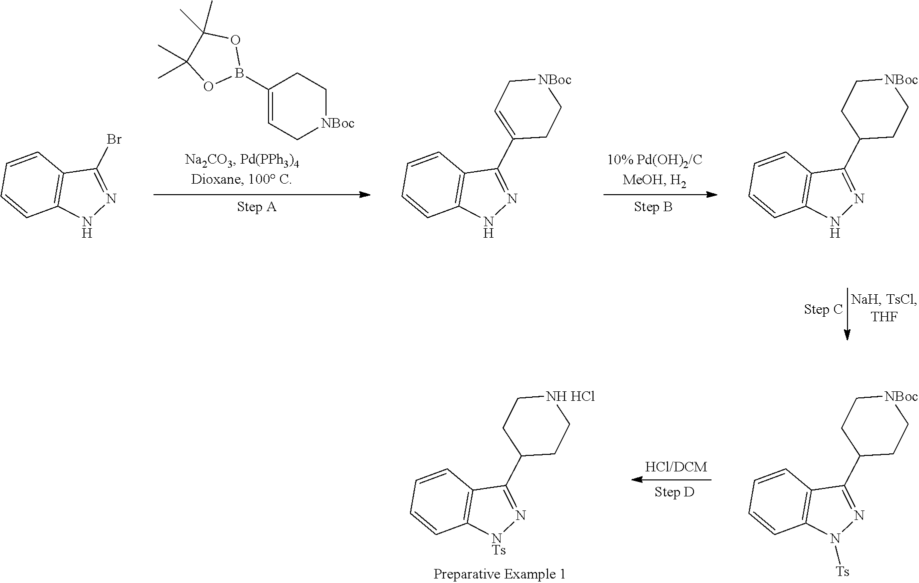

Step A

To a stirred solution of 3-bromo-1H-indazole (10 g, 50.20 mmol) in 1,4-dioxane (200 ml) was added tert-butyl 4-(4,4,5,5-tetramethyl-1,3,2-dioxaborolan-2-yl)-3,6-dihydro-2H-pyridine-1-carboxylate (18.6 g, 60.30 mmol) followed by the addition of 2.0 M Na₂CO₃ (75.4 ml) in a 500 ml multi-neck round bottom flask and purged with nitrogen for 20 minutes. Tetrakis(triphenylphosphine)palladium(0) (2.90 g, 25.10 mmol) was added, purged with nitrogen for 10 minutes and heated at 100° C. for 16 hours. Completion of the reaction was monitored by TLC. The reaction mixture was diluted with water (200 ml) and extracted with ethyl acetate (2×500 ml). The combined organic layers were dried over Na₂SO₄ and filtered. The reaction mixture was concentrated under reduced pressure. The residue was purified on 230-400 silica gel cartridge using a Biotage purification system by employing a petroleum ether/ethyl acetate gradient (100/0=>82/18) to afford the tert-butyl 4-(1H-indazol-3-yl)-3,6-dihydropyridine-1(2H)-carboxylate (14 g, 88.3%) as a yellow gummy solid. ¹H-NMR (400 MHz, DMSO-d₆): δ 13.00 (s, 1H), 7.99 (d, J=10.80 Hz, 1H), 7.53 (d, J=11.20 Hz, 1H), 7.36 (t, J=10.40 Hz, 1H), 7.14 (t, J=9.60 Hz, 1H), 6.54 (s, 1H), 4.10 (s, 2H), 3.58 (t, J=7.20 Hz, 2H), 2.69 (br, 2H), 1.44 (s, 9H). MS: 244.1 (M−tBu)⁺

Step B

To a stirred solution of the crude tert-butyl 4-(1H-indazol-3-yl)-3,6-dihydropyridine-1(2H)-carboxylate compound from Step A (14.5 g, 46.0 mmol) in methanol (MeOH, 150 ml) was added 10% Pd(OH)₂/C (1.45 g, 1.03 mmol). The reaction mixture was stirred under H₂ atmosphere at room temperature for 3 hours. The crude was filtered through celite followed by ethyl acetate wash (100 ml) and the filtrate was concentrated under reduced pressure. The residue was purified using a Biotage purification system by employing a petroleum ether/ethyl acetate gradient (100/0 to 75/35) to afford the tert-butyl 4-(1H-indazol-3-yl)piperidine-1-carboxylate compound (12.3 g, 84.7%) as a yellow solid. ¹H-NMR (400 MHz, CDCl₃): δ 7.79 (d, J=0.80 Hz, 1H), 7.48-7.49 (m, 1H), 7.41-7.43 (m, 1H), 7.17-7.19 (m, 1H), 4.27 (m, 2H), 3.24-3.30 (m, 1H), 2.97 (t, J=12.00 Hz, 2H), 1.93-2.09 (m, 4H), 1.49 (s, 9H). MS: 246.1 (M−tBu)⁺.

Step C

To a suspension of sodium hydride (NaH, 60% in paraffin oil, 3.2 g, 80 mmol) in tetrahydrofuran (150 ml) was added portion wise the tert-butyl 4-(1H-indazol-3-yl)piperidine-1-carboxylate compound from Step B at 0° C., and the reaction was stirred at room temperature for 60 minutes. Tosyl chloride (11.4 g, 60 mmol) was added at 0° C. dropwise (previously dissolved in THF 100 ml) and the reaction was stirred at room temperature for 2 hours. The reaction mixture was quenched with ice cold water slowly, and extracted using ethyl acetate (3×250 ml). The organic layer was separated, dried over sodium sulphate, and filtered. Then the solvent was removed under reduced pressure. The residue was purified on 60-120 silica gel cartridge using a Biotage purification system by employing a petroleum ether/ethyl acetate gradient (100/0 to 70/30) to afford the tert-butyl 4-(1-tosyl-1H-indazol-3-yl)piperidine-1-carboxylate compound (12.5 g, 67%) as a pale yellow solid. ¹H-NMR (400 MHz, CDCl₃): δ 8.21 (d, J=8.80 Hz, 1H), 7.84 (d, J=8.40 Hz, 2H), 7.67 (d, J=8.40 Hz, 1H), 7.53-7.57 (m, 1H), 7.29-7.34 (m, 1H), 7.24 (d, J=8.40 Hz, 2H), 4.18-4.21 (m, 2H), 3.17-3.20 (m, 1H), 2.90-2.95 (m, 2H), 2.37 (s, 3H), 1.93-1.94 (m, 4H), 1.46 (s, 9H). MS: 400.2 (M−tBu)⁺.

Step D

To a stirred solution of the tert-butyl 4-(1-tosyl-1H-indazol-3-yl)piperidine-1-carboxylate compound from Step C (12.5 g, 29.5 mmol) in dichloromethane (100 ml), 4N HCl in 1,4-dioxane was added (8 ml) at 0° C., then stirred for additional 1 hour at 0° C. and warmed up to room temperature. After completion of the reaction by TLC the reaction mixture was concentrated. Diethyl ether (50 ml) and petroleum ether (50 ml) were added, and the crude was stirred for 15 minutes at room temperature. The obtained solid was filtered, dried under reduced pressure to afford the 3-(piperidin-4-yl)-1-tosyl-1H-indazole hydrochloride (10 g, 93%) as an off-white solid. ¹H-NMR (300 MHz, DMSO-d₆): δ 8.10 (d, J=8.40 Hz, 1H), 7.98 (d, J=8.10 Hz, 1H), 7.76 (d, J=8.40 Hz, 2H), 7.66 (t, J=7.20 Hz, 1H), 7.36-7.45 (m, 3H), 3.56 (m, 1H), 3.42-3.46 (m, 4H), 3.31-3.35 (m, 2H), 3.02-3.05 (m, 2H), 2.31 (s, 3H). MS: 356.1 (M+H)⁺.

### Preparative Example 2: 3-(piperidin-4-yl)-1-tosyl-1H-indazole Hydrochloride

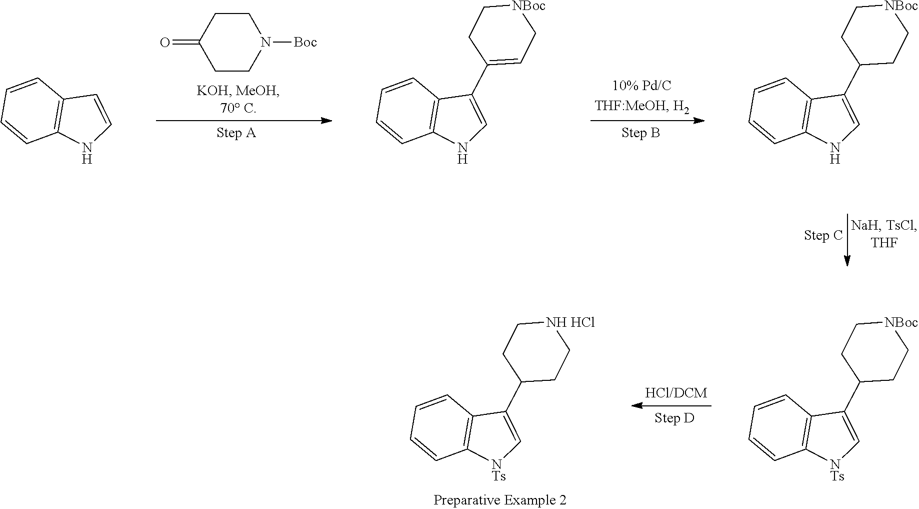

Step A

To a stirred solution of indole (5.0 g, 0.0427 mol) and tert-butyl 4-oxopiperidine-1-carboxylate (12.8 g, 0.0640 mol) in methanol (50 ml) was added potassium hydroxide (5.99 g, 0.107 mol). The mixture was then heated to 70° C. for 12 hours under nitrogen atmosphere. The reaction was monitored by TLC, the reaction mixture was then concentrated, and water (20 ml) was added to the crude mixture followed by extraction using DCM. The DCM layer was concentrated and to the crude was added petroleum ether (50 ml), the mixture was stirred for 30 minutes at room temperature. The slurry was filtered and dried under vacuum to afford a pale brown solid (12.1 g), which was directly taken to next step without further purification. MS: 299.2 (M+H)⁺.

Step B

To a solution of the crude title compound from Step A above (12 g) in THF/MeOH ( 1/1,150 ml) was added Pd/C (10% wet, 3.8 g). The reaction mixture was stirred at room temperature for 48 hours under hydrogen atmosphere (bladder pressure). The reaction was monitored by TLC, the reaction mixture was filtered through celite and the filtrate was concentrated under reduced pressure to afford the title compound as a white solid (10.3 g, 85%). MS: 201.0 (M−Boc)⁺.

Step C

To a suspension of sodium hydride (60% in paraffin oil, 1.53 g) in THF (10 ml) was added dropwise the title compound from Step B above (previously dissolved in THF 20 ml) at 0° C., and the reaction was stirred at room temperature for 60 minutes. Tosyl chloride (4.95 g, 0.0260 mol) was added at 0° C. dropwise (previously dissolved in THF 10 ml) and the reaction was stirred at room temperature for 3 hours. The reaction mixture was quenched with iced water followed by extraction using ethyl acetate (250 ml). The organic layer was separated, dried over sodium sulphate, filtered, and then concentrated under reduced pressure. Petroleum ether (50 ml) was added and the crude was stirred for 30 minutes at room temperature. The slurry was filtered and dried under vacuum to afford the title compound (5.8 g, 63%). ¹H-NMR (400 MHz, DMSO-d₆): δ 7.91 (d, J=8.40 Hz, 1H), 7.85 (d, J=8.40 Hz, 2H), 7.65 (d, J=8.00 Hz, 1H), 7.54 (s, 1H), 7.38-7.31 (m, 3H), 7.27-7.23 (m, 1H), 4.06 (d, J=11.60 Hz, 2H), 2.96-2.84 (m, 3H), 2.31 (s, 3H), 1.90 (d, J=12.80 Hz, 2H), 1.55-1.49 (m, 2H), 1.43 (s, 9H). MS: 355.1 (M−Boc)⁺.

Step D

To a stirred solution of the title compound from Step C above (5.2 g, 0.0114 mol) in dichloromethane (10 ml), 4N HCl in 1,4-dioxane (10 ml) was added at 0° C. The mixture was then stirred for an additional 3 hours at 0° C., then warmed up to room temperature. After completion of the reaction as monitored by TLC, the reaction mixture was concentrated to afford the title compound (4.5 g) as an off white solid. The solid was directly used for the next step without further purification. MS: 355.1 (M+H)⁺.

### Preparative Example 3: 1-methyl-3-(piperidin-4-yl)-1H-pyrazolo[3,4-b]pyridine Hydrochloride

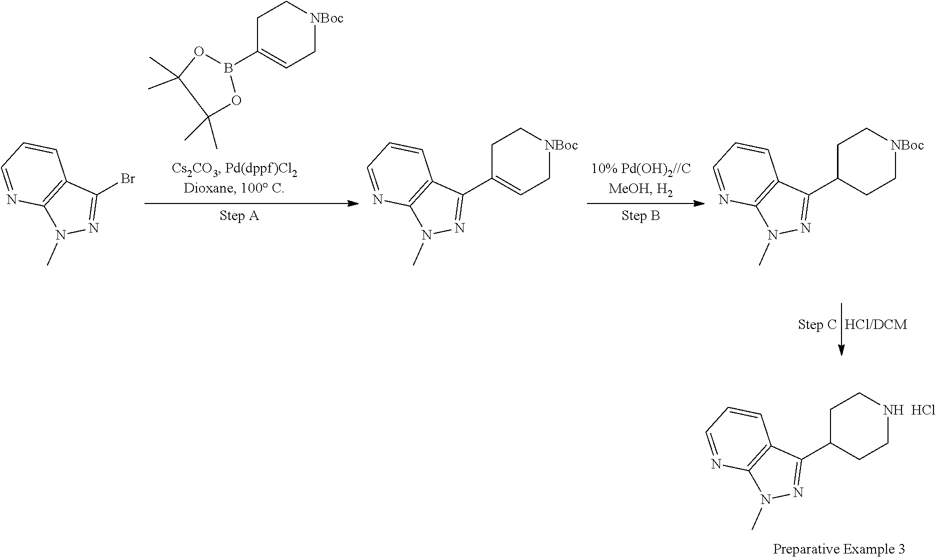

Step A

A stirred solution of 3-bromo-1-methyl-pyrazolo[3,4-b]pyridine (0.9 g, 4.24 mmol) and tert-butyl 4-(4,5,5-trimethyl-4-methyl-1,3,2-dioxaborolan-2-yl)-3,6-dihydro-2H-pyridine-1-carboxylate (1.57 g, 5.08 mmol) in 1,4-dioxane (25 ml) was purged with nitrogen for 15 minutes. Then Cs₂CO₃ (2.77 g, 8.49 mmol) in water (5 ml) was added and the nitrogen purging was continued for 5 minutes. Then Pd(dppf)Cl₂ (0.311 g, 0.42 mmol) was added. The sealed tube was closed and heated to 100° C. for 16 hours. Completion of reaction was monitored by TLC. The solvents were removed under reduced pressure and the residue was purified on a 230-400 silica gel cartridge using a Biotage Isolera One purification system by employing a petroleum ether/EtOAc gradient (100/0 to 80/20) to afford the title compound (1.35 g, 100%) as an off white solid. MS: 315.2 (M+H)⁺.

Step B

To a stirred solution of the title compound from Step A above (1.8 g, 5.73 mmol) in MeOH (50 ml), 10% Pd(OH)₂/C (0.180 g, 0.12 mmol) was added and the reaction mixture was stirred under hydrogen (H₂) atmosphere at room temperature for 4 hours. The reaction mixture was filtered through a celite pad and the filtrate was concentrated under reduced pressure. The residue was purified on a 230-400 silica gel cartridge using a Biotage Isolera One purification system by employing a petroleum ether/EtOAc gradient (100/0 to 80/20) to afford the title compound (1.4 g, 73.4%) as a transparent liquid.

Step C

To a stirred solution of the title compound from Step B above (1.4 g, 4.42 mmol) in 1,4-dioxane (6 ml), 4 M HCl in 1,4-dioxane (6 ml) was added at 0° C. The reaction mixture was stirred at room temperature for 1 hour. The reaction mixture was concentrated under reduced pressure and the solid was washed with diethyl ether, and then dried to afford the title compound (1.1 g, 96.4%) as a white solid. ¹H-NMR (400 MHz, DMSO-d₆): δ 9.17 (bs, 2H), 8.55-8.56 (m, 1H), 8.40-8.41 (m, 1H), 7.20-7.21 (m, 1H), 4.01 (s, 3H), 3.35-3.37 (m, 3H), 3.04-3.07 (m, 2H), 2.08-2.09 (m, 4H), 1.12 (s, 2H). MS: 217.0 (M+H)⁺.

### Preparative Example 4: 6-fluoro-3-(piperidin-4-yl)-1-tosyl-1H-pyrrolo[3,2-b]pyridine Hydrochloride

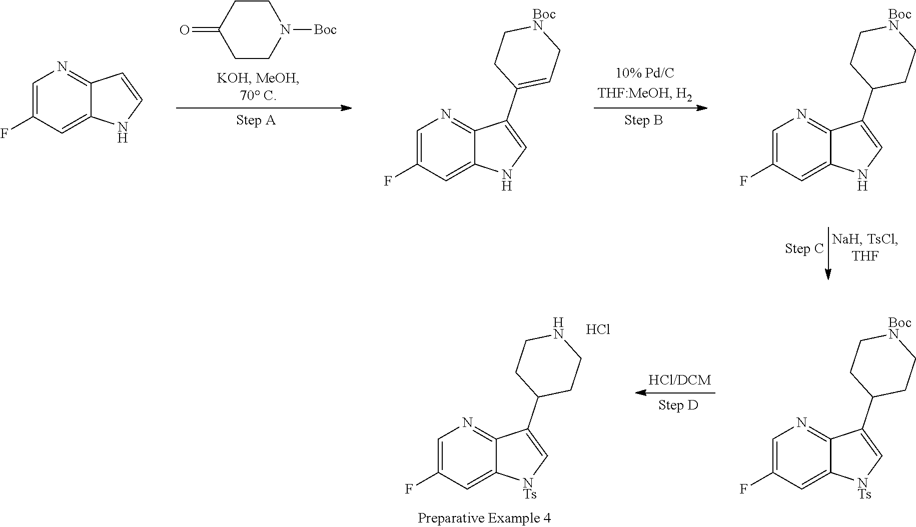

Step A

To a solution of 6-fluoro-1H-pyrrolo[3,2-b]pyridine (1.0 g, 7.35 mmol) in methanol (15 ml), was added tert-butyl 4-oxopiperidine-1-carboxylate (1.46 g, 7.35 mmol). To this was added KOH (1.45 g, 22.0 mmol). The resulting solution was stirred at 70° C. for 10 hours, following progress by TLC. Upon complete consumption of starting material, water (10 ml) was added. The resulting solid was filtered off, washed with water (100 ml) and the solid was dried under vacuum to afford tert-butyl 4-(6-fluoro-1H-pyrrolo[3,2-b]pyridin-3-yl)-3,6-dihydro-2H-pyridine-1-carboxylate (2.02 g, 98%) as a pale yellow solid. ¹H-NMR (400 MHz, DMSO-d₆): δ 11.40 (s, 1H), 8.37 (d, J=2.00 Hz, 1H), 7.67 (d, J=3.20 Hz, 2H), 7.10 (s, 1H), 4.04 (s, 2H), 3.56 (m, 2H), 3.32-3.33 (m, 2H), 1.43 (s, 9H). MS: 318.2 (M+H)⁺.

Step B

To a solution of the crude title compound from Step A above (2 g) in THF/MeOH ( 1/1,150 ml) was added Pd/C (10% wet, 600 mg). The reaction mixture was stirred at room temperature for 12 hours under hydrogen atmosphere (bladder pressure). The mixture was filtered through celite, and the filtrate was washed with MeOH (50 ml) and concentrated under reduced pressure. The crude was purified using 230-400 mesh in Biotage column chromatography (ethyl acetate/petroleum ether, 16/84) to afford the title compound tert-butyl 4-(6-fluoro-1H-indol-3-yl) piperidine-1-carboxylate as a white solid (1.52 g, 75.3%). ¹H-NMR (400 MHz, DMSO-d₆): δ 1.14 (s, 1H), 8.29-8.28 (m, 1H), 7.62-7.59 (m, 1H), 7.43 (d, J=2.00 Hz, 1H), 4.07-4.04 (m, 2H), 3.07-2.93 (m, 3H), 2.01-1.98 (m, 2H), 1.63-1.57 (m, 2H), 1.47 (s, 9H). MS: 320.3 (M+H)⁺.

Step C

To a suspension of sodium hydride (60% in paraffin oil, 0.396 g, 9.40 mmol) in THF (10.0 ml), the title compound from Step B above (previously dissolved in THF 20 ml) was added dropwise at 0° C. The reaction was stirred at room temperature for 60 minutes. Tosyl chloride (1.34 g, 7 mmol) previously dissolved in THF (10 ml) was added dropwise at 0° C. and then the reaction was stirred at room temperature for 3 hours. The reaction mixture was quenched with iced water and then was extracted using ethyl acetate (250 ml). The organic layers were separated, dried over sodium sulphate, filtered and then concentrated under reduced pressure. Petroleum ether (20 ml) was added and the crude was stirred for 30 minutes at room temperature. The slurry was filtered and dried under vacuum to afford the title compound (1.9 g, 86%). ¹H-NMR (400 MHz, DMSO-d₆): δ 8.55-8.54 (m, 1H), 8.18 (dd, J=2.80, 9.40 Hz, 1H), 7.99 (d, J=8.40 Hz, 2H), 7.91 (s, 1H), 7.41 (d, J=8.40 Hz, 2H), 4.06-4.01 (m, 2H), 3.04-2.98 (m, 1H), 2.85-2.84 (m, 2H), 2.34 (s, 3H), 1.95-1.92 (m, 2H), 1.65-1.58 (m, 2H), 1.42 (s, 9H). MS: 473.9 (M+H)⁺.

Step D

To a stirred solution of the title compound from Step C above (1.9 g, 4.02 mmol) in dichloromethane (15 ml), 4N HCl in 1,4-dioxane was added (10 ml) at 0° C. The mixture was stirred for 30 minutes at 0° C. and then was allowed to warm up to room temperature. After completion of the reaction by TLC, the reaction mixture was concentrated to afford the title compound (1.55 g) as an off white solid. The solid was directly used for the next step without further purification. MS: 373.9 (M+H)⁺.

### Preparative Example 5: 5-fluoro-1-methyl-3-(pyrrolidin-3-yl)-1H-pyrrolo[2,3-b]pyridine Hydrochloride

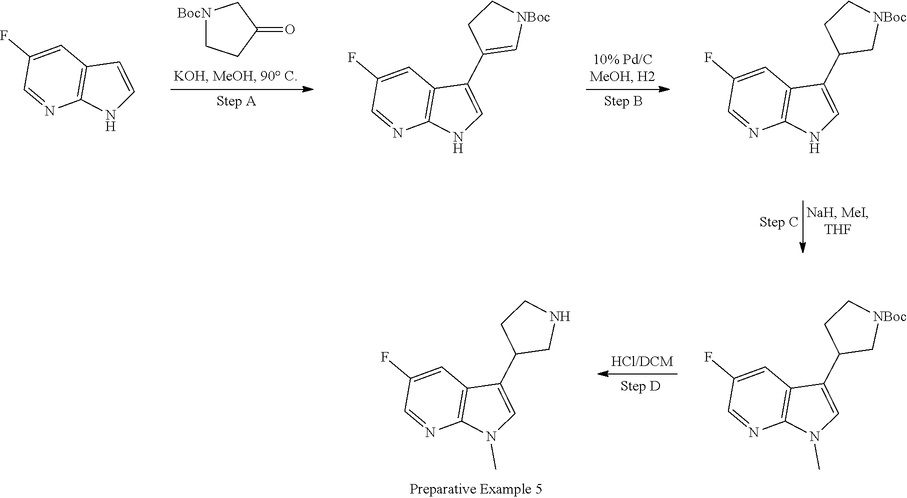 [SMILES: Fc1cnc2[nH]ccc2c1 ; CC(C)(C)OC(=O)N1CCC(=O)C1 ; CC(C)(C)OC(=O)N1C=C(c2c[nH]c3ncc(F)cc23)CC1 ; CC(C)(C)OC(=O)N1CCC(c2c[nH]c3ncc(F)cc23)C1 ; Cn1cc(C2CCNC2)c2cc(F)cnc21 ; Cn1cc(C2CCN(C(=O)OC(C)(C)C)C2)c2cc(F)cnc21]

Step A

To a solution of 5-fluoro-1H-pyrrolo[2,3-b]pyridine (1.0 g, 7.5 mmol) and tert-butyl 3-oxopyrrolidine-1-carboxylate (1.5 g, 8.10 mmol) in methanol (100 ml), potassium hydroxide (1.36 g, 24.3 mmol) was added. The suspension was stirred at 90° C. for 12 hours. The crude was cooled at room temperature, and then concentrated under reduced pressure. The mixture was purified on a HP-silica gel column by employing petroleum ether/ethyl acetate (20/80) to afford the title compound (0.660 g) as an off-white solid. The crude was directly used for the next step without further purification. MS: 304.1 (M+H)⁺.

Step B

To a solution of the title compound from Step A above (0.66 g, 2.18 mmol) in methanol (50 ml), Pd/C 10% (0.232 g, 2.18 mmol) was added. The suspension was stirred at 25° C. for 12 hours. The reaction mixture was filtered through celite, and then concentrated to afford the title compound (0.63 g, 72%) as an off white solid. MS: 306.1 (M+H)⁺.

Step C

To a solution of sodium hydride (60% in paraffin oil) (0.0339 g, 1.47 mmol) in tetrahydrofuran (25 ml) the title compound from Step B above (0.3 g, 0.982 mmol) in tetrahydrofuran (25 ml) was added dropwise at 0° C. The mixture was stirred for 1 hour at 25° C., and then methyl iodide (0.183 ml, 1.47 mmol) was added at 0° C. The mixture was stirred for an additional 2 hours at 25° C. The reaction mixture was diluted with water (50 ml) and the organic phase was separated. The aqueous phase was extracted twice with ethyl acetate (2×50 ml). The combined organic phases were dried over Na₂SO₄, filtered and the solvent was evaporated under reduced pressure. The crude product was purified on a silica gel column by employing petroleum ether/ethyl acetate (70/30) to afford the title compound (0.34 g, 84%) as an off white solid. MS: 320.2 (M+H)⁺.

Step D

To a solution of the title compound from Step C above (0.340 g, 1.06 mmol) in DCM (30 ml), 4 M hydrochloric acid in 1,4-dioxane (0.5 ml) was added dropwise at 0° C. The mixture was stirred at 25° C. for 2 hours. The reaction mixture was concentrated, washed with diethyl ether and filtered to afford the title compound (0.280 g, 96%) as an off white solid. MS: 220.2 (M+H)⁺.

### Preparative Example 6: 3-(pyrrolidin-3-yl)-1-tosyl-1H-indazole Hydrochloride

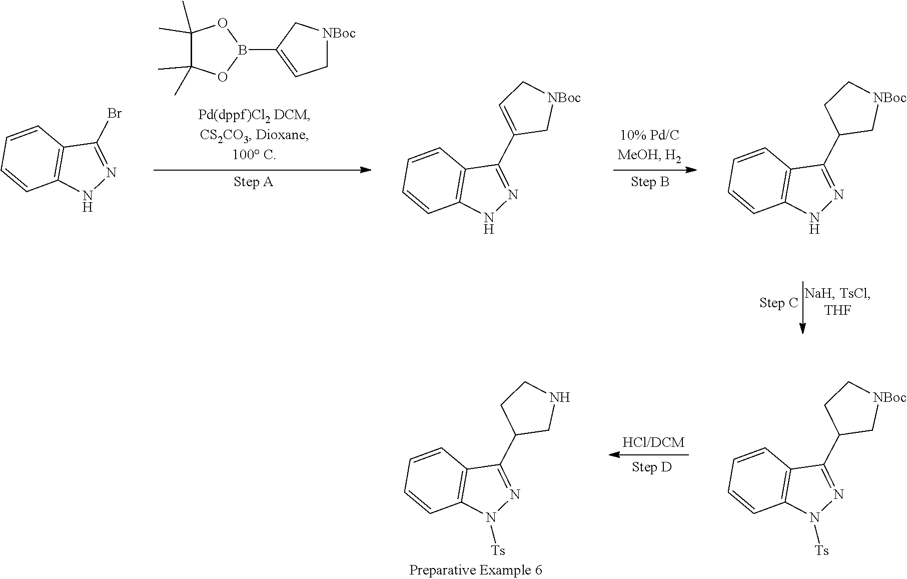 [SMILES: Brc1n[nH]c2ccccc12 ; CC(C)(C)OC(=O)N1CC=C(B2OC(C)(C)C(C)(C)O2)C1 ; CC(C)(C)OC(=O)N1CC=C(c2n[nH]c3ccccc23)C1 ; CC(C)(C)OC(=O)N1CCC(c2n[nH]c3ccccc23)C1 ; Cc1ccc(S(=O)(=O)n2nc(C3CCNC3)c3ccccc32)cc1 ; Cc1ccc(S(=O)(=O)n2nc(C3CCN(C(=O)OC(C)(C)C)C3)c3ccccc32)cc1]

Step A

3-bromo-1H-indazole (0.5 g 2.54 mmol) and tert-butyl 3-(4,4,5,5-tetramethyl-1,3,2-dioxaborolan-2-yl)-2,5-dihydropyrrole-1-carboxylate (0.824 g, 2.79 mmol) were added to a reaction vial followed by degassed 1,4-dioxane (6 ml) and water (2.0 ml). The vial was filled with argon gas and sealed. Then [1,1′-Bis(diphenylphosphino)ferrocene]dichloropalladium(II) complex with dichloromethane (0.186 g, 0.254 mmol) and cesium carbonate (2.48 g, 7.61 mmol) were added, and the solution was heated at 100° C. for 12 hours. After completion of the reaction as evidenced by TLC, the reaction mixture was filtered through celite, washed with a solution of DCM and MeOH, and concentrated under reduced pressure. The crude was purified on a silica gel column using Biotage Isolera One purification system employing an ethyl acetate/hexane gradient (30/70) to afford the title compound as a white solid (0.50 g, 54%). ¹H-NMR (400 MHz, DMSO-d₆): δ 13.16 (s, 1H), 8.03-8.04 (m, 1H), 7.57 (d, J=8.40 Hz, 1H), 7.39-7.40 (m, 1H), 7.19-7.20 (m, 1H), 6.64 (d, J=1.60 Hz, 1H), 4.54 (s, 2H), 4.31 (s, 2H), 1.42 (s, 9H). MS: 230.1 (M+H)⁺-t-butyl.

Step B

To a solution of the title compound from Step A above (0.45 g, 1.5 mmol) in methanol (50 ml), Pd/C 10% (0.079 g, 0.749 mmol) was added under nitrogen atmosphere. The reaction mixture was stirred at room temperature for 12 hours under bladder (hydrogen (H₂) atmosphere). The suspension was stirred at 25° C. for 12 hours. The reaction mixture was filtered through celite, washed with methanol, and concentrated to afford the title compound (0.54 g) as a yellow liquid. The crude was directly used for the next step without further purification. MS: 286.2 (M−H)⁻.

Step C

To a suspension of sodium hydride (60% in paraffin oil, 0.078 g, 1.97 mmol) in THF (10 ml) was added dropwise the title compound from Step B above (0.540 g, 1.32 mmol) previously dissolved in THF (20 ml) at 0° C. The mixture was stirred at room temperature for 30 minutes. A solution of tosyl chloride (0.752 g, 3.95 mmol) in THF (20 ml) was added dropwise at 0° C., and then the mixture was stirred at room temperature for 3 hours. The reaction mixture was quenched with iced water and extracted with ethyl acetate (100 ml). The organic layers were concentrated and purified by silica gel column chromatography using a petroleum ether/ethyl acetate gradient (70/30) to afford the title compound (0.350 g, 49%) as a pale gummy yellow solid. MS: 342.1 (M+H)⁺-Boc.

Step D

To a solution of the title compound from Step C above (0.35 g, 0.65 mmol) in DCM (30 ml) was added 4 M HCl in 1,4-dioxane (3.25 ml) dropwise at 0° C. The mixture was left to warm up and was stirred at 25° C. for 2 hours. The reaction mixture was concentrated, washed with diethyl ether, and filtered to afford the title compound (0.250 g) as a pale-yellow solid. The crude was directly used for the next step without further purification. MS: 342.1 (M+H)⁺.

### Preparative Example 7: 3-(piperidin-4-yl)imidazo[1,2-a]pyridine Hydrochloride

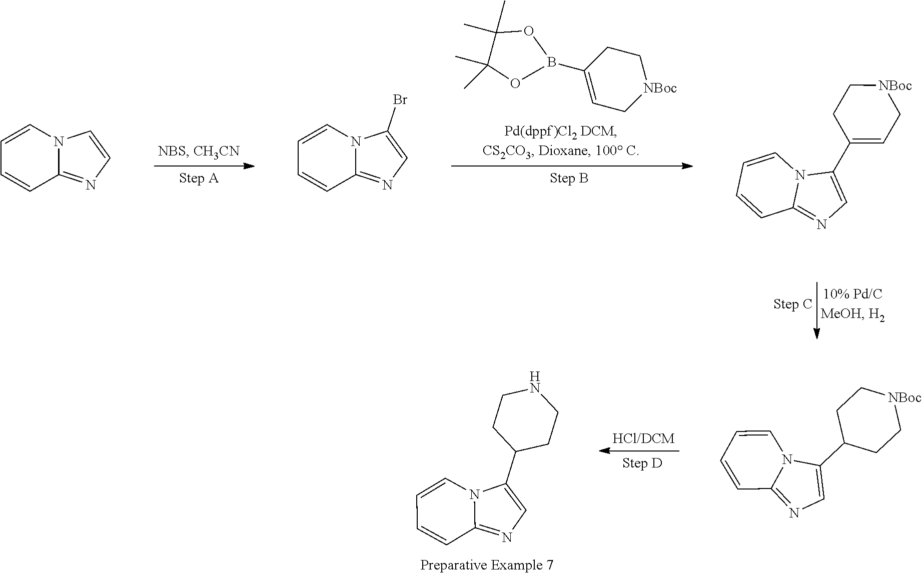 [SMILES: c1ccn2ccnc2c1 ; Brc1cnc2ccccn12 ; CC(C)(C)OC(=O)N1CC=C(B2OC(C)(C)C(C)(C)O2)CC1 ; CC(C)(C)OC(=O)N1CC=C(c2cnc3ccccn23)CC1 ; c1ccn2c(C3CCNCC3)cnc2c1 ; CC(C)(C)OC(=O)N1CCC(c2cnc3ccccn23)CC1]

Step A

To a stirred solution of imidazo[1,2-a]pyridine (3 g, 25.4 mmol) in acetonitrile (80 ml), NBS (5.42 g, 30.5 mmol) was added portion wise at 0° C. The mixture was stirred at room temperature for 1 hour.

Then the mixture was concentrated under reduced pressure The crude was purified on silica gel column using Biotage Isolera One purification system employing an ethyl acetate/petroleum ether gradient (30/70) to afford (1.3 g, 25.5%) as a light brown solid. ¹H-NMR (400 MHz, CDCl₃): δ 8.14-8.15 (m, 1H), 7.64-7.65 (m, 2H), 7.24-7.25 (m, 1H), 6.95-6.96 (m, 1H). MS: 198.8 (M+H)⁺.

Step B

A stirred solution of the title compound from Step A above (1.1 g, 5.58 mmol) and tert-butyl 4-(4,4,5,5-tetramethyl-1,3,2-dioxaborolan-2-yl)-3,6-dihydro-2H-pyridine-1-carboxylate (2.93 g, 9.49 mmol) in dioxane (50 m) was purged with nitrogen for 15 minutes. Cs₂CO₃ (3.64 g, 11.2 mmol) in water (5 ml) was then added. The nitrogen purging was continued for 5 minutes, and then 1,1-Bis(diphenylphosphino)ferrocene]dichloropalladium(II) complex with dichloromethane (0.456 g, 0.55 mmol) was added. The sealed tube was heated at 100° C. for 16 hours. The mixture was concentrated under reduced pressure. The crude was purified on a silica gel column using Biotage Isolera One purification system employing an ethyl acetate/petroleum ether gradient (60/40) to afford the title compound (1.3 g, 62.2%) as a brown gummy solid. ¹H-NMR (400 MHz, DMSO-d6): δ 8.62 (d, J=7.20 Hz, 1H), 7.59-7.61 (m, 2H), 7.24-7.25 (m, 1H), 6.92-6.93 (m, 1H), 6.17 (s, 1H), 4.10 (s, 2H), 3.58-3.60 (m, 2H), 2.50-2.51 (m, 2H), 1.46 (s, 9H). MS: 244.1 (M+H)⁺−t-butyl.

Step C

To a stirred solution of the title compound from Step B above (1.5 g, 3.51 mmol) in methanol (80 ml), Pd(OH)₂/C (0.2 g, 0.14 mmol) was added under nitrogen atmosphere. The reaction mixture was stirred at room temperature for 20 hours. Then the reaction mixture was filtered through celite, washed with MeOH. The filtrate was concentrated, and the crude was purified on a silica gel column using Biotage Isolera One purification system employing an ethyl acetate/petroleum ether gradient (60/40) to afford the title compound (0.5 g, 44%) as a brown sticky solid. The crude was directly used for the next step without further purification. MS: 302.2 (M+H)⁺.

Step D

To a solution of the title compound from Step C above (0.5 g, 1.54 mmol) in DCM (10 ml) was added 4 M HCl in 1,4-dioxane (10 ml) dropwise at 0° C. The mixture was then stirred at 25° C. for 1 hour. The reaction mixture was then concentrated, washed with diethyl ether, and filtered to afford the title compound (0.3 g, 73.6%) as a light-yellow solid. ¹H-NMR (400 MHz, DMSO-d6): δ 9.37 (s, 2H), 9.07 (d, J=6.80 Hz, 1H), 8.18 (s, 1H), 7.96-7.98 (m, 2H), 7.57 (t, J=6.80 Hz, 1H), 3.37-3.40 (m, 3H), 3.04-3.07 (m, 2H), 2.17-2.21 (m, 2H), 1.93-1.96 (m, 2H). MS: 202.2 (M+H)⁺.

### Preparative Example 8: 5-fluoro-1-methyl-3-(piperidin-4-yl)-1H-pyrrolo[2,3-b]pyridine Hydrochloride

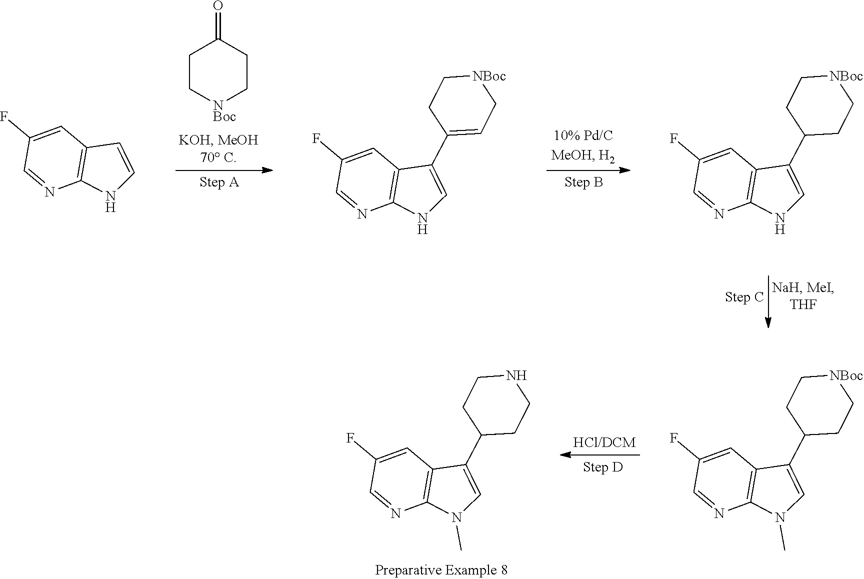 [SMILES: Fc1cnc2[nH]ccc2c1 ; CC(C)(C)OC(=O)N1CCC(=O)CC1 ; CC(C)(C)OC(=O)N1CC=C(c2c[nH]c3ncc(F)cc23)CC1 ; CC(C)(C)OC(=O)N1CCC(c2c[nH]c3ncc(F)cc23)CC1 ; Cn1cc(C2CCNCC2)c2cc(F)cnc21 ; Cn1cc(C2CCN(C(=O)OC(C)(C)C)CC2)c2cc(F)cnc21]

Step A

To a stirred solution of 5-fluoro-1H-pyrrolo[2,3-b]pyridine (60 g, 0.441 mol) in methanol (900 ml), KOH (49 g, 0.882 mol) and tert-butyl 4-oxopiperidine-1-carboxylate (96.6 g, 0.485 mol) were added. The reaction was heated at 70° C. for 12 hours under nitrogen atmosphere. The reaction mixture was then quenched with water (100 ml) and the formed precipitate was filtered through a sintered funnel. The filtrate was washed with water and petroleum ether, dried under vacuum, to afford the title compound (130 g, 85.5%) as a pale-yellow solid. ¹H-NMR (400 MHz, DMSO-d₆): δ 11.85 (s, 1H), 8.11-8.12 (m, 2H), 7.67 (s, 1H), 6.16 (s, 1H), 4.03 (s, 2H), 3.55-3.56 (m, 2H), 1.44 (s, 11H). MS: 318.2 (M+H)⁺.

Step B

To a stirred solution of the title compound from Step A above (50 g, 0.15 mol) in tetrahydrofuran (500 ml) was added 10% Pd/C (16 g). The reaction mixture was stirred at room temperature for 24 hours under hydrogen pressure (1 bar). The reaction mixture was filtered through celite and washed with methanol (1000 ml). The filtrate was concentrated under reduced pressure, recrystallized using a mixture of petroleum ether and methanol and filtered out by Buchner funnel to afford the title compound (43 g, 85%) as a black-brown solid. ¹H-NMR (400 MHz, DMSO-d6): δ 11.52 (s, 1H), 8.15-8.16 (m, 1H), 7.88-7.89 (m, 1H), 7.36 (s, 1H), 4.04-4.07 (m, 2H), 2.90-2.91 (m, 3H), 1.92-1.95 (m, 2H), 1.46-1.48 (m, 11H). MS: 320.3 (M+H)⁺.

Step C

To a suspension of sodium hydride (60% in paraffin oil, 16.3 g, 0.407 mol) in THF (300 ml) the title compound from Step B above (65 g, 0.204 mol) in THF (100 ml) was added dropwise at 0° C. The mixture was stirred at room temperature for 1 hour. Methyl Iodide (25 ml, 0.404 mol) was then added dropwise at 0° C. and the mixture was stirred at room temperature for 2 hours. The reaction was then quenched by pouring it into iced water, and then was extracted with ethyl acetate (3×500 ml). The combined organic layers were collected and washed with a brine solution, dried over sodium sulphate, filtered, and concentrated under reduced pressure. The crude was recrystallized using a mixture of petroleum ether and methanol and filtered out by Buchner funnel to afford the title compound (49 g, 72.2%) as a pale-yellow solid. ¹H-NMR (400 MHz, DMSO-d₆): δ 8.21-8.22 (m, 1H), 7.95 (dd, J=2.80, 9.60 Hz, 1H), 7.43 (s, 1H), 4.04-4.07 (m, 2H), 3.76 (s, 3H), 2.90-2.91 (m, 3H), 1.92-1.95 (m, 2H), 1.40-1.42 (m, 11H). MS: 334.3 (M+H)⁺.

Step D

To a stirred solution of the title compound from Step C above (49 g, 0.147 mol) in dichloromethane (500 ml), 4 M HCl in 1,4-dioxane (300 ml) was added at 0° C. The mixture was warmed up to room temperature and stirred for 4 hours. The reaction was concentrated under reduced pressure. The crude product was washed with diethyl ether (200 ml) and dried under vacuum to afford the title compound (38 g, 95.5%) as a brown solid. ¹H-NMR (400 MHz, DMSO-d6): δ 9.07 (s, 2H), 8.24-8.25 (m, 1H), 8.06-8.07 (m, 1H), 7.45 (s, 1H), 3.78 (s, 3H), 3.33-3.36 (m, 2H), 2.98-2.99 (m, 3H), 1.97-1.98 (m, 4H). MS: 234.3 (M+H)⁺.

### Preparative Example 9: 5-fluoro-3-(piperidin-4-yl)-1-tosyl-1H-pyrrolo[2,3-b]pyridine Hydrochloride

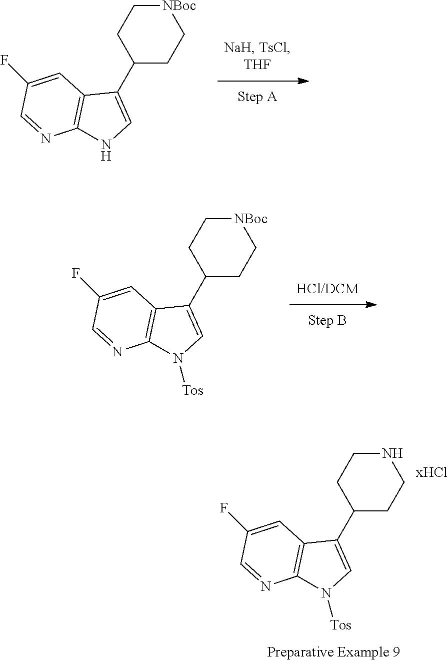

Step A

To a suspension of sodium hydride (60% in paraffin oil, 0.072 g, 0.003 mol) in THF (3 ml), butyl 4-(5-fluoro-1H-pyrrolo[2,3-b]pyridin-3-yl)piperidine-1-carboxylate (0.500 g, 0.00151 mol) previously dissolved in THF (4 ml) was added dropwise at 0° C. The mixture was warmed up to room temperature. Then tosyl chloride (0.345 g, 0.00181 mol) in THF (3 ml) was added at 0° C. The mixture was then warmed up to room temperature and stirred at room temperature for 1 hour. The reaction mixture was then quenched with iced water, and extracted using ethyl acetate (50 ml). The organic layers were separated, dried over sodium sulphate, filtered and then concentrated to get the title compound (500 mg) as an off white solid. The crude was directly used for the next step without further purification. MS: 474.2 (M+H)⁺.

Step B

To a stirred solution of the title compound from Step A above (0.49 g, 0.147 mol) in dichloromethane (5 ml), 4 M HCl in 1,4-dioxane (1 ml) was added at 0° C. The mixture was warmed up to room temperature and stirred for 2 hours. The reaction was concentrated under reduced pressure. The crude product was washed with diethyl ether, and dried under vacuum to afford the title compound (0.3 g) as a HCl salt. MS: 374.2 (M+H)⁺.

### Preparative Example 10: 3-(1H-pyrazol-4-yl)-1-tosyl-1H-indole

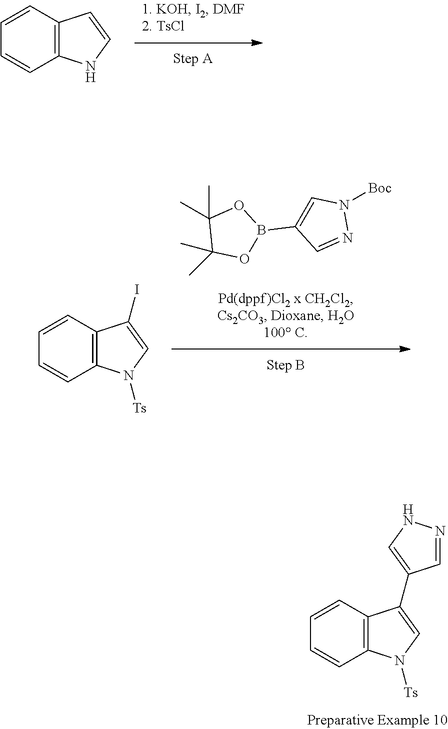 [SMILES: c1ccc2[nH]ccc2c1 ; CC(C)(C)OC(=O)n1cc(B2OC(C)(C)C(C)(C)O2)cn1 ; Cc1ccc(S(=O)(=O)n2cc(I)c3ccccc32)cc1 ; Cc1ccc(S(=O)(=O)n2cc(-c3cn[nH]c3)c3ccccc32)cc1]

Step A

To a stirred solution of 1H-indole (3 g, 25.6 mmol) in DMF (50 ml), KOH (3.59 g, 64.0 mmol) was added. The mixture was stirred at 25° C. for 30 minutes. A solution of iodine (6.82 g, 26.9 mmol) (dissolved in 25 ml of DMF) was added dropwise and the reaction was stirred at 25° C. for 1 hour. Then, KOH (3.59 g, 64.0 mmol) was added followed by tosyl chloride (7.81 g, 41.0 mmol). The reaction mixture was then stirred at 25° C. for 12 hours under nitrogen atmosphere. Then, the reaction was quenched with water (100 ml) followed by ethyl acetate (100 ml). The phases were separated, and the aqueous phases were extracted with dichloromethane (100 ml). The organic phases were combined, dried over Na₂SO₄, filtered, and the residual solvents were evaporated under reduced pressure. The crude purified on a HP-Sil column (Biotage) by employing a petroleum ether/ethyl acetate gradient (100/0→90/10). The crude was recrystallized using ethanol (20 ml), the solid was filtered and dried under vacuum to afford the title compound (4.1 g, 39%) as a pale brown solid. ¹H-NMR (400 MHz, CDCl₃): δ 7.97-7.98 (m, 1H), 7.80 (dd, J=2.00, 6.60 Hz, 2H), 7.72 (s, 1H), 7.39-7.40 (m, 2H), 7.33-7.35 (m, 1H), 7.25-7.26 (m, 1H). MS: 396.9 (M+H)⁺.

Step B

A mixture of the title compound from Step A above (1.5 g, 3.78 mmol), tert-butyl 4-(4,4,5,5-tetramethyl-1,3,2-dioxaborolan-2-yl)-1H-pyrazole-1-carboxylate (1.222 g, 4.15 mmol), PdCl₂(dppf) (0.138 g, 0.189 mmol), and cesium carbonate (3.08 g, 9.44 mmol) in 1,4-Dioxane (20 ml) and Water (3 ml) was degassed and purged using Nitrogen. The mixture was heated under nitrogen atmosphere at 110° C. overnight. The reaction mixture was filtered through a celite pad, washed with DCM and MeOH, and concentrated under reduced pressure. The crude was purified on a silica gel column using Biotage Isolera One purification system employing an EtOAc/hexane gradient (60/40) to afford the title compound (1.1 g, 85%) as a pale brown solid. ¹H-NMR 400 MHz, DMSO-d6: δ 13.05 (s, 1H), 8.31 (s, 1H), 7.97-8.00 (m, 3H), 7.87-7.89 (m, 3H), 7.30-7.32 (m, 4H), 2.31 (s, 3H). MS: 338.0 (M+H)⁺.

### Preparative Example 11: 3-(1H-pyrazol-4-yl)-1-tosyl-1H-indazole Hydrochloride

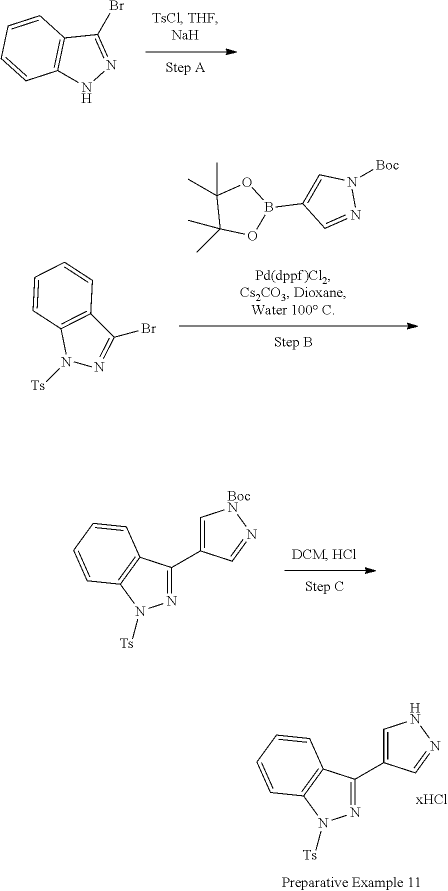 [SMILES: Brc1n[nH]c2ccccc12 ; CC(C)(C)OC(=O)n1cc(B2OC(C)(C)C(C)(C)O2)cn1 ; Cc1ccc(S(=O)(=O)n2nc(Br)c3ccccc32)cc1 ; Cc1ccc(S(=O)(=O)n2nc(-c3cnn(C(=O)OC(C)(C)C)c3)c3ccccc32)cc1 ; Cc1ccc(S(=O)(=O)n2nc(-c3cn[nH]c3)c3ccccc32)cc1]

Step A

To a suspension of sodium hydride (60% in paraffin oil, 0.609 g, 15.23 mmol) in THF (50 ml) was added 3-bromo-1H-indazole (1.0 g, 5.08 mmol). The reaction mixture was stirred for 30 minutes at 0° C. Then TsCl (1.451 g, 7.61 mmol) was added and the reaction mixture was stirred for 2 hours at 25° C. The reaction mixture was quenched with iced water and extracted with ethyl acetate (50 ml). The organic layers were separated, washed with brine solution, dried over sodium sulphate, filtered, and concentrated. The compound was recrystallized with petroleum ether to afford the title compound (1.7 g, 91%) as a pale-yellow solid. ¹H-NMR (400 MHz, DMSO-d₆): δ 8.19 (d, J=11.20 Hz, 1H), 7.77-7.78 (m, 4H), 7.51-7.52 (m, 1H), 7.42 (d, J=10.80 Hz, 2H), 2.34 (s, 3H). MS: 350.9 (M+H)⁺.

Step B

A mixture of the title compound from Step A above (1.7 g, 4.84 mmol), tert-butyl 4-(4,4,5,5-tetramethyl-1,3,2-dioxaborolan-2-yl)-1H-pyrazole-1-carboxylate (1.566 g, 5.32 mmol), PdCl₂(dppf) (0.177 g, 0.242 mmol) and cesium carbonate (3.94 g, 12.10 mmol) in 1,4-dioxane (15 ml) and water (2 ml) was degassed and purged using nitrogen. The mixture was heated under nitrogen atmosphere at 110° C. overnight. The reaction mixture was filtered through a celite pad, washed with DCM and MeOH, and concentrated under reduced pressure. The crude was purified on a silica gel column using Biotage Isolera One purification system employing an EtOAc/hexane gradient (70/30) to afford the title compound (0.9 g, 39%) as a pale brown solid. MS: 339.2 (M+H)⁺.

Step C

To a solution of title compound from the Step B above (900 mg, 2.052 mmol) in DCM (10 ml) was added at 0° C. a 4.0 M solution of HCl in 1,4-dioxane (0.5 ml). The reaction mixture was warmed up to room temperature and stirred for 2 hours. The reaction mixture was then evaporated under reduced pressure in the presence of diethyl ether to afford the title compound (700 mg, 1.903 mmol, 93%) as an off-white solid. ¹H-NMR (400 MHz, DMSO-d₆): δ 8.38 (s, 2H), 8.15-8.16 (m, 2H), 7.70-7.71 (m, 4H), 7.35-7.37 (m, 2H). MS: 339.0 (M+H)⁺.

### Preparative Example 12: 3-(1H-pyrazol-3-yl)-1-tosyl-1H-indazole

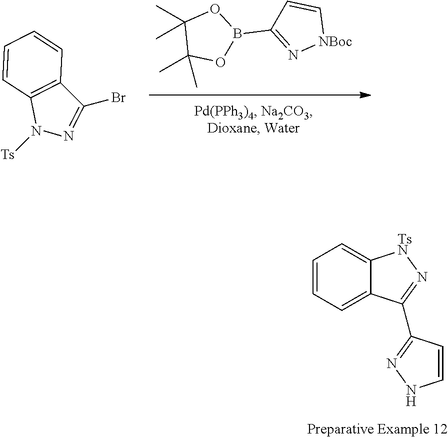 [SMILES: Cc1ccc(S(=O)(=O)n2nc(Br)c3ccccc32)cc1 ; CC(C)(C)OC(=O)n1ccc(B2OC(C)(C)C(C)(C)O2)n1 ; Cc1ccc(S(=O)(=O)n2nc(-c3cc[nH]n3)c3ccccc32)cc1]

In a sealed tube (50 ml), a solution of 3-bromo-1-tosyl-1H-indazole (1 g, 2.85 mmol) and tert-butyl 3-(4,4,5,5-tetramethyl-1,3,2-dioxaborolan-2-yl)-1H-pyrazole-1-carboxylate (0.838 g, 2.85 mmol) were dissolved in 1,4-dioxane (6 ml) and water (2 ml). Nitrogen gas was bubbled through the mixture for a period of 5 minutes. Then, Tetrakis(triphenylphosphine)palladium(0) (0.165 g, 0.142 mmol) and sodium carbonate (0.754 g, 7.12 mmol) were added under nitrogen atmosphere. The reaction mixture was heated at 100° C. for 16 hours. The mixture was concentrated under reduced pressure and was purified on silica gel column using Biotage Isolera One purification system eluting with EtOAc/petroleum ether (50/50) to afford the title compound (0.5 g, 49%) as a pale brown solid. MS: 339.3 (M+H)⁺.

### Preparative Example 13: 3-(1H-pyrazol-3-yl)-1-tosyl-1H-indole

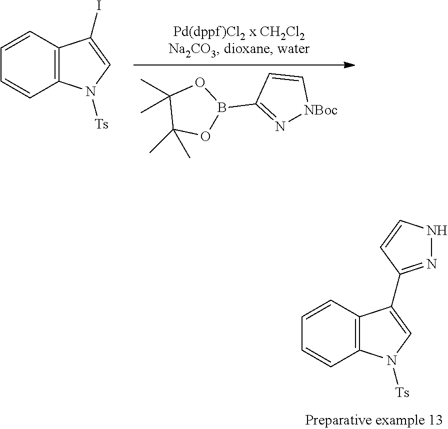

To a mixture of 3-iodo-1-tosyl-1H-indole (900 mg, 2.266 mmol) and tert-butyl 3-(4,4,5,5-tetramethyl-1,3,2-dioxaborolan-2-yl)-1H-pyrazole-1-carboxylate (666 mg, 2.266 mmol) in 1,4-dioxane (20 ml) and water (3 ml), was added 1,1′-Bis(diphenylphosphino)ferrocene]dichloropalladium(II) complex with dichloromethane (93 mg, 0.113 mmol) and cesium carbonate (1846 mg, 5.66 mmol). The mixture was degassed and filled with N₂ and stirred under N₂ atmosphere at 110° C. overnight. The reaction mixture was filtered through celite and washed with DCM (100 ml) and MeOH (20 ml) and concentrated under reduced pressure. The crude was purified on silica gel column using Biotage Isolera One purification system employing an EtOAc/hexane gradient (60/40) to afford the title compound (600 mg, 75%) as a pale brown solid. MS: 338.0 (M+H)⁺.

### Preparative Example 14: 3-(pyrrolidin-3-yl)-1-tosyl-1H-indole Hydrochloride

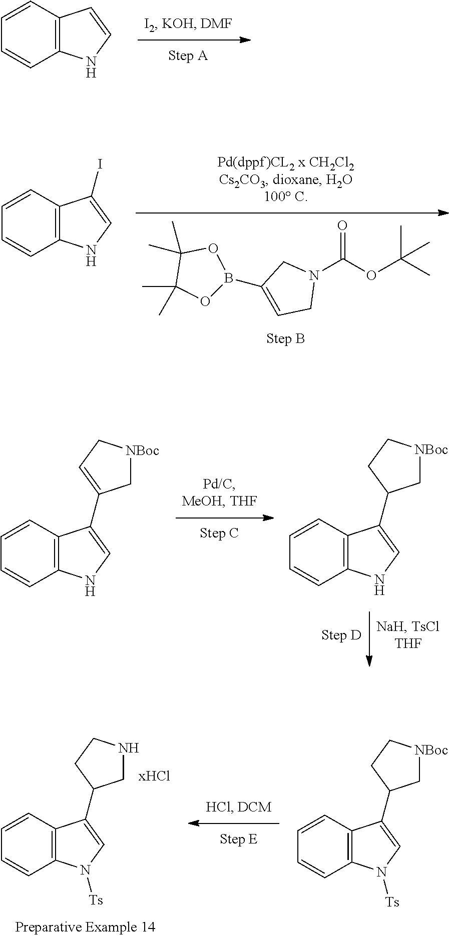

Step A

To a stirred solution of indole (2.0 g, 0.0171 mol) in DMF (30 ml), KOH (2.87 g, 0.0512 mol) was added. Then, iodine (4.33 g, 0.0171 mol) in DMF (30 ml) was added dropwise and the reaction was stirred at room temperature for 30 minutes under nitrogen atmosphere. The reaction mixture was poured into a mixture of iced water (400 ml), aqueous ammonia (2 ml) and sodium metabisulphite (100 mg). The formed solid was filtered, washed with cold water and dried to afford the title compound (3.50 g, 75%) as a white solid. ¹H-NMR (400 MHz, DMSO-d₆): δ 11.54 (s, 1H), 7.55 (s, 1H), 7.40-7.41 (m, 1H), 7.15-7.16 (m, 1H), 7.09-7.11 (m, 2H). MS: 241.9 (M−H)⁻.

Step B

The title compound from the Step A above (2.0 g, 7.32 mmol), and tert-butyl 3-(4,4,5,5-tetramethyl-1,3,2-dioxaborolan-2-yl)-2,5-dihydropyrrole-1-carboxylate (2.16 g, 7.32 mmol) were added into a reaction vial. Degassed 1,4-dioxane (20 ml) and water (30 ml) were added into the reaction vial. The vial was then filled with argon gas and sealed. Then, 1,1-Bis (diphenylphosphino)ferrocene]dichloropalladium(II) complex with dichloromethane (0.268 g, 0.366 mmol) and cesium carbonate (7.16 g, 2.20 mmol) were added and the resulting solution was heated at 100° C. for 4 hours. After completion of the reaction as evidenced by TLC, the reaction mixture was diluted with ethyl acetate (50 ml) and water (50 ml). The phases were separated and the aqueous phase was extracted with ethyl acetate (2×50 ml). The organic phases were then combined, dried over Na₂SO₄, filtered, and the solvent was evaporated under reduced pressure. The crude was purified on a HP-Sil column (Biotage) by employing an ethyl acetate/petroleum ether gradient (100/0→70/30) to afford the title compound (1.4 g, 48%). as a pale brown solid. MS: 283.1 (M−H)⁻.

Step C

To a solution of the title compound from Step B above (1.4 g, 4.23 mmol) in methanol (30 ml) and THF (20 ml), Pd/C (0.180 g, 1.69 mmol) was added under nitrogen atmosphere. The mixture was stirred at room temperature for 12 hours under hydrogen atmosphere. The reaction mixture was filtered through a celite pad, washed with methanol, and concentrated to afford the title compound (1.3 g, 95%) as a pale brown liquid. MS: 287.2 (M+H)⁺-Boc.

Step D

To a suspension of sodium hydride (60% in paraffin oil, 0.156 g, 3.93 mmol) in THF (10 ml), was added dropwise the title compound from Step C above (1.1 g, 2.64 mmol) in THF (20 ml) at 0° C. The mixture was stirred at room temperature for 30 minutes. Then, TsCl (1.5 g, 7.89 mmol) in THF (20 ml) was added dropwise at 0° C. Then the mixture was warmed up to room temperature and stirred for 3 hours. The reaction mixture was quenched with iced water and ethyl acetate (100 ml) was added. The organic layers were separated, dried over Na₂SO₄, and concentrated under reduced pressure. The crude was purified on a HP-Sil column (Biotage) by employing an ethyl acetate/petroleum ether gradient (100/0 to 70/30) to afford the title compound (1.2 g, 71%) as a pale-yellow solid. MS: 341.1 (M+H)⁺-Boc.

Step E

To a solution of title compound from the Step D above (0.7 g, 1.29 mmol) in DCM (10 ml), a 4.0 M solution of HCl in 1,4-dioxane (5 ml) was added at 0° C. The reaction mixture was warmed up to room temperature and stirred for 2 hours. The reaction mixture was evaporated under reduced pressure in the presence of diethyl ether to afford the title compound as a pale-yellow solid (0.250 g, 57%)¹H-NMR (400 MHz, DMSO-d⁶): δ 9.13 (s, 2H), 7.87-7.90 (m, 4H), 7.67 (d, J=9.60 Hz, 1H), 7.35-7.37 (m, 3H), 7.27-7.29 (m, 1H), 3.65-3.66 (m, 2H), 3.57-3.58 (m, 2H), 3.28-3.30 (m, 3H), 2.32 (s, 3H). MS: 341.1 (M+H)⁺.

### Preparative Example 15: 5-fluoro-3-(piperidin-4-yl)-1-tosyl-1H-indole Hydrochloride

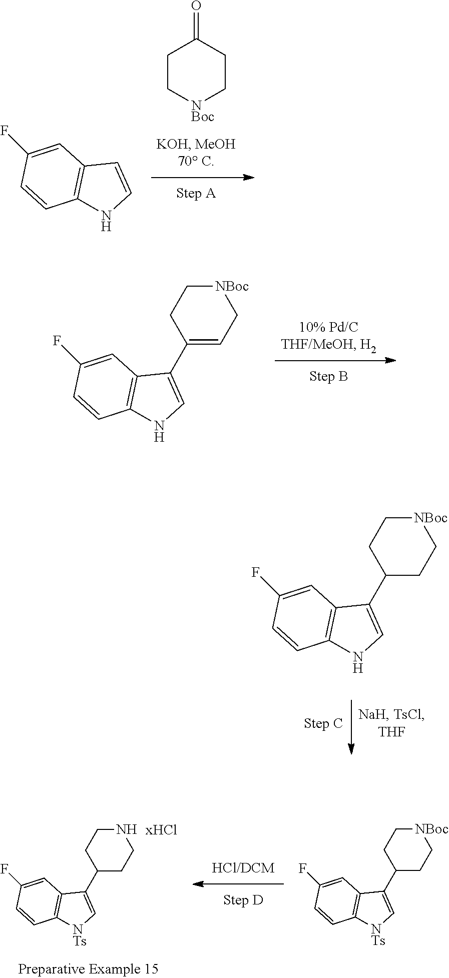 [SMILES: CC(C)(C)OC(=O)N1CCC(=O)CC1 ; Fc1ccc2[nH]ccc2c1 ; CC(C)(C)OC(=O)N1CC=C(c2c[nH]c3ccc(F)cc23)CC1 ; CC(C)(C)OC(=O)N1CCC(c2c[nH]c3ccc(F)cc23)CC1 ; Cc1ccc(S(=O)(=O)n2cc(C3CCNCC3)c3cc(F)ccc32)cc1 ; Cc1ccc(S(=O)(=O)n2cc(C3CCN(C(=O)OC(C)(C)C)CC3)c3cc(F)ccc32)cc1]

Step A

To a stirred solution of 5-fluoro-1H-indole (5.0 g, 0.0369 mol) and tert-butyl 4-oxopiperidine-1-carboxylate (14.7 g, 0.0739 mol) in methanol (50 ml), potassium hydroxide (6.2 g, 0.110 mol) was added. The reaction was heated to 70° C. and stirred at this temperature for 12 hours under nitrogen atmosphere. The mixture was concentrated. Then water (20 ml) was added, followed by DCM (20 ml). The layers were separated, and the organic phases were concentrated. Then, petroleum ether (10 ml) was added and the mixture was stirred for 30 minutes at room temperature. The slurry was filtered and dried under vacuum to afford the title compound (10.5 g) as a pale brown solid. The crude was directly used for the next step without further purification. MS: 314.9 (M−H)⁺.

Step B

To a solution of title compound from Step A above (10.5 g, 0.0331 mol) in THF/MeOH (1:1,100 ml) and Pd/C (10% wet, 1 g) was added. The reaction mixture was stirred at room temperature for 48 hours under a hydrogen atmosphere (bladder pressure). The reaction mixture was filtered through a celite pad. The filtrate was concentrated under reduced pressure to afford the title compound (10.0 g, crude) as a white solid. The crude was directly used for the next step without further purification. MS: 219.1 (M⁺+Boc).

Step C

To a suspension of sodium hydride (60% mineral oil, 1.35 g, 0.0565 mol) in THF (10 ml) was added the title compound from Step B above (6.0 g, 0.0188 mol) in THF (20 ml) at 0° C. Then the mixture was stirred at room temperature for 60 minutes. A solution of TsCl (4.31 g, 0.0226 mol) in THF (10 ml) was added dropwise at 0° C., and the mixture was stirred at room temperature for 3 hours. The reaction mixture was quenched with iced water and ethyl acetate (250 ml). The organic layers were separated, dried over sodium sulphate, filtered, and then concentrated under reduced pressure. Petroleum ether (20 ml) was added and the suspension was stirred for 30 minutes at room temperature. The slurry was filtered and dried under vacuum to afford the title compound (6.9 g, 77%). ¹H-NMR (400 MHz, DMSO-d₆): δ 7.85-7.87 (m, 3H), 7.63 (s, 1H), 7.50-7.51 (m, 1H), 7.38 (d, J=8.24 Hz, 2H), 7.16-7.18 (m, 1H), 4.04-4.06 (m, 2H), 2.86-2.89 (m, 3H), 2.32 (s, 3H), 1.87-1.90 (m, 2H), 1.43-1.46 (m, 11H). MS: 417.1 (M⁺-t-Butyl).

Step D

To a solution of title compound from Step C above (6 g, 0.0127 mol) in dichloromethane (60 ml) was added a 4N solution of HCl in 1,4-dioxane (15 ml) at 0° C. The mixture was stirred for 3 hours at 0° C. and finally warmed up to room temperature. After completion of the reaction (monitored by TLC), the mixture was concentrated, filtered, and washed with diethyl ether to afford the title compound (4.2 g) as an off white solid. The crude was directly used for the next step without further purification. MS: 373.2 (M⁺-HCl).

### Preparative Example 16: 3-ethynyl-5-fluoro-1-methyl-1H-pyrrolo[2,3-b]pyridine

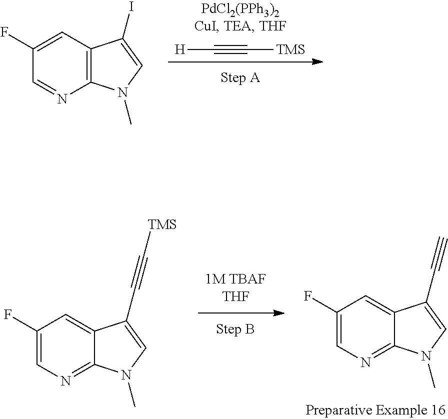

Step A

To a solution of 5-fluoro-3-iodo-1-methyl-1H-pyrrolo[2,3-b]pyridine (3 g, 10.87 mmol) in tetrahydrofuran (40 ml), TEA (7.57 ml, 54.3 mmol) was added and the mixture was purged with nitrogen for 15 minutes. Then, trimethylsilylacetylene (1.830 ml, 13.04 mmol), bis(triphenylphosphine)palladium(II) chloride (0.763 g, 1.087 mmol) and copper(I) iodide (0.207 g, 1.087 mmol) were added under an atmosphere of nitrogen. The resulting reaction mixture was stirred at 140° C. for 3 hours. The reaction mixture was filtered through a celite bed and the celite was washed with ethyl acetate (500 ml). The filtrate was concentrated under reduced pressure (bath temperature: 45° C.) and the resulting crude product was purified on a HP-Sil cartridge using a Biotage Isolera One purification system with a gradient of petroleum ether and ethyl acetate (80/20) to afford the title compound (2.3 g, 64.8%) as a yellow solid. ¹H-NMR (400 MHz, CDCl₃): δ 8.24-8.25 (m, 1H), 7.71 (dd, J=2.80, 8.40 Hz, 1H), 7.47 (s, 1H), 3.88 (s, 3H), 0.30 (s, 9H). MS: 247.1 (M+H)⁺.

Step B

To a cooled (0° C.) solution comprising the compound from Step A above (2.3 g, 9.34 mmol) in THF (10 ml), a 1 M solution of TBAF in THF (9.34 ml, 9.34 mmol) was added dropwise over a period of 5 minutes. The reaction mixture was stirred at 25° C. for 1 hour. Then, water (200 ml) and ethyl acetate (300 ml) were added and the phases were separated. The aqueous phase was extracted with ethyl acetate (2×300 ml); the combined organic layers were washed with brine (100 ml), dried over anhydrous sodium sulphate, filtered, and concentrated under reduced pressure (bath temperature: 45° C.). The crude was purified on a HP-Sil cartridge using a Biotage Isolera One purification system with a gradient of petroleum ether and ethyl acetate (70/30) to afford the title compound (0.4 g, 24.3%) as a brown solid. ¹H-NMR (400 MHz, CDCl₃): δ 8.26-8.27 (m, 1H), 7.73 (dd, J=2.80, 8.40 Hz, 1H), 7.51 (s, 1H), 3.90 (s, 3H), 3.22 (s, 1H). MS: 175.0 (M+H)⁺.

### Preparative Example 17: 3-(piperidin-4-yl)-1-tosyl-1H-pyrrolo[2,3-b]pyridine Hydrochloride

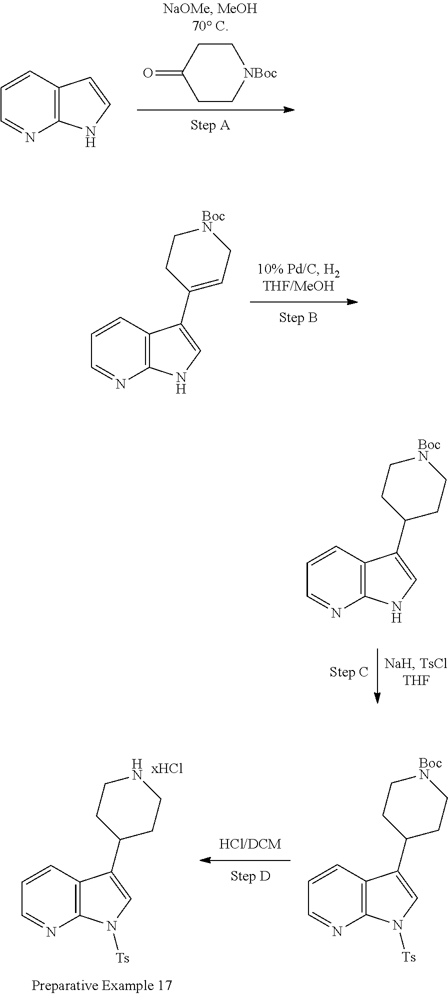 [SMILES: c1cnc2[nH]ccc2c1 ; CC(C)(C)OC(=O)N1CCC(=O)CC1 ; CC(C)(C)OC(=O)N1CC=C(c2c[nH]c3ncccc23)CC1 ; CC(C)(C)OC(=O)N1CCC(c2c[nH]c3ncccc23)CC1 ; Cc1ccc(S(=O)(=O)n2cc(C3CCNCC3)c3cccnc32)cc1 ; Cc1ccc(S(=O)(=O)n2cc(C3CCN(C(=O)OC(C)(C)C)CC3)c3cccnc32)cc1]

Step A

To a stirred solution of 1H-pyrrolo[2,3-b]pyridine (2 g, 16.9 mmol) in methanol (20 ml), sodium methoxide (1.8 g, 33.8 mmol) was added followed by tert-butyl 4-oxopiperidine-1-carboxylate (5.05 g, 25.39 mmol). The reaction mixture was heated at 70° C. for 12 hours under nitrogen atmosphere. The reaction mixture was quenched with water and then the precipitate was filtered through a sintered funnel, washed with water and petroleum ether, and then dried under reduced vacuum to afford the title compound (1.95 g, 39%) as a gummy brown solid. The crude was directly used for the next step without further purification. MS: 300.1 (M+H)⁺.

Step B

To a stirred solution of title compound from Step A above (1.95 g, 6.51 mmol) in THF (20 ml), 10% Pd/C (200 mg) was added. The mixture was stirred at room temperature for 24 hours under hydrogen pressure (1 bar). The reaction was monitored by LCMS. Then the reaction mixture was filtered through a celite pad and washed with methanol (20 ml). The filtrate was concentrated under reduced pressure to afford the title compound (1.85 g, 93%). The crude was directly used for the next step without further purification. MS: 201.2 (M+H)⁺-Boc.

Step C

To a suspension of sodium hydride (60% in paraffin oil, 0.12 g, 5.31 mmol) in DMF (3 ml), the title compound from Step B above (0.8 g, 2.65 mmol) dissolved in DMF (4 ml) was added dropwise at 0° C. The reaction mixture was stirred at room temperature for 30 minutes. Then, a solution of TsCl (0.75 g, 3.98 mmol) in DMF (3 ml) was added dropwise at 0° C. The reaction mixture was then stirred at room temperature for 1 hour. The reaction mixture was quenched with iced water and extracted with ethyl acetate (20 ml). The organic layer was separated, dried over sodium sulphate, filtered and then concentrated to afford the title compound (850 mg) as an off white solid. The crude was directly used for the next step without further purification. MS: 456.2 (M+H)⁺.

Step D

To a solution of title compound from Step C above (0.8 g, 1.75 mmol) in DCM (5 ml), a 4.0 M solution of HCl in 1,4-dioxane (1 ml) was added slowly at 0° C. The reaction mixture was stirred at 25° C. for 2 hours. The reaction mixture was then concentrated and washed with diethyl ether, the solid was filtered off and dried to afford the title crude compound (0.5 g) as HCl salt. MS: 355.9 (M+H)⁺.

### Preparative Example 18: 1-methyl-3-(piperidin-4-yl)-1H-pyrrolo[2,3-b]pyridine Hydrochloride

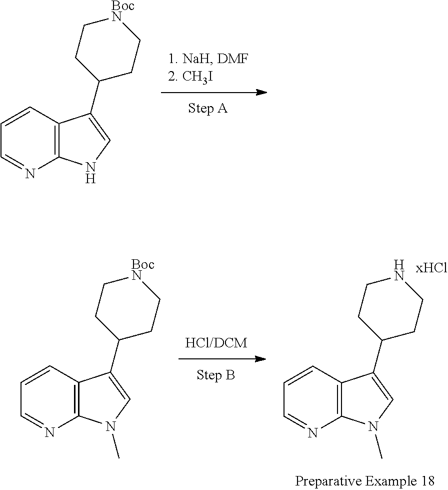 [SMILES: CC(C)(C)OC(=O)N1CCC(c2c[nH]c3ncccc23)CC1 ; Cn1cc(C2CCN(C(=O)OC(C)(C)C)CC2)c2cccnc21 ; Cn1cc(C2CCNCC2)c2cccnc21]

Step A

To a suspension of sodium hydride (0.12 g, 5.31 mmol) in DMF (3 ml) tert-butyl 4-(1H-pyrrolo[2,3-b]pyridin-3-yl)piperidine-1-carboxylate (0.9 g, 2.99 mmol) dissolved in DMF (4 ml) was added drop wise and the mixture was stirred at room temperature for 30 minutes. A solution of methyl iodide (0.85 g, 5.98 mmol) in DMF (3 ml) was added at 0° C. and the mixture was stirred at room temperature for 1 hour. The reaction mixture was quenched with iced water and extracted with ethyl acetate (20 ml). The organic layer was separated, dried over sodium sulphate, filtered and concentrated to afford the title compound (700 mg) as an off white solid. The crude was directly used in the next step without further purification. MS: 316.2 (M+H)⁺.

Step B

To a solution of title compound from the Step A above (0.7 g, 2.22 mmol) in DCM (5 ml), 4.0 M solution of HCl in 1,4-dioxane (1 ml) was added dropwise at 0° C. and the mixture was stirred at 25° C. for 2 hours The mixture was concentrated, the solid was washed with diethyl ether, dried to afford the title compound (0.5 g) as HCl salt. MS: 216.2 (M+H)⁺.

### Preparative Example 19: 3-(1,2,3,6-tetrahydropyridin-4-yl)-1-tosyl-1H-indolehydrochloride

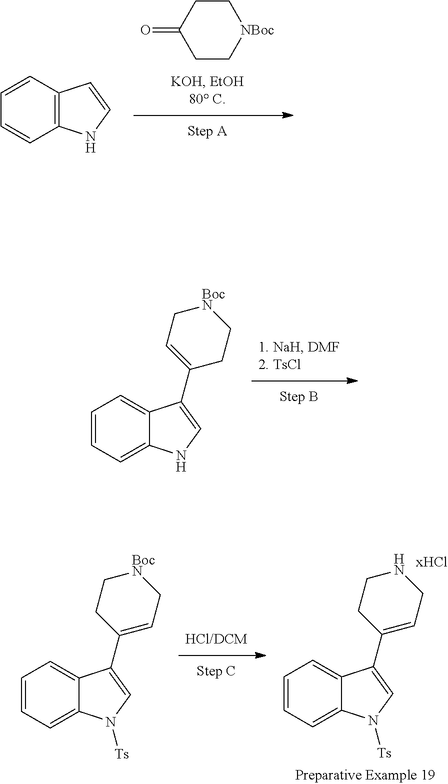 [SMILES: CC(C)(C)OC(=O)N1CCC(=O)CC1 ; c1ccc2[nH]ccc2c1 ; CC(C)(C)OC(=O)N1CC=C(c2c[nH]c3ccccc23)CC1 ; Cc1ccc(S(=O)(=O)n2cc(C3=CCN(C(=O)OC(C)(C)C)CC3)c3ccccc32)cc1 ; Cc1ccc(S(=O)(=O)n2cc(C3=CCNCC3)c3ccccc32)cc1]

Step A

To a mixture of 1H-indole (1 g, 8.54 mmol) and tert-butyl 4-oxopiperidine-1-carboxylate (1.701 g, 8.54 mmol) in ethanol (42.7 ml) was added potassium hydroxide (0.718 g, 12.80 mmol). The reaction mixture was stirred at 50° C. for 24 hours. Potassium hydroxide (0.718 g, 12.80 mmol) was added and the reaction mixture was stirred at 80° C. for 24 hours. The reaction mixture was filtered and the solid was washed with water and collected to afford the title compound (1.298 g, 51%) as a white powder ¹H-NMR (400 MHz, CDCl₃): δ 8.21 (s, 1H), 7.91 (t, J=6.3 Hz, 1H), 7.40 (t, J=6.6 Hz, 1H), 7.33-7.08 (m, 3H), 6.20 (s, 1H), 4.16 (d, J=4.4 Hz, 2H), 3.71 (q, J=5.5 Hz, 2H), 2.60 (s, 2H), 1.53 (s, 9H). MS: 299.2 (M+H)⁺.

Step B

To a stirred suspension of sodium hydride (60% in paraffin oil, 121 mg, 5.03 mmol) in dry THF (4 ml) at room temperature, a solution of title compound from Step A (500 mg, 1.676 mmol) in dry THF (4 ml) was added slowly and stirred at the same temperature for 30 minutes. Then a solution of 4-methylbenzene-1-sulfonyl chloride (327 mg, 1.718 mmol) in dry THF (1.7 ml) was added dropwise at room temperature and the reaction mixture was allowed to stir at room temperature for 1 hour 30 minutes. The reaction mixture was cooled to 0° C. and quenched with iced water, followed by extraction using ethyl acetate (2×20 ml). The combined organics were washed with brine, dried over Na₂SO₄, filtered and evaporated under reduced pressure. The crude product was purified on HP-Sil SNAP cartridges using a Biotage Isolera One purification system with a gradient of heptane and ethyl acetate (100/0 to 40/60). The fractions containing the compound were collected and concentrated under reduced pressure to afford the title compound (547 mg, 72%) as a beige solid. ¹H-NMR (80 MHz, CDCl₃): δ 8.09-7.93 (m, 1H), 7.85-7.77 (m, 1H), 7.77-7.65 (m, 2H), 7.50 (s, 1H), 7.40-7.28 (m, 2H), 7.25-7.09 (m, 2H), 6.28-6.08 (m, 1H), 4.20-4.02 (m, 2H), 3.67 (t, J=5.7 Hz, 2H), 2.67-2.38 (m, 2H), 2.34 (s, 3H), 1.50 (s, 9H). MS: 453.1 (M+H)⁺.

Step C

To a solution of title compound from Step B (497 mg, 1.098 mmol) in dioxane (6.5 ml) was added HCl in dioxane (4 M) (3.3 ml, 13.18 mmol) dropwise at room temperature. The reaction mixture was stirred at room temperature overnight. The reaction mixture was filtered and the solid was washed twice with ethyl acetate (2×50 ml) to afford the title compound (166 mg, 39%) as a yellow powder. ¹H-NMR (80 MHz, DMSO-d₆): δ 9.10 (s, 2H), 8.12-7.78 (m, 5H), 7.53-7.23 (m, 4H), 6.43-6.22 (m, 1H), 3.92-3.64 (m, 2H), 3.36-3.11 (m, 3H), 2.90-2.62 (m, 2H), 2.32 (s, 3H) MS: 353.1 (M+H)⁺.

### Preparative Example 20: 3-(piperidin-4-yl)-1-tosyl-1H-pyrrolo[3,2-c]pyridine Hydrochloride

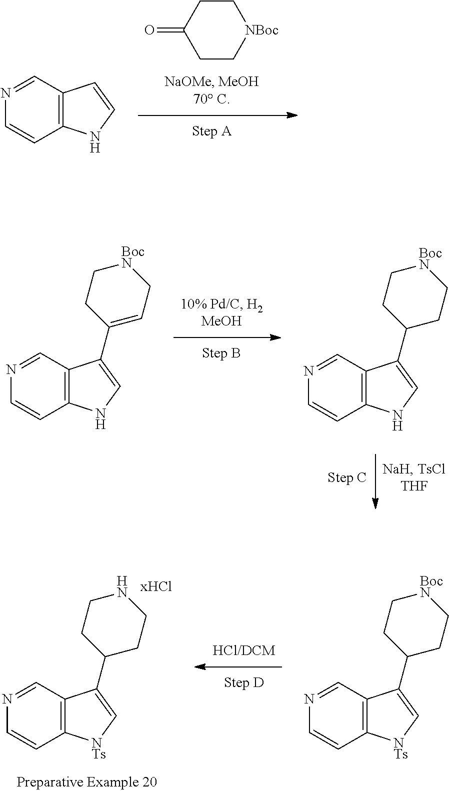 [SMILES: CC(C)(C)OC(=O)N1CCC(=O)CC1 ; c1cc2[nH]ccc2cn1 ; CC(C)(C)OC(=O)N1CC=C(c2c[nH]c3ccncc23)CC1 ; CC(C)(C)OC(=O)N1CCC(c2c[nH]c3ccncc23)CC1 ; Cc1ccc(S(=O)(=O)n2cc(C3CCNCC3)c3cnccc32)cc1 ; Cc1ccc(S(=O)(=O)n2cc(C3CCN(C(=O)OC(C)(C)C)CC3)c3cnccc32)cc1]

Step A

To a stirred solution of 1H-pyrrolo[3,2-c]pyridine (2.5 g, 21.2 mmol) in methanol (20 ml) sodium methoxide (2.75 g, 63.4 mmol) and tert-butyl 4-oxopiperidine-1-carboxylate (6.32 g, 31.7 mmol) were added and the mixture was heated to 70° C. for 12 hours under nitrogen atmosphere. The reaction mixture was quenched with water (20 ml) and the precipitate was filtered through a sintered funnel. The solid was washed with water (50 ml) then petroleum ether (50 ml) and dried under reduced pressure to afford the title compound (4.0 g, 65%) as a brown solid. ¹H-NMR (400 MHz, DMSO-d₆): δ 11.55 (s, 1H), 9.13 (s, 1H), 8.19 (d, J=5.60 Hz, 1H), 7.52 (d, J=2.00 Hz, 1H), 7.36-7.37 (m, 1H), 6.26 (s, 1H), 4.05 (s, 2H), 3.56-3.57 (m, 2H), 2.50-2.51 (m, 2H), 1.44 (s, 9H). MS: 300.2 (M+H)⁺.

Step B

To a solution of title compound from Step A (4.0 g, 12 mmol) in THF (40 ml), 10% Pd/C (400 mg) was added and the mixture was stirred at room temperature for 24 hours under hydrogen pressure (1 bar). The reaction mixture was filtered through celite and washed with methanol (50 ml). The filtrate was concentrated under reduced pressure to afford the title compound (3.5 g, 88%) MS: 302.2 (M+H)⁺.

Step C

To a suspension of sodium hydride (60% in paraffin oil, 1.1 g, 39.9 mmol) in THF (10 ml) a solution of title compound from Step B (3.5 g, 11.6 mmol) in THF (40 ml) was added dropwise at 0° C. and the mixture was stirred at room temperature for 30 minutes. A solution of TsCl (3.30 g, 17.3 mmol) in THF (15 ml) was added at 0° C. and the mixture was stirred at room temperature for 1 hour. The reaction mixture was quenched with iced water (10 ml) followed by extraction using ethyl acetate (50 ml). The organic layer was separated, dried over sodium sulphate, filtered and then concentrated under reduced pressure to afford the title compound (4 g, 75%) as an off white solid. MS: 456.2 (M+H)⁺.

Step D

To a solution of the title compound from Step C above (4 g, 8.75 mmol) in DCM (40 ml) a 4.0 M solution of hydrochloric acid in 1,4-dioxane (10 ml) was added slowly at 0° C. and the mixture was stirred at 25° C. for 2 hours. The reaction mixture was concentrated and washed with ether (50 ml) to afford the title compound (3 g, 87%) as HCl salt. MS: 355.9 (M+H)⁺.

### Preparative Example 21: 3-(piperazin-1-yl)-1-tosyl-1H-indole Hydrochloride

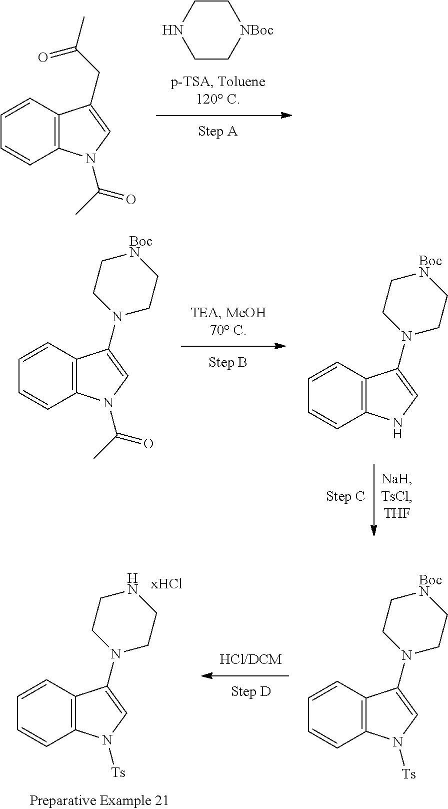 [SMILES: CC(=O)Cc1cn(C(C)=O)c2ccccc12 ; CC(C)(C)OC(=O)N1CCNCC1 ; CC(=O)n1cc(N2CCN(C(=O)OC(C)(C)C)CC2)c2ccccc21 ; CC(C)(C)OC(=O)N1CCN(c2c[nH]c3ccccc23)CC1 ; Cc1ccc(S(=O)(=O)n2cc(N3CCNCC3)c3ccccc32)cc1 ; Cc1ccc(S(=O)(=O)n2cc(N3CCN(C(=O)OC(C)(C)C)CC3)c3ccccc32)cc1]

Step A

To a stirred solution of 1-acetyl-1H-indol-3-yl acetate (2.5 g, 11.5 mmol) in toluene (25 ml), tert-butyl piperazine-1-carboxylate (10.71 g, 57.5 mmol) and p-toluene sulfonic acid (0.4 g, 2.1 mmol) were added. The resulting reaction mixture was heated to 120° C. for 12 hours under nitrogen atmosphere. The mixture was quenched with iced water (30 ml) followed by extraction using ethyl acetate (50 ml). The organic layer was separated, dried over sodium sulphate, filtered and then concentrated. The crude product was purified using column chromatography eluted with hexane/ethyl acetate (90/10) to afford the title compound (2.6 g, 65%) as a purple solid. ¹H-NMR (400 MHz, DMSO-d₆): δ 8.37 (d, J=8.40 Hz, 1H), 7.65 (d, J=7.60 Hz, 1H), 7.26-7.27 (m, 3H), 3.54 (d, J=4.80 Hz, 4H), 2.89-2.91 (m, 5H), 2.59 (s, 2H), 1.44 (s, 9H). MS: 344.1 (M+H)⁺.

Step B

To a solution of title compound from Step A above (2.6 g. 7.5 mmol) in methanol (20 ml), triethylamine (3.29 ml, 22.7 ml) was added. The reaction mixture was heated to 65° C. for 2 hours. The reaction mixture was diluted with water and then was extracted using ethyl acetate (50 ml). The organic layers were separated, dried over sodium sulphate, filtered and then concentrated. The crude product was purified using a column chromatography eluted with hexane/ethyl acetate (80/20) to afford the title compound (2.2 g, 96.4%) as a pink solid. ¹H-NMR (400 MHz, DMSO-d₆): δ 10.56 (s, 1H), 7.52 (d, J=8.00 Hz, 1H), 7.30 (d, J=8.00 Hz, 1H), 7.04-7.05 (m, 1H), 6.87-6.88 (m, 2H), 3.51-3.52 (m, 4H), 2.90-2.91 (m, 4H), 1.43 (s, 9H). MS: 302.0 (M+H)⁺.

Step C

To a suspension of sodium hydride (60% in paraffin oil, 1.5 g, 21.8 mmol) in THF (10 ml), a solution of title compound from Step B (2.2 g, 7.3 mmol) in THF (20 ml) was added dropwise at 0° C. The reaction mixture was stirred at room temperature for 30 minutes. Then a solution of tosyl chloride (2.09 g, 10.9 mmol) in THF (15 ml) was added at 0° C. The reaction mixture was stirred at room temperature for 1 hour. The reaction mixture was quenched with iced water, then extracted using ethyl acetate (50 ml). The organic layers were separated, dried over sodium sulphate, filtered and then concentrated to afford the title compound (3 g, 90%) as a brown solid. ¹H-NMR (400 MHz, DMSO-d₆): δ 7.94 (d, J=8.40 Hz, 1H), 7.79 (d, J=8.40 Hz, 2H), 7.60 (d, J=7.60 Hz, 1H), 7.32-7.33 (m, 3H), 7.22-7.24 (m, 1H), 7.16 (s, 1H), 3.50 (s, 4H), 2.96-2.97 (m, 4H), 2.30 (s, 3H), 1.43 (s, 9H). MS: 456.2 (M+H)⁺.

Step D

To a solution of title compound from Step C above (3 g, 6.5 mmol) in DCM (30 ml), a 4.0 M solution of hydrochloric acid in 1,4-dioxane (10 ml) was added at 0° C. The mixture was stirred at 25° C. for 2 hours. The reaction mixture was concentrated under reduced pressure, washed with diethyl ether to afford the tile compound (1.3 g) as HCl salt. The crude was directly used for the next step without further purification. MS: 356.0 (M+H)⁺.

### Preparative Example 22: 5-fluoro-1-methyl-3-(octahydrocyclopenta[c]pyrrol-5-yl)-1H-pyrrolo[2,3-b]pyridine Hydrochloride

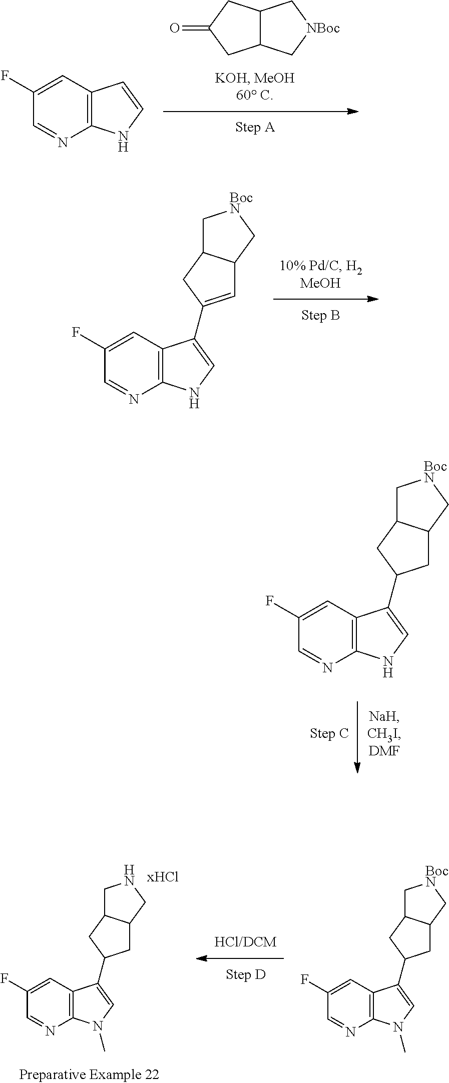 [SMILES: CC(C)(C)OC(=O)N1CC2CC(=O)CC2C1 ; Fc1cnc2[nH]ccc2c1 ; CC(C)(C)OC(=O)N1CC2C=C(c3c[nH]c4ncc(F)cc34)CC2C1 ; CC(C)(C)OC(=O)N1CC2CC(c3c[nH]c4ncc(F)cc34)CC2C1 ; Cn1cc(C2CC3CNCC3C2)c2cc(F)cnc21 ; Cn1cc(C2CC3CN(C(=O)OC(C)(C)C)CC3C2)c2cc(F)cnc21]

Step A

To a mixture of 5-fluoro-1H-pyrrolo[2,3-b]pyridine (3 g, 22.1 mmol), tert-butyl 5-oxohexahydrocyclopenta[c]pyrrole-2(1H)-carboxylate (5.46 g, 24.2 mmol) and potassium hydroxide (2.5 g, 7.28 mmol) and methanol (20 ml) were added. The reaction mixture was stirred at 60° C. for 16 hours. Water (20 ml) was added to the reaction mixture. The reaction mixture was stirred for 10 minutes then filtered to afford the title compound (2.5 g, 24%) as a brown solid. MS: 344.2 (M+H)⁺.

Step B

To a solution of title compound from Step A (2.5 g, 7.3 mmol) in methanol (15 ml), 10% Pd/C (250 mg) was added and the reaction mixture was stirred at room temperature for 12 hours under hydrogen pressure. The reaction mixture was filtered through celite and the filtrate was concentrated under vacuum to afford the title compound (2 g) as an off white solid. The crude was directly used for the next step without further purification. MS: 246.2 (M+H)⁺-Boc.

Step C

To a suspension of sodium hydride (60% in paraffin, 0.399 g, 17.4 mmol) in THF (10 ml), a solution of title compound from Step B (2.0 g, 5.8 mmol) in THF (20 ml) was added dropwise at 0° C. The reaction mixture was stirred at room temperature for 60 minutes. A solution of methyl iodide (0.54 mL, 8.7 mmol) in THF (2 ml) was added at 0° C. and the mixture was stirred at room temperature for 3 hours. The reaction mixture was quenched with iced water slowly and then ethyl acetate (25 ml) was added. The organic phases were separated and the aqueous phases were extracted with ethyl acetate two more times. The combined organic phases were dried over Na₂SO₄, filtered and the solvent was evaporated under reduced pressure. The crude product was purified on a HP-Sil column (Biotage) by employing a petroleum ether/ethyl acetate gradient (50/50) to afford the title compound (1.5 g, 71%) as a brown solid. MS: 304.1 (M+H)⁺-t-butyl.

Step D

To a solution of title compound from Step C above (1.5 g, 4.2 mmol) in DCM (10 ml), a 4.0 M solution of hydrochloric acid in 1,4-dioxane (5 ml) was added at 0° C. The reaction mixture was stirred at 25° C. for 2 hours. The reaction mixture was concentrated under reduced pressure and washed with diethyl ether to afford the title compound (1.0 g, 81%) as an off white solid. ¹H-NMR (400 MHz, DMSO-d₆): δ 9.52 (bs, 2H), 8.21-8.22 (m, 1H), 8.10 (dd, J=2.80, 10.00 Hz, 1H), 7.45 (s, 1H), 3.76 (s, 3H), 3.12-3.13 (m, 5H), 2.89-2.90 (m, 2H), 2.25-2.27 (m, 2H), 1.63-1.65 (m, 2H). MS: 260.0 (M+H)⁺.

### Preparative Example 23: 5-fluoro-3-(piperidin-4-yl)-1-tosyl-1H-indazole Hydrochloride

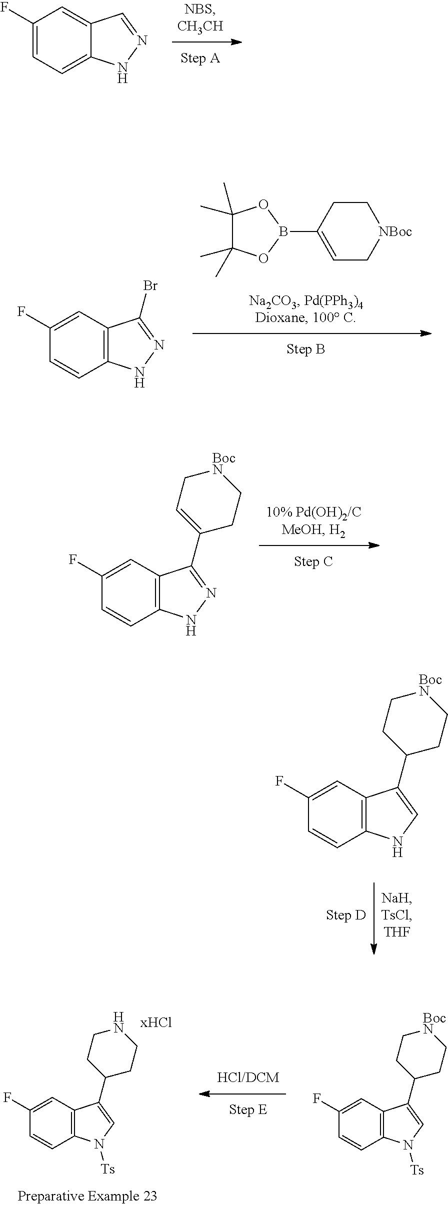

Step A

To a stirred solution of 5-fluoro-1H-indazole (2 g, 14.7 mmol) in acetonitrile (60.00 ml), NBS (2.61 g, 14.7 mmol) was added portion wise at 0° C. The reaction mixture was stirred at room temperature for 2 hours under nitrogen atmosphere. The reaction mixture was concentrated and purified on a HP-Sil column (Biotage) by employing a petroleum ether/ethyl acetate gradient (100/0 to 90/10) to the title compound (3.1 g, 98.1%) as a pale-yellow solid. ¹H-NMR (400 MHz, DMSO-d₆): δ 13.57 (s, 1H), 7.62-7.63 (m, 1H), 7.34-7.37 (m, 1H). MS: 214.9 (M+H)⁺.

Step B

To a stirred solution of 3-bromo-5-fluoro-1H-indazole compound from Step A (1 g, 4.62 mmol) in 1,4-dioxane (60 ml) in a sealed tube, tert-butyl 4-(4,4,5,5-tetramethyl-1,3,2-dioxaborolan-2-yl)-3,6-dihydro-2H-pyridine-1-carboxylate (1.43 g, 4.62 mmol) was added, followed by a 2.0 M solution of Na₂CO₃ (6.94 ml). Then the sealed tube was purged with nitrogen for 20 minutes. Tetrakis(triphenylphosphine)palladium(0) (0.534 g, 0.46 mmol) was added, and the sealed tube was purged with nitrogen for 5 minutes. The reaction mixture was heated at 100° C. for 16 hours. Completion of reaction was monitored by TLC. The reaction mixture was diluted with water (100 ml) and extracted with ethyl acetate (2×200 ml). The combined organic layers were dried over Na₂SO₄, filtered and concentrated under reduced pressure. The residue was purified on a 230-400 silica gel cartridge using a Biotage purification system by employing a petroleum ether/ethyl acetate gradient (100/0 to 80/20) to afford the title compound (1.45 g, 97.8%) as a yellow solid. MS: 318.1 (M+H)⁺.

Step C

To a stirred solution of the title compound from Step B above (14.5 g, 46.0 mmol) in methanol (15 ml), 10% Pd(OH)₂/C (0.2 g, 0.18 mmol) was added. The reaction mixture was stirred under H₂ atmosphere at room temperature for 5 hours. The crude was filtered through a celite pad which was washed with methanol. The filtrate was concentrated under reduced pressure. The residue was purified on a chromatography column using a Biotage purification system by employing a petroleum ether/ethyl acetate gradient (100/0 to 70/30) to afford the title compound (0.8 g, 63.6%) as an off white solid. MS: 220.1 (M+H)⁺-Boc.

Step D

To a suspension of sodium hydride (60% in paraffin oil, 0.198 g, 4.95 mmol) in tetrahydrofuran (15 ml), the title compound from Step C above (0.8 g, 2.47 mmol) was added portion wise at 0° C. The reaction mixture was stirred at room temperature for 60 minutes. Tosyl chloride (0.566 g, 2.97 mmol) was then added dropwise (previously dissolved in THF 10 ml) at 0° C. The reaction mixture was then stirred at room temperature for 2 hours. The reaction mixture was quenched slowly with ice cold water and extracted using ethyl acetate (2×50 ml). The organic layers were separated, dried over sodium sulphate, and filtered. Then the solvent was removed under reduced pressure to afford the title compound (1.25 g, 95.4%) as a pale-yellow solid. MS: 474.2 (M+H)⁺.

Step E

To a stirred solution of the title compound from Step D above (1.25 g, 2.35 mmol) in dichloromethane (10 ml), a 4N HCl solution in 1,4-dioxane (7 ml) was added at 0° C. The reaction mixture was stirred for 1 hour at 0° C. before to being allowed to warm up to room temperature. After completion of the reaction by TLC, the reaction mixture was concentrated. Diethyl ether and petroleum ether were added, and the crude mixture was stirred for 15 minutes at room temperature. The obtained solid was filtered, dried under reduced pressure to afford the title compound (0.850 g, 84.6%) as a white solid. ¹H-NMR (400 MHz, DMSO-d₆): δ 8.85 (bs, 1H), 8.68 (bs, 1H), 8.11-8.12 (m, 1H), 7.87 (dd, J=2.00, 8.60 Hz, 1H), 7.77 (d, J=8.40 Hz, 2H), 7.55-7.56 (m, 1H), 7.39 (d, J=8.00 Hz, 2H), 3.35-3.38 (m, 2H), 2.97-3.00 (m, 3H), 2.33 (s, 3H), 2.05-2.08 (m, 2H), 1.94-1.97 (m, 2H). MS: 374.1 (M+H)⁺.

### Preparative Example 24: 1-methyl-3-(piperidin-4-yl)-1H-indole Hydrochloride

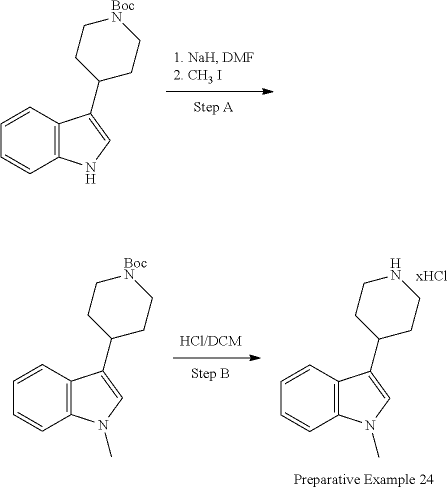 [SMILES: CC(C)(C)OC(=O)N1CCC(c2c[nH]c3ccccc23)CC1 ; Cn1cc(C2CCN(C(=O)OC(C)(C)C)CC2)c2ccccc21 ; Cn1cc(C2CCNCC2)c2ccccc21]

Step A

To a suspension of sodium hydride (60% mineral oil) (1.53 g, 0.0399 mol) in THF (10 ml), a solution of butyl 4-(1H-indol-3-yl)piperidine-1-carboxylate (title compound from Step B of Preparative Example 2, 6.0 g, 0.020 mol) in THF (20 ml) was added dropwise at 0° C. The reaction mixture was stirred at room temperature for 60 minutes. A solution of methyl iodide (4.23 g, 0.03 mol) in THF (10 ml) was added at 0° C. and the reaction mixture was stirred at room temperature for 3 hours. The reaction mixture was then quenched slowly with iced water, followed by addition of ethyl acetate (250 ml). The organic phases were separated and the aqueous phases were extracted with ethyl acetate two more times. The combined organic phases were dried over Na₂SO₄, filtered and the solvent were evaporated under reduced pressure. The slurry was filtered and dried under vacuum to afford the title compound (5.5 g, 87%). MS: 215.1 (M⁺-Boc).

Step B

To a stirred solution of the title compound from Step A above (5.5 g, 0.175 mol) in DCM (10 ml), a 4.0 M HCl in 1,4-dioxane solution (10 ml) was added at 0° C. The reaction mixture was stirred at room temperature for 3 hours. The reaction mixture was concentrated under reduced pressure to afford the title compound (4.5 g) as an off white solid. The crude was directly used for the next step without further purification. MS: 215.1 (M⁺-HCl).

### Preparative Example 25: 5-bromo-2-morpholinooxazolo[5,4-b]pyridine

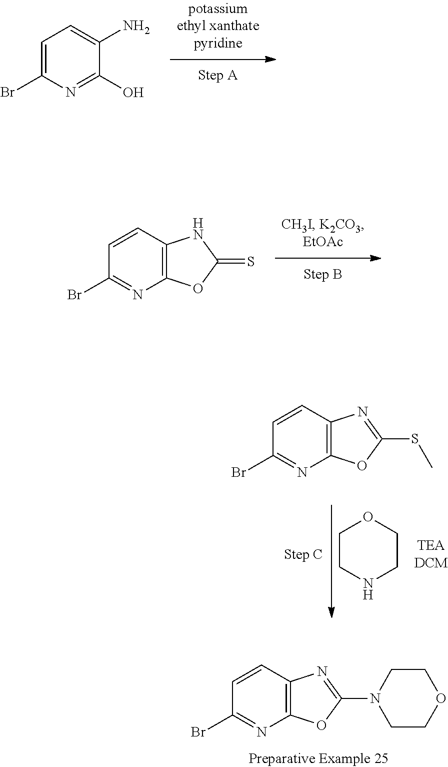 [SMILES: Nc1ccc(Br)nc1O ; S=c1[nH]c2ccc(Br)nc2o1 ; CSc1nc2ccc(Br)nc2o1 ; C1COCCN1 ; Brc1ccc2nc(N3CCOCC3)oc2n1]

Step A

To a stirred solution of 3-amino-6-bromo-pyridin-2-ol (1.0 g, 5.24 mmol) in pyridine (15 ml), potassium ethyl xanthate (0.924 g, 5.76 mmol) was added and the mixture was heated to 120° C. for 12 hours. The reaction mixture was acidified with 1.5N HCl, extracted with ethyl acetate (30 ml) and water (30 ml). The organic phase was separated and the aqueous phase was extracted with ethyl acetate two more times (2×30 ml). The combined organic phases were dried over Na₂SO₄, filtered and the solvents were evaporated under reduced pressure to afford the title compound (0.6 g, 48%) as a pale-yellow solid. MS: 228.9 (M−2H)⁺.

Step B

To a stirred solution of the title compound from Step A above (0.6 g, 2.56 mmol) in ethyl acetate (30 ml) potassium carbonate (0.496 g, 5.59 mmol) and methyl iodide (0.02 ml, 3.82 mmol) were added and the mixture was stirred at 25° C. for 12 hours. The reaction mixture was extracted with ethyl acetate (30 ml), washed with water (30 ml) and brine solution (30 ml). The organic layers were combined and concentrated under vacuum to afford the title compound (0.6 g, 84%) as a pale-yellow solid. MS: 247.1 (M+2H)⁺.

Step C

To a title compound from Step B above (0.6 g, 2.15 mmol) was added morpholine (3.77 ml) and the mixture was heated to 80° C. for 12 hours. The mixture was concentrated and purified by silica gel column chromatography using petroleum ether/ethyl acetate (70/30) to afford the title compound (0.45 g, 73%) as an off-white solid. MS: 284.0 (M+H)⁺.

### Preparative Example 26: 4-(6-chlorothiazolo[5,4-b]pyridin-2-yl)morpholine

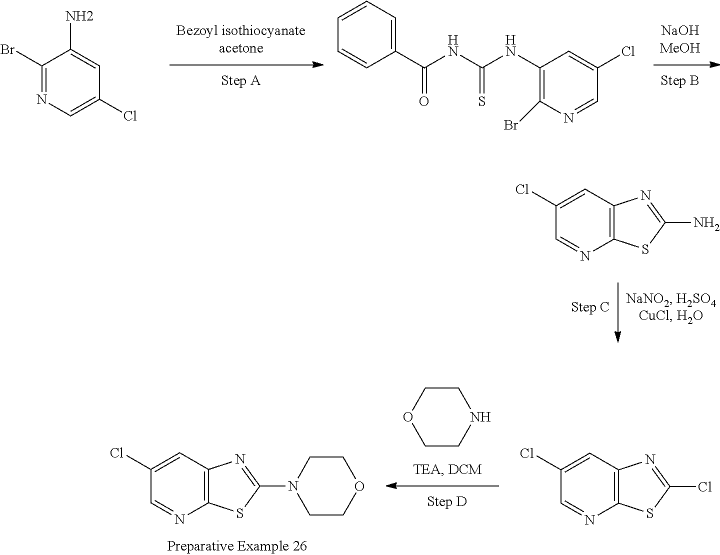 [SMILES: Nc1cc(Cl)cnc1Br ; O=C(NC(=S)Nc1cc(Cl)cnc1Br)c1ccccc1 ; Nc1nc2cc(Cl)cnc2s1 ; C1COCCN1 ; Clc1cnc2sc(N3CCOCC3)nc2c1 ; Clc1cnc2sc(Cl)nc2c1]

Step A

A solution of 2-bromo-5-chloropyridin-3-amine (10 g, 0.0482 mol) and benzoyl isothiocyanate (8.43 ml, 0.0675 mol) in acetone (150 ml) was stirred at room temperature for 18 hours. After completion of the reaction (monitored by TLC), the reaction mixture was evaporated under reduced pressure and the solid was filtered, washed with n-hexane (200 ml) and dried to give the title compound (7.1 g, 86.6%) as a white solid. ¹H-NMR (400 MHz, CDCl3): δ 13.01 (s, 1H), 9.21 (s, 1H), 9.09 (s, 1H), 8.26 (s, 1H), 7.96 (d, J=10.40 Hz, 2H), 7.28-7.57 (m, 3H). MS: 367.9 (M−2H)⁺.

Step B

A suspension of the title compound from Step A above (15 g, 0.0404 mol) in 3.0N solution of NaOH (200 ml) and MeOH (100 ml) was refluxed for 1 hour. The reaction mixture was cooled to 0° C. and the resulting precipitate was filtered off and dried to afford the title compound as brown solid (7 g, 93%). MS: 186.1 (M+H)⁺.

Step C

To a stirred solution of the title compound from Step B above (1 g, 5.38 mmol) in 3.0N solution of H₂SO₄ (100 ml), sodium nitrate (0.52 g, 7.52 mmol) in water (10 ml) was added dropwise at 0° C. and the mixture was stirred at 0° C. for 30 minutes. Then, copper (1) chloride (1.01 g, 0.0754 mol) in concentrated HCl (10 ml) was added dropwise at 0° C. The reaction mixture was allowed to warm to room temperature and stirred for 6 hours. After completion of the reaction (monitored by TLC), the reaction mixture was diluted with water (50 ml) and extracted with ethyl acetate (3×20 ml). The combined organics were washed with brine (10 ml), dried over Na₂SO₄, filtered and concentrated under reduced pressure. The crude material was purified by silica-gel (60-120) column chromatography eluting with ethyl acetate and petroleum ether (20/80) to afford the title compound (700 mg, 63%) as a brown solid. ¹H-NMR (400 MHz, CDCl₃): δ 8.58 (s, 1H), 8.20 (s, 1H). MS: 205.1 (M+H)⁺.

Step D

To a stirred solution of the title compound from Step C above (700 mg, 3.41 mmol) in dry DCM (10 ml), morpholine (356 mg, 4.09 mmol) and triethylamine (0.95 ml, 6.6 mmol) were added and the mixture was stirred at room temperature for 16 hours. The reaction mixture was concentrated to afford the title compound (900 mg, 96.8%) as a brown solid. ¹H-NMR (400 MHz, CDCl3): δ 8.18 (s, 1H), 7.72 (s, 1H), 3.85-3.86 (m, 4H), 3.68-3.69 (m, 4H). MS: 256.1 (M+H)⁺.

### Preparative Example 27: 4-(5-chlorothiazolo[4,5-b]pyridin-2-yl)morpholine

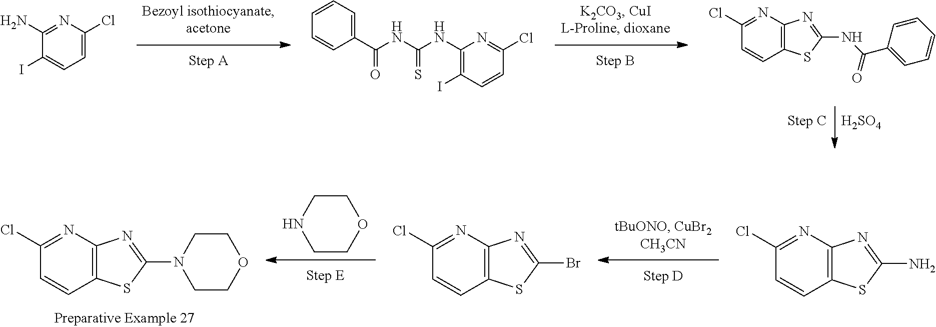

Step A

A solution of 6-chloro-3-iodopyridin-2-amine (5 g, 0.0196 mol) and benzoyl isothiocyanate (3.86 g, 0.0255 mol) in acetone (25 ml) was stirred at 60° C. for 12 hours. After completion of the reaction (monitored by TLC), the reaction mixture was evaporated under reduced pressure and the solid was filtered, washed with n-hexane (200 ml) and dried to give the title compound (7.1 g, 86.6%) as a pale brown solid. MS: 418.0 (M+H)⁺.

Step B

To a stirred solution of the title compound from Step A above (7.1 g, 0.0170 mol) in 1,4-dioxane (25 ml) at 25° C., potassium carbonate (4.4 g, 0.0323 mol), L-proline (0.39 g, 0.0034 mol) and copper(I) iodide (0.324 g, 0.0017 mol) were added and the resulting mixture was stirred at 80° C. for 16 hours, After completion of the reaction (monitored by TLC), the reaction mixture was poured into water (100 ml) and aqueous saturated NH₄Cl (100 ml) and stirred at 25° C. for 1 hour. The solid thus obtained was filtered, washed with aqueous saturated NH₄Cl (2×25 ml), water (2×25 ml) and dried to give the title compound (4.9 g crude) as an off white solid. MS: 290.0 (M+H)⁺.

Step C

A suspension of title compound from Step B above (4.9 g, 0.0166 mol) in H₂SO₄ (70%, 27 ml) was heated at 120° C. for 4 hours. After completion of the reaction (monitored by TLC), the reaction mixture was cooled to 25° C. and slowly poured into 100 ml of iced cold water. Then, the reaction mixture was basified using aqueous NaOH (50%) and extracted with ethyl acetate (6×25 ml). The combined organic layers were dried over Na₂SO₄, filtered and concentrated under reduced pressure to give the title compound (2.3 g, 70%) as a light-yellow solid. MS: 186.1 (M+H)⁺.

Step D

To a suspension of title compound from Step C above (2.3 g, 0.0124 mol) in acetonitrile (20 ml) at 0° C., was added tert-butyl nitrite (2.2 ml, 0.0186 mol) over a period of 10 minutes with a syringe. Then, copper (II) bromide (3.33 g, 0.0149 mol) was added portion wise at 0° C. and stirring was continued for 30 minutes. The reaction mixture was allowed to warm to 25° C. and stirred for 6 hours. After completion of the reaction (monitored by TLC), the solvent was evaporated under reduced pressure to yield the residue which was diluted with water (20 ml) and extracted with DCM/MeOH (95/5) (20 ml×3). The combined organics were washed with brine (10 ml), dried over Na₂SO₄, filtered and concentrated under reduced pressure. The crude material was purified by silica-gel (60-120) column chromatography using DCM/MeOH (99/1) to afford the title compound (2.7 g, 90%) as an off-white solid. MS: 248.9 (M+H)⁺.

Step E

A solution of title compound from Step D above (2.7 g, 0.0108 mol) in morpholine (30 mg) was heated to 80° C. for 12 hours. After completion of the reaction (monitored by TLC), the reaction mixture was concentrated under reduced pressure. The crude product was purified by silica-gel (60-120 mesh) column chromatography using hexane/EtOAc (70/30) to afford the title compound (2.13 g, 76%) as a pale brown solid. MS: 255.9 (M+H)⁺

### Preparative Example 28: 4-(6-chlorothiazolo[5,4-c]pyridin-2-yl)morpholine

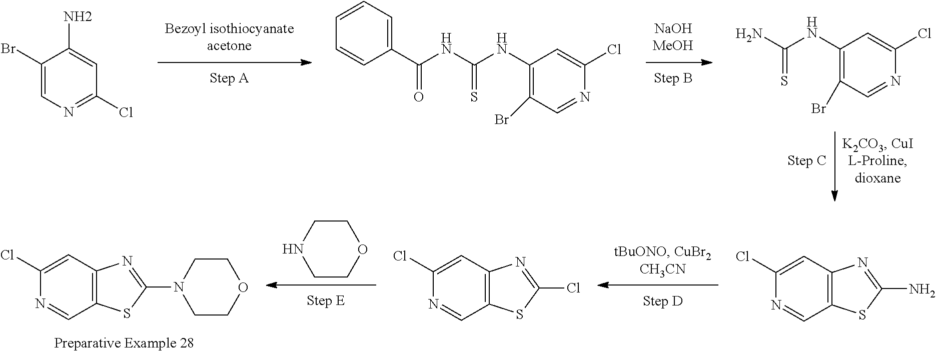

Step A

A solution of 5-bromo-2-chloropyridin-4-amine (5 g, 0.0241 mol) and benzoyl isothiocyanate (7.88 g, 0.0483 mol) in acetone (30 ml) was stirred at 60° C. for 12 hours. After completion of the reaction (monitored by TLC), the reaction mixture was evaporated under reduced pressure and the solid was filtered, washed with n-hexane (200 ml) and dried to give the title compound (3 g, 33%) as a pale brown solid. MS: 371.9 (M+H)⁺.

Step B

A suspension of the title compound from Step A above (3 g, 0.0080 mol) in 6.0N NaOH (15 ml) and MeOH (30 ml) was refluxed for 4 hours. The reaction mixture was cooled to room temperature and saturated NH₄Cl solution was added until the solid precipitated out. The solid was filtered and washed with water (20 ml) and DCM (20 ml) and dried to afford title compound as brown solid (1.6 g, 76.19%). MS: 267.9 (M+H)⁺.

Step C

To a suspension of title compound from Step B above (1.6 g, 0.006 mol) in DMSO (15 ml) at 25° C., Cesium carbonate (3.96 g, 0.012 mol), L-proline (0.139 g, 0.0012 mol) and copper(I) iodide (0.114 g, 0.0063 mmol) were added and the resulting mixture was stirred at 70° C. for 16 hours. After completion of the reaction (monitored by TLC), the reaction mixture was poured into water (100 ml), the resulting precipitate was filtered off and dried to give the title compound (0.5 g, 45%) as a brown solid. MS: 186.1 (M+H)⁺.

Step D

To a suspension of the title compound from Step C above (500 mg, 2.69 mmol) and copper chloride (346 mg, 3.50 mmol) in acetonitrile (15 ml) at 0° C., was added isoamyl nitrite (430 mg, 4.0409 mmol) and the resulting mixture was stirred for 30 minutes, then was allowed to warm to 25° C. and stirred for an additional 4 hours. After completion of the reaction (monitored by TLC), the solvent was evaporated under reduced pressure to afford the title compound (290 mg, crude) as a brown solid. MS: 207.0 (M+H)⁺.

Step E

A solution of the title compound from Step D above (290 mg, 1.4706 mmol) in morpholine (10 ml) was heated to 80° C. for 12 hours. After completion of the reaction (monitored by TLC), the reaction mixture was concentrated under reduced pressure to yield the crude product which was purified by silica-gel (60-120 mesh) column chromatography using hexane/EtOAc (70/30) to afford the title compound (130 mg, 36%) as a pale brown solid. MS: 256.0 (M+H)⁺:

### Preparative Example 29: 6-chloro-2-morpholinobenzo[d]oxazole

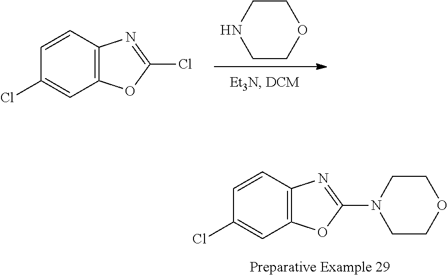 [SMILES: Clc1ccc2nc(Cl)oc2c1 ; C1COCCN1 ; Clc1ccc2nc(N3CCOCC3)oc2c1]

To a solution of 2,6-dichlorobenzo[d]oxazole (5 g, 26.8 mmol) in dry dichloromethane (50 ml), morpholine (3.50 g, 40.3 mmol) was added. The reaction mixture was cooled to 0° C. To this cold reaction mixture triethylamine (4.0 g, 39.6 mmol) was added dropwise. After the addition was completed, the reaction mixture was allowed to stir at room temperature for 4 hours. After the completion of the reaction, the reaction mixture was treated with water (2×20 ml) and extracted with dichloromethane. The organic layers were separated, dried over Na₂SO₄, filtered and evaporated to afford a white solid which was triturated with diethyl ether to afford the title compound (5 g, 78%). ¹H-NMR (400 MHz, DMSO-d₆) δ=7.59 (d, J=2.80 Hz, 1H), 7.30 (d, J=11.20 Hz, 1H), 7.21 (dd, J=2.80, 11.20 Hz, 1H), 3.71-3.74 (m, 4H), 3.57-3.60 (m, 4H). MS: 239.2 (M+H)⁺.

### Preparative Example 30: 5-chloro-2-morpholinobenzo[d]oxazole

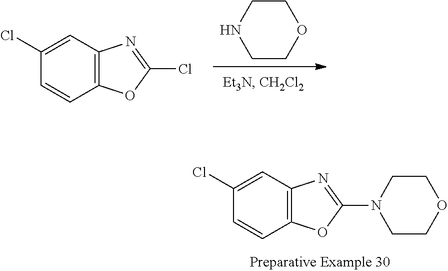 [SMILES: Clc1ccc2oc(Cl)nc2c1 ; C1COCCN1 ; Clc1ccc2oc(N3CCOCC3)nc2c1]

To a solution of 2,5-dichlorobenzo[d]oxazole (5 g, 26.8 mmol) in dry dichloromethane (50 ml), morpholine (3.50 g, 40.3 mmol) was added. The reaction mixture was cooled to 0° C. To this cold reaction mixture triethylamine (4.0 g, 39.6 mmol) was added dropwise. After the addition was completed, the reaction mixture was allowed to stir at room temperature for 4 hours. After the completion of the reaction, the reaction mixture was treated with water (2×20 ml) and extracted with dichloromethane. The organic layers were separated, dried over Na₂SO₄, filtered and evaporated to afford a white solid which was triturated with diethyl ether to afford the title compound (5.2 g, 81%). ¹H-NMR (400 MHz, DMSO-d₆) δ=7.44 (d, J=8.40 Hz, 1H), 7.36 (d, J=2.40 Hz, 1H), 7.06 (dd, J=2.00, 8.40 Hz, 1H), 3.71-3.73 (m, 4H), 3.59-3.61 (m, 4H). MS: 239.2 (M+H)⁺.

### Preparative Example 31: 3-(5-chlorobenzo[d]oxazol-2-yl)-6-oxa-3-azabicyclo[3.1.1]heptane

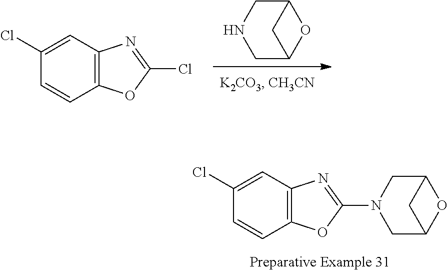 [SMILES: Clc1ccc2oc(Cl)nc2c1 ; C1NCC2CC1O2 ; Clc1ccc2oc(N3CC4CC(C3)O4)nc2c1]

To a solution of 6-oxa-3-azabicyclo[3.1.1]heptane (1 g, 10.09 mmol) and 2,5-dichlorobenzo[d]oxazole (1.897 g, 10.09 mmol) in acetonitrile (35 ml), K2CO3 (2.79 g, 20.17 mmol) was added and the mixture was refluxed at 90° C. overnight. The reaction mixture was filtered through celite and the filtrate was concentrated. The residue was purified by column chromatography using ethyl acetate in petroleum ether as eluent to afford title compound (1.68 g, 60%). ¹H-NMR (400 MHz, DMSO-d₆) δ=7.47 (d, J=8.40 Hz, 1H), 7.38-7.35 (m, 1H), 7.05 (dd, J=2.00, 8.60 Hz, 1H), 4.71 (d, J=6.40 Hz, 2H), 3.85-3.79 (m, 4H), 3.19-3.15 (m, 1H), 1.97 (d, J=9.20 Hz, 1H). MS: 251.3 (M+H)⁺.

### Preparative Example 32

Following the procedure described in Preparative example 31 the following compounds were prepared.

| | | | | 1. Yield; % |
|---|---|---|---|---|
| Preparative | Chloro | Amine | | 2. ¹H-NMR |
| example | Derivative | Derivative | Product | 3. MH⁺ (ESI) |
| Prep. Ex 32 5-chloro-2-(4- methoxypiperidin- 1- yl)benzo[d]oxazole | 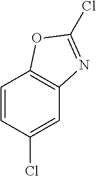 [SMILES: Clc1ccc2oc(Cl)nc2c1] | 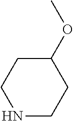 | 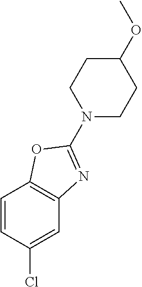 [SMILES: COC1CCN(c2nc3cc(Cl)ccc3o2)CC1] | 1. 97%. 2. ¹H-NMR (80 MHz, DMSO-d₆) δ 7.40 (d, J = 8.40 Hz, 1H), 7.32 (d, J = 2.40 Hz, 1H), 7.02 (dd, J = 2.00, 8.40 Hz, 1H), 3.86-3.80 (m, 2H), 3.49-3.42 (m, 3H), 3.29 (s, 3H), 1.96-1.91 (m, 2H), 1.59-1.52 (m, 2H), 3. 267.2 |

### Preparative Example 33: 4-(6-chlorobenzo[d]thiazol-2-yl)morpholine

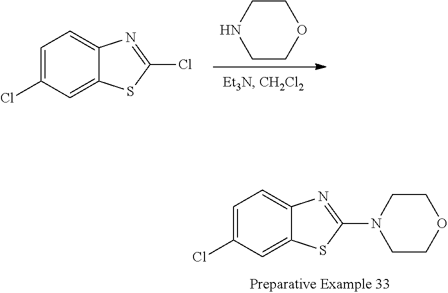 [SMILES: Clc1ccc2nc(Cl)sc2c1 ; C1COCCN1 ; Clc1ccc2nc(N3CCOCC3)sc2c1]

To a stirred solution of commercially available 2,6-dichlorobenzo[d]thiazole (500 g, 2.45 mol) in dichloromethane (4000 ml), triethylamine (1031 ml, 7.35 mol) and morpholine (290 ml, 3.67 mol) were added at 0° C. Then the reaction mixture was stirred at 25° C. for 48 hours. After completion of the reaction (monitored by TLC), water (3000 ml) was added to the reaction mixture, and then the reaction mixture was extracted using dichloromethane (2×2500 ml). The organic layers were dried over Na₂SO₄, filtered and evaporated under reduced pressure to afford the crude product. To the crude material was added methyl tert-butyl ether (1000 ml), and the mixture was stirred for 2 hours. The solid was collected by filtration and dried under vacuum for 6 hours to afford the title compound (530 g, 85%) as a pale brown solid. ¹H-NMR (400 MHz, DMSO-d₆) δ=7.93-7.94 (m, 1H), 7.43-7.44 (m, 1H), 7.28-7.29 (m, 1H), 3.72-3.74 (m, 4H), 3.54-3.55 (m, 4H). MS: 255.1 (M+H)⁺.

### Preparative Example 34: 4-(5-chlorobenzo[d]thiazol-2-yl)morpholine

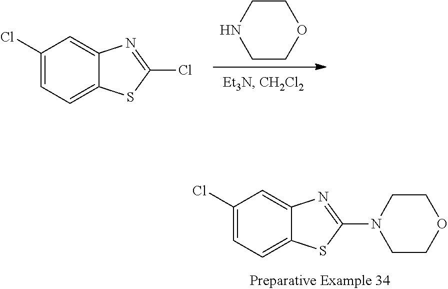 [SMILES: Clc1ccc2sc(Cl)nc2c1 ; C1COCCN1 ; Clc1ccc2sc(N3CCOCC3)nc2c1]

To a solution of 2,5-dichlorobenzo[d]thiazole (5 g, 24.5 mmol) in dry dichloromethane (50 ml) was added morpholine (3.19 g, 36.6 mmol) and the reaction mixture was cooled to 0° C. To this cold reaction mixture, was added triethylamine (3.71 g, 36.7 mmol) dropwise and the reaction mixture was allowed to stir at room temperature for 4 hours. The reaction mixture was treated with water (2×20 ml) and extracted with dichloromethane. The organic layer was separated, dried over Na₂SO₄, filtered and evaporated under reduced pressure to afford a white solid which was triturated with diethyl ether, filtered and dried to afford the title compound (4.5 g, 86%). ¹H-NMR (400 MHz, DMSO-d₆): δ=7.82 (d, J=8.00 Hz, 1H), 7.50 (d, J=2.00 Hz, 1H), 7.11-7.12 (m, 1H), 3.72-3.73 (m, 4H), 3.55-3.56 (m, 4H). MS: 255.4 (M+H)⁺.

### Preparative Example 35: 4-(6-bromothiazolo[4,5-b]pyridin-2-yl)morpholine

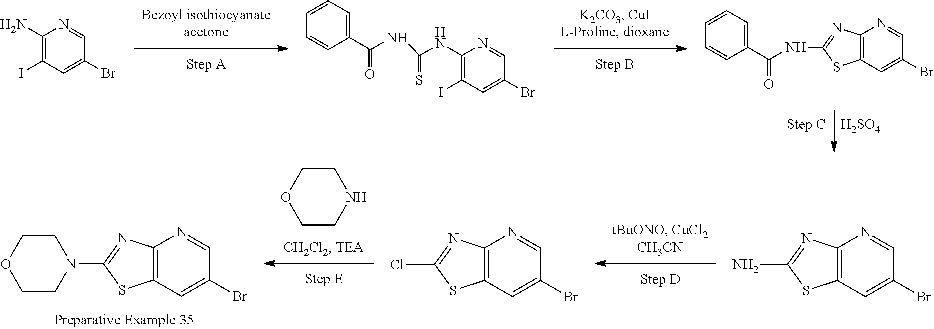

Step A

A solution of 5-bromo-3-iodopyridin-2-amine (5 g, 16.72 mmol) and benzoyl isothiocyanate (3.29, 20.07 mmol) in acetone (10 ml) was stirred at 60° C. for 12 hours, following progress by TLC. The solvent was evaporated and the solid was filtered, washed with n-hexane (200 ml) and dried to give the title compound as an off-white solid (4 g, 52%). ¹H-NMR (400 MHz, DMSO-d₆) δ=12.35 (s, 1H), 11.86 (s, 1H), 8.64-8.65 (m, 2H), 7.98-7.99 (m, 2H), 7.67 (s, 1H), 7.56 (d, J=9.40 Hz, 2H). MS: 461.5 (M+H)⁺.

Step B

To a solution of the title compound from Step A above (4 g, 12.1 mmol) in 1,4-dioxane (60 ml), was added potassium carbonate (2.5 g, 18.15 mmol), L-proline (0.28 g, 2.43 mmol) and copper(I) iodide (0.462 g, 2.43 mmol). Then, the reaction mixture was stirred at 80° C. for 16 hours, following progress by TLC. The reaction mixture was poured into 1.0 L of water and 1.0 L of aqueous saturated solution of NH₄Cl. The suspension was stirred at room temperature for 1 hour. The solid was filtered off, washed with aqueous saturated solution of NH₄Cl (2×300 ml) and water (2×300 ml) and dried to give the title compound (2.5 g, 62%) as an off-white solid. MS: 334.51 (M+H)⁺.

Step C

A suspension of the title compound from Step B above (2 g, 5.98 mmol) in 70% H₂SO₄ (20 ml) was heated at 120° C. for 2 hours. The reaction mixture was cooled to room temperature and the reaction mixture was slowly poured into 100 ml of cold water (0° C.). Then, the reaction mixture was adjusted to basic pH by addition of 50% aqueous NaOH. Then, the compound was extracted with EtOAc (6×150 ml). The combined organic layers were dried over with Na₂SO₄, filtered and concentrated under reduced pressure to afford the title compound (0.3 g, 23%) as a light yellow solid. ¹H-NMR (400 MHz, DMSO-d₆): δ=8.27-8.31 (m, 2H), 8.11 (s, 2H). MS: 230.4 (M)⁺.

Step D

To a suspension of the title compound from Step C above (0.3 mg, 1.3 mmol) in acetonitrile (5 ml) at 0° C. was added tert-butyl nitrite (0.2 ml, 1.95 mmol) over a period of 10 minutes with a syringe. Then, copper (II) chloride (0.2 g, 1.56 mmol) was added portion wise. After 30 minutes at 0° C., the reaction mixture was allowed to warm to room temperature for 1 hour and then was heated to 65° C. and stirred for 4 hours. The progress of the reaction was monitored by TLC. After completion of the reaction, the solvent was evaporated, and the product was diluted with water (20 ml) and 5% MeOH/DCM (3×20 ml). The combined organics were washed with brine (10 ml), dried over Na₂SO₄, filtered and concentrated under reduced pressure. The crude compound was purified by silica gel (60-120) column chromatography, eluted with 1% MeOH/DCM to afford the title compound (0.15 g, 46%) as an off white solid. ¹H-NMR (400 MHz, DMSO-d₆): δ=8.91 (d, J=2.40 Hz, 1H), 8.82 (d, J=1.60 Hz, 1H). MS: 250.9 (M+H)⁺.

Step E

To a solution of the title compound from Step D above (0.18 g, 0.72 mmol) in dry dichloromethane (5 ml), was added triethylamine (0.3 ml, 2.16 mmol) and morpholine (0.074 g, 0.86 mmol) and the mixture was stirred at room temperature for 6 hours. The reaction mixture was concentrated under reduced pressure. The crude compound was purified by silica gel (60-120) column chromatography, eluting with petroleum ether/ethyl acetate to afford the title compound (0.18 g, 83%) as an off yellow solid. ¹H-NMR (400 MHz, DMSO-d₆): δ=8.49 (d, J=2.00 Hz, 1H), 8.38 (d, J=1.60 Hz, 1H), 3.72-3.74 (m, 4H), 3.61-3.62 (m, 4H)·MS: 300.0 (M+H)⁺.

### Preparative Example 36: 4-(5-chlorothiazolo[5,4-b]pyridin-2-yl)morpholine

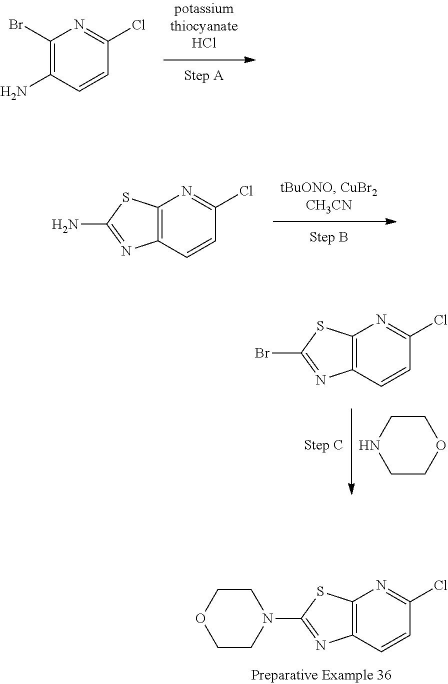

Step A

A solution of 2-bromo-6-chloropyridin-3-amine (5 g, 24.1 mmol) and potassium thiocyanate (7 g, 72.3 mmol) in ethanol (50 ml) and conc. hydrochloric acid (37%, 100 ml) was stirred at 100° C. for 45 hours The completion of the reaction was confirmed by TLC. The reaction mixture was cooled down to room temperature and concentrated to provide a brown solid, which was partitioned between dichloromethane (150 ml) and aqueous 1N NaOH (50 ml). The solid was filtered off and dried to afford the title compound (3.5 g, 79%) as a light yellow solid. MS: 186.1 (M+H)⁺.

Step B

To a suspension of the title compound from Step A above (1.5 g, 8.08 mmol) in acetonitrile (25 ml) at 0° C. was added tert-butyl nitrite (1.4 ml, 12.12 mmol) over a period of 10 minutes with a syringe. Then, copper (II) bromide (2.16 g, 9.69 mmol) was added portion wise. After 30 minutes at 0° C., the reaction mixture was allowed to warm to room temperature and stirred for 2 hours. The progress of the reaction was monitored by TLC. After completion of the reaction, the solvent was evaporated and the mixture was diluted with water (20 ml) and 5% MeOH/DCM (3×20 ml). The combined organics were washed with brine (10 ml), dried over Na₂SO₄, filtered and concentrated under reduced pressure. The crude compound was purified by silica gel (60-120) column chromatography, eluting with 1% MeOH/DCM to afford the title compound (0.65 g, 32%) as a pale-yellow solid. MS: 248.5 (M+H)⁺.

Step C

To a solution of the title compound from Step B above (0.65 g, 2.61 mmol) in dry dichloromethane (5 ml), was added triethylamine (1.1 ml, 7.83 mmol) and morpholine (0.34 g, 3.91 mmol) and the mixture was stirred at room temperature for 6 hours. The reaction mixture was concentrated under vacuum. The crude compound was purified by silica gel (60-120) column chromatography, eluting with petroleum ether/ethyl acetate to afford the title compound (0.6 g, 90%) as an off yellow solid. ¹H-NMR (400 MHz, DMSO-d₆) δ=7.83 (d, J=8.40 Hz, 1H), 7.41 (d, J=8.44 Hz, 1H), 3.72-3.74 (m, 2H), 3.59-3.60 (m, 2H). MS: 256.0 (M+H)⁺.

### Preparative Example 37: 4-(6-chlorothiazolo[4,5-c]pyridin-2-yl)morpholine

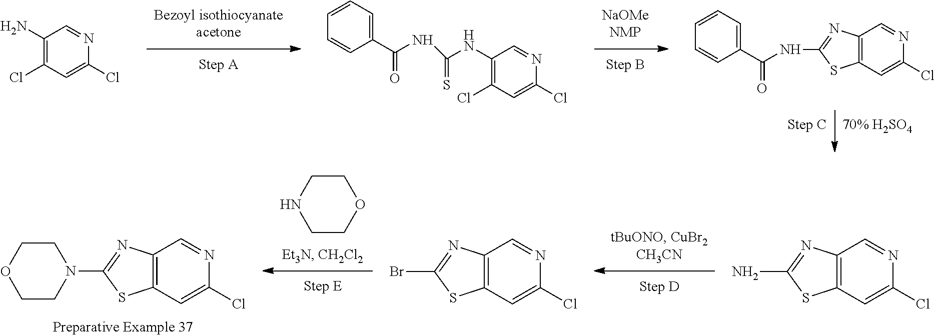

Step A

A solution of commercially available 4,6-dichloropyridin-3-amine (8.0 g, 49.07 mmol) and benzoyl isothiocyanate (7.3 ml, 53.98 mmol) in acetone (120 ml) was stirred at 60° C. for 3 hours. The reaction was monitored by the TLC. After completion, the solvent was evaporated and the solid was filtered off, washed with n-hexane (100 ml) and dried to give the title compound as an off-white solid (14.0 g, 87%). ¹H-NMR (400 MHz, DMSO-d₆): δ=12.39 (s, 1H), 12.02 (s, 1H), 8.74 (s, 1H), 7.98-7.99 (m, 3H), 7.67-7.68 (m, 1H), 7.56 (t, J=7.60 Hz, 2H). MS: 328.0 (M+H)⁺.

Step B

To a solution of the title compound from Step A above (14.0 g, 42.94 mmol) in N-methyl-2-pyrrolidone (70 ml) was added sodium methoxide (4.6 g, 85.88 mmol) at 0° C. The mixture was then heated to 120° C. and stirring was continued for 4 hours. The reaction was monitored by TLC. After completion, the reaction mixture was poured into cold water (300 ml) and a white precipitate was obtained. The solid was filtered off, washed with water (300 ml) and n-hexane (200 ml). The compound was dried under vacuum for 6 hours to give the title compound as a white solid (14.0 g, 100%). MS: 290.0 (M+H)⁺

Step C

A suspension of the title compound from Step B above (14.0 g, 48.4 mmol) in 70% H₂SO₄ (50 ml) was heated at 110° C. for 4 hours. The reaction mixture was cooled to room temperature and the reaction mixture was slowly poured into 200 mL of cold water (0° C.). Then, the reaction mixture was adjusted to basic pH by addition of solid 50% aq. NaOH. Then, the compound was extracted with EtOAc (6×100 ml). The combined organic layers were dried over with Na₂SO₄ and filtered, then the solvent was concentrated to give the title compound as a light-yellow solid (6 g, 67%). ¹H-NMR (400 MHz, DMSO-d₆): δ=8.30 (s, 1H), 7.86 (s, 1H). MS: 186.1 (M+H)⁺:

Step D

To a suspension of the title compound from Step C above (5.0 g, 27.02 mmol) in acetonitrile (120 mL) at 0° C. was added tert-butyl nitrite (4.8 ml, 40.54 mmol) over a period of 10 minutes with a syringe. Then, copper(II) bromide (9.0 g, 40.54 mmol) was added portion wise. After 30 minutes at 0° C., the reaction mixture was allowed to warm to room temperature for 2.5 hours, the progress of the reaction was monitored by TLC. After completion of the reaction, solvent was evaporated and the crude residue was partitioned between water (200 ml) and 5% MeOH/DCM (200 ml). The aqueous phase was separated, extracted further with 5% MeOH/DCM (2×200 ml). The combined organics were washed with brine (50 ml), dried over Na₂SO₄, filtered and concentrated under reduced pressure to afford the title compound as a white solid (6.5 g). The product was taken as such for the next step. MS: 250.9 (M+H)⁺:

Step E

To a solution of the title compound from Step D above (6.5 g, 26.09 mmol) in dry DCM (100 ml) was added triethylamine (11.2 ml, 81.5 mmol) and morpholine (2.8 ml, 28.13 mmol). The reaction mixture was stirred at room temperature for 4 hours. The reaction mixture was concentrated under reduced pressure. The crude reaction mixture was purified on a silica gel column using a Biotage Isolera One purification system eluting with a gradient of EtOAc/hexane gradient (10/80 to 80/20) to afford the title compound as a pale yellow solid (4.7 g, 71%). ¹H-NMR (400 MHz, DMSO-d₆): δ=8.48 (s, 1H), 8.05 (s, 1H), 3.73-3.74 (m, 4H), 3.60-3.61 (m, 4H). MS: 256.1 (M+H)⁺.

### Preparative Example 38: 5-bromo-2-morpholinobenzo[d]oxazole

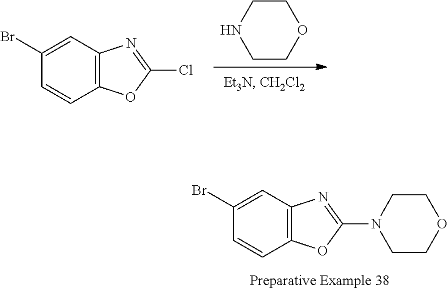 [SMILES: Clc1nc2cc(Br)ccc2o1 ; C1COCCN1 ; Brc1ccc2oc(N3CCOCC3)nc2c1]

To a solution of 5-bromo-2-chlorobenzo[d]oxazole (1 g, 4.30 mmol) in dry DCM (10 ml) at 0° C., morpholine (0.56 g, 6.42 mmol) and Et₃N (1.7 ml, 12.9 mmol) were added, and the resulting mixture was stirred at 25° C. for 4 hours. After completion of the reaction (monitored by TLC), the reaction mixture was diluted with H₂O (10 ml) and extracted with DCM (10 ml×2). The combined organic extracts were dried over Na₂SO₄, filtered and evaporated under reduced pressure to yield the crude product which was triturated with diethyl ether (100 ml), filtered, washed with diethyl ether (5 ml) and dried to afford the title compound (0.85 g, 71%) as an off-white solid. ¹H-NMR (400 MHz, DMSO-d₆) δ=7.48 (d, J=2.40 Hz, 1H), 7.34-7.38 (m, 1H), 7.16-7.17 (m, 1H), 3.70-3.72 (m, 4H), 3.58-3.59 (m, 4H) MS: 283.0 (M+H)⁺.

### Preparative Example 39: 6-chloro-2-morpholinooxazolo[4,5-c]pyridine

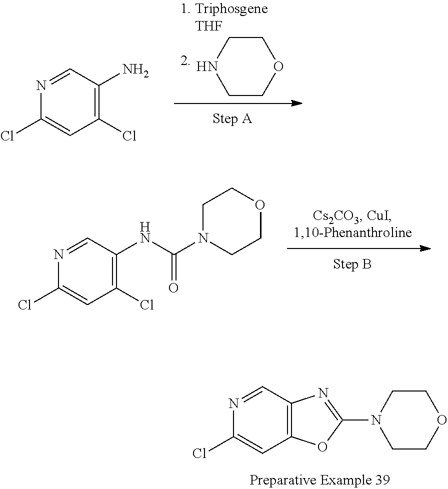 [SMILES: Nc1cnc(Cl)cc1Cl ; C1COCCN1 ; O=C(Nc1cnc(Cl)cc1Cl)N1CCOCC1 ; Clc1cc2oc(N3CCOCC3)nc2cn1]

Step A

To a stirred solution of 4,6-dichloropyridin-3-amine (2.5 g, 15.3 mmol) in THF (50 ml) was added triphosgene (4.55 g, 15.3 mmol) in THF dropwise, followed by triethylamine (4.28 ml, 30.7 mmol) and the resulting mixture was heated to reflux for 2 hours The reaction mixture was concentrated under vacuum. The residue was dissolved in acetonitrile (50 ml) and toluene (50 ml) and morpholine (1.34 g, 15.3 mmol) was added, then the mixture was heated at 110° C. for 12 hours, following progress by TLC. Upon completion of the reaction, the crude mixture was concentrated and purified directly by silica gel column chromatography using petroleum ether/EtOAc (20/80) to afford the title compound (3.0 g, 70.1%) as a white solid. ¹H-NMR (400 MHz, DMSO-d₆): δ=8.55 (s, 1H), 8.38-8.40 (m, 1H), 7.78-7.79 (m, 1H), 3.57-3.58 (m, 4H), 3.40-3.42 (m, 4H). MS: 276.0 (M+H)⁺.

Step B

To a solution of the title compound from Step A above (3.0 g, 10.8 mmol) in 1,4-dioxane (5 ml) was added Cs₂CO₃ (10.5 g, 32.4 mmol), 1,10-phenanthroline (0.972 g, 5.40 mol) and copper iodide (1.03 g, 5.40 mmol) then the resulting mixture was heated at 120° C. for 12 hours. The reaction mixture was filtered through celite and washed with DCM/MeOH, concentrated and was purified on silica gel column using Biotage Isolera One purification system eluting with an EtOAc/hexane (40/60) to afford the title compound (0.150 g, 6%) as an off-white solid. MS: 240.1 (M+H)⁺.

### Preparative Example 40: 1-(2-morpholinobenzo[d]oxazol-5-yl)piperidin-4-one

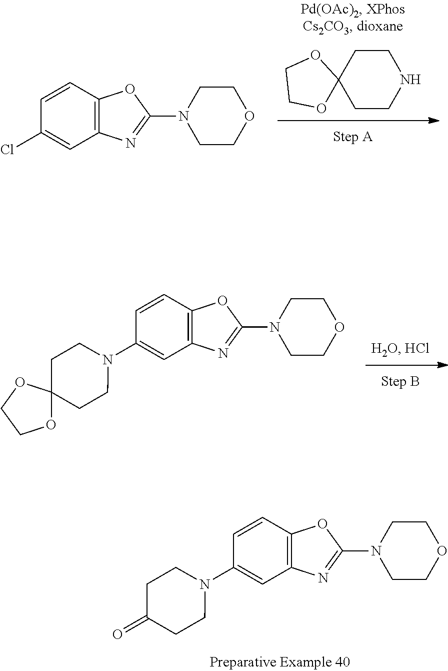

Step A

Palladium (II) acetate (0.188 g, 0.83 mmol) and 2-dicyclohexylphosphino-2′,4′,6′-triisopropylbiphenyl (XPhos; 1.20 g, 2.51 mmol) were added to a reaction vial and degassed 1,4-dioxane (80 ml) was added. The reaction mixture was degassed for 10 minutes under N₂ atmosphere. The suspension was heated at 100° C. for 10 minutes then 5-chloro-2-morpholino-1,3-benzoxazole (2 g, 8.38 mmol), 1,4-dioxa-8-azaspiro[4.5]decane (1.32 g, 9.22 mmol) and Cs₂CO₃ (8.19 g, 25.1 mmol) were added and the solution was heated at 100° C. for 12 hours. The reaction mixture was filtered through celite and the filtrate was concentrated under reduced pressure. The crude was purified on HP-Sil column (Biotage), eluting with a gradient of petroleum ether/ethyl acetate (100/0 to 20/80) to afford the title compound (2.3 g, 67.2%) as an off white solid. ¹H-NMR (400 MHz, DMSO-d⁶): δ 7.23 (d, J=8.80 Hz, 1H), 6.92 (d, J=2.40 Hz, 1H), 6.64-6.65 (m, 1H), 3.91 (s, 4H), 3.71 (t, J=5.20 Hz, 4H), 3.55 (t, J=4.40 Hz, 4H), 3.18 (t, J=5.60 Hz, 4H), 1.73 (t, J=5.60 Hz, 4H). MS: 346.2 (M+H)⁺.

Step B

To a stirred solution of the title compound from Step B above (2.3 g, 5.66 mmol) in water (15 ml), concentrated HCl was added (15 ml) and the resulting mixture was heated at 100° C. for 2 hours. The reaction mixture was basified using NaOH solution and the crude was extracted with ethyl acetate (2×500 ml). The organic layers were collected, dried over Na₂SO₄, filtered and concentrated under reduced pressure. The crude was purified on HP-Sil column (Biotage), eluting with a gradient of petroleum ether/ethyl acetate (100/0 to 20/80) to afford the title compound (1.1 g, 56.6%) as an off white solid. ¹H-NMR (400 MHz, DMSO-d₆): δ 7.27 (d, J=11.60 Hz, 1H), 7.01 (d, J=3.20 Hz, 1H), 6.71-6.72 (m, 1H), 3.57-3.63 (m, 4H), 3.33-3.50 (m, 8H), 2.43-2.45 (m, 4H). MS: 302.1 (M+H)⁺.

### Preparative Example 41: 2-morpholinobenzo[d]oxazol-5-amine

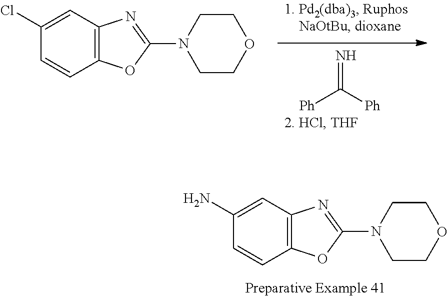 [SMILES: Clc1ccc2oc(N3CCOCC3)nc2c1 ; N=C(c1ccccc1)c1ccccc1 ; Nc1ccc2oc(N3CCOCC3)nc2c1]

5-chloro-2-morpholino-1,3-benzoxazole (2.0 g, 8.38 mmol) and diphenylmethanimine (1.67 g, 9.22 mmol) were added to a reaction vial containing degassed 1,4-dioxane (40 ml). To this, sodium tert-butoxide (2.42 g, 25.1 mmol), 2-dicyclohexylphosphino-2′,6′-di-i-propoxy-1,1′-biphenyl (Ruphos) (0.391 g, 0.838 mmol) and tris(dibenzylideneacetone)dipalladium(0) (0.241 g, 0.419 mmol) were added and the solution was heated at 100° C. for 16 hours in a sealed tube. The reaction mixture was filtered through celite and the filtrate was concentrated under reduced pressure. THF (10 ml) and 1.5N HCl (20 ml) were added to the crude and the resulting mixture was stirred for 3 hours at room temperature. Water was added (20 ml) to the reaction mixture followed by ethyl acetate (20 ml) and the phases were separated. The aqueous layer was basified with 10% NaOH solution. The precipitated solid was filtered, washed with water (20 ml) and dried under vacuum to afford the title compound (1.3 g, 68.6%) as a brown solid. 1H-NMR (400 MHz, DMSO-d⁶): δ 7.03 (d, J=11.20 Hz, 1H), 6.50 (s, 1H), 6.25 (t, J=8.80 Hz, 1H), 4.80 (s, 2H), 3.69 (t, J=6.40 Hz, 4H), 3.51 (t, J=6.00 Hz, 4H). MS: 220.1 (M+H)⁺.

### Preparative Example 42: 5-bromo-2-morpholinooxazolo[4,5-b]pyridine

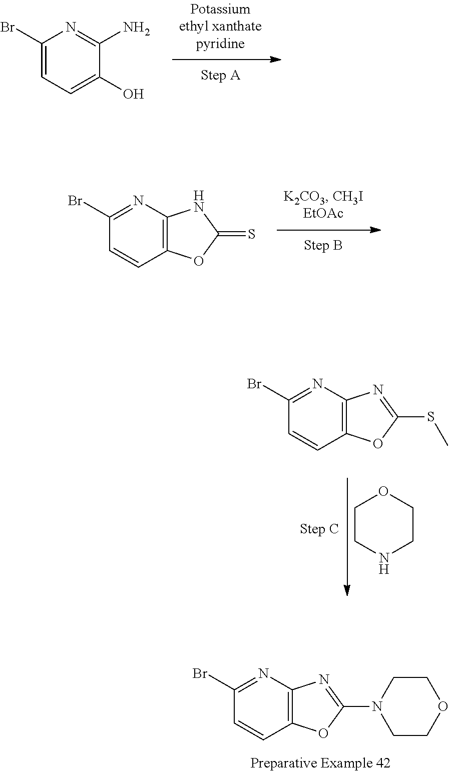 [SMILES: Nc1nc(Br)ccc1O ; S=c1[nH]c2nc(Br)ccc2o1 ; CSc1nc2nc(Br)ccc2o1 ; C1COCCN1 ; Brc1ccc2oc(N3CCOCC3)nc2n1]

Step A

To a stirred solution of 2-amino-6-bromopyridin-3-ol (2.5 g, 0.0133 mol) in pyridine (27 ml), was added potassium ethyl xanthate (6.39 g, 0.0399 mol) and the mixture was heated to 120° C. for 12 hours. The reaction mixture was acidified with a solution of 1.5N HCl and the precipitated solid was filtered and dried under vacuum to afford the title compound (2.4 g, 80%) as a pale yellow solid. The crude was directly used for the next step without further purification. MS: 231.0 (M+H)⁺.

Step B

To a stirred solution of the title compound from Step A above (2.4 g, 0.0104 mol) in ethyl acetate (20 ml), was added potassium carbonate (2.01 g, 0.0161 mol) followed by methyl iodide (1.0 ml, 0.0146 mol). The resulting mixture was stirred at 25° C. for 12 hours. To the crude mixture was added water (20 ml), and the phases were separated; the aqueous phase was extracted with ethyl acetate (2×30 ml), the combined organics were washed with water and brine, dried over Na₂SO₄, filtered and concentrated under reduced pressure to afford the title compound (2.3 g, 92%) as a pale-yellow solid. MS: 231.0 (M+H)⁺.

Step C

To the title compound from Step B above (2.3 g, 0.0094 mol) was added morpholine (20 ml) and the mixture was heated to 80° C. for 12 hours. The reaction mixture was poured into water, the precipitated solid was filtered off and dried under vacuum to afford the title compound (2.0 g, 76%) as an off-white solid. MS: 284.0 (M+H)⁺.

### Preparative Example 43: 6-bromo-2-morpholinooxazolo[5,4-b]pyridine

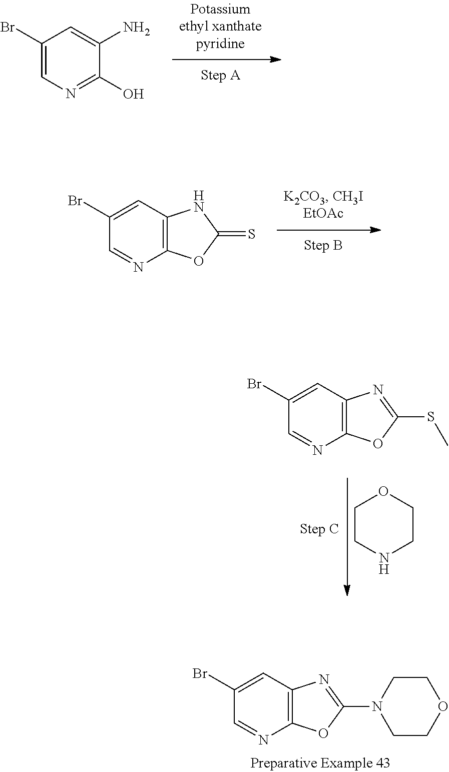 [SMILES: Nc1cc(Br)cnc1O ; S=c1[nH]c2cc(Br)cnc2o1 ; CSc1nc2cc(Br)cnc2o1 ; C1COCCN1 ; Brc1cnc2oc(N3CCOCC3)nc2c1]

Step A

To a stirred solution of 3-amino-5-bromo-pyridin-2-ol (2.0 g, 0.0105 mol) in pyridine (30 ml) was added potassium ethyl xanthate was added (1.85 g, 0.0115 mol) and the mixture was heated to 120° C. for 12 hours. The reaction mixture was acidified with a solution of 1.5N HCl and the precipitated solid was filtered and dried under vacuum to afford the title compound (1.5 g, 61%) as a pale yellow solid. MS: 228.9 (M−2H)⁺.

Step B

To a stirred solution of the title compound from Step A above (1.5 g, 0.00643 mol) in ethyl acetate (30 ml) was added potassium carbonate (1.24 g, 9 mmol mol) and methyl iodide (0.06 ml, 9.5 mmol) and the mixture was stirred at 25° C. for 12 hours. To the reaction mixture was added water (30 ml), the phases were separated; the aqueous phase was extracted with ethyl acetate, washed with water and brine. The combined organic layers were concentrated under vacuum to afford the title compound (1.5 g, 88%) as a pale yellow solid. ¹H-NMR (400 MHz, DMSO-d₆): δ 8.36-8.37 (m, 2H), 2.78 (s, 3H). MS: 245.0 (M+H)⁺.

Step C

To the title compound from Step B above (1.5 g, 5.69 mmol) was added morpholine (9.96 ml) and the mixture was heated to 80° C. for 12 hours. The reaction mixture was poured into water (10 ml) and the precipitated solid was filtered and dried under vacuum to afford the title compound (1.2 g, 72%) as an off-white solid. ¹H NMR (400 MHz, DMSO-d₆): δ 8.00 (s, 1H), 7.91 (s, 1H), 3.72-3.73 (m, 4H), 3.63-3.64 (m, 4H). MS: 286.1 (M+2H)⁺.

### Preparative Example 44: 6-bromo-2-morpholinooxazolo[4,5-b]pyridine

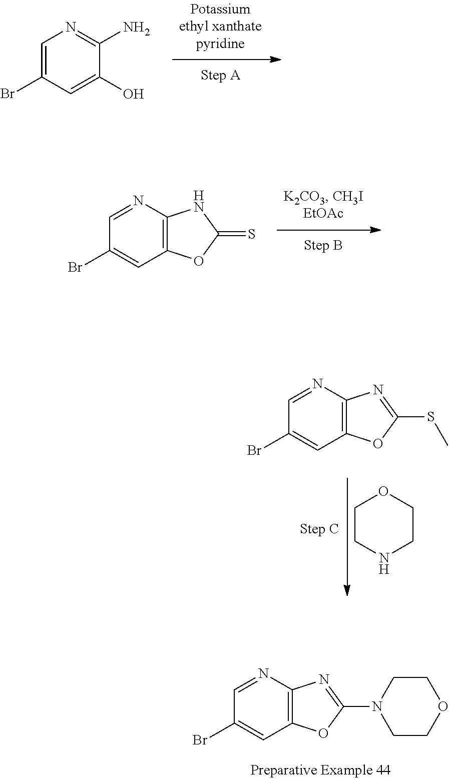 [SMILES: Nc1ncc(Br)cc1O ; S=c1[nH]c2ncc(Br)cc2o1 ; CSc1nc2ncc(Br)cc2o1 ; C1COCCN1 ; Brc1cnc2nc(N3CCOCC3)oc2c1]

Step A

To a stirred solution of 2-amino-5-bromopyridin-3-ol (3.0 g, 0.0159 mol) in pyridine (30 ml), was added potassium ethyl xanthate (7.7 g, 0.0498 mol) and the mixture was then heated to 120° C. for 12 hours. The reaction mixture was acidified with a 1.5N solution of HCl and the precipitated solid was filtered off and dried under vacuum to afford the title compound (2.65 g, 72%) as a pale yellow solid. ¹H-NMR (400 MHz, DMSO-d₆): δ 8.36 (s, 1H), 8.26 (s, 1H). MS: 233.0 (M+2H)⁺.

Step B

To a stirred solution of the title compound from Step A above (2.65 g, 0.0115 mol) in ethyl acetate (30 ml), was added potassium carbonate (2.22 g, 0.0161 mol) and methyl iodide (1.04 ml, 0.0161 mol) and the mixture was stirred at 25° C. for 12 hours. The reaction mixture was extracted with ethyl acetate (2×20 ml), washed with water (20 ml) and brine (20 ml). The organic layers were combined and concentrated under reduced pressure to afford the title compound (2.4 g, 86%) as a pale-yellow solid. MS: 245.0 (M+H)⁺.

Step C

To the title compound from Step B above (2.4 g, 0.0099 mol), was added morpholine (20 ml) and the mixture was heated to 80° C. for 12 hours. The reaction mixture was poured into water (20 ml), the precipitated solid was filtered off and dried under vacuum to afford the title compound (2.2 g, 78%) as an off-white solid. ¹H-NMR (400 MHz, DMSO-d₆): δ 8.26 (s, 1H), 8.12 (s, 1H), 3.73-3.74 (m, 4H), 3.64-3.65 (m, 4H). MS: 284.0 (M+H)⁺.

### Preparative Example 45: 6-bromo-2-morpholinooxazolo[5,4-c]pyridine

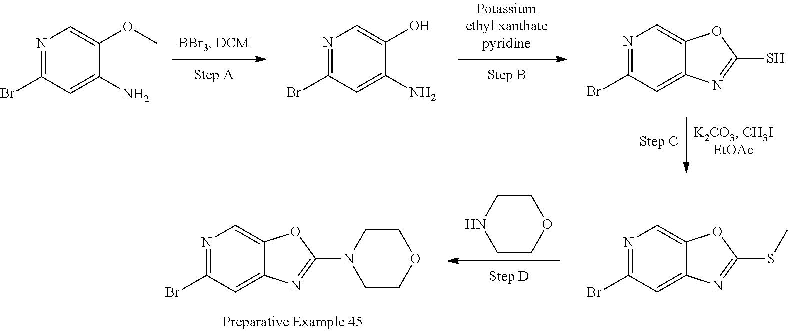

Step A

A solution of 2-bromo-5-methoxy-pyridin-4-amine (1.80 g, 0.00878 mol) in dichloromethane (50 ml) was cooled to −78° C. and BBr₃ (52.7 ml, 0.0527 mol) was added slowly and the mixture was stirred at room temperature for 12 hours. Then, the reaction mixture was concentrated, neutralized with a 10% aqueous solution of sodium bicarbonate at 0° C. and extracted with ethyl acetate (50 ml). The organic layer was washed with brine and concentrated to afford the title compound (1.0 g, 58%) as an off-white solid. ¹H NMR (400 MHz, DMSO-d₆): δ 9.57 (s, 1H), 7.46 (s, 1H), 6.61 (s, 1H), 5.85 (s, 2H). MS: 188.9 (M+H)⁺.

Step B

To a stirred solution of the title compound from Step A above (1.0 g, 5.13 mmol) in pyridine (15 ml), was added potassium ethyl xanthate (0.905 g, 5.65 mmol) and the mixture was heated to 120° C. for 12 hours. The reaction mixture was acidified with 1.5N solution of HCl, partitioned between ethyl acetate (30 ml) and water (30 ml). The organic phase was separated and the aqueous phase was extracted with ethyl acetate (2×50 ml). The combined organics were dried over Na₂SO₄, filtered and the solvents were evaporated under reduced pressure to afford the title compound (0.4 g, 33%) as a pale-yellow solid. ¹H-NMR (400 MHz, DMSO-d₆): δ 8.51 (s, 1H), 7.51 (s, 1H). MS: 230.9 (M+H)⁺.

Step C

To a stirred solution of the title compound from Step B above (0.4 g, 0.0017 mol) in ethyl acetate (10 ml), was added potassium carbonate (0.47 g, 0.0036 mol) and methyl iodide (0.1 ml, 0.0025 mol) and the mixture was stirred at 25° C. for 12 hours. The reaction mixture was partitioned between ethyl acetate (30 ml) and water (30 ml). The organic phase was separated, and the aqueous phase was extracted with ethyl acetate (2×50 ml). The combined organics were dried over Na₂SO₄, filtered and concentrated under reduced pressure to afford the title compound (0.4 g, 94%) as a pale-yellow solid. MS: 245.0 (M+H)⁺.

Step D

To the title compound from Step C above (0.4 g, 1.6 mmol) was added morpholine (2.8 ml and the mixture was heated to 80° C. for 12 hours. The reaction mixture was concentrated and purified directly by silica gel column chromatography eluting with petroleum ether/ethyl acetate (70/30) to afford the title compound (0.3 g, 65%) as an off-white solid. ¹H-NMR (400 MHz, CDCl₃): δ 8.42 (s, 1H), 7.52 (s, 1H), 3.68-3.69 (m, 8H). MS: 286.0 (M+2H)⁺.

### Preparative Example 46: 4-(6-chlorothiazolo[5,4-b]pyridin-2-yl)morpholine

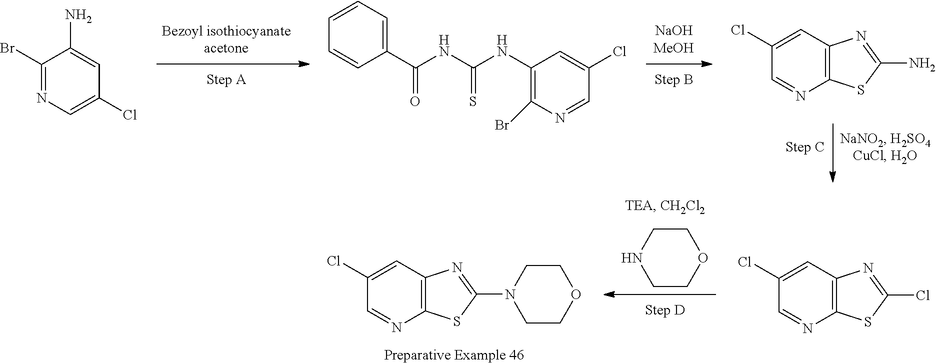

Step A

A solution of 2-bromo-5-chloropyridin-3-amine (10 g, 0.0482 mol) and benzoyl isothiocyanate (8.43 ml, 0.0675 mol) in acetone (150 ml) was stirred at room temperature for 18 hours. After completion of the reaction (monitored by TLC), the reaction mixture was evaporated under reduced pressure and the solid was filtered off, washed with n-hexane (200 ml) and dried under vacuum to give the title compound (7.1 g, 86.6%) as a white solid. ¹H-NMR (400 MHz, CDCl3): δ 13.01 (s, 1H), 9.21 (s, 1H), 9.09 (s, 1H), 8.26 (s, 1H), 7.96 (d, J=10.40 Hz, 2H), 7.28-7.57 (m, 3H). MS: 367.9 (M−2H)⁺.

Step B

To a suspension of the title compound from Step A above (15 g, 0.0404 mol) in 3.0N solution of NaOH (200 ml) and MeOH (100 ml) was refluxed for 1 hour. The reaction mixture was cooled to 0° C. and the precipitated solid was filtered off and dried to under vacuum to afford the title compound (7 g, 93%) as a brown solid. MS: 186.1 (M+H)⁺.

Step C

To a stirred solution of the title compound from Step B above (1 g, 5.38 mmol) in 3.0N solution of H₂SO₄ (100 ml), sodium nitrate (0.52 g, 7.52 mmol) in water (10 ml) was added dropwise at 0° C. and the mixture was stirred at 0° C. for 30 minutes. Then, copper chloride (1.01 g, 7.54 mmol) in concentrated HCl (10 ml) was added dropwise at 0° C. The reaction mixture was allowed to warm to room temperature and stirred for 6 hours. After completion of the reaction (monitored by TLC), the reaction mixture was diluted with water (50 ml) and extracted with ethyl acetate (20 ml×3). The combined organics were washed with brine (10 ml), dried over Na₂SO₄, filtered and concentrated under reduced pressure. The crude material was purified by silica-gel (60-120) column chromatography eluting with ethyl acetate/petroleum ether (20/80) to afford the title compound (700 mg, 63%) as a brown solid. ¹H-NMR (400 MHz, CDCl3): δ 8.58 (s, 1H), 8.20 (s, 1H). MS: 205.1 (M+H)⁺.

Step D

To a stirred solution of the title compound from Step C above (700 mg, 0.00341 mol) in dry DCM (10 ml), morpholine (356 mg, 0.00409 mol) and triethylamine (0.95 ml, 0.00662 mmol) were added and the mixture was stirred at room temperature for 16 hours. The reaction mixture was concentrated to afford the title compound (900 mg, 96.8%) as a brown solid. ¹H-NMR (400 MHz, CDCl3): δ 8.18 (s, 1H), 7.72 (s, 1H), 3.85-3.86 (m, 4H), 3.68-3.69 (m, 4H). MS: 256.1 (M+H)⁺.

### Preparative Example 47: 2-morpholino-5-(4,4,5,5-tetramethyl-1,3,2-dioxaborolan-2-yl)benzo[d]oxazole

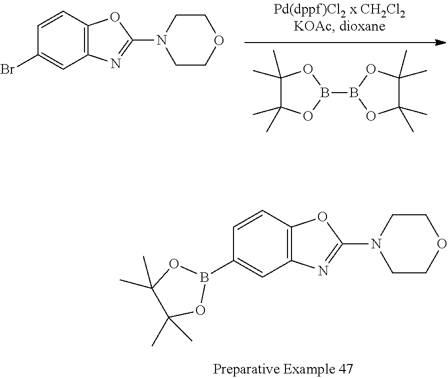

5-bromo-2-morpholinobenzo[d]oxazole (2.2 g, 7.77 mmol) and 4,4,4′,4′,5,5,5′,5′-octamethyl-2,2′-bi(1,3,2-dioxaborolane) (2.171 g, 8.55 mmol) were added to a sealed tube and degassed 1,4-dioxane (20 ml) was added. Then, [1,1′-bis(diphenylphosphino)ferrocene]dichloropalladium(II) (0.569 g, 0.777 mmol) and potassium acetate (2.288 g, 23.31 mmol) were added and the solution was purged with nitrogen and heated at 80° C. for 12 hours. After completion of the reaction as evidenced by TLC, the reaction mixture was filtered through celite, washed with DCM/MeOH (1/1, 20 ml) solution and concentrated under reduced pressure. The crude was purified on silica gel column using Biotage Isolera One purification system, eluting with ethyl acetate/hexane (75/25) to afford the title compound (2.3 g, 73%) as a brown solid. MS: 331.1 (M+H)⁺.

### Preparative Example 48: 5-ethynyl-2-morpholinobenzo[d]oxazole

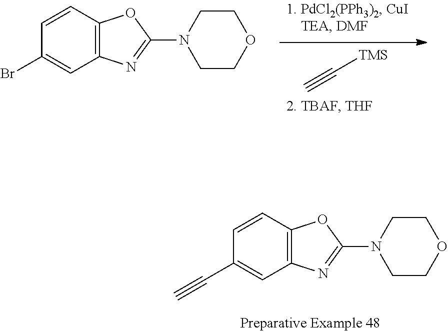

To a solution of 5-bromo-2-morpholinobenzo[d]oxazole (2 g, 7.06 mmol) in DMF (20 ml), triethylamine (0.715 g, 7.06 mmol) and trimethylsilylacetylene (0.694 g, 7.06 mmol) were added and the reaction was purged with nitrogen for 5 minutes. Then, bis(triphenylphosphin)palladium(II)-dichloride (0.49 g, 0.706 mmol) and copper iodide (0.135 g, 0.706 mmol) were added and the reaction was heated at 80° C. for 16 hours. The reaction mixture was filtered through celite and the filtrate was concentrated under reduced pressure. The crude material was dissolved in THF (15 ml) and TBAF (1.392 g, 5.33 mmol) was added at 0° C. and the resulting mixture was stirred for 15 minutes. Water (30 ml) and ethyl acetate (30 ml) were added, the organic layer was separated, dried over Na₂SO₄ and concentrated under reduced pressure. The crude compound was purified on HP-Sil cartridge using a Biotage Isolera One purification system eluting with petroleum ether/ethyl acetate (25/75) to afford the title compound (0.95 g, 78%) 1H-NMR (400 MHz, DMSO-d₆): δ 7.39-7.40 (m, 2H), 7.16 (dd, J=1.60, 8.40 Hz, 1H), 4.08 (s, 1H), 3.72-3.73 (m, 4H), 3.59-3.60 (m, 4H). MS: 229.0 (M+H)⁺.

### Preparative Example 49: 4-fluoro-3-(piperidin-4-yl)-1-tosyl-1H-indazole Hydrochloride

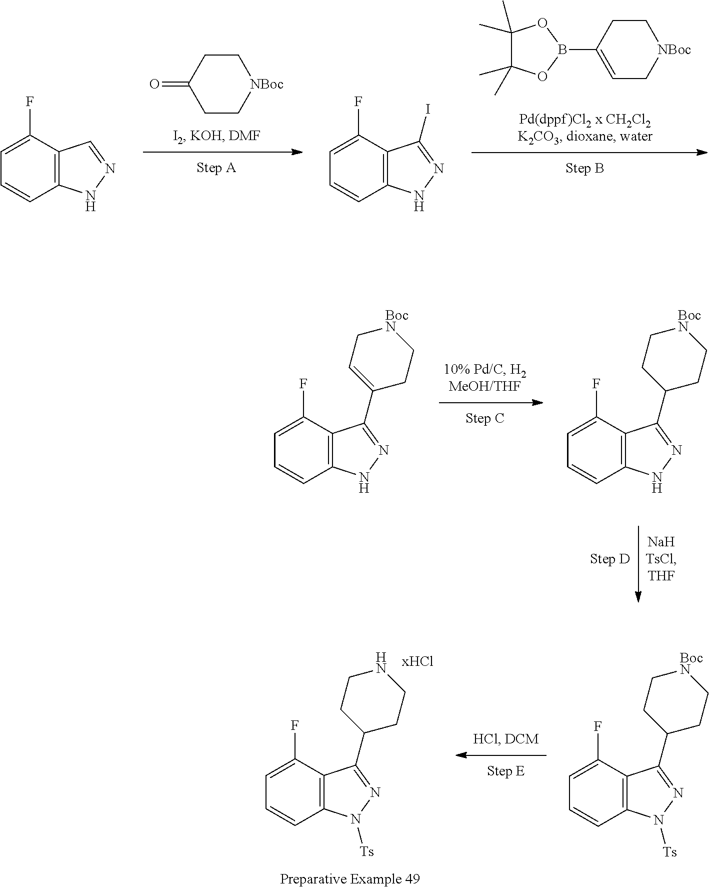

Step A

To a solution of 6-fluoro-1H-indazole (3 g, 22 mmol) in DMF (30 ml), KOH (4.64 g, 83 mmol) and iodine (8.39 g, 33.1 mmol) were added at 0° C. and the mixture was stirred at room temperature overnight. The mixture was poured into a saturated solution of sodium thiosulphate (10 ml). The solid formed was filtered using a sintered funnel and the filtrate was extracted with EtOAc (2×100 ml). The combined extracts were concentrated under reduced pressure to afford the title compound (3 g, 52%). ¹H-NMR (400 MHz, DMSO-d₆): δ 7.36-7.38 (m, 2H), 6.88-6.90 (m, 1H). MS: 261.0 (M+H)⁺.

Step B

In a sealed tube, compound from Step A above (3 g, 11.45 mmol) and tert-butyl 4-(4,4,5,5-tetramethyl-1,3,2-dioxaborolan-2-yl)-3,6-dihydropyridine-1(2H)-carboxylate (3.54 g, 11.45 mmol) were dissolved in 1,4-dioxane (30 ml) and water (7.5 ml). Then, K₂CO₃ (3.16 g, 22.90 mmol) and 1,1′-bis(diphenylphosphino)ferrocenedichloro palladium(II) dichloromethane complex (0.838 g, 1.14 mmol) were added under nitrogen atmosphere. The resulting reaction mixture was stirred at 100° C. for 12 hours. The mixture was filtered through celite and the filtrate was concentrated under reduced pressure; the crude residue was purified on HP-Sil column (Biotage), eluting with a gradient of petroleum ether/ethyl acetate (100/0 to 80/20) to afford the title compound (2.4 g, 65.4%). ¹H-NMR (400 MHz, CDCl₃): δ 7.28-7.29 (m, 2H), 6.83-6.85 (m, 1H), 6.57 (s, 1H), 4.12-4.14 (m, 2H), 3.70-3.71 (m, 2H), 2.80 (s, 2H), 2.80 (s, 9H). MS: 316.2 (M−H)⁻.

Step C

To a solution of compound from Step B above (2.4 g, 7.56 mmol) in methanol (30 ml) was added palladium hydroxide on carbon (0.8 g, 5.70 mmol). The reaction mixture was stirred under hydrogen gas in a miniclave (5 bar) at room temperature for 12 hours. The mixture was purged with nitrogen and filtered through celite. The filtrate was concentrated under reduced pressure to afford the title compound (2.2 g, 73%). 1H-NMR (400 MHz, CDCl₃): δ 7.25-7.29 (m, 2H), 6.77-6.79 (m, 1H), 4.26 (s, 2H), 3.33-3.34 (m, 1H), 2.93-2.96 (m, 6H), 1.48 (s, 9H). MS: 264.1 (M+H)⁺-t-butyl.

Step D

To a suspension of sodium hydride (60% in paraffin oil, 0.416 g, 17.33 mmol) in THF (60 ml), a solution of the title compound from Step C above (2.2 g, 6.93 mmol) in THF (50 ml) was added dropwise at 0° C. and the mixture was stirred at room temperature for 30 minutes under nitrogen atmosphere. Then a solution of TsCl (1.718 g, 9.01 mmol) in THF (20 ml) was added dropwise at 0° C. and the reaction mixture was further stirred at room temperature for 30 minutes. The mixture was filtered through celite and the filtrate was concentrated under reduced pressure to afford the title compound (2.4 g, 73%). MS: 374.1 (M+H)⁺-Boc.

Step E

To a solution of the title compound from Step D above (2.4 g, 5.07 mmol) in DCM (25 ml) a 4.0 M solution of HCl in 1,4-dioxane (10 ml) was added dropwise over a period of 5 minutes at 0° C. The mixture was stirred at 25° C. for 1 hour. After completion of the reaction (as monitored by TLC), the crude mixture was concentrated under reduced pressure and the mixture was basified with triethyl amine. Water (50 ml) and DCM (50 ml) were added and the phases were separated. The organic layer was dried over Na₂SO₄ and the solvent was removed under reduced pressure to afford the title compound (2 g, 100%). MS: 374.2 (M+H)⁺.

### Preparative Example 50: 6-fluoro-3-(piperidin-4-yl)-1-tosyl-1H-indazole Hydrochloride

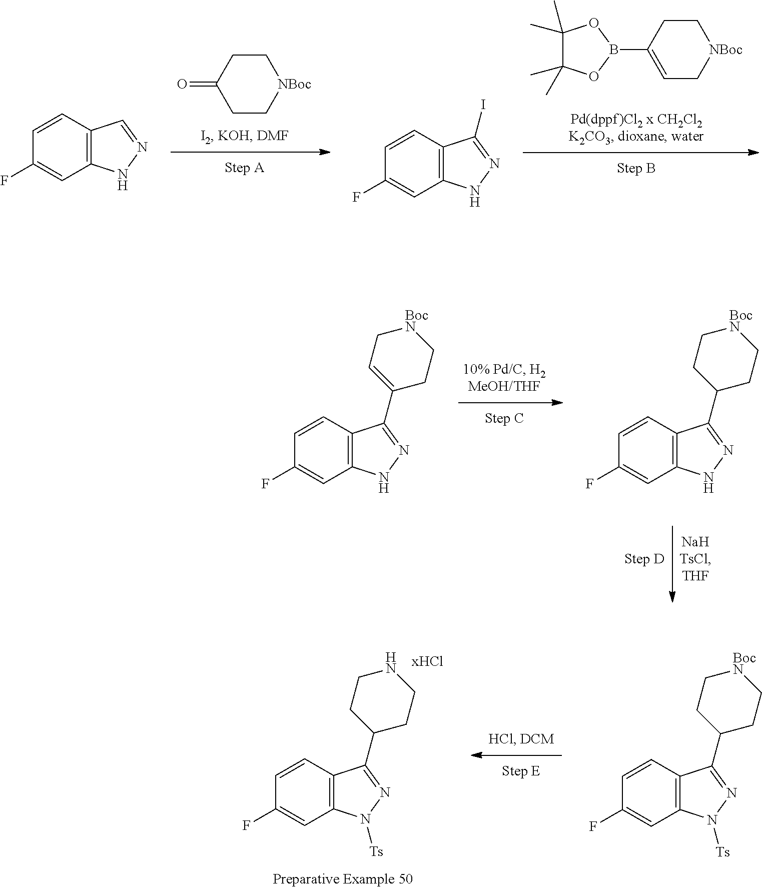

Step A

To a solution of 6-fluoro-1H-indazole (3 g, 22.04 mmol) in DMF (30 ml), KOH (4.64 g, 83 mmol) and iodine (8.39 g, 33.1 mmol) were added at 0° C. and the mixture was stirred at room temperature overnight. The mixture was poured into a saturated solution of sodium thiosulphate (20 ml). The solid formed was filtered using a sintered funnel and the filtrate was extracted with EtOAc (2×100 ml). The combined organic extracts were concentrated under reduced pressure to afford the title compound (5.2 g, 89%). ¹H-NMR (400 MHz, DMSO-d₆): δ 7.39-7.43 (m, 2H), 7.06 (t, J=12.00 Hz, 1H). MS: 262.9 (M+H)⁺.

Step B

In a sealed tube, the title compound from Step A above (5.1 g, 19.7 mmol) and tert-butyl 4-(4,4,5,5-tetramethyl-1,3,2-dioxaborolan-2-yl)-3,6-dihydropyridine-1(2H)-carboxylate (6.11 g, 19.77 mmol) were dissolved in 1,4-dioxane (50 ml) and water (12.5 ml). Then, K₂CO₃ (5.4 g, 39.5 mmol) and 1,1′-bis(diphenylphosphino)ferrocenedichloro palladium(II) dichloromethane complex (1.446 g, 1.977 mmol) were added under nitrogen atmosphere. The resulting reaction mixture was stirred at 100° C. for 4 hours. The mixture was filtered through celite and the filtrate was concentrated under reduced pressure and the crude was purified on HP-Sil column (Biotage), eluting with a gradient of petroleum ether/ethyl acetate (100/0 to 80/20) to afford the title compound (4.35 g, 67%). ¹H-NMR (400 MHz, DMSO-d₆): δ 13.05 (s, 1H), 8.01-8.02 (m, 1H), 7.30 (dd, J=2.00, 9.40 Hz, 1H), 6.99-7.00 (m, 1H), 6.54 (s, 1H), 4.09 (s, 2H), 3.56-3.58 (m, 2H), 2.53-2.54 (m, 2H), 1.43 (s, 9H). MS: 316.0 (M−H)⁺.

Step C

To a solution of the title compound from Step B above (4.33 g, 13.6 mmol) in methanol (50 ml) was added palladium hydroxide on carbon (1.91 g, 13.63 mmol). The reaction mixture was stirred under hydrogen pressure (5 bar) at room temperature for 12 hours. The mixture was purged with nitrogen then was filtered through celite. The filtrate was concentrated under reduced pressure to afford the title compound (3.67 g, 84%). MS: 264.2 (M+H)⁺-t-butyl.

Step D

To a suspension of sodium hydride (60% in paraffin oil, 1.158 g, 29.0 mmol) in THF (50 ml), a solution of the title compound from Step C above (3.676 g, 11.58 mmol) was added dropwise at 0° C. and then the reaction mixture was stirred at room temperature for 2 hours under nitrogen atmosphere. Then a solution of tosyl-chloride (2.87 g, 15.06 mmol) in THF (10 ml) was added dropwise at 0° C. and the reaction mixture was further stirred at room temperature for 3 hours. The mixture was filtered through celite and the filtrate was concentrated under reduced pressure to afford the title compound (3.88 g, 70%). ¹H-NMR (400 MHz, DMSO-d₆): δ 7.97-7.99 (m, 1H), 7.80-7.82 (m, 3H), 7.39 (d, J=8.00 Hz, 2H), 7.32 (t, J=9.20 Hz, 1H), 3.96-4.00 (m, 2H), 2.92 (bs, 2H), 2.33 (bs, 4H), 1.87-1.90 (m, 2H), 1.53-1.56 (m, 2H), 1.39 (s, 9H). MS: 374.1 (M+H)⁺-Boc.

Step E

To a solution of the title compound from Step D above (3.88 g, 8.19 mmol) in DCM (35 ml), a 4.0 M solution of HCl in 1,4-dioxane (10 ml) was added dropwise over a period of 5 minutes at 0° C. The mixture was stirred at 25° C. for 1 hour. After completion of the reaction (as monitored by TLC), the mixture was concentrated under reduced pressure and was basified with triethylamine. Water (50 ml) and DCM (50 ml) were added and the phases were separated. The organic layer was dried over Na₂SO₄ and the solvent was removed under reduced pressure to afford the title compound (3.3 g, 100%). MS: 374.3 (M+H)⁺.

### Preparative Example 51: 7-fluoro-3-(piperidin-4-yl)-1H-indazole Hydrochloride

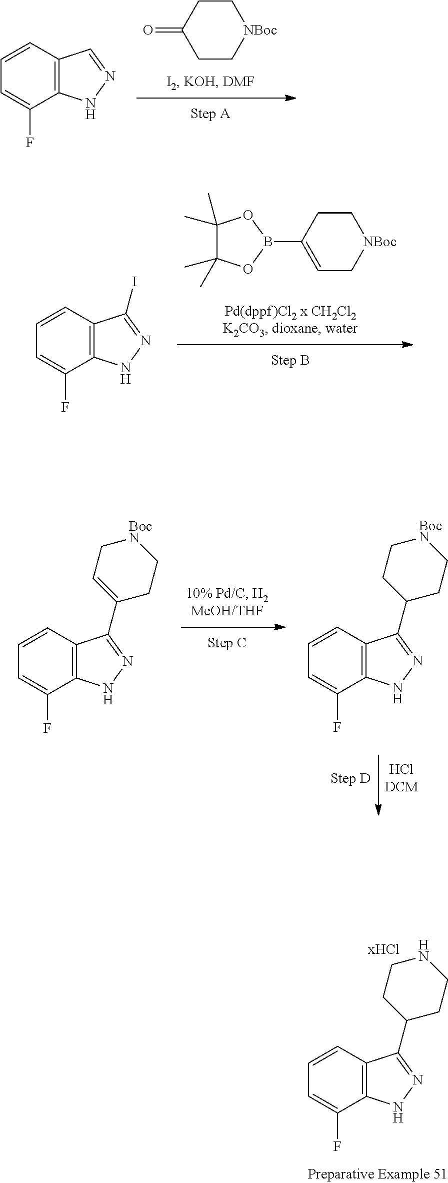

Step A

To a solution of 7-fluoro-1H-indazole (2 g, 14.69 mmol) in DMF (20 ml), KOH (3.05 g, 54.4 mmol) and iodine (5.59 g, 22.04 mmol) were added at 0° C. The mixture was stirred at 25° C. for 1 hour and then concentrated under reduced pressure. The crude was diluted with a solution of saturated sodium thiosulphate (200 ml) and extracted with ethyl acetate (2×400 ml). The combined organic extracts were washed with brine (200 ml), dried over anhydrous Na₂SO₄, filtered and concentrated under reduced pressure. The crude was purified on HP-Sil column (Biotage), eluting with a gradient of petroleum ether/ethyl acetate (100/0 to 80/20) to afford the title compound (3.8 g, 98%) as an off white solid. MS: 261.0 (M−H)⁻.

Step B

In a sealed tube, the title compound from Step A above (3.8 g, 14.50 mmol), tert-butyl 4-(4,4,5,5-tetramethyl-1,3,2-dioxaborolan-2-yl)-3,6-dihydropyridine-1(2H)-carboxylate (4.48 g, 14.50 mmol) in 1,4-dioxane (30 ml), K₂CO₃ (4.01 g, 29.0 mmol) dissolved in water (7.50 ml) and 1,1′-bis(diphenylphosphino)ferrocenedichloro palladium(II) dichloromethane complex (1.061 g, 1.450 mmol) were added under nitrogen atmosphere. The resulting reaction mixture was stirred at 100° C. for 16 hours. The mixture was diluted with water (100 ml) and extracted with ethyl acetate (2×200 ml). The combined organic extracts were washed with brine (200 ml), dried over anhydrous Na₂SO₄, filtered and concentrated under reduced pressure. The crude was purified on HP-Sil column (Biotage), eluting with a gradient of petroleum ether/ethyl acetate (100/0 to 70/30) to afford the title compound (3.5 g, 75%) as a yellow solid. ¹H-NMR (400 MHz, DMSO-d₆): δ 13.56 (s, 1H), 7.83 (d, J=8.00 Hz, 1H), 7.12-7.13 (m, 2H), 6.57 (s, 1H), 4.10 (s, 2H), 3.58-3.59 (m, 2H), 2.69 (bs, 2H), 1.42 (s, 9H). MS: 262.0 (M+H)⁺-t-butyl.

Step C

In an autoclave, a solution of the title compound from Step B above (3.6 g, 11.34 mmol) was dissolved in MeOH (80 ml) and placed under nitrogen atmosphere. Then, palladium hydroxide on carbon (0.797 g, 1.134 mmol) was added. The reaction mixture was stirred under hydrogen gas pressure (5 bar) at room temperature for 16 hours. The mixture was purged with nitrogen and filtered through celite. The filtrate was concentrated under reduced pressure to afford the title compound (3.6 g, 99%) as a yellow solid. MS: 264.1 (M+H)⁺-t-butyl.

Step D

To a solution of the title compound from Step C above (1 g, 3.13 mmol) in DCM (5 ml), a 4.0 M solution of HCl in 1,4-dioxane (7.83 ml, 31.3 mmol) was added dropwise over a period of 5 minutes at 0° C. The mixture was stirred at 25° C. for 4 hours. The reaction was concentrated under reduced pressure to afford the title compound (0.560 g, 81%) as an off white solid. ¹H-NMR (400 MHz, DMSO-d₆): δ 9.17 (bs, 2H), 7.71 (d, J=8.40 Hz, 1H), 7.09-7.10 (m, 2H), 3.57 (s, 1H), 3.38-3.39 (m, 2H), 3.05-3.08 (m, 2H), 2.10-2.11 (m, 4H). MS: 220.1 (M+H)⁺.

### Preparative Example 52: 5-methyl-3-(piperidin-4-yl)-1-tosyl-1H-indazole Hydrochloride

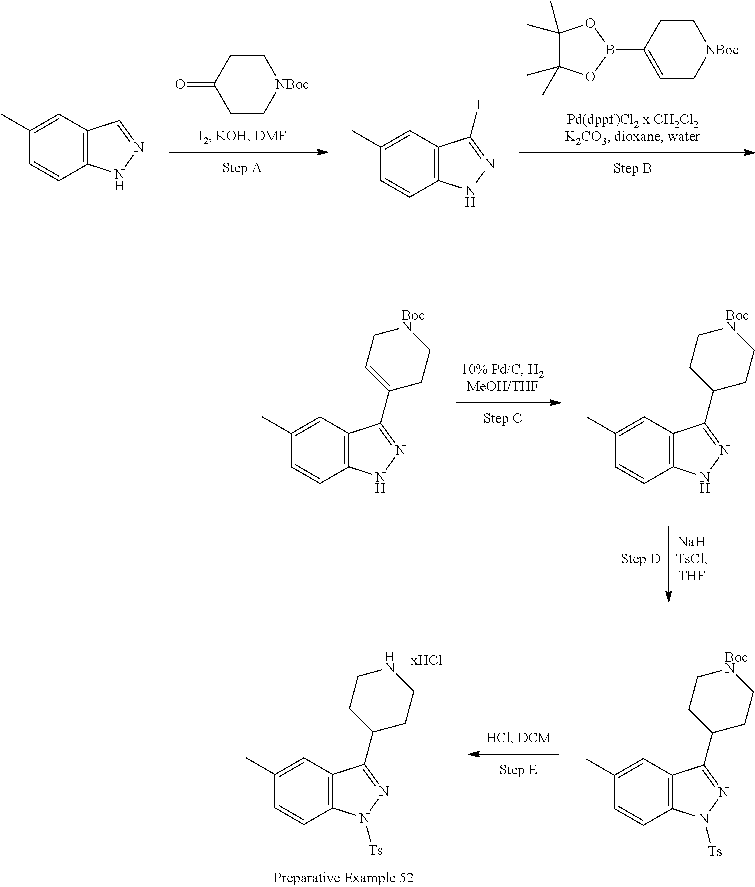

Step A

To a solution of 5-methyl-1H-indazole (3 g, 22.70 mmol) in DMF (50 ml), KOH (11.5 g, 205 mmol) was added. The reaction mixture was stirred at room temperature for 30 minutes. Then, iodine (4.5 g, 17.73 mmol) was added at 0° C. and the reaction mixture was stirred overnight at room temperature. The mixture was poured into a saturated solution of sodium thiosulphate (20 ml). The solid formed was filtered off, washed and dried to afford the title compound (4.6 g, 78%). ¹H-NMR (400 MHz, DMSO-d₆): δ 13.32 (s, 1H), 7.45 (d, J=8.80 Hz, 1H), 7.26-7.27 (m, 1H), 7.19 (s, 1H), 2.43 (s, 3H). MS: 257.0 (M−H)⁺.

Step B

In a sealed tube, a solution of the title compound from Step A above (4.5 g, 17.44 mmol) in 1,4-dioxane (80 ml), tert-butyl 4-(4,4,5,5-tetramethyl-1,3,2-dioxaborolan-2-yl)-3,6-dihydropyridine-1(2H)-carboxylate (5.39 g, 17.44 mmol), K₂CO₃ (4.82 g, 34.9 mmol) dissolved in water (12.5 ml). and PdCl₂(dppf) (1.276 g, 1.744 mmol) were added under continuous bubbling of nitrogen. The resulting reaction mixture was stirred at 100° C. for 12 hours and the crude was filtered through celite and concentrated under reduced pressure. The crude residue was purified on HP-Sil column (Biotage) eluting with a gradient of petroleum ether/ethyl acetate (100/0 to 70/30) to afford the title compound (5.2 g, 95%) as a white solid. ¹H-NMR (400 MHz, DMSO-d₆): δ 12.86 (s, 1H), 7.78 (s, 1H), 7.41 (d, J=11.20 Hz, 1H), 7.19 (d, J=11.20 Hz, 1H), 6.52 (s, 1H), 3.65-3.93 (m, 2H), 3.56-3.57 (m, 2H), 2.67-2.68 (m, 2H), 2.42 (s, 3H), 1.39 (s, 9H). MS: 258.2 (M+H)⁺-t-butyl.

Step C

To a solution of the title compound from Step B above (5 g, 15.95 mmol) in MeOH (150 ml), palladium hydroxide on carbon (1.5 g, 10.68 mmol) was added and the mixture was stirred under hydrogen pressure (1 bar) at room temperature overnight. The reaction was filtered through celite and the filtrate was concentrated under reduced pressure to afford the title compound (5 g, 98%). MS: 260.2 (M+H)⁺-t-butyl.

Step D

To a suspension of sodium hydride (60% in paraffin oil, 0.342 g, 14.27 mmol) in THF (80 ml), a solution of the title compound from Step C above (1.5 g, 4.76 mmol) was added dropwise at 0° C. and the mixture was stirred at room temperature for 2 hours under nitrogen atmosphere. Then a solution of tosyl-chloride (2.67 g, 14.01 mmol) in THF (10 ml) was added dropwise at 0° C. and the reaction mixture was further stirred at room temperature for 3 hours. The mixture was quenched with iced cold water (70 ml) and was extracted with ethyl acetate (2×100 ml). The combined organics were washed with brine (50 ml), dried over anhydrous Na₂SO₄, filtered and concentrated under reduced pressure. The crude was purified on HP-Sil column (Biotage) eluting with a gradient of petroleum ether/ethyl acetate (100/0 to 70/30) to afford the title compound (2 g, 87%) as a white solid. ¹H-NMR (400 MHz, DMSO-d₆): δ 7.96 (d, J=8.40 Hz, 1H), 7.69-7.70 (m, 3H), 7.47 (d, J=9.20 Hz, 1H), 7.35 (d, J=8.40 Hz, 2H), 3.97-4.00 (m, 2H), 3.22-3.24 (m, 1H), 2.92-2.95 (m, 2H), 2.43 (s, 3H), 2.34 (s, 3H), 1.87-1.90 (m, 2H), 1.58-1.61 (m, 2H), 1.43 (s, 9H). MS: 370.2 (M+H)+−Boc.

Step E

To a solution of the title compound from Step D above (300 mg, 0.639 mmol) in DCM (15 ml), a 4.0 M solution of HCl in 1,4-dioxane (0.019 ml, 0.639 mmol) was added dropwise over a period of 5 minutes at 0° C. The mixture was stirred at 25° C. for 4 hours. The reaction was concentrated under reduced pressure. The solid was then dissolved in DCM (15 ml) and the reaction mixture was basified using triethylamine (2 ml). The mixture was then diluted with water (10 ml) and extracted with DCM (2×30 ml). The combined organics were washed with brine (10 ml), dried over anhydrous Na₂SO₄, filtered and concentrated under reduced pressure to afford the title product (230 mg, 97%). ¹H-NMR (400 MHz, DMSO-d₆): δ 7.95 (d, J=8.40 Hz, 1H), 7.68-7.70 (m, 3H), 7.44-7.45 (m, 1H), 7.34-7.36 (m, 2H), 3.02-3.05 (m, 3H), 2.55-2.62 (m, 2H), 2.47 (s, 3H), 2.44 (s, 3H), 1.68-1.69 (m, 4H). MS: 370.1 (M+H)⁺.

### Preparative Example 53: 6-methyl-3-(piperidin-4-yl)-1-tosyl-1H-indazole Hydrochloride

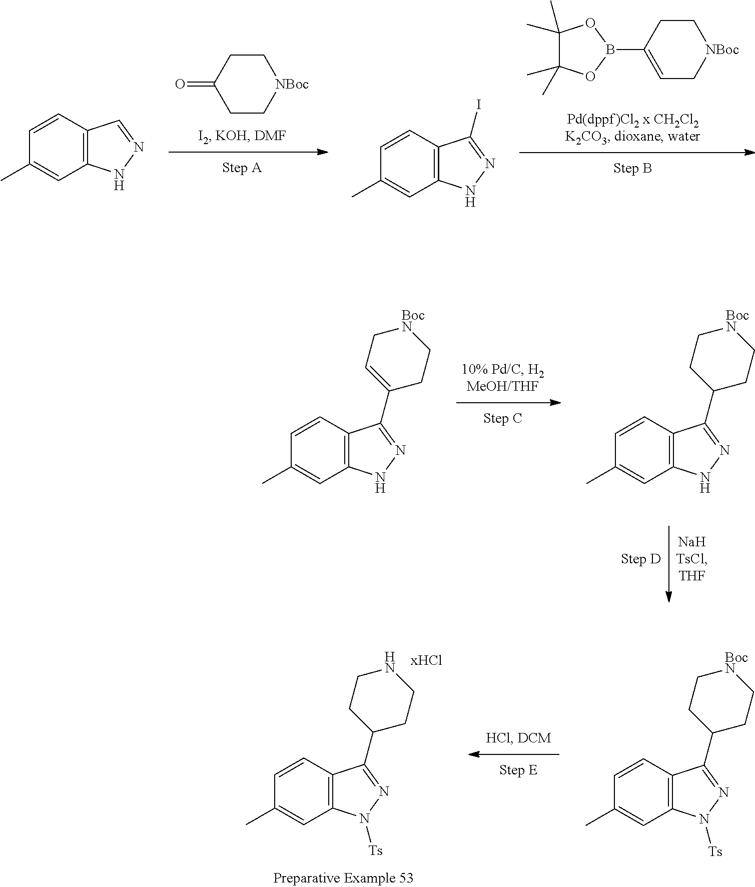

Step A

To a solution of 6-methyl-1H-indazole (3 g, 22.7 mmol) in DMF (30 ml), KOH (78 g, 85 mmol) was added and the mixture was stirred at room temperature for 30 minutes. Then, iodine (8.64 g, 34 mmol) was added at 0° C. and the mixture was stirred overnight at room temperature. The mixture was poured onto a saturated solution of sodium thiosulphate (20 ml). The solid formed was filtered using a sintered funnel and the filtrate was extracted with EtOAc (2×200 ml). The combined organics were concentrated under reduced pressure to afford the title compound (4.3 g, 73%). ¹H-NMR (400 MHz, DMSO-d₆): δ 13.39 (s, 1H), 7.30 (d, J=13.20 Hz, 2H), 7.03 (d, J=10.40 Hz, 2H), 2.51 (s, 3H). MS: 259.0 (M+H)⁺.

Step B

To a solution of the title compound from Step A above (4.3 g, 16.7 mmol) in 1,4-dioxane (50 ml), tert-butyl 4-(4,4,5,5-tetramethyl-1,3,2-dioxaborolan-2-yl)-3,6-dihydropyridine-1(2H)-carboxylate (5.19 g, 16.79 mmol), K₂CO₃ (4.64 g, 33.6 mmol) dissolved in water (12.5 ml). and 1,1′-bis(diphenylphosphino)ferrocenedichloro palladium(II) dichloromethane complex (1.228 g, 1.679 mmol) were added under continuous bubbling of nitrogen. The resulting reaction mixture was stirred at 100° C. for 4 hours then was filtered through celite and concentrated under reduced pressure to afford the title compound (5.7 g, >100%). MS: 258.2 (M+H)⁺-t-butyl.

Step C

To a solution of the title compound from Step B above (3.4 g, 10.85 mmol) in MeOH (50 ml), palladium hydroxide on carbon (1.524 g, 10.85 mmol) was added and the mixture was stirred under hydrogen gas atmosphere (5 bar) using a miniclave for 12 hours. The reaction was filtered through celite and the filtrate was concentrated to afford the title compound (3.2 g, 88%). MS: 260.2 (M+H)⁺-t-butyl.

Step D

To a suspension of sodium hydride (60% in paraffin oil, 0.776 g, 32.3 mmol) in THF (10 ml), a solution of the title compound from Step C above in THF (10 ml) was added dropwise at 0° C. and the mixture was stirred at room temperature for 30 minutes. Then a solution of tosyl-chloride (2.67 g, 14.01 mmol) in THF (10 ml) was added dropwise at 0° C. and the reaction mixture was further stirred at room temperature for 1 hour. The mixture was quenched with ice and extracted with ethyl acetate. The organic layer was collected, dried over anhydrous Na₂SO₄, filtered and concentrated under reduced pressure. The crude was purified on HP-Sil column (Biotage) eluting with a gradient of petroleum ether/ethyl acetate (100/0 to 80/20) to afford the title compound (3.4 g, 65.6%). 1H-NMR (400 MHz, MeOD): δ 7.97 (s, 1H), 7.77 (d, J=8.40 Hz, 2H), 7.67 (d, J=8.40 Hz, 1H), 7.31 (d, J=8.00 Hz, 2H), 7.22-7.23 (m, 1H), 4.11-4.13 (m, 2H), 3.24-3.25 (m, 1H), 3.15-3.16 (m, 2H), 2.56 (s, 3H), 2.36 (s, 3H), 1.91-1.92 (m, 2H), 1.79-1.79 (m, 2H), 1.51 (s, 9H). MS: 370.2 (M+H)+−Boc.

Step E

To a solution of the title compound from Step D above (3.2 g, 6.81 mmol) in DCM (30 ml), a 4.0 M solution of HCl in 1,4-dioxane (10 ml) was added dropwise over a period of 5 minutes at 0° C. The mixture was stirred at 25° C. for 1 hour. The reaction was concentrated under reduced pressure, basified with triethylamine, water was added the mixture was extracted with DCM (2×50 ml). The combined organics were dried over anhydrous Na₂SO₄, filtered and concentrated under reduced pressure to afford the title product (1.5 g, 52%). MS: 370.3 (M+H)⁺.

### Preparative Example 54: 7-methyl-3-(piperidin-4-yl)-1H-indazole Hydrochloride

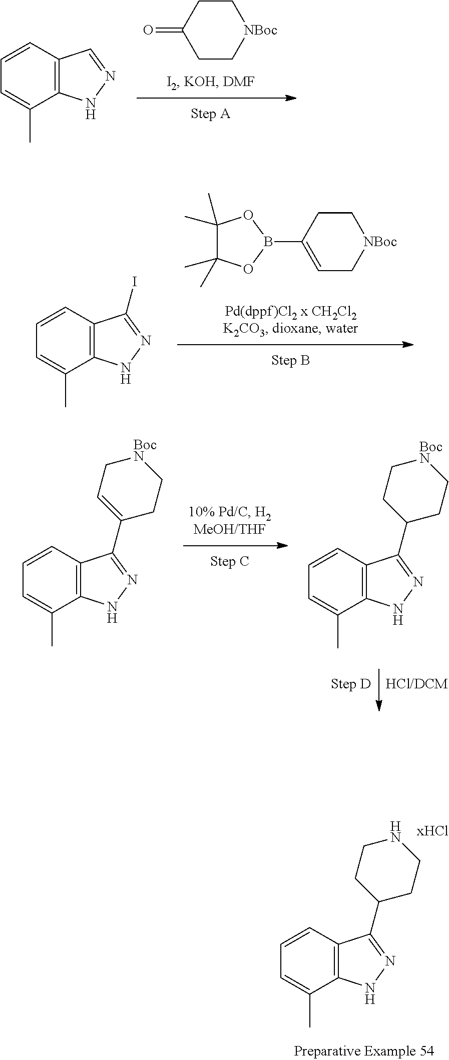

Step A

To a solution of 7-methyl-1H-indazole (2 g, 15.13 mmol) in MeOH (30 ml) and water (1.5 ml), NaOH (0.605 g, 15.13 mmol) was added and the mixture was stirred for 10 minutes until NaOH dissolved completely. Then, iodine (3.84 g, 15.13 mmol) and potassium iodide (2.51 g, 15.13 mmol) were added at 0° C. The mixture was stirred then allowed to warm to room temperature and stirred at 25° C. for 8 hours. The reaction mixture was concentrated under reduced pressure, diluted with a saturated solution of sodium thiosulphate (200 ml) and extracted with ethyl acetate (2×400 ml). The combined organic extracts were washed with brine (200 ml), dried over anhydrous sodium sulphate, filtered and concentrated under reduced pressure. The crude was purified on HP-Sil column (Biotage) eluting with a gradient of petroleum ether/ethyl acetate (100/0 to 80/20) to afford the title compound (2.5 g, 63.8%) as an off white solid. ¹H-NMR (400 MHz, DMSO-d₆): δ 7.23 (t, J=11.20 Hz, 1H), 7.10 (t, J=9.20 Hz, 1H), 2.52 (s, 3H). MS: 259.0 (M+H)⁺.

Step B

To a solution of the title compound from Step A above (2.5 g, 9.69 mmol) in 1,4-dioxane (30 ml), tert-butyl 4-(4,4,5,5-tetramethyl-1,3,2-dioxaborolan-2-yl)-3,6-dihydropyridine-1(2H)-carboxylate (3.00 g, 9.69 mmol), K₂CO₃ (2.68 g, 19.38 mmol) dissolved in water (7.5 ml) and 1,1′-bis(diphenylphosphino)ferrocenedichloro palladium(II) dichloromethane complex (0.709 g, 0.969 mmol) were added under nitrogen atmosphere. The mixture was stirred at 100° C. for 16 hours. The reaction was diluted with water (100 ml) and extracted with ethyl acetate (2×200 ml). The combined organic extracts were washed with brine (200 ml), dried over anhydrous sodium sulphate, filtered and concentrated under reduced pressure. The crude was purified on HP-Sil column (Biotage) eluting with a gradient of petroleum ether/ethyl acetate (100/0 to 70/30) to afford the title compound (2.9 g, 87%) as a yellow solid. 1H-NMR (400 MHz, DMSO-d6): δ 13.05 (s, 1H), 7.80 (d, J=8.00 Hz, 1H), 7.03-7.05 (m, 2H), 6.53 (s, 1H), 4.10 (s, 2H), 3.58-3.59 (m, 2H), 2.59-2.70 (m, 2H), 2.58 (s, 3H), 1.45 (s, 9H). MS: 258.2 (M+H)⁺-t-butyl.

Step C

A solution of the title compound from Step B above (2.9 g, 9.25 mmol) in MeOH (80 ml) was bubbled with nitrogen gas for a period of 10 minutes. Then, palladium hydroxide on carbon (0.325 g, 0.463 mmol) was added under nitrogen atmosphere. The mixture was stirred under hydrogen gas pressure (1 bar) at 25° C. temperature for 20 hours. The reaction was bubbled with nitrogen gas for 10 minutes and filtered through celite and was washed with ethyl acetate (500 ml). The filtrate was concentrated under reduced pressure to afford the title compound (2.6 g, 83%) as an off white solid. MS: 260.2 (M+H)+−t-butyl.

Step D

To a solution of the title compound from Step C above (0.5 g, 1.58 mmol) in DCM (10 ml) a 4.0 M solution of HCl in 1,4-dioxane (3.96 ml, 15.85 mmol) was added dropwise over a period of 5 minutes at 0° C. The mixture was stirred at 25° C. for 1 hour. The reaction was concentrated under reduced pressure to afford the title compound (0.34 g, 98%) as a yellow solid. 1H-NMR (400 MHz, DMSO-d6): δ 12.53 (s, 1H), 7.59 (d, J=8.00 Hz, 1H), 7.07 (d, J=6.40 Hz, 1H), 6.95 (t, J=8.00 Hz, 1H), 3.05-3.08 (m, 3H), 2.58-2.62 (m, 3H), 2.47 (s, 3H), 1.72-1.75 (m, 4H). MS: 216.2 (M+H)⁺.

### Preparative Example 55: 3-(piperazin-1-yl)-1-tosyl-1H-indazole Hydrochloride

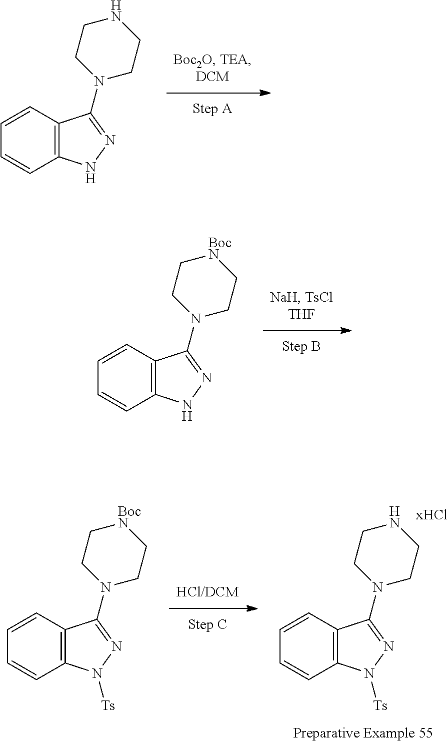 [SMILES: c1ccc2c(N3CCNCC3)n[nH]c2c1 ; CC(C)(C)OC(=O)N1CCN(c2n[nH]c3ccccc23)CC1 ; Cc1ccc(S(=O)(=O)n2nc(N3CCN(C(=O)OC(C)(C)C)CC3)c3ccccc32)cc1 ; Cc1ccc(S(=O)(=O)n2nc(N3CCNCC3)c3ccccc32)cc1]

Step A

To a stirred solution 3-(piperazin-1-yl)-1H-indazole (0.6 g, 2.97 mmol) in DCM (20 ml), TEA (1.240 ml, 8.90 mmol) and Boc-anhydride (0.827 ml, 3.56 mmol) were added at 25° C. The reaction mixture was stirred at 25° C. for 2 hours then was diluted with water (200 ml) and extracted with DCM (2×300 ml). The combined organics were washed with brine (100 ml), dried over anhydrous sodium sulphate, filtered and concentrated under reduced pressure to afford the title compound (0.9 g, 100%) as a yellow solid. 1H-NMR (400 MHz, DMSO-d₆): δ 12.04 (s, 1H), 7.76 (d, J=8.40 Hz, 1H), 7.29-7.30 (m, 2H), 6.97-6.99 (m, 1H), 3.52-3.53 (m, 4H), 3.26-3.27 (m, 4H), 1.44 (s, 9H). MS: 303.2 (M+H)⁺.

Step B

To a suspension of sodium hydride (60% in paraffin oil, 0.265 g, 6.61 mmol) in THF (10 ml), a solution of the title compound from Step A above in THF (10 ml) was added dropwise at 0° C. and the mixture was stirred at room temperature for 30 minutes. Then a solution of tosyl-chloride (0.757 g, 3.97 mmol) in THF (10 ml) was added dropwise at 0° C. and the reaction mixture was further stirred at room temperature for 1 hour. The mixture was quenched with ice cold water (100 ml) and was extracted with ethyl acetate (2×200 ml). The combined organic extracts were washed with brine (100 ml), dried over anhydrous sodium sulphate, filtered, and concentrated under reduced pressure. The crude was purified on HP-Sil column (Biotage), eluting with a gradient of petroleum ether/ethyl acetate (100/0 to 70/30) to afford the title compound (1.5 g, 98%) as an off white solid. ¹H-NMR (400 MHz, CDCl₃): δ 8.21 (d, J=8.40 Hz, 1H), 7.75-7.76 (m, 2H), 7.63 (d, J=8.00 Hz, 1H), 7.51-7.53 (m, 1H), 7.26-7.28 (m, 1H), 7.19 (d, J=8.00 Hz, 2H), 3.57-3.58 (m, 4H), 3.45-3.46 (m, 4H), 2.36 (s, 3H), 1.51 (s, 9H). MS: 457.2 (M+H)⁺.

Step C

To a solution of the title compound from Step B above (1.5 g, 3.29 mmol) in DCM (10 ml) a 4.0 M solution of HCl in 1,4-dioxane (8.21 ml, 32.9 mmol) was added dropwise over a period of 5 minutes at 0° C. The mixture was stirred at 25° C. for 1 hour. The reaction was concentrated under reduced pressure to afford the title compound (1.25 g, 95%) as an off white solid. ¹H-NMR (400 MHz, DMSO-d6): δ 9.11 (s, 2H), 8.02 (d, J=8.40 Hz, 1H), 7.88 (d, J=8.00 Hz, 1H), 7.55-7.56 (m, 2H), 7.25-7.27 (m, 2H), 3.50-3.55 (m, 4H), 3.17 (s, 4H), 2.26 (s, 3H). MS: 357.2 (M+H)⁺.

### Preparative Example 56: 7-methoxy-3-(piperidin-4-yl)-1H-indazole Hydrochloride

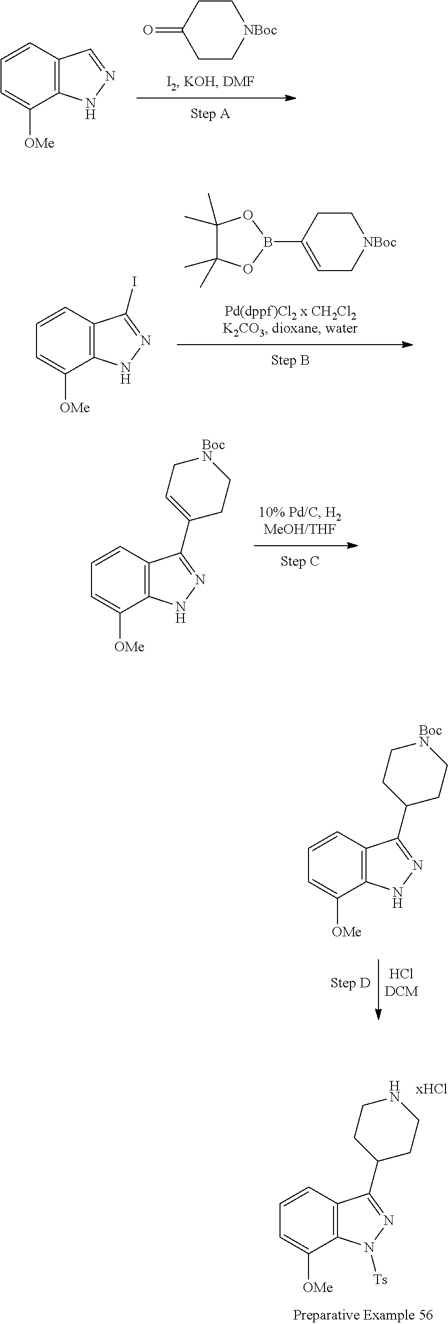

Step A

To 7-methoxy-1H-indazole (2 g, 13.50 mmol) in a 100 ml round bottom flask was added DMF (20 ml). To this reaction mixture, KOH (2.80 g, 49.9 mmol) and iodine (5.14 g, 20.25 mmol) were added at 0° C. The reaction mixture was stirred at 25° C. for 1 hour, following progress by TLC. The reaction mixture was concentrated under reduced pressure then was diluted with a saturated solution of sodium thiosulphate (200 ml) and extracted with ethyl acetate (2×400 ml). The combined organics were washed with brine (200 ml), dried over anhydrous sodium sulphate, filtered and concentrated under reduced pressure to afford the title compound (4.1 g, 100%) as a brown liquid. ¹H-NMR (400 MHz, DMSO-d₆): δ 10.96 (s, 1H), 7.18-7.09 (m, 2H), 6.81 (d, J=7.20 Hz, 1H), 4.00 (s, 3H). MS: 275.0 (M+H)⁺.

Step B

In a 100 ml sealed tube, the title compound from Step A above (4.2 g, 13.90 mmol), tert-butyl 4-(4,4,5,5-tetramethyl-1,3,2-dioxaborolan-2-yl)-3,6-dihydropyridine-1(2H)-carboxylate (4.30 g, 13.90 mmol) in 1,4-dioxane (30 ml), K₂CO₃ (3.84 g, 27.8 mmol) dissolved in water (7.50 ml) and 1,1′-bis(diphenylphosphino)ferrocenedichloro palladium(II) dichloromethane complex (1.017 g, 1.390 mmol) were added under continuous bubbling of nitrogen. The resulting reaction mixture was stirred at 100° C. for 16 hours, following progress by TLC. The reaction mixture was diluted with water (100 ml) and extracted with ethyl acetate (2×200 ml). The combined organics were washed with brine (200 ml), dried over anhydrous sodium sulphate, filtered and concentrated under reduced pressure. The crude was purified by Biotage isolera with silica-gel cartridge (50 g-size) eluting with a gradient of petroleum ether/ethyl acetate (75/25 to 70/30) to give the title compound (4.3 g, 92%) as a yellow solid. ¹H-NMR (400 MHz, DMSO-d₆): δ 13.21 (s, 1H), 7.53 (d, J=8.40 Hz, 1H), 7.06 (t, J=8.40 Hz, 1H), 6.85 (d, J=7.60 Hz, 1H), 6.51 (s, 1H), 4.09 (s, 2H), 3.95 (s, 3H), 3.58 (s, 2H), 2.68 (s, 2H), 1.44 (s, 9H). MS: 330.2 (M+H)⁺.

Step C

In a mini clave, a solution of title compound from Step B above (4.3 g, 13.05 mmol) was dissolved in MeOH (80 ml) and bubbled through with nitrogen gas for a period of 10 minutes. To this reaction mixture, palladium hydroxide on carbon (0.917 g, 0.653 mmol) was added under nitrogen atmosphere. The reaction mixture was stirred under hydrogen pressure (5 bar) at 25° C. for 16 hours. The reaction mixture was purged with nitrogen gas for 10 minutes and then filtered through celite and washed with ethyl acetate (500 ml). The filtrate was concentrated under reduced pressure to afford the tile compound (4 g, 89%) as an off white solid. ¹H-NMR (400 MHz, DMSO-ds): 12.88 (s, 1H), 7.30 (d, J=10.40 Hz, 1H), 6.98 (t, J=10.80 Hz, 1H), 6.80 (d, J=10.00 Hz, 1H), 4.06-4.01 (m, 2H), 3.94 (s, 3H), 3.18 (t, J=9.60 Hz, 1H), 2.94 (s, 2H), 1.96-1.92 (m, 2H), 1.74-1.66 (m, 2H), 1.49 (s, 9H). MS: 332.3 (M+H)⁺

Step D

In a 100 ml round bottom flask, the title compound from Step C above (1.2 g, 3.62 mmol) was dissolved in DCM (10 ml). To this reaction mixture, 4 M HCl in 1,4-dioxane (9.05 ml, 36.2 mmol) was added dropwise over a period of 10 minutes at 0° C. The resulting reaction mixture was stirred at 25° C. for 2 hours, following progress by TLC. Upon completion, the mixture was concentrated to give the title compound (0.7 g, 81%) as an off white solid. MS: 232.1 (M+H)⁺.

### Preparative Example 57: 4-methyl-3-(piperidin-4-yl)-1-tosyl-1H-indazole Hydrochloride

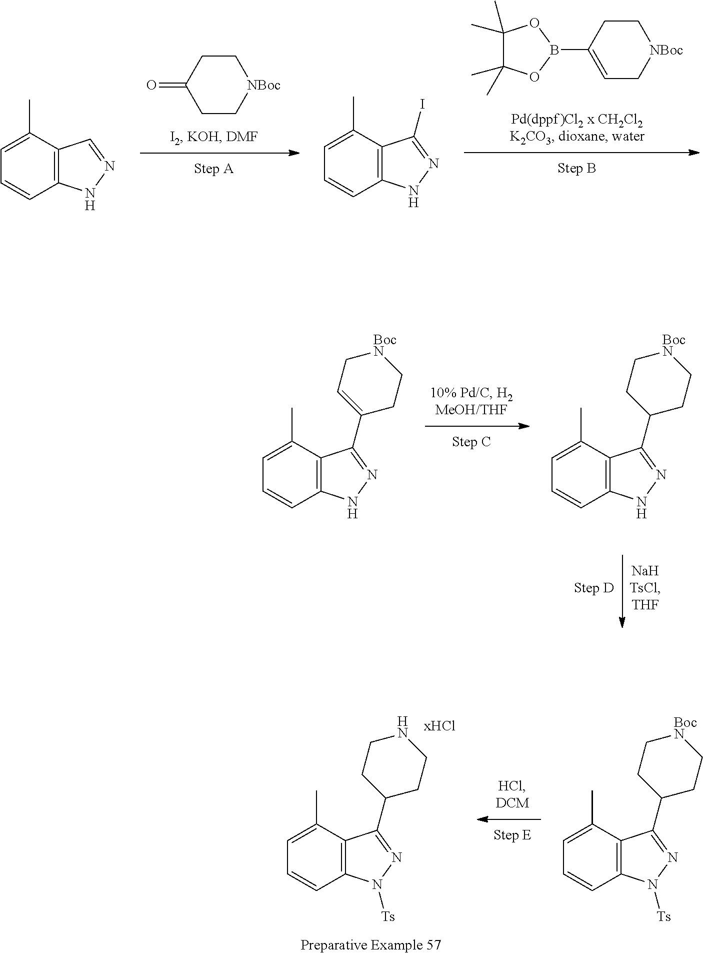

Step A

To a solution of 4-methyl-1H-indazole (3 g, 22.70 mmol) in methanol (35 ml), NaOH (5.27 g, 34 mmol) and iodine (8.64 g, 34 mmol) were added at 0° C. The mixture was stirred for 48 hours at room temperature. After completion of reaction, the reaction mixture was poured into a saturated solution of sodium thiosulphate (10 ml). The solid formed was then filtered using a sintered funnel and the filtrate was extracted with EtOAc (2×100 ml). The combined organic extracts were concentrated under reduced pressure to afford the title compound (5.08 g, 84%). ¹H-NMR (400 MHz, DMSO-d₆): δ 13.43 (s, 1H), 7.42 (d, J=8.40 Hz, 1H), 7.25 (t, J=8.00 Hz, 1H), 6.90 (d, J=6.80 Hz, 1H), 2.77 (s, 3H). MS: 259.0 (M+H)⁺.

Step B

To a mixture of the title compound from Step A above (2.5 g, 9.69 mmol) and 2-methyl-1-(4-(4,4,5,5-tetramethyl-1,3,2-dioxaborolan-2-yl)-3,6-dihydropyridin-1(2H)-yl)propan-2-yl formate (3 g, 9.69 mmol) in dioxane (15 ml) and water (4 ml), was added PdCl2(dppf) (709 mg, 0.969 mmol) and cesium carbonate (947 mg, 2.91 mmol). The mixture was degassed and stirred under N₂ atmosphere at 110° C. overnight in a sealed tube. The mixture was filtered through celite and washed with EtOAc (20 ml) and water (20 ml) and was concentrated to afford the title compound (1.7 g, 54%). MS: 314.1 (M+H)⁺.

Step C

To a solution of the title compound from Step B above (1.7 g, 5.42 mmol) in methanol (50 ml), palladium hydroxide on carbon (0.762 g, 5.42 mmol) was added. The reaction mixture was then hydrogenated using a miniclave (5 bar hydrogen pressure) for 12 hours. The reaction mixture was filtered through celite and the filtrate was concentrated to afford the title compound (1.4 g, 74%). ¹H-NMR (400 MHz, DMSO-d₆): δ 12.67 (s, 1H), 7.27 (d, J=8.40 Hz, 1H), 7.17 (t, J=8.00 Hz, 1H), 6.81 (d, J=6.80 Hz, 1H), 4.10-4.01 (m, 2H), 3.95 (s, 1H), 2.97-2.94 (m, 2H), 2.65 (s, 3H), 1.95-1.92 (m, 2H), 1.72-1.64 (m, 2H), 1.43 (s, 9H). MS: 260.1 (M+H)⁺-t-butyl.

Step D

In a 3-neck round bottom flask, sodium hydride (60% in paraffin oil, 1.414 g, 4.48 mmol) was dissolved in THF at 0° C. The title compound from Step C above (0.269 g, 11.21 mmol) dissolved in THF (10 ml) was added and reaction mixture was stirred for 30 min. Then, tosyl-Cl (1.111 g, 5.83 mmol) in THF (10 ml) was added. The reaction mixture was stirred for 30 minutes at room temperature. The mixture was filtered through celite and concentrated under reduced pressure to afford the title compound (1.15 g, 48%). 1H-NMR (400 MHz, DMSO-d6): δ 7.92 (d, J=11.20 Hz, 1H), 7.73 (d, J=11.20 Hz, 1H), 7.49 (t, J=11.20 Hz, 1H), 7.35 (d, J=10.80 Hz, 1H), 7.15 (d, J=9.60 Hz, 1H), 4.04-4.00 (m, 2H), 3.40 (s, 1H), 2.92 (s, 2H), 2.62 (s, 3H), 2.30 (s, 3H), 1.91-1.87 (m, 2H), 1.65-1.51 (m, 2H), 1.48 (s, 9H). MS: 370 (M+H)⁺-Boc.

Step E

To a solution of the title compound from Step D above (1.01 g, 2.447 mmol) dissolved in DCM (20 ml) was added HCl in 1,4-dioxane (10 ml) at 0° C. and the reaction mixture was stirred for 1 hour. The crude solution was concentrated under reduced pressure, basified with triethylamine, then partitioned between water (20 ml) and DCM (20 ml); the phases were separated, the aqueous phase was extracted with DCM (2×15 ml), the combined organics were washed with water (15 ml), dried over anhydrous sodium sulfate, filtered and concentrated under reduced pressure to afford the title compound (0.8 g, 86%). MS: 370.1 (M+H)+

### Preparative Example 58: 3-(1,2,3,6-tetrahydropyridin-4-yl)-1-tosyl-1H-indazole Hydrochloride

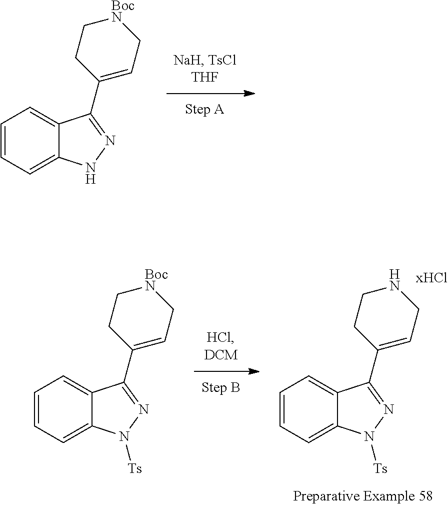 [SMILES: CC(C)(C)OC(=O)N1CC=C(c2n[nH]c3ccccc23)CC1 ; Cc1ccc(S(=O)(=O)n2nc(C3=CCN(C(=O)OC(C)(C)C)CC3)c3ccccc32)cc1 ; Cc1ccc(S(=O)(=O)n2nc(C3=CCNCC3)c3ccccc32)cc1]

Step A

To a stirred suspension of sodium hydride (60% in paraffin oil, 0.481 g, 20.04 mmol) in dry tetrahydrofuran (5 ml) at room temperature, a solution of title compound from Step A of Preparative Example 1 above (3 g, 10.02 mmol)) in dry tetrahydrofuran (25 ml) was added slowly at 0° C. and stirred at the same temperature for 30 minutes. Then a solution of 4-methylbenzene-1-sulfonyl chloride (2.87 g, 15.03 mmol) in dry tetrahydrofuran (10 ml) was added dropwise at room temperature and the reaction mixture was allowed to stir at room temperature for 2 hours. The reaction mixture was cooled to 0° C. and quenched with iced water, followed by extraction using ethyl acetate (2×20 ml). The combined organic extracts were washed with brine, dried over Na₂SO₄, filtered and evaporated under reduced pressure. The crude product was purified on HP-Sil SNAP cartridges using a Biotage Isolera One purification system, eluting with a gradient of heptane/ethyl acetate (100/0 to 40/60). The fractions containing the compound were collected and concentrated under reduced pressure to afford the title compound (12.5 g, 56.2%) as a beige solid. ¹H-NMR (400 MHz, DMSO-d₆): δ 8.16 (d, J=8.40 Hz, 1H), 8.10 (d, J=8.40 Hz, 1H), 7.80 (d, J=8.40 Hz, 2H), 7.70-7.66 (m, 1H), 7.46-7.37 (m, 3H), 6.77 (s, 1H), 4.10 (s, 2H), 3.59-3.56 (m, 2H), 2.62 (s, 2H), 2.32 (s, 3H), 1.44 (s, 9H). MS: 398.0 (M+H)+−t Butyl.

Step B

To a solution of the title compound from Step A above (3 g, 6.61 mmol) in DCM (10 ml) was added 4 M HCl in 1,4-dioxane (10 ml, 40.0 mmol) dropwise at 0° C. and the mixture was stirred at room temperature overnight. The reaction mixture was concentrated and the residue was washed with ether (20 ml) and dried to afford the title compound (2.30 g, 84%) as a yellow solid. 1H-NMR (400 MHz, DMSO-d6): δ 8.95 (bs, 2H), 8.19 (d, J=8.40 Hz, 1H), 8.11 (d, J=8.40 Hz, 1H), 7.82 (d, J=8.00 Hz, 2H), 7.71 (t, J=8.00 Hz, 1H), 7.49 (t, J=8.00 Hz, 1H), 7.40 (d, J=8.00 Hz, 2H), 6.81 (s, 1H), 3.97 (s, 1H), 3.86 (s, 2H), 2.81 (s, 2H), 2.33 (s, 4H). MS: 354.0 (M+H)⁺.

### Preparative Example 59: 3-(3-fluoropiperidin-4-yl)-1-tosyl-1H-indazole Hydrochloride

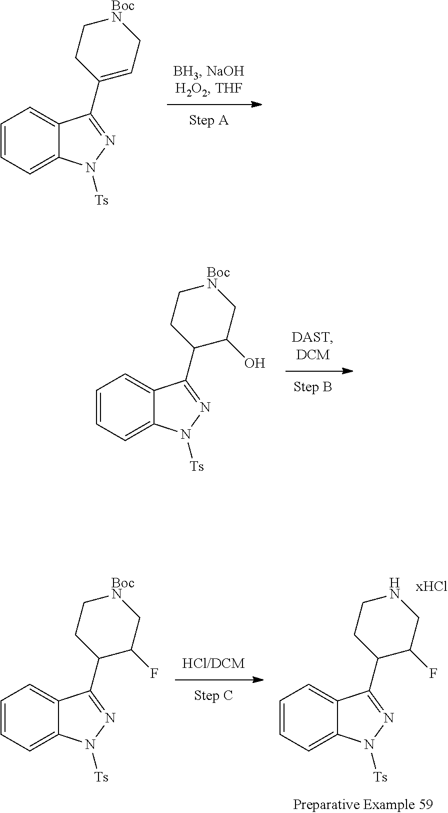 [SMILES: Cc1ccc(S(=O)(=O)n2nc(C3=CCN(C(=O)OC(C)(C)C)CC3)c3ccccc32)cc1 ; Cc1ccc(S(=O)(=O)n2nc(C3CCN(C(=O)OC(C)(C)C)CC3O)c3ccccc32)cc1 ; Cc1ccc(S(=O)(=O)n2nc(C3CCN(C(=O)OC(C)(C)C)CC3F)c3ccccc32)cc1 ; Cc1ccc(S(=O)(=O)n2nc(C3CCNCC3F)c3ccccc32)cc1]

Step A

To a solution of title compound from Step B or Preparative Example 1 above (3 g, 6.61 mmol) in THF (30 ml), BH₃·THF (26.5 ml, 26.5 mmol) was added at 0° C. and the reaction mixture was stirred at room temperature for 12 hours. Then, NaOH (9.92 ml, 19.84 mmol) was added dropwise at 0° C., followed by H₂O₂(1.892 ml, 18.52 mmol). The addition was done while keeping the internal temperature below 10° C. The reaction was then stirred at room temperature for 1 hour. The reaction mixture was quenched using 10% sodium thiosulphate solution at 0° C. and extracted with ethyl acetate (2×100 ml). The organic layers were collected, dried over anhydrous sodium sulfate and the solvent was evaporated under reduced pressure to afford the title compound (0.8 g, 77%). MS: 372.1 (M+H)⁺-boc.

Step B

To a solution of title compound from Step A above (1.8 g, 3.82 mmol) in DCM (30 ml), DAST (2.017 ml, 15.27 mmol) was added at −65° C. Then the reaction mixture was warmed up to room temperature within 2 hours. The reaction mixture was quenched with a solution of sodium carbonate, and extracted with DCM (2×50 ml). The organic layers were collected, dried and the solvent was evaporated under reduced pressure. The crude was purified by chromatography column using ethyl acetate and petroleum ether (20/80) as eluents to afford the title compound (0.8 g, 42.7%). MS: 374.2 (M+H)⁺-boc.

Step C

To a solution of title compound from Step B above (0.8 g, 1.689 mmol) in DCM (30 ml), 4 M HCl in 1,4-dioxane (3 ml, 12.00 mmol) was added at 0° C. The reaction mixture was stirred at room temperature for 1 hour. The reaction mixture was then concentrated under reduced pressure to afford the title compound (0.69 g, 95%). ¹H-NMR (400 MHz, DMSO-ds): 9.53-9.43 (m, 2H), 8.13 (d, J=8.80 Hz, 1H), 7.98 (d, J=8.00 Hz, 1H), 7.83-7.78 (m, 2H), 7.72-7.68 (m, 1H), 7.48-7.40 (m, 2H), 5.31-5.18 (m, 1H), 3.84-3.62 (m, 1H), 3.34-3.22 (m, 4H), 2.46 (s, 3H), 2.33-2.15 (m, 2H). MS: 374.1 (M+H)⁺.

### Preparative Examples 60-61

Following the Buchwald coupling procedure as described in Preparative Example 59 the below compounds were prepared.

| | | | | 1. Yield; % |
|---|---|---|---|---|
| Preparative | Amine | Chloro | | 2. ¹H-NMR |
| Example | Derivative | Derivative | Product | 3. MH⁺ (ESI) |
| Prep. Ex. 60 4-(5-(4-(1-tosyl-1H- indazol-3- yl)piperidin-1- yl)thiazolo[5,4- b]pyridin-2- yl)morpholine | 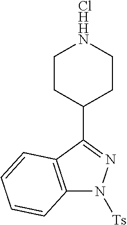 | 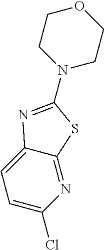 [SMILES: Clc1ccc2nc(N3CCOCC3)sc2n1] | 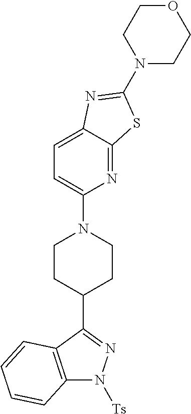 [SMILES: Cc1ccc(S(=O)(=O)n2nc(C3CCN(c4ccc5nc(N6CCOCC6)sc5n4)CC3)c3ccccc32)cc1] | 1. 29% 2. ¹H-NMR (80 MHz, DMSO-d₆) δ = 8.01 (dd, J = 13.0, 7.9 Hz, 2H), 7.84 - 7.57 (m, 3H), 7.57 - 7.22 (m, 3H), 7.18 - 6.77 (m, 2H), 4.46 - 3.96 (m, 2H), 3.85 - 3.59 (m, 4H), 3.59 - 3.40 (m, 4H), 3.11 - 2.72 (m, 3H), 2.28 (s, 3H), 2.10 - 1.53 (m, 4H). 3. 575.3 |
| Prep. Ex. 61 2-morpholino-5-(4- (1-tosyl-1H-indol- 3-yl)-3,6- dihydropyridin- 1(2H)- yl)benzo[d]oxazole | 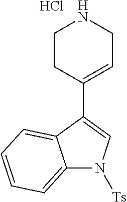 [SMILES: Cl ; Cc1ccc(S(=O)(=O)n2cc(C3=CCNCC3)c3ccccc32)cc1] | 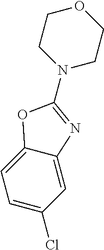 [SMILES: Clc1ccc2oc(N3CCOCC3)nc2c1] | 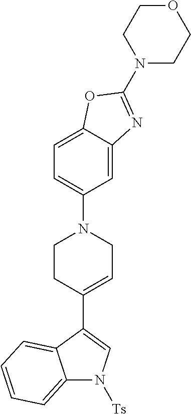 [SMILES: Cc1ccc(S(=O)(=O)n2cc(C3=CCN(c4ccc5oc(N6CCOCC6)nc5c4)CC3)c3ccccc32)cc1] | 1. 88%. 2. ¹H-NMR (80 MHz, DMSO-d₆) δ 8.16 - 7.70 (m, 4H), 7.62 - 7.10 (m, 5H), 7.30 - 6.80 (m, 2H), 6.72 (d, J = 8.6 Hz, 1H), 6.42 (s, 1H), 3.62 (d, J = 8.6 Hz, 12H), 2.68 (s, 2H), 2.31 (s, 3H). 3. 555.2 |

### Preparative Example 62: 5-fluoro-1-methyl-3-(1,2,3,6-tetrahydropyridin-4-yl)-1H-pyrrolo[2,3-b]pyridine Hydrochloride

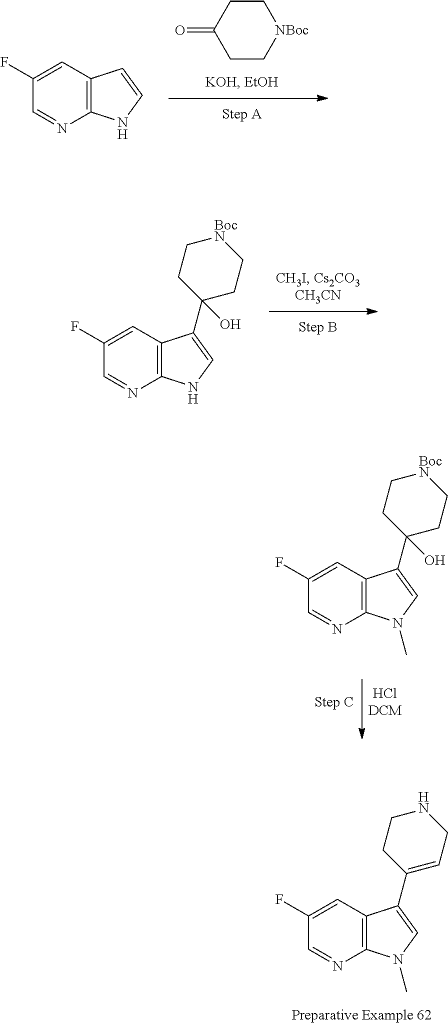 [SMILES: CC(C)(C)OC(=O)N1CCC(=O)CC1 ; Fc1cnc2[nH]ccc2c1 ; CC(C)(C)OC(=O)N1CCC(O)(c2c[nH]c3ncc(F)cc23)CC1 ; Cn1cc(C2(O)CCN(C(=O)OC(C)(C)C)CC2)c2cc(F)cnc21 ; Cn1cc(C2=CCNCC2)c2cc(F)cnc21]

Step A

To a stirred solution of 5-fluoro-1H-pyrrolo[2,3-b]pyridine (3 g, 0.022 mol) in MeOH (50 ml), KOH (3.7 g, 0.066 mol) and tert-butyl 4-oxopiperidine-1-carboxylate (8.77 g, 0.0444 mol) were added and the mixture was stirred at room temperature for 48 hours. The reaction mixture was quenched with water (30 ml) and the formed precipitate was filtered, washed with water (20 ml) and dried under vacuum to afford the title compound. (1.4 g, 27%). ¹H-NMR (400 MHz, DMSO-d₆): δ 11.57 (s, 1H), 8.25-8.24 (m, 1H), 7.87-7.84 (m, 1H), 7.62 (d, J=3.60 Hz, 1H), 6.47 (d, J=3.20 Hz, 1H), 3.83 (s, 2H), 3.63-3.60 (m, 4H), 2.50-2.34 (m, 2H), 1.43 (s, 9H). MS: 336.2 (M+H)⁺.

Step B

To a stirred solution of the title compound from Step A above (1.0 g, 2.98 mmol) in acetonitrile (10 ml), cesium carbonate (2.9 g, 8.94 mmol) and methyl iodide (0.63 g, 4.47 mmol) were added and the mixture was stirred at room temperature for 16 hours. The reaction mixture was filtered through celite and concentrated to afford the title compound (0.8 g, 77%). MS: 250.2 (M+H)⁺-Boc.

Step C

To a cooled solution (0° C.) of the title compound from Step B above (0.8 g, 2.28 mmol) in dry DCM (15 ml), 4.0 M HCl in 1,4-dioxane (2 ml) was added and stirred for 30 minutes. The reaction mixture was concentrated and triturated with diethyl ether, the solid was filtered off, dried under vacuum to afford the title compound (0.6 g, 98%). MS: 232.2 (M+H)⁺.

### Preparative Example 63: 3-bromo-5-fluoro-1-methyl-1H-pyrrolo[2,3-b]pyridine

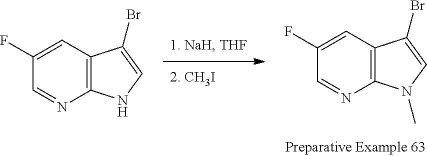

Step A

In a 100 ml round bottom flask purged with nitrogen, sodium hydride (60% in paraffin oil, 0.558 g, 13.95 mmol) was dissolved in THF (20 ml), cooled to 0° C., and the solution was stirred for 10 min. To this reaction mixture, 3-bromo-5-fluoro-1H-pyrrolo[2,3-b]pyridine (1 g, 4.65 mmol) in THF (10 ml) was added dropwise at 0° C. over 1 minute. The reaction mixture was then stirred at 25° C. for 1 hour under nitrogen atmosphere. To this reaction mixture was added methyl iodide (0.349 ml, 5.58 mmol) dropwise over a period of 2 minutes. The resulting reaction mixture was stirred at 0° C. for 1 hour under nitrogen atmosphere. The reaction mixture was extracted with ethyl acetate (2×200 ml) and washed with water (100 ml). The combined organics were concentrated under reduced pressure to afford the title compound (1.14 g, 98%). ¹H-NMR (400 MHz, CDCl₃): δ 8.21-8.23 (m, 1H), 7.43-7.44 (m, 1H), 7.35 (s, 1H), 3.91 (s, 3H). MS: 231.0 (M+H)⁺.

### Preparative Example 64: 3-(piperidin-4-yl)-1-tosyl-1H-pyrrolo[2,3-c]pyridine Hydrochloride

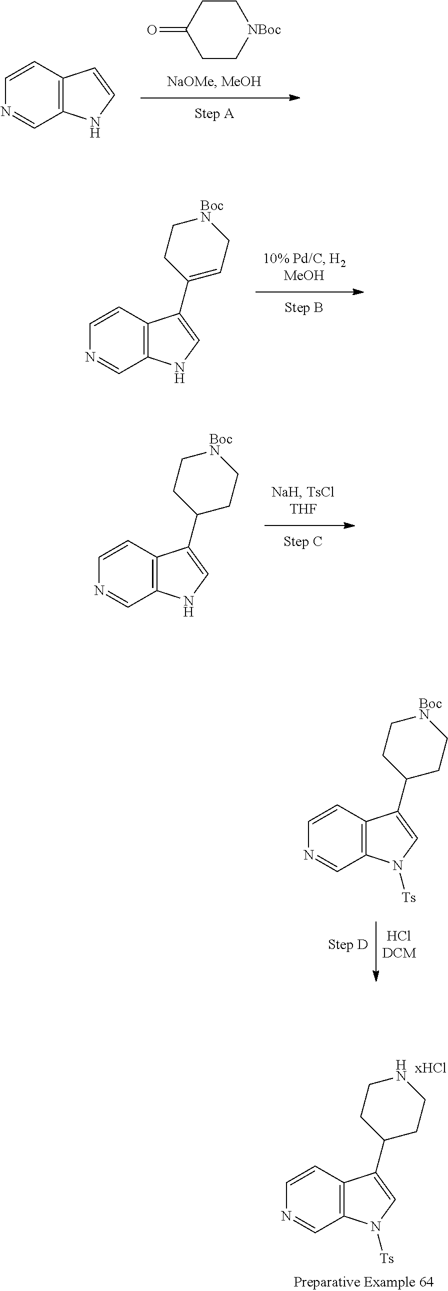 [SMILES: CC(C)(C)OC(=O)N1CCC(=O)CC1 ; c1cc2cc[nH]c2cn1 ; CC(C)(C)OC(=O)N1CC=C(c2c[nH]c3cnccc23)CC1 ; CC(C)(C)OC(=O)N1CCC(c2c[nH]c3cnccc23)CC1 ; Cc1ccc(S(=O)(=O)n2cc(C3CCN(C(=O)OC(C)(C)C)CC3)c3ccncc32)cc1 ; Cc1ccc(S(=O)(=O)n2cc(C3CCNCC3)c3ccncc32)cc1]

Step A

To a stirred solution of 1H-pyrrolo[3,2-c]pyridine (2.5 g, 21.2 mmol) in methanol (20 ml), sodium methoxide (2.75 g, 63.4 mmol) and tert-butyl 4-oxopiperidine-1-carboxylate (6.32 g, 31.7 mmol) were added and the mixture was heated to 70° C. for 12 hours under nitrogen atmosphere. The reaction mixture was quenched by water (20 ml) and the precipitate was filtered through a sintered funnel. The solid was washed with water (20 ml), petroleum ether (15 ml) and dried under vacuum to afford the title compound (4.5 g, 70%) as a gummy brown solid. ¹H NMR (400 MHz, DMSO-d₆): δ 11.67 (s, 1H), 8.74 (t, J=5.20 Hz, 1H), 8.13 (d, J=5.60 Hz, 1H), 7.79 (d, J=5.60 Hz, 1H), 7.68 (d, J=2.40 Hz, 1H), 6.18 (s, 1H), 4.05 (s, 2H), 3.57 (t, J=5.60 Hz, 2H), 2.50-2.51 (m, 2H), 1.44 (s, 9H). MS: 300.3 (M+H)⁺.

Step B

To a solution of the title compound from Step A above (4.5 g, 15 mmol) in THF (50 ml), 10% Pd/C (400 mg) was added and the mixture was stirred at room temperature for 24 h under hydrogen pressure (1 bar). The reaction mixture was filtered through celite and washed with methanol (50 ml). The filtrate was concentrated under reduced pressure to afford the title compound (4 g, 88%). MS: 302.2 (M+H)⁺.

Step C

To a suspension of sodium hydride (60% in paraffin oil, 1.1 g, 39.9 mmol) in THF (10 ml), was added dropwise at 0° C. a solution of the title compound from Step B above (4 g, 13.3 mmol) in THF (40 ml) and the resulting mixture was stirred at room temperature for 30 minutes. A solution of tosyl chloride (3.30 g, 17.3 mmol) in THF (15 ml) was added at 0° C. and the mixture was stirred at room temperature for 1 hour. The reaction mixture was quenched with iced water (10 ml) followed by extraction using ethyl acetate (50 ml). The organic layer was separated, dried over sodium sulphate, filtered and then concentrated under reduced pressure to afford the title compound (4 g, 66%) as an off-white solid. ¹H NMR (400 MHz, DMSO-d₆): δ 9.17 (s, 1H), 8.37 (d, J=5.20 Hz, 1H), 7.96 (d, J=8.80 Hz, 2H), 7.81 (s, 1H), 7.71 (d, J=6.00 Hz, 1H), 7.40 (d, J=9.20 Hz, 2H), 4.04-4.08 (m, 2H), 2.74-2.75 (m, 4H), 2.33 (s, 3H), 1.50-1.54 (m, 3H), 1.43 (s, 9H). MS: 456.2 (M+H)⁺.

Step D

To a solution of the title compound from Step C above (4 g, 8.75 mmol) in DCM (40 ml), was added slowly at 0° C. 4.0 M HCl in 1,4-dioxane (10 ml) and the mixture was stirred at 25° C. for 2 hours. The reaction mixture was concentrated, the solid washed with diethyl ether (10 ml), and dried under vacuum to afford the title compound (3 g, 96%). MS: 355.9 (M+H)⁺.

### Preparative Example 65: 5-fluoro-1-methyl-3-(piperidin-4-yl)-1H-indole Hydrochloride

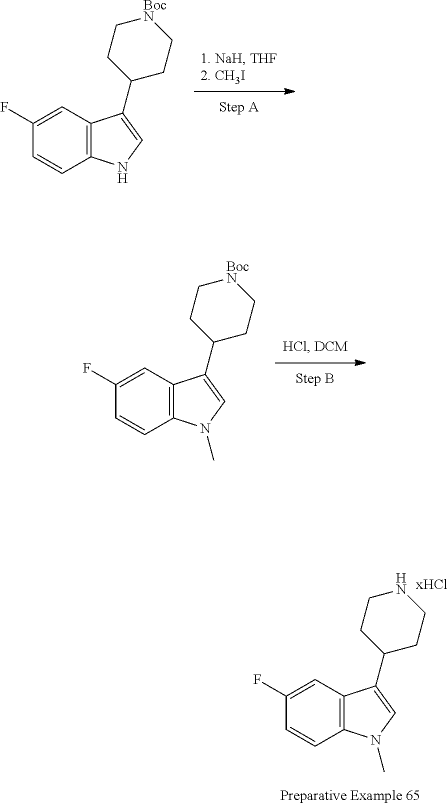 [SMILES: CC(C)(C)OC(=O)N1CCC(c2c[nH]c3ccc(F)cc23)CC1 ; Cn1cc(C2CCN(C(=O)OC(C)(C)C)CC2)c2cc(F)ccc21 ; Cn1cc(C2CCNCC2)c2cc(F)ccc21]

Step A

To a suspension of sodium hydride (60% in paraffin oil, 0.24 g, 5.03 mmol) in THF (5 ml), was added dropwise at 0° C. a solution of title compound from Step B of Preparative Example 15 above (4 g, 13.3 mmol) in THF (40 ml) and the resulting mixture was stirred at room temperature for 30 minutes. A solution of methyl iodide (0.5 ml, 3.2 mmol) was added at 0° C. and the mixture was stirred at room temperature for 1 hour. The reaction mixture was quenched with iced water (10 ml) followed by extraction using ethyl acetate (50 ml). The organic layer was separated, dried over sodium sulphate, filtered and then concentrated under reduced pressure to afford the title compound (0.75 g, 90%) as an off white solid. MS: 333.1 (M+H)⁺.

Step B

To a solution of the title compound from Step A above (0.75 g, 2.24 mmol) in DCM (40 ml), was added slowly at 0° C. 4.0 M HCl in 1,4-dioxane (10 ml) and the mixture was stirred at 25° C. for 2 hours. The reaction mixture was concentrated and washed with diethyl ether to afford the title compound (0.5 g, 96%) MS: 233.3 (M+H)⁺.

### SYNTHESIS OF THE EXAMPLES

### Example 1: Synthesis of Compound 1 5-(4-(1H-indazol-3-yl)piperidin-1-yl)-2-morpholinobenzo[d]oxazole

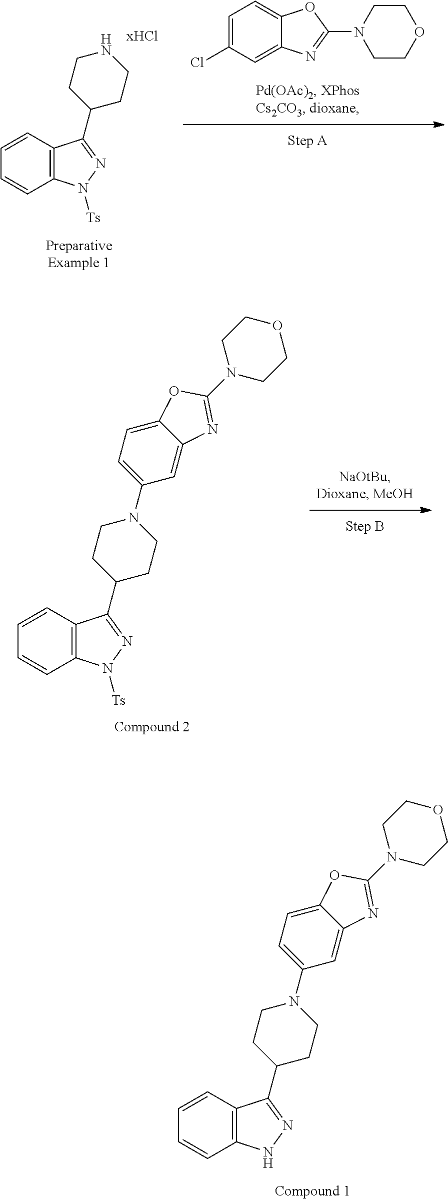 [SMILES: Cc1ccc(S(=O)(=O)n2nc(C3CCNCC3)c3ccccc32)cc1 ; Clc1ccc2oc(N3CCOCC3)nc2c1 ; Cc1ccc(S(=O)(=O)n2nc(C3CCN(c4ccc5oc(N6CCOCC6)nc5c4)CC3)c3ccccc32)cc1 ; c1ccc2c(C3CCN(c4ccc5oc(N6CCOCC6)nc5c4)CC3)n[nH]c2c1]

Step A

Pd(OAc)₂ (0.426 g, 1.90 mmol) and 2-dicyclohexylphosphino-2′,4′,6′-triisopropylbiphenyl (Xphos) (1.81 g, 3.80 mmol) were added to a reaction vial and 1,4-dioxane (200 ml) was added, degassed with nitrogen for 10 minutes. The vial was filled with nitrogen gas and sealed. The suspension was heated at 100° C. for 10 minutes then 5-chloro-2-morpholino-1,3-benzoxazole (3.32 g, 13.9 mmol) followed by Preparative Example 1 (4.50 g, 12.7 mmol), and Cs₂CO₃ (10.3 g, 31.6 mmol) were added, and the solution was heated at 100° C. for 20 hours. The reaction was monitored by Liquid Chromatograph-Mass Spectrometry (LCMS). The reaction mixture was filtered through celite and the filtrate was concentrated under reduced pressure. The residue was purified on HP-Sil column using a Biotage purification system by employing a petroleum ether/ethyl acetate gradient (100/0 to 80/20) to afford the 2-morpholino-5-(4-(1-tosyl-1H-indazol-3-yl)piperidin-1-yl)benzo[d]oxazole compound (Compound 2, 2.3 g, 67.2%) as an off white solid. ¹H-NMR (300 MHz, DMSO-d₆): δ 8.09 (d, J=8.40 Hz, 1H), 7.94 (d, J=7.80 Hz, 1H), 7.75 (d, J=8.40 Hz, 2H), 7.62-7.68 (m, 1H), 7.38-7.43 (m, 1H), 7.34 (d, J=8.10 Hz, 2H), 7.26 (d, J=8.70 Hz, 1H), 6.95 (d, J=2.40 Hz, 1H), 6.69 (dd, J=2.40, 8.70 Hz, 1H), 3.70-3.73 (m, 4H), 3.54-3.64 (m, 6H), 3.25 (m, 1H), 2.81-2.88 (m, 2H), 2.29 (s, 3H), 1.96-2.04 (m, 4H). MS: 558.2 (M+H)⁺.

Step B

To a stirred solution of the crude title compound from Step A above (5.5 g, 9.86 mmol) in 1.4 dioxane (50 ml) and MeOH (50 ml), sodium tert-butoxide (NaOtBu, 5.6 g, 59.10 mmol) was added and the reaction mixture was stirred at 80° C. for 16 hours. Completion of reaction was monitored by TLC. The reaction mixture was concentrated, and the crude was triturated with water at room temperature for 1 hour. The solid obtained was washed with water and dried. The solid was washed with methanol (50 ml) and diethyl ether (50 ml) and dried to afford to the 5-(4-(1H-indazol-3-yl)piperidin-1-yl)-2-morpholinobenzo[d]oxazole (Compound 1, 2.65 g, 71%) as a pale yellow solid. ¹H-NMR (400 MHz, DMSO-d₆): δ 12.67 (s, 1H), 7.81 (d, J=8 Hz, 1H), 7.48 (d, J=8.40 Hz, 1H), 7.30-7.34 (m, 1H), 7.26 (d, J=8.80 Hz, 1H), 7.05-7.09 (m, 1H), 6.97 (d, J=2.40 Hz, 1H), 6.72 (dd, J=2.40, 8.80 Hz, 1H), 3.70-3.73 (m, 6H), 3.55-3.58 (m, 4H), 3.17-3.21 (m, 1H), 2.83-2.89 (m, 2H), 2.00-2.09 (m, 4H). MS: 404.2 (M+H)⁺.

### Example 3: 5-(5-fluoro-1-methyl-1H-pyrrolo[2,3-b]pyridin-3-yl)-2-morpholinobenzo[d]oxazole

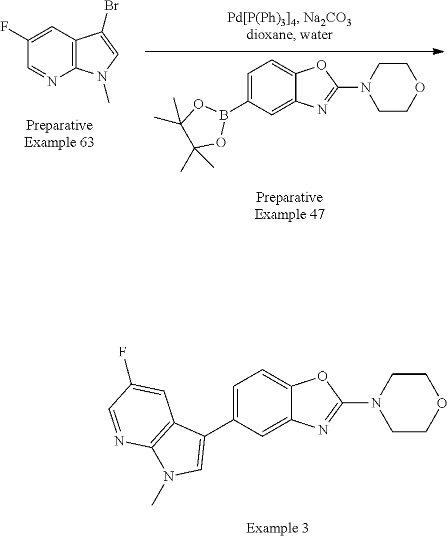 [SMILES: Cn1cc(Br)c2cc(F)cnc21 ; CC1(C)OB(c2ccc3oc(N4CCOCC4)nc3c2)OC1(C)C ; Cn1cc(-c2ccc3oc(N4CCOCC4)nc3c2)c2cc(F)cnc21]

In a 50 ml sealed tube, the title compound from Preparative Example 63 (0.300 g, 1.310 mmol) and the title compound from Preparative Example 47 (0.432 g, 1.310 mmol) were dissolved in 1,4-dioxane (10 ml) and water (2 ml) and the mixture was purged with nitrogen. To this were added sodium carbonate (0.416 g, 3.93 mmol) and tetrakis(triphenylphosphine)palladium (0.076 g, 0.065 mmol). The reaction mixture was heated at 100° C. for 1 hour. The mixture was then allowed to cool down to room temperature and diluted with ethyl acetate and water. The organic layer was separated and the aqueous layer was extracted twice with ethyl acetate. The combined organic layers were dried over Na₂SO₄, filtered and concentrated under reduced pressure. The crude compound was purified on HP-Sil cartridge using a Biotage Isolera One purification system with a gradient of ethyl acetate/petroleum ether (100/0 to 80/20). Fractions containing the compound were collected and concentrated under reduced pressure to afford the title compound as a white solid (0.066 g, 14%). ¹H NMR (400 MHz, DMSO-d₆): δ 8.32 (s, 1H), 8.13 (dd, J=2.40, 9.80 Hz, 1H), 8.00 (s, 1H), 7.58 (s, 1H), 7.48 (d, J=8.40 Hz, 1H), 7.35 (dd, J=1.60, 8.40 Hz, 1H), 3.88 (s, 3H), 3.75 (t, J=5.20 Hz, 4H), 3.62 (t, J=4.40 Hz, 4H). MS: 353.2 (M+H).

### Example 4

Following the Sukuzi coupling procedure as described in Example 3, the following compound was prepared.

| | | | | 1. Yield; % |
|---|---|---|---|---|
| | Bromo | Boronate | | 2. ¹H-NMR |
| Example | Derivative | Derivative | Product | 3. MH⁺ (ESI) |
| Example 4 5-(1H-indazol-3- yl)-2- morpholinobenzo [d]oxazole | 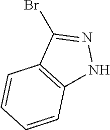 | 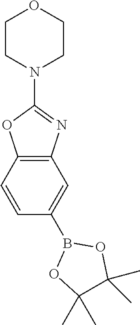 [SMILES: CC1(C)OB(c2ccc3oc(N4CCOCC4)nc3c2)OC1(C)C] | 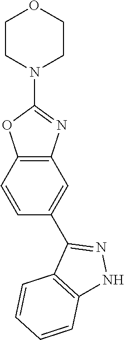 [SMILES: c1ccc2c(-c3ccc4oc(N5CCOCC5)nc4c3)n[nH]c2c1] | 1. 16.6% 2. 1H-NMR (400 MHz, DMSO-d6): δ 13.17 (s, 1H), 8.05 (d, J = 8.00 Hz, 1H), 7.84 (s, 1H), 7.67 (dd, J = 2.00, 8.20 Hz, 1H), 7.57-7.55 (m, 2H), 7.39-7.38 (m, 1H), 7.19- 7.18 (m, 1H), 3.76 (t, J = 5.20 Hz, 4H), 3.64 (t, J = 4.40 Hz, 4H) 3. 321.1 |

### Example 5: 5-((1H-indazol-3-yl)ethynyl)-2-morpholinobenzo[d]oxazole

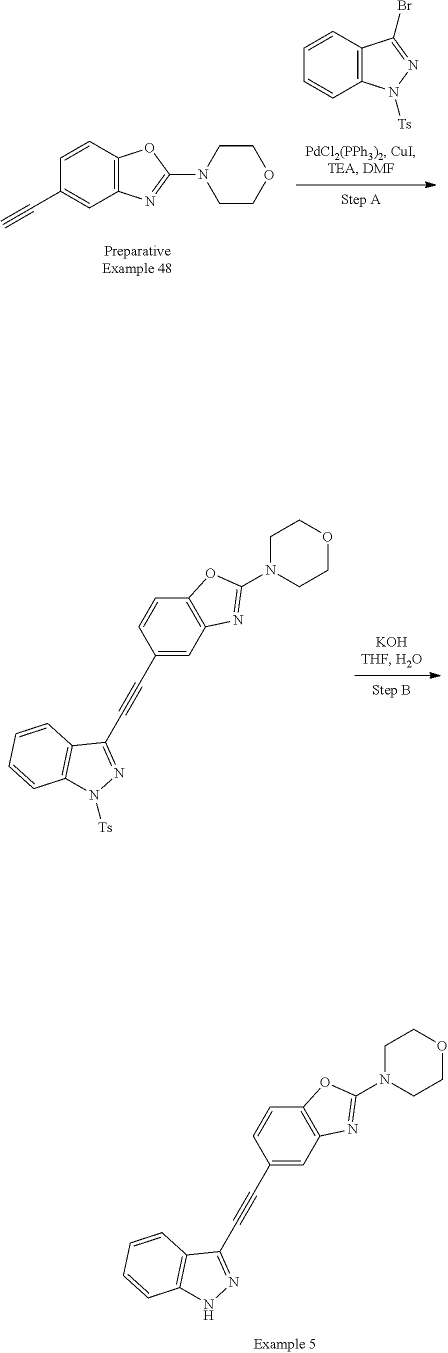 [SMILES: Cc1ccc(S(=O)(=O)n2nc(Br)c3ccccc32)cc1 ; C#Cc1ccc2oc(N3CCOCC3)nc2c1 ; Cc1ccc(S(=O)(=O)n2nc(C#Cc3ccc4oc(N5CCOCC5)nc4c3)c3ccccc32)cc1 ; C(#Cc1n[nH]c2ccccc12)c1ccc2oc(N3CCOCC3)nc2c1]

Step A

To a solution of 3-bromo-1-tosyl-1H-indazole (0.358 g, 1.314 mmol) in DMF (20 ml), were added triethylamine (15 ml, 0.857 mmol) and Preparative Example 48 (0.3 g, 1.314 mmol) and the mixture was purged with nitrogen for 5 minutes. Bis(triphenylphosphin)palladium(II)-dichloride (0.922 g, 1.314 mmol) and copper (I) iodide (0.250 g, 1.314 mmol) were added and the mixture was stirred at room temperature for 12 hours. The reaction mixture was filtered through celite and the filtrate was concentrated. The residue was recrystallized from a mixture of ethyl acetate and DCM to afford 2-morpholino-5-((1-tosyl-1H-indazol-3-yl)ethynyl)benzo[d]oxazole (0.22 g, 33.5%). ¹H NMR (400 MHz, DMSO-d₆): δ 8.21 (d, J=8.80 Hz, 1H), 8.01 (d, J=8.00 Hz, 1H), 7.89 (d, J=8.00 Hz, 2H), 7.77 (t, J=7.60 Hz, 1H), 7.68 (s, 1H), 7.56-7.52 (m, 2H), 7.44 (d, J=8.40 Hz, 3H), 3.75-3.73 (m, 4H), 3.64-3.62 (m, 4H), 2.35 (s, 3H). MS: 499.0 (M+H)⁺.

Step B

To a solution of 2-morpholino-5-((1-tosyl-1H-indazol-3-yl)ethynyl)benzo[d]oxazole (0.18 g, 0.361 mmol) from Step A in THF (2.5 ml) and water (1 ml), was added KOH (101 mg, mg, 1.805 mmol) and the resulting mixture was heated at 55° C. for 12 hours. The crude mixture was concentrated and purified by preparative HPLC to afford 5-((1H-indazol-3-yl)ethynyl)-2-morpholinobenzo[d]oxazole (0.05 g, 40.2%) 1H-NMR (400 MHz, DMSO-d6): δ 13.49 (s, 1H), 7.87 (d, J=8.00 Hz, 1H), 7.59-7.60 (m, 2H), 7.51-7.53 (m, 1H), 7.45 (t, J=8.00 Hz, 1H), 7.36 (d, J=8.40 Hz, 1H), 7.26 (t, J=7.60 Hz, 1H), 3.73-3.74 (m, 4H), 3.62-3.63 (m, 4H) MS: 345 (M+H)⁺.

### Example 6

Following the Sukuzi coupling, procedure as described in Example 5 the following compound was prepared.

| | | | | 1. Yield; % |
|---|---|---|---|---|
| | Bromo | Alkyne | | 2. ¹H-NMR |
| Example | Derivative | Derivative | Product | 3. MH⁺ (ESI) |
| Example 6 5-((1H-indol-3- yl)ethynyl)-2- morpholinobenzo [d]oxazole | 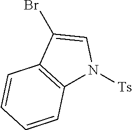 [SMILES: Cc1ccc(S(=O)(=O)n2cc(Br)c3ccccc32)cc1] | 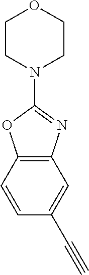 [SMILES: C#Cc1ccc2oc(N3CCOCC3)nc2c1] | 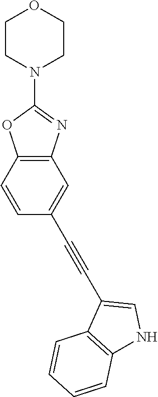 [SMILES: C(#Cc1c[nH]c2ccccc12)c1ccc2oc(N3CCOCC3)nc2c1] | 1. 31% 2. ¹H-NMR (400 MHz, DMSO-d6): δ 11.54 (s, 1H), 7.76 (d, J = 2.40 Hz, 1H), 7.68 (d, J = 7.60 Hz, 1H), 7.47-7.45 (m, 3H), 7.25-7.18 (m, 3H), 3.75-3.73 (m, 4H), 3.63-3.61 (m, 4H). 3. 344.3 |

### Example 7: N-(1H-indol-3-yl)-2-morpholinobenzo[d]oxazole-5-carboxamide

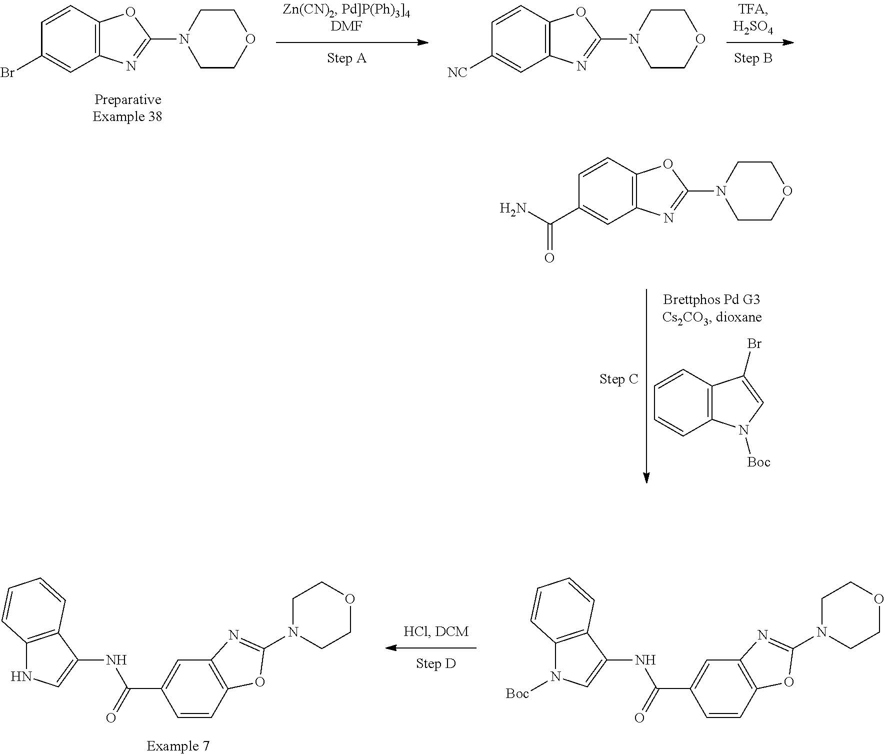 [SMILES: Brc1ccc2oc(N3CCOCC3)nc2c1 ; N#Cc1ccc2oc(N3CCOCC3)nc2c1 ; NC(=O)c1ccc2oc(N3CCOCC3)nc2c1 ; CC(C)(C)OC(=O)n1cc(Br)c2ccccc21 ; O=C(Nc1c[nH]c2ccccc12)c1ccc2oc(N3CCOCC3)nc2c1 ; CC(C)(C)OC(=O)n1cc(NC(=O)c2ccc3oc(N4CCOCC4)nc3c2)c2ccccc21]

Step A

To a sealed tube were added 5-bromo-2-morpholino-1,3-benzoxazole (Preparative example 38, 600 mg, 2.12 mmol) and Zn(CN)₂ (298.62 mg, 2.54 mmol, 161.42 ml) in DMF (10 ml). Then Pd(PPh₃)₄ (1.22 g, 1.06 mmol) was added under N₂. The mixture was heated to 80° C. and stirred for 3 hours. The reaction mixture was poured into saturated EDTA solution (80 ml) and followed by ethyl acetate (30 ml). The solution was stirred at 20° C. for 2 hours and the aqueous phase was separated and extracted with ethyl acetate (2×20 ml). The combined organic layers were washed successively with water (2×20 ml) and brine (20 ml), dried over anhydrous Na₂SO₄, filtered, and concentrated. The residue was purified by silica gel chromatography (column height: 250 mm, diameter: 100 mm, 100-200 mesh silica gel, Petroleum ether/Ethyl acetate=20/1 to 5/1) to afford 2-morpholinobenzo[d]oxazole-5-carbonitrile (440 mg, 90.57%) as a white solid. MS: 230.0 (M+H)⁺.

Step B

A mixture of compound from Step A above (400 mg, 1.74 mmol), TFA (2 ml) and H₂SO₄ (2 ml) was heated to 60° C. and stirred for 6 hours. The reaction mixture was cooled to 20° C., then added to 2.0 M solution of aqueous HCl. The solution was extracted with ethyl acetate (3×30 ml), and the combined organic layers were washed with H₂O (3×20 ml) and dried over Na₂SO₄ to obtain 2-morpholinobenzo[d]oxazole-5-carboxamide (300 mg, crude) as a brown solid. MS: 248.0 (M+H)⁺.

Step C

To a sealed tube were added tert-butyl 3-bromoindole-1-carboxylate (210.82 mg, 711.83 mmol), compound from Step B above (110 mg, 444.89 mmol) in 1,4-dioxane (2 ml). Then Cs₂CO₃ (289.91 mg, 889.79 mmol) and BrettPhos Pd G3 (40.33 mg, 44.49 mmol) were added under N₂ atmosphere. The mixture was stirred heated to 100° C. and stirred for 3 hours. The reaction mixture was poured into 80 ml saturated EDTA solution followed by 30 ml of ethyl acetate. The solution was stirred at 20° C. for 2 hours. After that, the aqueous phase was separated and extracted with ethyl acetate (2×20 ml). The combined organic layers were washed successively with water (2×20 ml) and brine (20 ml), dried over anhydrous Na₂SO₄, filtered, and concentrated under reduced pressure. The residue was purified by prep-TLC (SiO2, Petroleum ether/Ethyl acetate=1/1) to afford tert-butyl 3-(2-morpholinobenzo[d]oxazole-5-carboxamido)-1H-indole-1-carboxylate (130 mg, 74%) as a yellow solid.

Step D

A solution of compound from Step C above (110 mg, 237.84 mmol) in HCl/ethyl acetate (4 M, 59.46 ml) was stirred for 1 hour at 20° C. The solid was filtered and concentrated under reduced pressure.

The crude was purified by prep-HPLC (column: Phenomenex Gemini-NX 80*40 mm*3 μm; mobile phase: [water (10 mM NH4HCO3)-ACN]; B %: 15%-45%, 8 min neutral), to afford the title compound (26.48 mg, 31%) as a white solid. MS: 363.1 (M+H)⁺.

### Example 8: 5-(4-(1H-indazol-3-yl)-1H-pyrazol-1-yl)-2-morpholinobenzo[d]oxazole

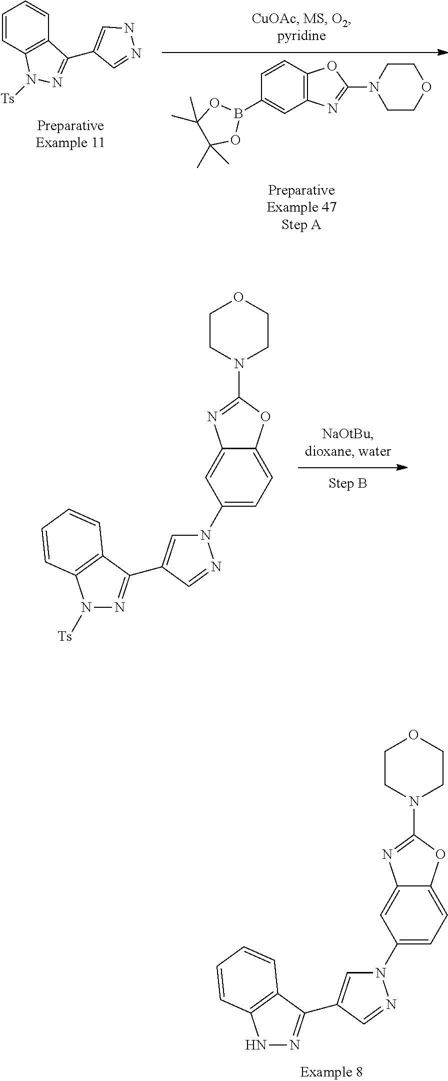 [SMILES: Cc1ccc(S(=O)(=O)n2nc(-c3cn[nH]c3)c3ccccc32)cc1 ; CC1(C)OB(c2ccc3oc(N4CCOCC4)nc3c2)OC1(C)C ; Cc1ccc(S(=O)(=O)n2nc(-c3cnn(-c4ccc5oc(N6CCOCC6)nc5c4)c3)c3ccccc32)cc1 ; c1ccc2c(-c3cnn(-c4ccc5oc(N6CCOCC6)nc5c4)c3)n[nH]c2c1]

Step A

Preparative example 11 (400 mg, 1.186 mmol) and Preparative example 47 (431 mg, 1.304 mmol) were dissolved in pyridine (20 ml). Then, copper(II)acetate (538 mg, 2.96 mmol) and molecular sieves (1.186 mmol) were added and the resulting mixture was heated at 100° C. for 12 hours. The reaction mixture was concentrated, ethyl acetate (50 ml) and water (50 ml) were added. The phases were separated, the organic layer was washed with brine and dried over sodium sulphate then filtered and concentrated. The crude compound was purified on HP-Sil cartridge using a Biotage Isolera One purification system with a gradient of petroleum ether and ethyl acetate (100/0 to 55/45) to afford 2-morpholino-5-(4-(1-tosyl-1H-indol-3-yl)-1H-pyrazol-1-yl)benzo[d]oxazole (250 mg, 36%) as a brown gummy solid. MS: 540.1 (M+H)⁺.

Step B

To a solution of 2-morpholino-5-(4-(1-tosyl-1H-indol-3-yl)-1H-pyrazol-1-yl)benzo[d]oxazole from Step A (250 mg, 0.463 mmol) in 1,4-dioxane (5 ml) and MeOH (5 ml), sodium tert-butoxide (267 mg, 2.78 mmol) was added and the reaction mixture was heated at 70° C. for 12 hours, following progress by TLC. After completion of the reaction, the crude mixture was concentrated and purified on silica gel column using Biotage Isolera One purification system eluting with ethyl acetate/hexane (90/10). The residue was recrystallized from diethyl ether and methanol to afford the title compound (50 mg, 27%) as a white solid. 2H-NMR (400 MHz, DMSO-d₆): δ 13.08 (s, 1H), 9.09 (s, 1H), 8.24-8.26 (m, 2H), 7.96 (d, J=2.40 Hz, 1H), 7.68-7.69 (1, 1H), 7.56 (d, J=8.40 Hz, 2H), 7.41 (t, J=7.60 Hz, 1H), 7.21 (t, J=7.20 Hz, 1H), 3.74-3.76 (m, 4H), 3.63-3.64 (n, 4H). MS: 387.1 (M+H)⁺.

### Example 9

Following the coupling and deprotection procedures as described in Example 8 the following compounds were prepared.

| | | | | 1. Yield; % |
|---|---|---|---|---|
| | Amine | Boronate | | 2. ¹H-NMR |
| Example | Derivative | Derivative | Product | 3. MH⁺ (ESI) |
| Example 9 5-(4-(1H- indol-3-yl)- 1H-pyrazol-1- yl)-2- morpholino- benzo[d]oxa- zole | 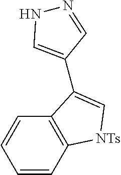 [SMILES: Cc1ccc(S(=O)(=O)n2cc(-c3cn[nH]c3)c3ccccc32)cc1] | 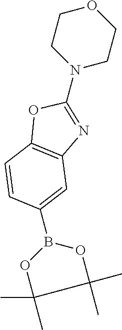 | 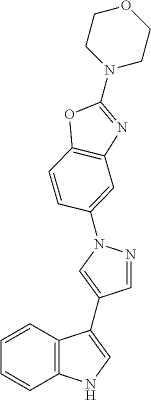 [SMILES: c1ccc2c(-c3cnn(-c4ccc5oc(N6CCOCC6)nc5c4)c3)c[nH]c2c1] | 1. 32% 2. 1H-NMR (400 MHz, DMSO- d6) : δ 11.23 (s, 1H), 8.80 (s, 1H), 8.12 (s, 1H), 7.99 (d, J = 8.00 Hz, 1H), 7.89 (s, 1H), 7.70 (d, J = 2.40 Hz, 1H), 7.64 (dd, J = 2.00, 8.60 Hz, 1H), 7.54 (d, J = 8.80 Hz, 1H), 7.43 (d, J = 8.00 Hz, 1H), 7.10-7.08 (m, 2H), 3.76-3.74 (m, 4H), 3.64-3.63 (m, 4H). 3. 386.1 |
| Example 10 5-(3-(1H- indazol-3-yl)- 1H-pyrazol-1- yl)-2- morpholino- benzo[d]oxa- zole | 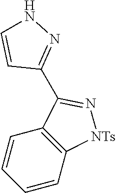 [SMILES: Cc1ccc(S(=O)(=O)n2nc(-c3cc[nH]n3)c3ccccc32)cc1] | 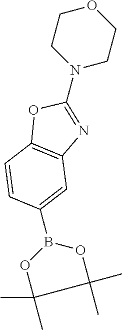 | 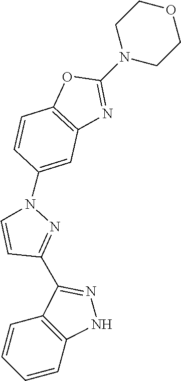 [SMILES: c1ccc2c(-c3ccn(-c4ccc5oc(N6CCOCC6)nc5c4)n3)n[nH]c2c1] | 1. 21% 2. 1H-NMR (400 MHz, DMSO- d6): δ : 13.19 (s, 1H), 8.61 (d, J = 2.80 Hz, 1H), 8.43 (d, J = 8.00 Hz, 1H), 7.93 (s, 1H), 7.72-7.69 (m, 1H), 7.59 (d, J = 8.40 Hz, 2H), 7.43 (t, J = 7.60 Hz, 1H), 7.26 (t, J = 7.20 Hz, 1H), 7.01 (d, J = 2.40 Hz, 1H), 3.77-3.75 (m, 4H), 3.66-3.64 (m, 4H). 3. 387.3 |
| Example 11 5-(3-(1H- indol-3-yl)- 1H-pyrazol-1- yl)-2- morpholino- benzo[d]oxa- zole | 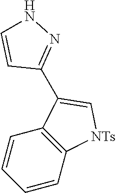 [SMILES: Cc1ccc(S(=O)(=O)n2cc(-c3cc[nH]n3)c3ccccc32)cc1] | 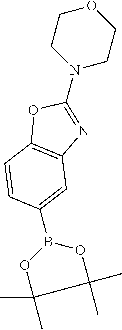 | 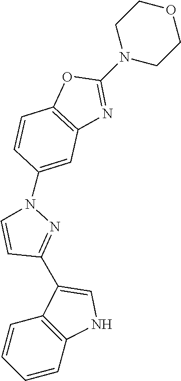 [SMILES: c1ccc2c(-c3ccn(-c4ccc5oc(N6CCOCC6)nc5c4)n3)c[nH]c2c1] | 1. 25% 2. 1H NMR (400 MHz, DMSO- d6): δ 11.33 (s, 1H), 8.50 (d, J = 2.00 Hz, 1H), 8.33 (d, J = 6.80 Hz, 1H), 7.87 (s, 2H), 7.65 (d, J = 8.40 Hz, 1H), 7.55 (d, J = 8.40 Hz, 1H), 7.44 (d, J = 6.80 Hz, 1H), 7.16 (t, J = 3.60 Hz, 2H), 6.88 (s, 1H), 3.76-3.75 (m, 4H), 3.65-3.64 (m, 4H). 3. 386.1 |

### Example 12: N-(2-morpholinobenzo[d]oxazol-5-yl)-1H-indole-3-carboxamide

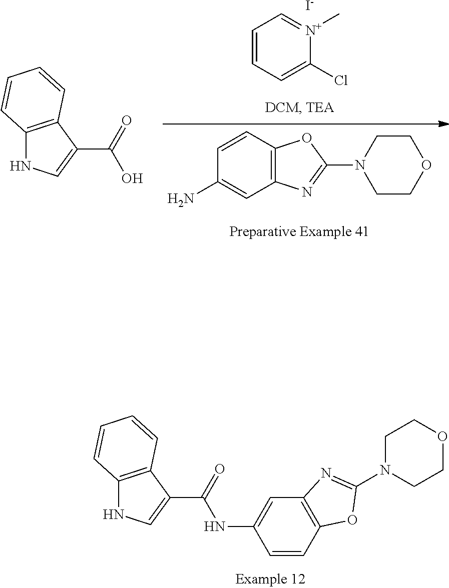

To a stirred solution of 1-phenylpyrrole-3-carboxylic acid (0.25 g, 0.00134 mol) and 2-morpholino-1,3-benzoxazol-5-amine (Preparative example 41, 0.327 g, 0.00147 mol) in DCM (10 ml), triethylamine (0.55 ml) was added at 0° C. and the mixture was stirred at room temperature for 10 minutes. Then, 2-Chloro-N-methylpyridinium iodide (Mukaiyama reagent) (0.512 g, 0.002 mol) was added and the reaction mixture was stirred for 12 hours at room temperature. After completion of the reaction as evidenced by LCMS, the reaction mixture was diluted with dichloromethane (2×30 ml) and water (30 ml). The phases were separated and the aqueous phase was extracted with dichloromethane twice (2×30 ml). The combined organics were dried over Na₂SO₄, filtered and the solvents were evaporated under reduced pressure. The crude was purified on HP-Sil column (Biotage), eluting with a gradient of DCM/MeOH (100/0 to 96/04) to afford the title compound as a white solid (0.075 g, 15%). 1H-NMR (400 MHz, DMSO-d6): δ 11.20 (s, 1H), 9.69 (s, 1H), 8.28 (d, J=2.80 Hz, 1H), 8.20 (d, J=7.60 Hz, 1H), 7.78 (s, 1H), 7.47 (d, J=7.60 Hz, 1H), 7.35-7.41 (m, 2H), 7.19-7.21 (m, 2H), 3.74 (t, J=5.20 Hz, 4H), 3.60 (t, J=4.40 Hz, 4H). MS: 363.2 (M+H)⁺.

Following the amide coupling procedure as described in Example 12, the following compounds were prepared.

| | | | | 1. Yield; % |
|---|---|---|---|---|
| | Acid | Amino | | 2. ¹H-NMR |
| Example | Derivative | Derivative | Product | 3. MH⁺ (ESI) |
| Example 13 N-(2- morpholino- benzo[d]oxazol- 5-yl)-1H- pyrrolo[2,3- b]pyridine-3- carboxamide | 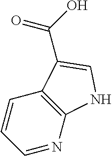 [SMILES: O=C(O)c1c[nH]c2ncccc12] | 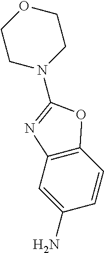 [SMILES: Nc1ccc2oc(N3CCOCC3)nc2c1] | 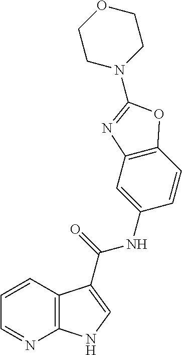 [SMILES: O=C(Nc1ccc2oc(N3CCOCC3)nc2c1)c1c[nH]c2ncccc12] | 1. 22% 2. 1H-NMR (400 MHz, DMSO- d6): δ 12.25 (s, 1H), 9.80 (s, 1H), 8.50 (dd, J = 1.60, 7.60 Hz, 1H), 8.41 (d, J = 2.80 Hz, 1H), 8.31 (dd, J = 1.60, 4.80 Hz, 1H), 7.77 (s, 1H), 7.38 (s, 2H), 7.22 (dd, J = 4.80, 8.00 Hz, 1H), 3.74 (t, J = 5.20 Hz, 4H), 3.60 (t, J = 4.40 Hz, 4H). 3. 364.2 |
| Example 14 N-(2- morpholinoben- zo[d]oxazol- 6-yl)-1H- indole-3- carboxamide | 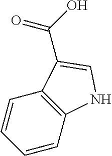 [SMILES: O=C(O)c1c[nH]c2ccccc12] | 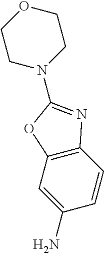 [SMILES: Nc1ccc2nc(N3CCOCC3)oc2c1] | 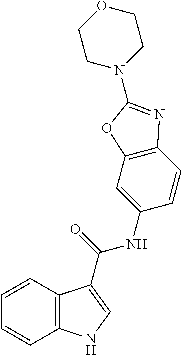 [SMILES: O=C(Nc1ccc2nc(N3CCOCC3)oc2c1)c1c[nH]c2ccccc12] | 1. 61% 2. 1H-NMR (400 MHz, DMSO-d6) : δ 11.73 (s, 1H), 9.77 (s, 1H), 8.28 (d, J = 2.80 Hz, 1H), 8.20 (d, J = 7.20 Hz, 1H), 8.05 (s, 1H), 7.47-7.48 (m, 1H), 7.40-7.41 (m, 1H), 7.27 (d, J = 8.40 Hz, 1H), 7.21-7.22 (m, 2H), 3.73-3.74 (m, 4H), 3.57-3.58 (m, 4H). 3. 363.2 |
| Example 15 N-(2- morpholinoben- zo[d]oxazol- 6-yl)-1H- pyrrolo[2,3- b]pyridine-3- carboxamide | 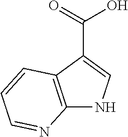 [SMILES: O=C(O)c1c[nH]c2ncccc12] | 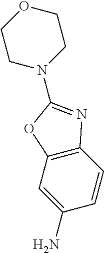 [SMILES: Nc1ccc2nc(N3CCOCC3)oc2c1] | 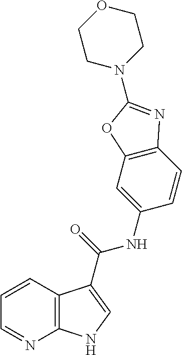 [SMILES: O=C(Nc1ccc2nc(N3CCOCC3)oc2c1)c1c[nH]c2ncccc12] | 1. 15% 2. 1H NMR (400 MHz, DMSO- d6): δ 12.22 (bs, 1H), 9.93 (s, 1H), 8.51-8.49 (m, 1H), 8.44 (s, 1H), 8.32-8.30 (m, 1H), 8.04 (s, 1H), 7.44-7.41 (m, 1H), 7.27 (d, J = 8.40 Hz, 1H), 7.23-7.20 (m, 1H), 3.75-3.72 (m, 4H), 3.60-3.57 (m, 4H). 3. 364.2 |
| Example 16 N-(2- morpholinoben- zo[d]oxazol- 6-yl)-1H- indazole-3- carboxamide | 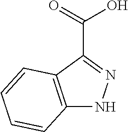 [SMILES: O=C(O)c1n[nH]c2ccccc12] | 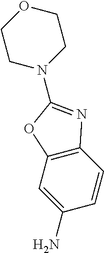 [SMILES: Nc1ccc2nc(N3CCOCC3)oc2c1] | 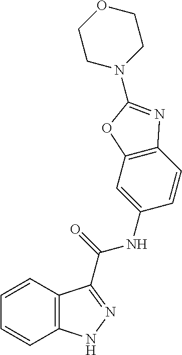 [SMILES: O=C(Nc1ccc2nc(N3CCOCC3)oc2c1)c1n[nH]c2ccccc12] | 1. 12% 2. 1H NMR (400 MHz, DMSO- d6) δ 10.40 (s, 1H), 8.23 (d, J = 8.00 Hz, 1H), 8.11 (s, 1H), 7.69-7.65 (m, 2H), 7.47-7.43 (m, 1H), 7.29 (t, J = 8.40 Hz, 2H), 3.75-3.73 (m, 4H), 3.60- 3.58 (m, 4H). 3. 364.1 |
| Example 17 5-fluoro-N-(2- morpholinoben- zo[d]thiazol- 6-yl)-1H- indole-3- carboxamide | 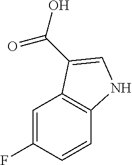 [SMILES: O=C(O)c1c[nH]c2ccc(F)cc12] | 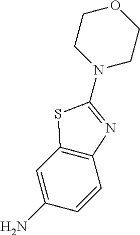 [SMILES: Nc1ccc2nc(N3CCOCC3)sc2c1] | 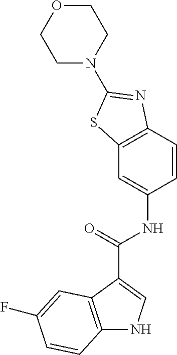 [SMILES: O=C(Nc1ccc2nc(N3CCOCC3)sc2c1)c1c[nH]c2ccc(F)cc12] | 1. 7% 2. 1H NMR (400 MHz, DMSO- d6) δ 11.83 (s, 1H), 9.79 (s, 1H), 8.43 - 8.26 (m, 2H), 7.87 (dd, J = 10.2, 2.6 Hz, 1H), 7.55 - 7.40 (m, 3H), 7.04 (td, J = 9.1, 2.7 Hz, 1H), 3.74 (dd, J = 5.9, 3.9 Hz, 4H), 3.53 (dd, J = 5.8, 4.0 Hz, 4H). 3. 397.17 |
| Example 18 N-(2- morpholinoben- zo[d]oxazol- 5-yl)-1H- indazole-3- carboxamide | 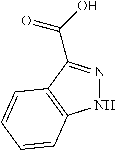 [SMILES: O=C(O)c1n[nH]c2ccccc12] | 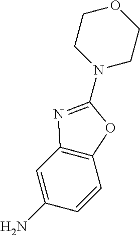 [SMILES: Nc1ccc2oc(N3CCOCC3)nc2c1] | 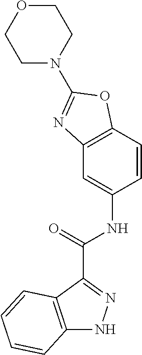 [SMILES: O=C(Nc1ccc2oc(N3CCOCC3)nc2c1)c1n[nH]c2ccccc12] | 1. 28% 2. 1H NMR (400 MHz, DMSO- d6) δ = 13.75 (br s, 1H), 10.28 (s, 1H), 8.24 (d, J = 8.1 Hz, 1H), 7.89 (d, J = 2.0 Hz, 1H), 7.67 (d, J = 8.4 Hz, 1H), 7.58 (dd, J = 2.1, 8.7 Hz, 1H), 7.46 (t, J = 7.3 Hz, 1H), 7.38 (d, J = 8.6 Hz, 1H), 7.30 (t, J = 7.5 Hz, 1H), 3.79 - 3.69 (m, 4H), 3.66 - 3.57 (m, 4H). 3. 364.0 |

### Examples 19-103

The Examples of this invention were prepared following the general procedures for the Buchwald and Sonogashira couplings. The specific procedures used are:

Procedure 1:

To a stirred solution of the amine derivative (0.15 g, 1 eq.) in dry 1,4-dioxane (5 ml), was added the corresponding bromo or chloro derivative (1 eq.) as indicated in Table 1, and sodium tert.-butoxide (3 eq.). The reaction mixture was degassed for 10 min under N₂ atmosphere. Then tris(dibenzylideneacetone)dipalladium(0) (Pd₂(dba)₃; 0.05 eq.) and 2-dicyclohexylphosphino-2′,6′-diisopropoxybiphenyl (Ru-Phos; 0.1 eq.) were added and the reaction mixture was heated to 10000 following progress by LCMS. After completion of the reaction, the mixture was filtered through celite and washed with ethyl acetate. The filtrate was concentrated under reduced pressure to yield the crude product. The crude material was purified by flash column chromatography or preparative HPLC to afford the tosyl protected compound. To a solution of tosyl compound (1.0 eq.) in 1,4-dioxane:MeOH (1:1, 10 vol), was added NaOtBu (3 eq.) and the resulting mixture was heated at 70° C. for 6 hours. The reaction mixture was concentrated under vacuum and the crude material was purified by flash column chromatography or preparative HPLC to afford the final compound as indicated in Table 1.

Procedure 2:

To a stirred solution of the amine derivative (0.15 g, 1 eq.) in dry 1,4-dioxane (5 ml), was added the corresponding bromo or chloro derivative (1 eq.) as indicated in Table 1, and sodium tert.-butoxide (3 eq.). The reaction mixture was degassed for 10 minutes under N₂ atmosphere. Then tris(dibenzylideneacetone)dipalladium(0) (Pd₂(dba)₃; 0.05 eq.) and 2-dicyclohexylphosphino-2′,6′-diisopropoxybiphenyl (Ru-Phos; 0.1 eq.) were added and the reaction mixture was heated at 100° C. following progress by LCMS. After completion of the reaction, the mixture was filtered through celite and washed with ethyl acetate. The filtrate was concentrated under reduced pressure to yield the crude product. The crude material was purified by flash column chromatography or preparative HPLC to afford the final compounds as indicated in Table 1.

Procedure 3:

Pd(OAc)₂ (0.1 eq) and Xphos (0.3 eq.) were added to a reaction vial and degassed 1,4-dioxane (4 ml) was added. The vial was filled with argon gas and sealed. The suspension was heated at 110° C. for 1 minute then the amine derivative (70 mg, 1 eq.), bromo or chloro derivative (1.1 eq.) and Cs₂CO₃ (3.5 eq.) were added and the solution was heated at 100° C. for 18 hours. The reaction mixture was diluted with ethyl acetate (30 ml) and water (30 ml). The phases were separated and the aqueous phase was extracted with ethyl acetate (2×30 ml). The combined organics were dried over Na₂SO₄, filtered and the solvents were evaporated under reduced pressure. The crude was purified on HP-Sil column (Biotage), eluting with a gradient of DCM/MeOH (100/0 to 95/05) to afford the tosyl protected compound.

To a solution of the tosyl compound (1.0 eq) in 1,4-dioxane:MeOH (1:1, 10 vol), was added NaOtBu (3 eq) and the resulting mixture was heated at 70° C. for 6 hours. The reaction mixture was concentrated under vacuum and the crude product was purified by flash column chromatography or preparative HPLC, followed, when appropriate, by chiral supercritical fluid (SFC) chromatography to obtain the final compound as indicated in Table 1.

Procedure 4:

To a stirred solution of the amine derivative (0.15 g, 1 eq.) in dry 1,4-dioxane (5 ml), was added the corresponding bromo or chloro derivative (1 eq.) as indicated in Table 1, and Cs₂CO₃ (3 eq.). The reaction mixture was degassed for 10 min under N₂ atmosphere. Then Pd(OAc)₂ (0.1 eq) and 2-Dicyclohexylphosphino-2′,4′,6′-triisopropylbiphenyl (XPhos; 0.3 eq) were added and the reaction mixture was heated at 100° C. following progress by LCMS. After completion of the reaction, the crude mixture was filtered through celite and washed with ethyl acetate (30 ml). The filtrate was concentrated under reduced pressure to yield the crude product. The crude material was purified by flash column chromatography or preparative HPLC to afford the tosyl protected compound. To a solution of the tosyl compound (1.0 eq) in 1,4-dioxane/MeOH (1/1, 10 vol), was added NaOtBu (3 eq) and the resulting mixture was heated at 70° C. for 6 hours. The reaction mixture was concentrated under vacuum and the crude product was purified by flash column chromatography or preparative HPLC to afford the final compound as indicated in Table 1.

Procedure 5

To a stirred solution of the amine derivative (150 mg, 1 eq) in dry 1,4-dioxane (5 ml), was added the corresponding bromo or chloro derivative (1 eq), and Sodium Tert-Butoxide (3 eq). The reaction was degassed for 10 min under N₂ atmosphere. To this reaction mixture was added Ruphos G4 Pd (0.3 eq) and heated at 100° C. following progress by LCMS. After completion of the reaction, the crude mixture was filtered through celite, washed with ethyl acetate (30 ml). The filtrate was concentrated under reduced pressure and the crude was purified by column chromatography or Prep HPLC, followed, when appropriate, by chiral supercritical fluid chromatography (SFC) to obtain the final compound as indicated in Table 1.

Procedure 6

To a stirred solution of the amine derivative (150 mg, 1 eq.) in dry 1,4-dioxane (5 ml), was added the corresponding bromo or chloro derivative (1 eq.), Potassium phosphate (3 eq.) and degassed for 10 min under N₂ atmosphere. To this reaction mixture was added tris(dibenzylideneacetone)dipalladium(0) (Pd₂(dba)₃; 0.15 eq.) and 2-Dicyclohexylphosphino-2′,4′,6′-triisopropylbiphenyl (XPhos, 0.4 eq), heated to 100° C. until completion of the reaction. After completion of the reaction, the reaction mixture was filtered through celite, washed with EtOAc. The filtrate was concentrated under reduced pressure and the crude material was purified by column chromatography or Prep HPLC to afford the final compound as indicated in Table 1 Procedure 7 To a stirred solution of ethynyl derivative (1 eq) and bromo or chloro derivative (1 eq) in DMF (10 ml), CuI (0.05 eq), PdCl₂(PPh₃)₂(0.1 eq) and triethylamine (5 eq.) were added. The reaction mixture was heated in a microwave reactor at 120° C. for 1 hour or at room temperature for 16 hours. After the completion of the reaction (monitored by LCMS), the reaction mixture was filtered through celite and washed with ethyl acetate (30 ml). The filtrate was concentrated under reduced pressure to yield the crude product. The crude material was purified by flash column chromatography or preparative HPLC, followed by, when appropriate, chiral SFC separation, to afford the final compounds as indicated in Table 1.

TABLE 1

| | | | | 1. Yield; % |
|---|---|---|---|---|
| | | | | 2. ¹H-NMR |
| | Amine | Halo | | 3. MH⁺ (ESI) |
| Example | Derivative | Derivative | Product | 4. Synthesis procedure |
| Example 19 5-(4-(1H-indol-3- yl)piperidin-1- yl)-2- morpholinobenzo [d]oxazole | 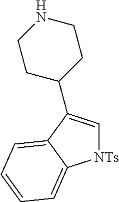 [SMILES: Cc1ccc(S(=O)(=O)n2cc(C3CCNCC3)c3ccccc32)cc1] | 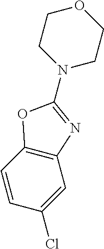 [SMILES: Clc1ccc2oc(N3CCOCC3)nc2c1] | 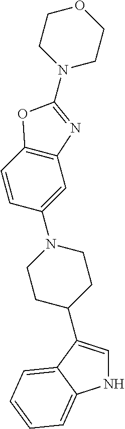 [SMILES: c1ccc2c(C3CCN(c4ccc5oc(N6CCOCC6)nc5c4)CC3)c[nH]c2c1] | 1. 68% 2. ¹H-NMR (400 MHz, DMSO- d₆): δ 10.81 (s, 1H), 7.59 (d, J = 8.00 Hz, 1H), 7.35 (d, J = 8.00 Hz, 1H), 7.26 (d, J = 8.80 Hz, 1H), 7.13 (d, J = 2.00 Hz, 1H), 7.09-7.05 (m, 1H), 6.99-6.96 (m, 2H), 6.71 (dd, J = 2.40, 8.80 Hz, 1H), 3.72-3.67 (m, 6H), 3.56 (t, J = 4.80 Hz, 4H), 2.93-2.78 (m, 3H), 2.04 (t, J = 20.80 Hz, 2H), 1.89-1.79 (m, 2H) 3. 403.1 4. Procedure 3 |
| Example 20 5-(4-(1H- pyrrolo[2,3- b]pyridin-3- yl)piperidin-1- yl)-2- morpholinobenzo [d]oxazole | 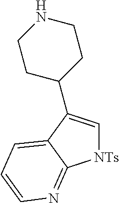 [SMILES: Cc1ccc(S(=O)(=O)n2cc(C3CCNCC3)c3cccnc32)cc1] | 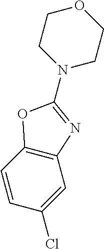 [SMILES: Clc1ccc2oc(N3CCOCC3)nc2c1] | 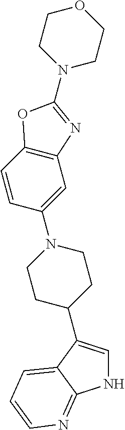 [SMILES: c1cnc2[nH]cc(C3CCN(c4ccc5oc(N6CCOCC6)nc5c4)CC3)c2c1] | 1. 35% 2. ¹H-NMR (400 MHz, DMSO- d₆): δ 11.35 (s, 1H), 8.18-8.17 (m, 1H), 8.02 (d, J = 7.20 Hz, 1H), 7.26-7.25 (m, 2H), 7.01- 7.02 (m, 1H), 6.95 (d, J = 2.00 Hz, 1H), 6.71-6.70 (m, 1H), 3.72-3.67 (m, 6H), 3.56-3.55 (m, 4H), 2.92-2.91 (m, 1H), 2.81 (t, J = 10.80 Hz, 2H), 2.08-2.05 (m, 2H), 1.84-1.83 (m, 2H), . 3. 404.2 4. Procedure 3 |
| Example 21 5-(4-(1-methyl- 1H-pyrrolo[2,3- b]pyridin-3- yl)piperidin-1- yl)-2- morpholinobenzo [d]oxazole | 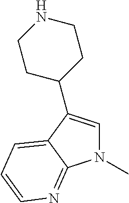 [SMILES: Cn1cc(C2CCNCC2)c2cccnc21] | 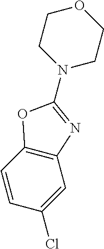 [SMILES: Clc1ccc2oc(N3CCOCC3)nc2c1] | 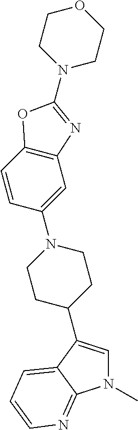 [SMILES: Cn1cc(C2CCN(c3ccc4oc(N5CCOCC5)nc4c3)CC2)c2cccnc21] | 1. 31% 2. ¹H-NMR (400 MHz, DMSO- d₆): δ 8.24-8.23 (m, 1H), 8.04 (dd, J = 1.20, 7.80 Hz, 1H), 7.33 (s, 1H), 7.26 (d, J = 8.80 Hz, 1H), 7.05-7.04 (m, 1H), 6.96 (d, J = 2.40 Hz, 1H), 6.71 (dd, J = 2.40, 8.60 Hz, 1H), 3.82 (s, 3H), 3.71- 3.68 (m, 5H), 3.56-3.55 (m, 4H), 2.82-2.79 (m, 3H), 2.08-2.05 (m, 2H), 1.82-1.81 (m, 2H). 3. 418.2 4. Procedure 3 |
| Example 22 5-(4-(5-fluoro- 1H-pyrrolo[2,3- b]pyridin-3- yl)piperidin-1- yl)-2- morpholinobenzo [d]oxazole | 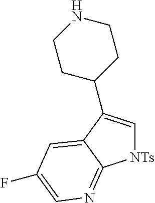 [SMILES: Cc1ccc(S(=O)(=O)n2cc(C3CCNCC3)c3cc(F)cnc32)cc1] | 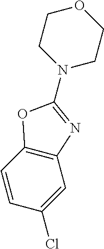 [SMILES: Clc1ccc2oc(N3CCOCC3)nc2c1] | 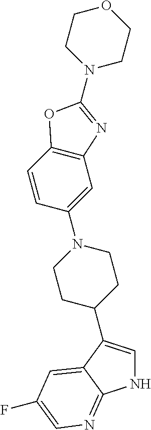 [SMILES: Fc1cnc2[nH]cc(C3CCN(c4ccc5oc(N6CCOCC6)nc5c4)CC3)c2c1] | 1. 23% 2. ¹H-NMR (400 MHz, DMSO- d₆): δ 11.52 (s, 1H), 8.17 (s, 1H), 7.93 (d, J = 8.88 Hz, 1H), 7.38 (s, 1H), 7.26 (d, J = 8.92 Hz, 1H), 6.95 (s, 1H), 6.71 (d, J = 8.92 Hz, 1H), 3.56-3.42 (m, 9H), 2.77-2.80 (m, 4H), 2.07-2.04 (m, 2H), 1.83-1.80 (m, 2H) 3. 422.2 4. Procedure 3 |
| Example 23 5-(4-(5-fluoro-1- methyl-1H- pyrrolo[2,3- b]pyridin-3- yl)piperidin-1- yl)-2- morpholinobenzo [d]oxazole | 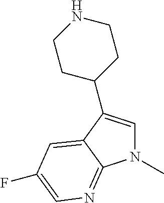 [SMILES: Cn1cc(C2CCNCC2)c2cc(F)cnc21] | 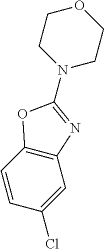 [SMILES: Clc1ccc2oc(N3CCOCC3)nc2c1] | 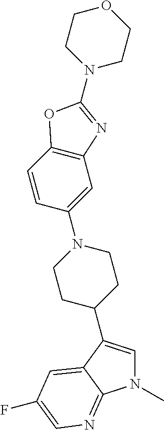 [SMILES: Cn1cc(C2CCN(c3ccc4oc(N5CCOCC5)nc4c3)CC2)c2cc(F)cnc21] | 1. 23% 2. ¹H-NMR (400 MHz, DMSO- d₆): δ 8.23 (s, 1H), 7.96-7.95 (m, 1H), 7.45 (d, J = 3.08 Hz, 1H), 7.25-7.24 (m, 1H), 6.95 (d, J = 2.52 Hz, 1H), 6.70-6.69 (m, 1H), 3.71-3.67 (m, 9H), 3.57-3.56 (m, 4H), 2.81-2.78 (m, 3H), 2.07- 2.04 (m, 2H), 1.78-1.75 (m, 2H 3. 436.0 4. Procedure 3 |
| Example 24 4-(6-(4-(1H- indol-3- yl)piperidin-1- yl)thiazolo[4,5- c]pyridin-2- yl)morpholine | 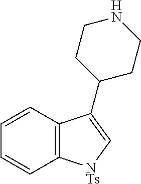 [SMILES: Cc1ccc(S(=O)(=O)n2cc(C3CCNCC3)c3ccccc32)cc1] | 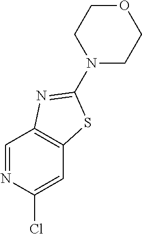 [SMILES: Clc1cc2sc(N3CCOCC3)nc2cn1] | 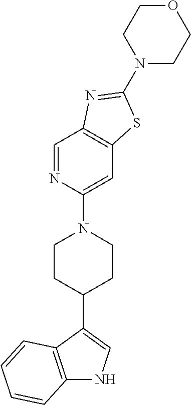 [SMILES: c1ccc2c(C3CCN(c4cc5sc(N6CCOCC6)nc5cn4)CC3)c[nH]c2c1] | 1. 48% 2. ¹H-NMR (400 MHz, DMSO- d₆): δ 10.81 (s, 1H), 8.34 (d, J = 0.80 Hz, 1H), 7.58 (d, J = 8.00 Hz, 1H), 7.35 (t, J = 8.00 Hz, 2H), 7.11-7.04 (m, 2H), 6.99- 6.95 (m, 1H), 4.36 (d, J = 12.80 Hz, 2H), 3.73 (t, J = 5.20 Hz, 4H), 3.51 (t, J = 4.80 Hz, 4H), 3.05-2.92 (m, 3H), 2.04 (d, J = 11.20 Hz, 2H), 1.75-1.65 (m, 2H). 3. 419.9 4. Procedure 2 |
| Example 25 5-(4- (imidazo[1,2- a]pyridin-3- yl)piperidin-1- yl)-2- morpholinobenzo [d]oxazole | 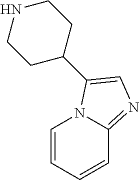 [SMILES: c1ccn2c(C3CCNCC3)cnc2c1] | 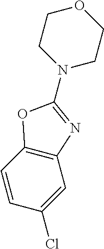 [SMILES: Clc1ccc2oc(N3CCOCC3)nc2c1] | 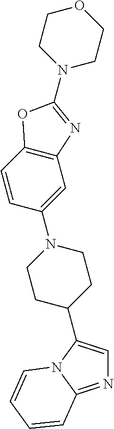 [SMILES: c1ccn2c(C3CCN(c4ccc5oc(N6CCOCC6)nc5c4)CC3)cnc2c1] | 1. 30% 2. ¹H-NMR (400 MHz, DMSO- d₆): δ 8.48 (d, J = 6.80 Hz, 1H), 7.57 (d, J = 8.80 Hz, 1H), 7.44 (s, 1H), 7.22-7.23 (m, 2H), 6.93- 6.92 (m, 2H), 6.72 (dd, J = 2.40, 8.80 Hz, 1H), 3.69-3.71 (m, 6H), 3.56-3.55 (m, 4H), 3.17-3.16 (m, 1H), 2.87-2.85 (m, 2H), 2.15- 2.11 (m, 2H), 1.81-1.80 (m, 2H). 3. 404.2 4. Procedure 2 |
| Example 26 4-(6-(4-(5-fluoro- 1-methyl-1H- pyrrolo[2,3- b]pyridin-3- yl)piperidin-1- yl)thiazolo[4,5- b]pyridin-2- yl)morpholine | 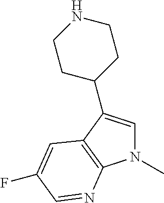 [SMILES: Cn1cc(C2CCNCC2)c2cc(F)cnc21] | 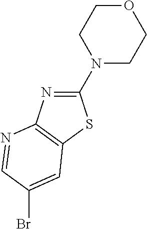 [SMILES: Brc1cnc2nc(N3CCOCC3)sc2c1] | 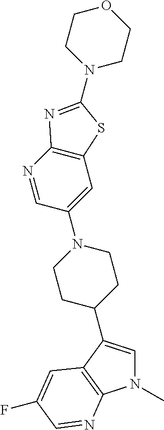 [SMILES: Cn1cc(C2CCN(c3cnc4nc(N5CCOCC5)sc4c3)CC2)c2cc(F)cnc21] | 1. 17% 2. ¹H-NMR (400 MHz, DMSO- d₆): δ 8.23 (s, 1H), 8.14 (d, J = 2.80 Hz, 1H), 7.97-7.96 (m, 1H), 7.94 (d, J = 2.80 Hz, 1H), 7.46 (s, 1H), 3.74-3.73 (m, 9H), 3.55- 3.54 (m, 4H), 2.84-2.87 (m, 3H), 2.09-2.07 (m, 2H), 1.77-1.75 (m, 2H). 3. 453.2 4. Procedure 3 |
| Example 27 4-(6-(4-(5-fluoro- 1-methyl-1H- pyrrolo[2,3- b]pyridin-3- yl)piperidin-1- yl)thiazolo[4,5- c]pyridin-2- yl)morpholine | 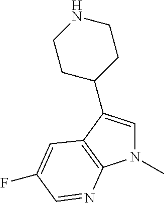 [SMILES: Cn1cc(C2CCNCC2)c2cc(F)cnc21] | 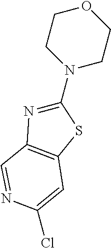 [SMILES: Clc1cc2sc(N3CCOCC3)nc2cn1] | 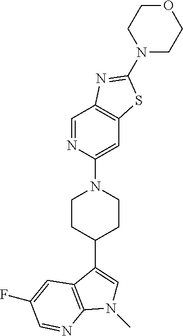 [SMILES: Cn1cc(C2CCN(c3cc4sc(N5CCOCC5)nc4cn3)CC2)c2cc(F)cnc21] | 1. 10% 2. ¹H-NMR (400 MHz, DMSO- d₆): δ 8.33 (d, J = 0.40 Hz, 1H), 8.23-8.22 (m, 1H), 7.96 (dd, J = 2.80, 9.60 Hz, 1H), 7.42 (s, 1H), 7.38 (s, 1H), 4.35 (d, J = 13.20 Hz, 2H), 3.75 (d, J = 8.00 Hz, 3H), 3.72 (d, J = 4.80 Hz, 4H), 3.51 (t, J = 4.40 Hz, 4H), 3.02- 2.90 (m, 3H), 2.04 (d, J = 11.20 Hz, 2H), 1.70-1.64 (m, 2H). 3. 453.2 4. Procedure 3 |
| Example 28 4-(6-(4-(1H- indol-3- yl)piperidin-1- yl)thiazolo[4,5- b]pyridin-2- yl)morpholine | 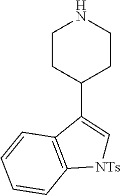 [SMILES: Cc1ccc(S(=O)(=O)n2cc(C3CCNCC3)c3ccccc32)cc1] | 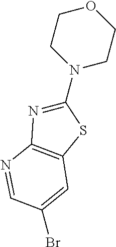 [SMILES: Brc1cnc2nc(N3CCOCC3)sc2c1] | 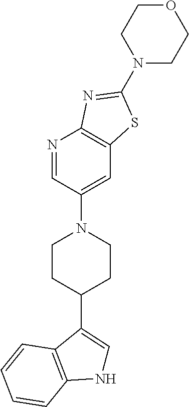 [SMILES: c1ccc2c(C3CCN(c4cnc5nc(N6CCOCC6)sc5c4)CC3)c[nH]c2c1] | 1. 52% 2. ¹H-NMR (400 MHz, DMSO- d₆): δ 10.81 (s, 1H), 8.15 (d, J = 3.20 Hz, 1H), 7.94 (d, J = 2.80 Hz, 1H), 7.59 (d, J = 8.00 Hz, 1H), 7.35 (d, J = 8.00 Hz, 1H), 7.14 (d, J = 2.00 Hz, 1H), 7.09- 7.05 (m, 1H), 7.00-6.96 (m, 1H), 3.77-3.73 (m, 6H), 3.58-3.54 (m, 4H), 2.94-2.86 (m, 3H), 2.08 (d, J = 11.60 Hz, 2H), 1.86-1.82 (m, 2H). 3. 420.0 4. Procedure 1 |
| Example 30 4-(6-(4-(6-fluoro- 1H-pyrrolo[3,2- b]pyridin-3- yl)piperidin-1- yl)benzo[d]thia- zol-2- yl)morpholine | 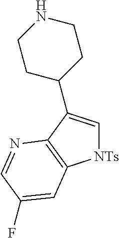 [SMILES: Cc1ccc(S(=O)(=O)n2cc(C3CCNCC3)c3ncc(F)cc32)cc1] | 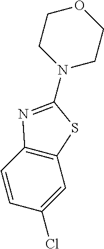 [SMILES: Clc1ccc2nc(N3CCOCC3)sc2c1] | 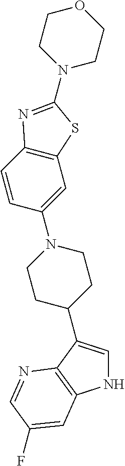 [SMILES: Fc1cnc2c(C3CCN(c4ccc5nc(N6CCOCC6)sc5c4)CC3)c[nH]c2c1] | 1. 20% 2. ¹H-NMR (400 MHz, DMSO- d6) : δ 8.42 (d, J = 1.20 Hz, 1H), 8.08 (d, J = 2.00 Hz, 1H), 7.84- 7.77 (m, 2H), 7.61 (dd, J = 3.20, 8.40 Hz, 1H), 7.50 (dd, J = 2.40, 8.60 Hz, 1H), 3.75 (t, J = 4.80 Hz, 4H), 3.58 (t, J = 4.40 Hz, 4H), 3.24-3.14 (m, 2H), 2.96- 2.87 (m, 2H), 2.16 (d, J = 12.00 Hz, 2H), 1.88-1.80 (m, 4H) 3. 438.2 4. Procedure 3 |
| Example 31 5-(4-(1-methyl- 1H-pyrazolo[3,4- b]pyridin-3- yl)piperidin-1- yl)-2- morpholinobenzo [d]oxazole | 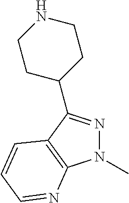 [SMILES: Cn1nc(C2CCNCC2)c2cccnc21] | 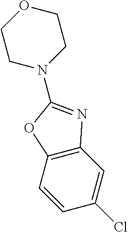 [SMILES: Clc1ccc2oc(N3CCOCC3)nc2c1] | 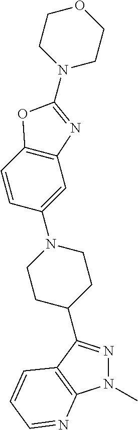 [SMILES: Cn1nc(C2CCN(c3ccc4oc(N5CCOCC5)nc4c3)CC2)c2cccnc21] | 1. 60% 2. ¹H-NMR (400 MHz, DMSO- d6): δ 8.54 (dd, J = 1.60, 4.40 Hz, 1H), 8.34 (dd, J = 1.60, 8.00 Hz, 1H), 7.26 (d, J = 8.80 Hz, 1H), 7.18 (dd, J = 4.40, 8.00 Hz, 1H), 6.96 (d, J = 2.40 Hz, 1H), 6.72 (dd, J = 2.40, 8.80 Hz, 1H), 4.01 (s, 3H), 3.72 (t, J = 5.20 Hz, 6H), 3.56 (t, J = 4.40 Hz, 4H), 3.23-3.18 (m, 1H), 2.89-2.82 (m, 2H), 2.12-1.99 (m, 4H). 3. 419.1 4. Procedure 2 |
| Example 32 5-(4-(5-fluoro-1- methyl-1H- pyrrolo[2,3- b]pyridin-3-yl)- 3,6- dihydropyridin- 1(2H)-yl)-2- morpholinobenzo [d]oxazole | 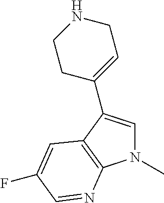 [SMILES: Cn1cc(C2=CCNCC2)c2cc(F)cnc21] | 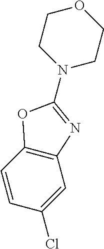 [SMILES: Clc1ccc2oc(N3CCOCC3)nc2c1] | 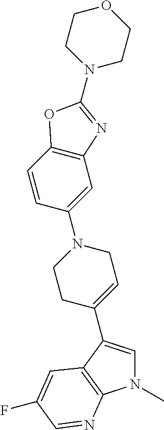 [SMILES: Cn1cc(C2=CCN(c3ccc4oc(N5CCOCC5)nc4c3)CC2)c2cc(F)cnc21] | 1. 16% 2. ¹H-NMR (400 MHz, DMSO- d₆): δ 8.29-8.28 (m, 1H), 8.19 (dd, J = 2.40, 10.20 Hz, 1H), 7.76 (s, 1H), 7.28 (d, J = 8.40 Hz, 1H), 6.98 (s, 1H), 6.72-6.71 (m, 1H), 6.31 (s, 1H), 3.84-3.82 (m, 5H), 3.72-3.71 (m, 4H), 3.57- 3.55 (m, 4H), 3.44-3.43 (m, 2H), 2.64 (bs, 2H). 3. 434.2 4. Procedure 2 |
| Example 33 5-(3-(5-fluoro-1- methyl-1H- pyrrolo[2,3- b]pyridin-3- yl)pyrrolidin-1- yl)-2- morpholinobenzo [d]oxazole Enantiopure 1 | 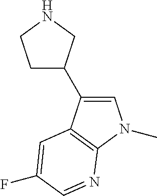 [SMILES: Cn1cc(C2CCNC2)c2cc(F)cnc21] | 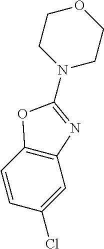 [SMILES: Clc1ccc2oc(N3CCOCC3)nc2c1] | 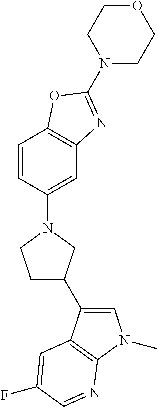 [SMILES: Cn1cc(C2CCN(c3ccc4oc(N5CCOCC5)nc4c3)C2)c2cc(F)cnc21] | 1. 44% 2. ¹H-NMR (400 MHz, DMSO- d₆): δ 8.24-8.25 (m, 1H), 8.01 (dd, J = 2.80, 9.60 Hz, 1H), 7.55 (s, 1H), 7.22 (d, J = 8.40 Hz, 1H), 6.54 (d, J = 2.40 Hz, 1H), 6.27 (dd, J = 2.40, 8.80 Hz, 1H), 3.70- 3.71 (m, 9H), 3.54-3.55 (m, 5H), 3.41-3.42 (m, 2H), 3.24-3.26 (m, 1H), 2.10-2.21 (m, 1H) 3. 422.0 4. Procedure 5 |
| Example 34 5-(3-(5-fluoro-1- methyl-1H- pyrrolo[2,3- b]pyridin-3- yl)pyrrolidin-1- yl)-2- morpholinobenzo [d]oxazole Enantiopure 2 | 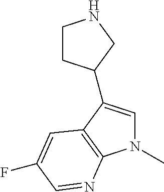 [SMILES: Cn1cc(C2CCNC2)c2cc(F)cnc21] | 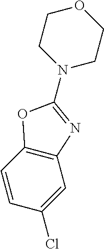 [SMILES: Clc1ccc2oc(N3CCOCC3)nc2c1] | 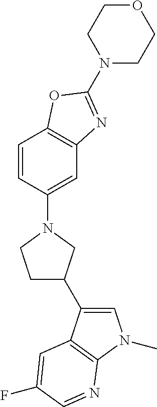 [SMILES: Cn1cc(C2CCN(c3ccc4oc(N5CCOCC5)nc4c3)C2)c2cc(F)cnc21] | 1. 24% 2. ¹H-NMR (400 MHz, DMSO- d₆): δ 8.25-8.24 (m, 1H), 8.01 (dd, J = 2.80, 9.60 Hz, 1H), 7.55 (s, 1H), 7.22 (d, J = 8.80 Hz, 1H), 6.54 (d, J = 2.40 Hz, 1H), 6.27 (dd, J = 2.40, 8.80 Hz, 1H), 3.77 (s, 3H), 3.70-3.71 (m, 6H), 3.56- 3.55 (m, 4H), 3.41-3.40 (m, 2H), 3.26 (t, J = 7.60 Hz, 1H), 2.10- 2.21 (m, 1H) 3. 422.0 4. Procedure 5 |
| Example 35 4-(6-(4-(1H- indazol-3- yl)piperidin-1- yl)thiazolo[4,5- c]pyridin-2- yl)morpholine | 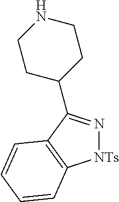 [SMILES: Cc1ccc(S(=O)(=O)n2nc(C3CCNCC3)c3ccccc32)cc1] | 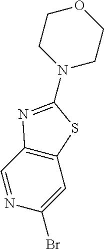 [SMILES: Brc1cc2sc(N3CCOCC3)nc2cn1] | 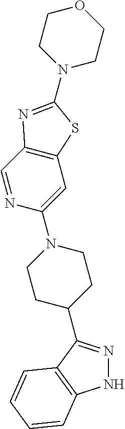 [SMILES: c1ccc2c(C3CCN(c4cc5sc(N6CCOCC6)nc5cn4)CC3)n[nH]c2c1] | 1. 6% 2. ¹H-NMR (400 MHz, DMSO- d₆): δ 8.34 (s, 1H), 7.79 (d, J = 8.00 Hz, 1H), 7.47 (d, J = 8.40 Hz, 1H), 7.40 (s, 1H), 7.34-7.29 (m, 1H), 7.04-7.08 (m, 1H), 4.35 (d, J = 12.80 Hz, 2H), 3.74-3.72 (m, 4H), 3.50-3.53 (m, 5H), 3.01 (t, J = 10.40 Hz, 2H), 2.06-2.03 (m, 2H), 1.87-1.91 (m, 2H). 3. 421.1 4. Procedure 5 |
| Example 36 N-(1H-indazol-3- yl)-2- morpholinobenzo [d]oxazol-5- amine | 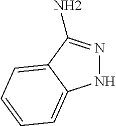 | 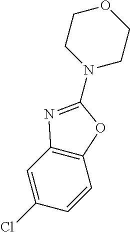 [SMILES: Clc1ccc2oc(N3CCOCC3)nc2c1] | 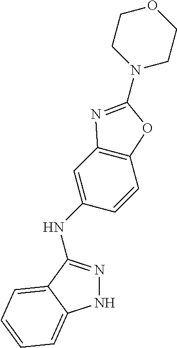 [SMILES: c1ccc2c(Nc3ccc4oc(N5CCOCC5)nc4c3)n[nH]c2c1] | 1. 5% 2. ¹H-NMR (400 MHz, DMSO- d₆): δ 7.83 (d, J = 8.00 Hz, 1H), 7.66 (d, J = 8.40 Hz, 1H), 7.51 (d, J = 8.40 Hz, 2H), 7.50-7.36 (m, 1H), 7.30 (dd, J = 2.00, 8.60 Hz, 1H), 7.08 (t, J = 7.20 Hz, 1H), 5.86 (s, 2H), 3.75 (t, J = 5.20 Hz, 4H), 3.63 (t, J = 4.40 Hz, 4H). 3. 336.1 4. Procedure 6 |
| Example 37 5-(3-(1H- indazol-3- yl)pyrrolidin-1- yl)-2- morpholinobenzo [d]oxazole Enantiopure 1 | 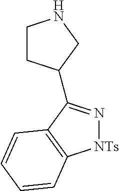 [SMILES: Cc1ccc(S(=O)(=O)n2nc(C3CCNC3)c3ccccc32)cc1] | 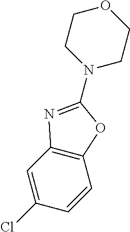 [SMILES: Clc1ccc2oc(N3CCOCC3)nc2c1] | 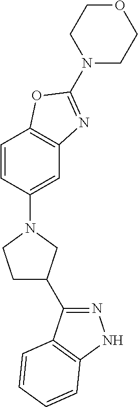 [SMILES: c1ccc2c(C3CCN(c4ccc5oc(N6CCOCC6)nc5c4)C3)n[nH]c2c1] | 1. 10% 2 ¹H-NMR (400 MHz, DMSO- d₆): δ 12.75 (s, 1H), 7.82 (d, J = 8.00 Hz, 1H), 7.49 (d, J = 8.40 Hz, 1H), 7.34 (t, J = 7.60 Hz, 1H), 7.22 (d, J = 8.80 Hz, 1H), 7.09 (t, J = 7.20 Hz, 1H), 6.54 (d, J = 2.40 Hz, 1H), 6.28 (dd, J = 2.40, 8.80 Hz, 1H), 3.99 (t, J = 8.80 Hz, 1H), 3.78 (t, J = 8.00 Hz, 1H), 3.71 (t, J = 5.20 Hz, 5H), 3.56-3.53 (m, 6H), 3.41- 3.47 (m, 2H). 3. 390.2 4. Procedure 3 |
| Example 38 5-(3-(1H- indazol-3- yl)pyrrolidin-1- yl)-2- morpholinobenzo [d]oxazole Enantiopure 2 | 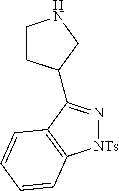 [SMILES: Cc1ccc(S(=O)(=O)n2nc(C3CCNC3)c3ccccc32)cc1] | 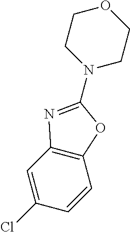 [SMILES: Clc1ccc2oc(N3CCOCC3)nc2c1] | 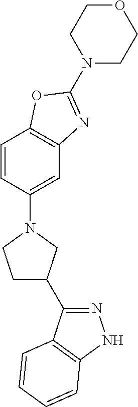 [SMILES: c1ccc2c(C3CCN(c4ccc5oc(N6CCOCC6)nc5c4)C3)n[nH]c2c1] | 1. 10% 2. ¹H-NMR (400 MHz, DMSO- d₆): δ 12.75 (s, 1H), 7.82 (d, J = 8.00 Hz, 1H), 7.49 (d, J = 8.40 Hz, 1H), 7.36-7.32 (m, 1H), 7.22 (d, J = 8.80 Hz, 1H), 7.11-7.07 (m, 1H), 6.54 (d, J = 2.40 Hz, 1H), 6.28 (dd, J = 2.40, 8.80 Hz, 1H), 4.01-3.97 (m, 1H), 3.78 (t, J = 8.00 Hz, 1H), 3.71 (t, J = 5.20 Hz, 5H), 3.57-3.53 (m, 6H), 3.47-3.41 (m, 2H). 3. 390.2 4. Procedure 3 |
| Example 39 5-(3-(1H-indol-3- yl)pyrrolidin-1- yl)-2- morpholinobenzo [d]oxazole Enantiopure 1 | 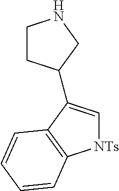 [SMILES: Cc1ccc(S(=O)(=O)n2cc(C3CCNC3)c3ccccc32)cc1] | 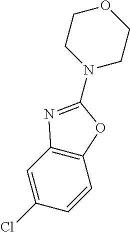 [SMILES: Clc1ccc2oc(N3CCOCC3)nc2c1] | 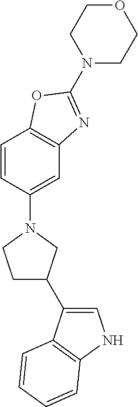 [SMILES: c1ccc2c(C3CCN(c4ccc5oc(N6CCOCC6)nc5c4)C3)c[nH]c2c1] | 1. 19% 2. ¹H-NMR (400 MHz, DMSO- d₆): δ 10.89 (s, 1H), 7.62 (d, J = 8.00 Hz, 1H), 7.36 (d, J = 8.00 Hz, 1H), 7.21 (d, J = 8.40 Hz, 2H), 7.10-7.07 (m, 1H), 7.01- 6.97 (m, 1H), 6.52 (d, J = 2.00 Hz, 1H), 6.26 (dd, J = 2.00, 8.60 Hz, 1H), 3.75-3.70 (m, 6H), 3.56-3.54 (m, 4H), 3.44-3.34 (m, 3H), 2.21-2.16 (m, 2H). 3. 389.2 4. Procedure 3 |
| Example 40 5-(3-(1H-indol-3- yl)pyrrolidin-1- yl)-2- morpholinobenzo [d]oxazole Enantiopure 2 | 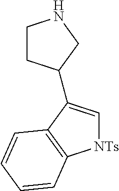 [SMILES: Cc1ccc(S(=O)(=O)n2cc(C3CCNC3)c3ccccc32)cc1] | 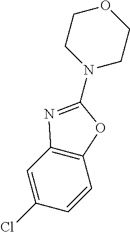 [SMILES: Clc1ccc2oc(N3CCOCC3)nc2c1] | 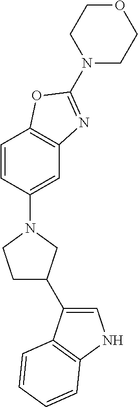 [SMILES: c1ccc2c(C3CCN(c4ccc5oc(N6CCOCC6)nc5c4)C3)c[nH]c2c1] | 1. 14% 2. ¹H-NMR (400 MHz, DMSO- d₆): δ 10.89 (s, 1H), 7.62 (d, J = 7.60 Hz, 1H), 7.36 (d, J = 8.00 Hz, 1H), 7.21 (d, J = 8.80 Hz, 2H), 7.09 (t, J = 7.20 Hz, 1H), 6.99 (t, J = 7.20 Hz, 1H), 6.52 (d, J = 2.40 Hz, 1H), 6.26 (dd, J = 2.40, 8.80 Hz, 1H), 3.89-3.70 (m, 6H), 3.61-3.54 (m, 4H), 3.48- 3.41 (m, 3H), 2.30-2.14 (m, 2H). 3. 389.2 4. Procedure 3 |
| Example 41 4-(6-(4-(1H- indazol-3- yl)piperidin-1- yl)thiazolo[4,5- b]pyridin-2- yl)morpholine | 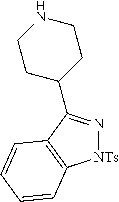 [SMILES: Cc1ccc(S(=O)(=O)n2nc(C3CCNCC3)c3ccccc32)cc1] | 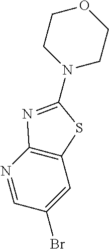 [SMILES: Brc1cnc2nc(N3CCOCC3)sc2c1] | 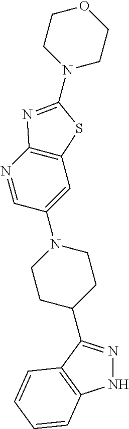 [SMILES: c1ccc2c(C3CCN(c4cnc5nc(N6CCOCC6)sc5c4)CC3)n[nH]c2c1] | 1. 26% 2. ¹H-NMR (400 MHz, DMSO- d6): δ 12.68 (s, 1H), 8.15 (d, J = 2.80 Hz, 1H), 795 (d, J = 2.80 Hz, 1H), 7.81 (d, J = 8.00 Hz, 1H), 7.48 (d, J = 8.40 Hz, 1H), 7.35-7.30 (m, 1H), 7.10-7.06 (m, 1H), 3.79-3.73 (m, 6H), 3.54- 3.57 (m, 4H), 3.33-3.20 (m, 1H), 2.96-2.90 (m, 2H), 2.12-2.05 (m, 4H) 3. 421.2 4. Procedure 5 |
| Example 42 4-(6-(4-(1H- indazol-3- yl)piperidin-1- yl)thiazolo[5,4- b]pyridin-2- yl)morpholine | 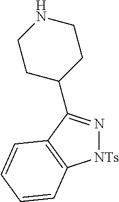 [SMILES: Cc1ccc(S(=O)(=O)n2nc(C3CCNCC3)c3ccccc32)cc1] | 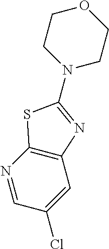 [SMILES: Clc1cnc2sc(N3CCOCC3)nc2c1] | 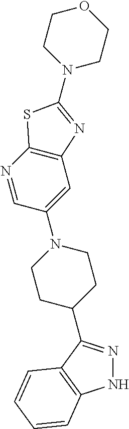 [SMILES: c1ccc2c(C3CCN(c4cnc5sc(N6CCOCC6)nc5c4)CC3)n[nH]c2c1] | 1. 57% 2. ¹H-NMR (400 MHz, DMSO- d6): δ 12.68 (s, 1H), 8.05 (d, J = 2.40 Hz, 1H), 7.80 (d, J = 8.00 Hz, 1H), 7.48 (d, J = 8.40 Hz, 1H), 7.43 (d, J = 2.80 Hz, 1H), 7.34-7.30 (m, 1H), 7.09-7.05 (m, 1H), 3.86 (d, J = 12.40 Hz, 2H), 3.74 (t, J = 5.20 Hz, 4H), 3.57 (t, J = 4.80 Hz, 4H), 3.28-3.23 (m, 1H), 3.00-2.93 (m, 2H), 2.10- 1.99 (m, 4H). 3. 421.2 4. Procedure 6 |
| Example 43 5-(4-(1H- indazol-3- yl)piperidin-1- yl)-2- morpholinooxa- zolo[4,5- b]pyridine | 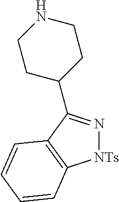 [SMILES: Cc1ccc(S(=O)(=O)n2nc(C3CCNCC3)c3ccccc32)cc1] | 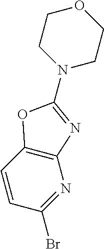 [SMILES: Brc1ccc2oc(N3CCOCC3)nc2n1] | 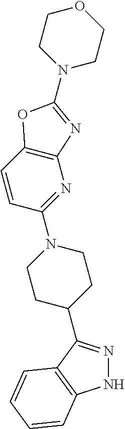 [SMILES: c1ccc2c(C3CCN(c4ccc5oc(N6CCOCC6)nc5n4)CC3)n[nH]c2c1] | 1. 52% 2. ¹H-NMR (400 MHz, DMSO- d6): δ 12.66 (s, 1H), 7.78 (d, J = 8.00 Hz, 1H), 7.58 (d, J = 8.80 Hz, 1H), 7.47 (d, J = 8.40 Hz, 1H), 7.33-7.29 (m, 1H), 7.08- 7.04 (m, 1H), 6.47 (d, J = 8.80 Hz, 1H), 4.34 (d, J = 16.00 Hz, 2H), 3.73 (t, J = 5.20 Hz, 4H), 3.60 (t, J = 4.40 Hz, 4H), 3.31- 3.29 (m, 1H), 3.00 (t, J = 10.80 Hz, 2H), 2.04-2.02 (m, 2H), 1.93-1.83 (m, 2H). 3. 405.2 4. Procedure 5 |
| Example 44 4-(6-(4-(1H- indazol-3- yl)piperidin-1- yl)benzo[d]thia- zol-2- yl)morpholine | 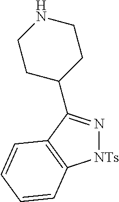 [SMILES: Cc1ccc(S(=O)(=O)n2nc(C3CCNCC3)c3ccccc32)cc1] | 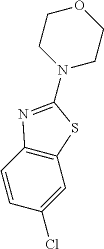 [SMILES: Clc1ccc2nc(N3CCOCC3)sc2c1] | 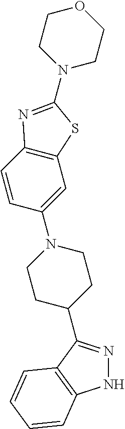 [SMILES: c1ccc2c(C3CCN(c4ccc5nc(N6CCOCC6)sc5c4)CC3)n[nH]c2c1] | 1. 35% 2. ¹H-NMR (400 MHz, DMSO- d6): δ 12.67 (s, 1H), 7.81 (d, J = 8.40 Hz, 1H), 7.44-7.49 (m, 2H), 7.30 (m, 2H), 7.02-7.07 (m, 2H), 4.04-4.03 (m, 6H), 3.77-3.72 (m, 4H), 3.22-3.18 (m, 1H), 2.91- 2.85 (m, 2H), 2.11-2.00 (m, 4H). 3. 420.2 4. Procedure 5 |
| Example 45 6-(4-(1H- indazol-3- yl)piperidin-1- yl)-2- morpholinooxa- zolo[5,4- c]pyridine | 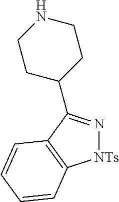 [SMILES: Cc1ccc(S(=O)(=O)n2nc(C3CCNCC3)c3ccccc32)cc1] | 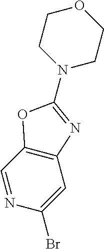 [SMILES: Brc1cc2nc(N3CCOCC3)oc2cn1] | 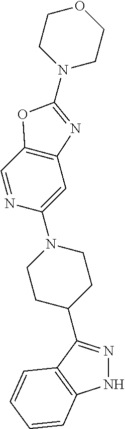 [SMILES: c1ccc2c(C3CCN(c4cc5nc(N6CCOCC6)oc5cn4)CC3)n[nH]c2c1] | 1. 7% 2. ¹H-NMR (400 MHz, DMSO- d6): δ 12.73 (s, 1H), 8.16 (s, 1H), 7.78 (d, J = 8.40 Hz, 1H), 7.47 (d, J = 8.40 Hz, 1H), 7.31 (t, J = 8.00 Hz, 1H), 7.06 (t, J = 7.60 Hz, 1H), 6.78 (s, 1H), 4.34 (d, J = 12.80 Hz, 2H), 3.71-3.70 (m, 4H), 3.63-3.62 (m, 4H), 3.25-3- 34 (m, 1H), 2.98 (t, J = 11.20 Hz, 2H), 2.04-2.01 (m, 2H), 1.87- 1.84 (m, 2H). 3. 405.0 4. Procedure 5 |
| Example 46 5-((5-fluoro-1- methyl-1H- pyrrolo[2,3- b]pyridin-3- yl)ethynyl)-2- morpholinobenzo [d]oxazole | 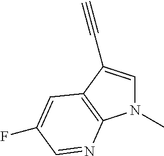 | 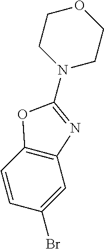 [SMILES: Brc1ccc2oc(N3CCOCC3)nc2c1] | 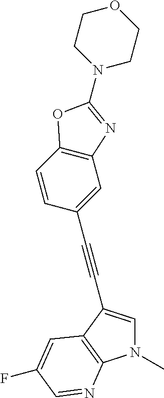 [SMILES: Cn1cc(C#Cc2ccc3oc(N4CCOCC4)nc3c2)c2cc(F)cnc21] | 1. 22% 2. ¹H-NMR (400 MHz, DMSO- d6): δ 8.37-8.36 (m, 1H), 8.04- 8.03 (m, 2H), 7.51-7.50 (m, 1H), 7.46-7.45 (m, 1H), 7.27-7.25 (m, 1H), 3.86 (s, 3H), 3.74-3.73 (m, 4H), 3.62-3.61 (m, 4H). 3. 377.3 4. Procedure 7 |
| Example 47 6-(4-(1H- indazol-3- yl)piperidin-1- yl)-2- morpholinooxa- zolo[4,5- b]pyridine | 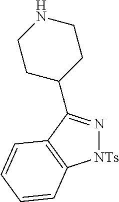 [SMILES: Cc1ccc(S(=O)(=O)n2nc(C3CCNCC3)c3ccccc32)cc1] | 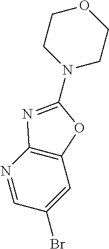 [SMILES: Brc1cnc2nc(N3CCOCC3)oc2c1] | 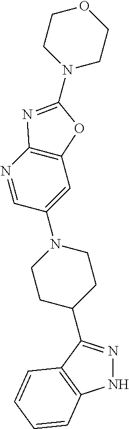 [SMILES: c1ccc2c(C3CCN(c4cnc5nc(N6CCOCC6)oc5c4)CC3)n[nH]c2c1] | 1. 8% 2. ¹H-NMR (400 MHz, DMSO- d6): δ 12.83 (s, 1H), 7.95 (d, J = 2.80 Hz, 1H), 7.81 (d, J = 8.00 Hz, 1H), 7.61 (d, J = 2.40 Hz, 1H), 7.48 (d, J = 8.40 Hz, 1H), 7.32 (t, J = 7.60 Hz, 1H), 7.08 (t, J = 7.20 Hz, 1H), 3.74-3.72 (m, 6H), 3.60-3.59 (m, 4H), 3.19- 3.18 (m, 1H), 2.91-2.89 (m, 2H), 2.03-2.02 (m, 4H). 3. 405.0 4. Procedure 2 |
| Example 48 6-(4-(1H-indol-3- yl)piperidin-1- yl)-2- morpholinooxa- zolo[5,4- c]pyridine | 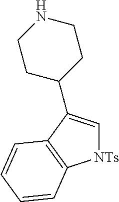 [SMILES: Cc1ccc(S(=O)(=O)n2cc(C3CCNCC3)c3ccccc32)cc1] | 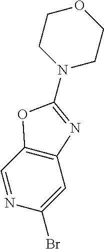 [SMILES: Brc1cc2nc(N3CCOCC3)oc2cn1] | 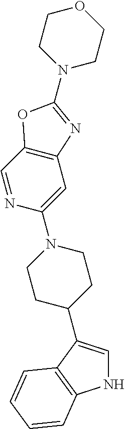 [SMILES: c1ccc2c(C3CCN(c4cc5nc(N6CCOCC6)oc5cn4)CC3)c[nH]c2c1] | 1. 22% 2. ¹H-NMR (400 MHz, DMSO- d6): δ 10.78 (s, 1H), 8.16 (s, 1H), 7.57 (d, J = 7.60 Hz, 1H), 7.34 (d, J = 8.00 Hz, 1H), 7.07 (d, J = 6.80 Hz, 2H), 6.96-6.95 (m, 1H), 6.76 (s, 1H), 4.35 (d, J = 12.00 Hz, 2H), 3.68 (d, J = 4.40 Hz, 8H), 2.95-2.92 (m, 3H), 2.03 (d, J = 12.40 Hz, 2H), 1.68- 1.65 (m, 2H). 3. 404.2 4. Procedure 2 |
| Example 49 6-(4-(1H- indazol-3- yl)piperidin-1- yl)-2- morpholinooxa- zolo[5,4- b]pyridine | 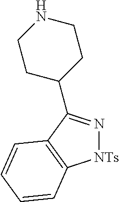 [SMILES: Cc1ccc(S(=O)(=O)n2nc(C3CCNCC3)c3ccccc32)cc1] | 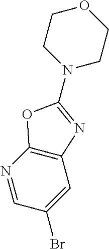 [SMILES: Brc1cnc2oc(N3CCOCC3)nc2c1] | 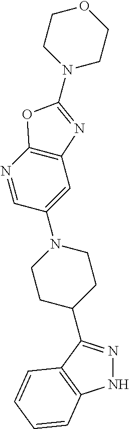 [SMILES: c1ccc2c(C3CCN(c4cnc5oc(N6CCOCC6)nc5c4)CC3)n[nH]c2c1] | 1. 15% 2. ¹H-NMR (400 MHz, DMSO- d6): δ 12.68 (s, 1H), 7.81 (d, J = 8.00 Hz, 1H), 7.64 (s, 1H), 7.48 (d, J = 8.40 Hz, 1H), 7.41 (d, J = 2.80 Hz, 1H), 7.33-7.31 (m, 1H), 7.08-7.06 (m, 1H), 3.61- 3.60 (m, 6H), 3.38-3.34 (m, 5H), 2.92-2.90 (m, 2H), 2.07-2.05 (m, 4H) 3. 405.0 4. Procedure 6 |
| Example 50 5-(4-(1H- indazol-3-yl)- 3,6- dihydropyridin- 1(2H)-yl)-2- morpholinobenzo [d]oxazole | 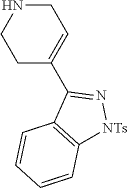 [SMILES: Cc1ccc(S(=O)(=O)n2nc(C3=CCNCC3)c3ccccc32)cc1] | 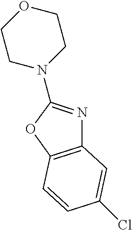 [SMILES: Clc1ccc2oc(N3CCOCC3)nc2c1] | 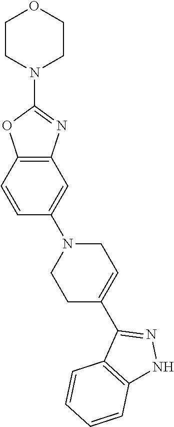 [SMILES: C1=C(c2n[nH]c3ccccc23)CCN(c2ccc3oc(N4CCOCC4)nc3c2)C1] | 1. 37% 2. ¹H-NMR (400 MHz, DMSO- d6): δ 13.00 (s, 1H), 8.03 (d, J = 8.40 Hz, 1H), 7.54 (d, J = 8.40 Hz, 1H), 7.37 (t, J = 8.00 Hz, 1H), 7.29 (d, J = 8.80 Hz, 1H), 7.16 (t, J = 7.60 Hz, 1H), 7.00 (d, J = 2.40 Hz, 1H), 6.74 (dd, J = 2.40, 8.80 Hz, 1H), 6.68 (s, 1H), 3.91 (bs, 2H), 3.72-3.71 (m, 4H), 3.57-3.56 (m, 4H), 3.46 (t, J = 5.60 Hz, 2H), 2.85 (bs, 2H). 3. 402.1 4. Procedure 5 |
| Example 51 5-(4-(5-fluoro-1- methyl-1H- indol-3- yl)piperidin-1- yl)-2- morpholinobenzo [d]oxazole | 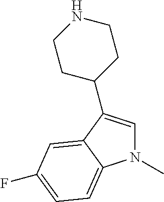 [SMILES: Cn1cc(C2CCNCC2)c2cc(F)ccc21] | 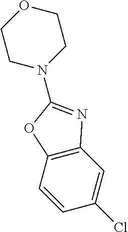 [SMILES: Clc1ccc2oc(N3CCOCC3)nc2c1] | 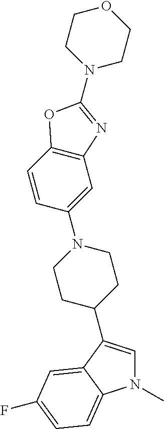 [SMILES: Cn1cc(C2CCN(c3ccc4oc(N5CCOCC5)nc4c3)CC2)c2cc(F)ccc21] | 1. 45% 2. ¹H-NMR (400 MHz, DMSO- d6): δ 7.39-7.38 (m, 2H), 7.21- 7.24 (m, 2H), 6.96-6.95 (m, 2H), 6.70-6.69 (m, 1H), 3.71-3.67 (m, 9H), 3.56-3.55 (m, 4H), 2.82- 2.79 (m, 3H), 2.02-2.00 (m, 2H), 1.79-1.77 (m, 2H) 3. 435.3 4. Procedure 5 |
| Example 52 6-(4-(1H-indol-3- yl)piperidin-1- yl)-2- morpholinobenzo [d]oxazole | 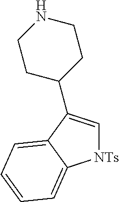 [SMILES: Cc1ccc(S(=O)(=O)n2cc(C3CCNCC3)c3ccccc32)cc1] | 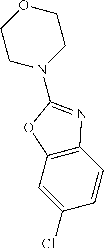 [SMILES: Clc1ccc2nc(N3CCOCC3)oc2c1] | 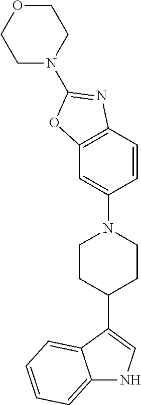 [SMILES: c1ccc2c(C3CCN(c4ccc5nc(N6CCOCC6)oc5c4)CC3)c[nH]c2c1] | 1. 50% 2. ¹H-NMR (400 MHz, DMSO- d6): δ 10.80 (s, 1H), 7.59 (d, J = 8.00 Hz, 1H), 7.35 (d, J = 8.00 Hz, 1H), 7.12-7.12 (m, 3H), 7.07 (t, J = 7.20 Hz, 1H), 6.97 (t, J = 7.20 Hz, 1H), 6.87 (dd, J = 2.00, 8.60 Hz, 1H), 3.71-3.68 (m, 6H), 3.53-3.52 (m, 4H), 2.83-2.82 (m, 3H), 2.08-2.05 (m, 2H), 1.83- 1.82 (m, 2H) 3. 403.2 4. Procedure 1 |
| Example 53 5-(4-(6-fluoro- 1H-pyrrolo[3,2- b]pyridin-3- yl)piperidin-1- yl)-2- morpholinobenzo [d]oxazole | 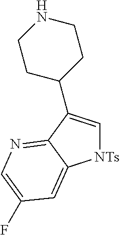 [SMILES: Cc1ccc(S(=O)(=O)n2cc(C3CCNCC3)c3ncc(F)cc32)cc1] | 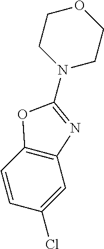 [SMILES: Clc1ccc2oc(N3CCOCC3)nc2c1] | 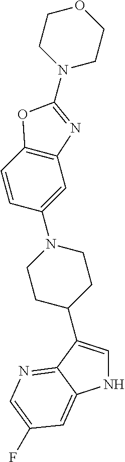 [SMILES: Fc1cnc2c(C3CCN(c4ccc5oc(N6CCOCC6)nc5c4)CC3)c[nH]c2c1] | 1. 23% 2. ¹H-NMR (400 MHz, DMSO- d6) : δ 11.14 (s, 1H), 8.29-8.28 (m, 1H), 7.61 (dd, J = 2.80, 10.00 Hz, 1H), 7.45 (d, J = 2.40 Hz, 1H), 7.25 (d, J = 8.40 Hz, 1H), 6.95 (s, 1H), 6.70 (dd, J = 2.40, 8.80 Hz, 1H), 3.69-3.66 (m, 6H), 3.56-3.55 (m, 4H), 3.00- 2.98 (m, 1H), 2.79-2.77 (m, 2H), 2.12-2.09 (m, 2H), 1.90-1.89 (m, 2H). 3. 421.9 4. Procedure 3 |
| Example 54 5-(4-(1H- pyrrolo[2,3- c]pyridin-3- yl)piperidin-1- yl)-2- morpholinobenzo [d]oxazole | 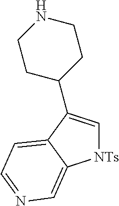 [SMILES: Cc1ccc(S(=O)(=O)n2cc(C3CCNCC3)c3ccncc32)cc1] | 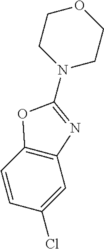 [SMILES: Clc1ccc2oc(N3CCOCC3)nc2c1] | 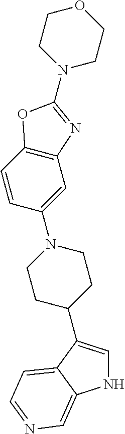 [SMILES: c1cc2c(C3CCN(c4ccc5oc(N6CCOCC6)nc5c4)CC3)c[nH]c2cn1] | 1. 64% 2. ¹H-NMR (400 MHz, DMSO- d6) : δ 11.39 (s, 1H), 8.72 (s, 1H), 8.07 (d, J = 5.60 Hz, 1H), 7.60 (d, J = 5.20 Hz, 1H), 7.44 (s, 1H), 7.26 (d, J = 8.80 Hz, 1H), 6.96 (s, 1H), 6.70-6.72 (m, 1H), 3.71- 3.67 (m, 6H), 3.56-3.55 (m, 4H), 2.82-2.79 (m, 3H), 2.07-2.04 (m, 2H), 1.85-1.84 (m, 2H). 3. 404.1 4. Procedure 3 |
| Example 55 5-(4-(1H- pyrrolo[3,2- c]pyridin-3- yl)piperidin-1- yl)-2- morpholinobenzo [d]oxazole | 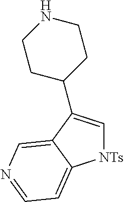 [SMILES: Cc1ccc(S(=O)(=O)n2cc(C3CCNCC3)c3cnccc32)cc1] | 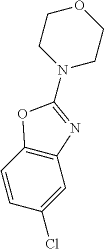 [SMILES: Clc1ccc2oc(N3CCOCC3)nc2c1] | 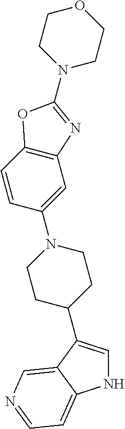 [SMILES: c1cc2[nH]cc(C3CCN(c4ccc5oc(N6CCOCC6)nc5c4)CC3)c2cn1] | 1. 8% 2. ¹H-NMR (400 MHz, DMSO- d6): δ 11.39 (s, 1H), 9.02 (s, 1H), 8.24 (d, J = 5.60 Hz, 1H), 7.60 (d, J = 8.80 Hz, 1H), 7.50-7.49 (m, 2H), 7.44-7.43 (m, 1H), 7.20- 7.20 (m, 1H), 3.66-3.65 (m, 8H), 3.12-3.08 (m, 3H), 2.69-2.68 (m, 2H), 2.08-2.03 (m, 2H), 1.73- 1.72 (m, 2H). 3. 404.0 4. Procedure 5 |
| Example 56 5-(4-(1H-indol-3- yl)piperazin-1- yl)-2- morpholinobenzo [d]oxazole | 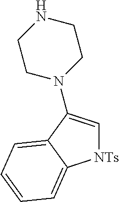 [SMILES: Cc1ccc(S(=O)(=O)n2cc(N3CCNCC3)c3ccccc32)cc1] | 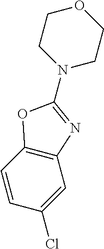 [SMILES: Clc1ccc2oc(N3CCOCC3)nc2c1] | 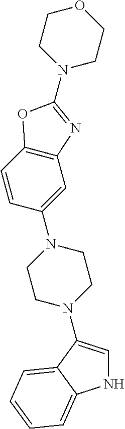 [SMILES: c1ccc2c(N3CCN(c4ccc5oc(N6CCOCC6)nc5c4)CC3)c[nH]c2c1] | 1. 10% 2. ¹H-NMR (400 MHz, DMSO- d6): δ 10.55 (s, 1H), 7.55 (d, J = 8.00 Hz, 1H), 7.30 (dd, J = 8.80, 13.20 Hz, 2H), 7.07-7.05 (m, 1H), 6.94-6.93 (m, 2H), 6.90 (d, J = 2.40 Hz, 1H), 6.72-6.71 (m, 1H), 3.73-3.71 (m, 4H), 3.57- 3.56 (m, 4H), 3.30-3.28 (m, 4H), 3.13-3.12 (m, 4H). 3. 404.0 4. Procedure 1 |
| Example 57 4-(5-(4-(5-fluoro- 1-methyl-1H- pyrrolo[2,3- b]pyridin-3- yl)piperidin-1- yl)thiazolo[5,4- b]pyridin-2- yl)morpholine | 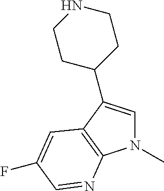 [SMILES: Cn1cc(C2CCNCC2)c2cc(F)cnc21] | 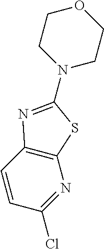 [SMILES: Clc1ccc2nc(N3CCOCC3)sc2n1] | 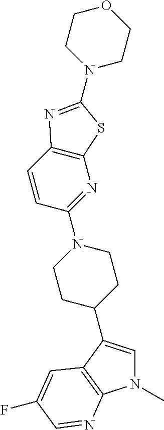 [SMILES: Cn1cc(C2CCN(c3ccc4nc(N5CCOCC5)sc4n3)CC2)c2cc(F)cnc21] | 1. 14% 2. ¹H-NMR (400 MHz, DMSO- d6): δ 8.22-8.21 (m, 1H), 7.97- 7.96 (m, 1H), 7.64 (d, J = 8.80 Hz, 1H), 7.43 (s, 1H), 6.89 (d, J = 9.20 Hz, 1H), 4.35-4.31 (m, 2H), 3.73-3.72 (m, 7H), 3.48- 3.47 (m, 4H), 2.96-2.93 (m, 3H), 2.06-2.03 (m, 2H), 1.64-1.63 (m, 2H). 3. 453.2 4. Procedure 2 |
| Example 58 6-(4-(1H-indol-3- yl)piperidin-1- yl)-2- morpholinooxa- zolo[4,5- c]pyridine | 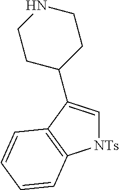 [SMILES: Cc1ccc(S(=O)(=O)n2cc(C3CCNCC3)c3ccccc32)cc1] | 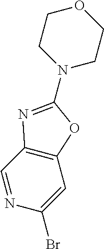 [SMILES: Brc1cc2oc(N3CCOCC3)nc2cn1] | 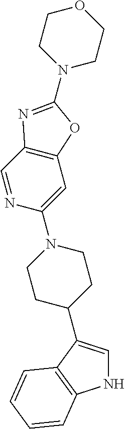 [SMILES: c1ccc2c(C3CCN(c4cc5oc(N6CCOCC6)nc5cn4)CC3)c[nH]c2c1] | 1. 28% 2. ¹H-NMR (400 MHz, DMSO- d6) : δ 10.78 (s, 1H), 8.14 (s, 1H), 7.57 (d, J = 8.00 Hz, 1H), 7.34 (d, J = 8.00 Hz, 1H), 7.06- 7.04 (m, 3H), 6.96-6.95 (m, 1H), 4.37-4.34 (m, 2H), 3.73-3.71 (m, 4H), 3.55-3.54 (m, 4H), 3.01- 3.00 (m, 3H), 2.05-2.02 (m, 2H), 1.71-1.70 (m, 2H). 3. 404.2 4. Procedure 1 |
| Example 59 6-(4- (imidazo[1,2- a]pyridin-3- yl)piperidin-1- yl)-2- morpholinobenzo [d]oxazole | 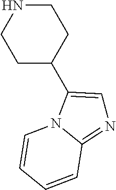 [SMILES: c1ccn2c(C3CCNCC3)cnc2c1] | 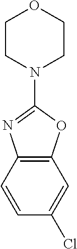 [SMILES: Clc1ccc2nc(N3CCOCC3)oc2c1] | 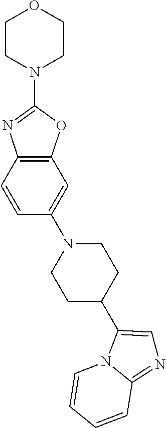 [SMILES: c1ccn2c(C3CCN(c4ccc5nc(N6CCOCC6)oc5c4)CC3)cnc2c1] | 1. 10% 2. ¹H-NMR (400 MHz, DMSO- d6): δ 8.47 (d, J = 6.80 Hz, 1H), 7.56 (d, J = 8.80 Hz, 1H), 7.43 (s, 1H), 7.18-7.14 (m, 3H), 6.89- 6.88 (m, 2H), 3.71-3.70 (m, 6H), 3.53-3.52 (m, 4H), 3.16-3.15 (m, 1H), 2.88-2.85 (m, 2H), 2.14- 2.11 (m, 2H), 1.81-1.80 (m, 2H). 3. 404.0 4. Procedure 2 |
| Example 60 5-(5-(5-fluoro-1- methyl-1H- pyrrolo[2,3- b]pyridin-3- yl)hexahydrocy- clopenta[c]pyr- rol-2(1H)-yl)-2- morpholinobenzo [d]oxazole | 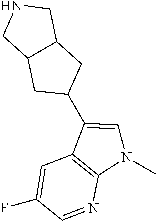 [SMILES: Cn1cc(C2CC3CNCC3C2)c2cc(F)cnc21] | 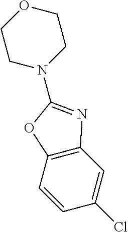 [SMILES: Clc1ccc2oc(N3CCOCC3)nc2c1] | 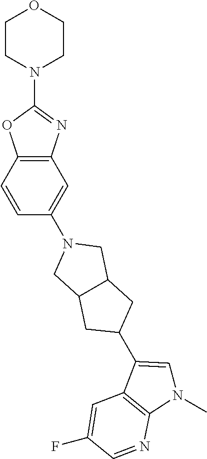 [SMILES: Cn1cc(C2CC3CN(c4ccc5oc(N6CCOCC6)nc5c4)CC3C2)c2cc(F)cnc21] | 1. 50% 2. ¹H-NMR (400 MHz, DMSO- d6): δ 8.21-8.20 (m, 1H), 7.88 (dd, J = 2.80, 9.60 Hz, 1H), 7.45 (s, 1H), 7.21 (d, J = 8.80 Hz, 1H), 6.63 (s, 1H), 6.37-6.36 (m, 1H), 3.71-3.70 (m, 7H), 3.55-3.54 (m, 4H), 3.21-3.20 (m, 5H), 2.88- 2.87 (m, 2H), 2.46-2.34 (m, 2H), 1.52-1.51 (m, 2H). 3. 462.2 4. Procedure 3 |
| Example 61 5-(4-(5-fluoro- 1H-indazol-3- yl)piperidin-1- yl)-2- morpholinobenzo [d]oxazole | 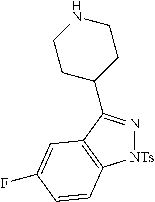 [SMILES: Cc1ccc(S(=O)(=O)n2nc(C3CCNCC3)c3cc(F)ccc32)cc1] | 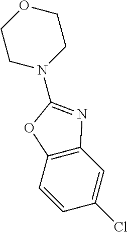 [SMILES: Clc1ccc2oc(N3CCOCC3)nc2c1] | 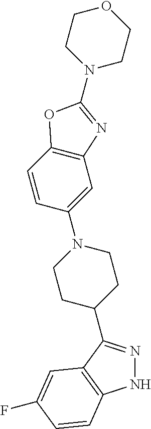 [SMILES: Fc1ccc2[nH]nc(C3CCN(c4ccc5oc(N6CCOCC6)nc5c4)CC3)c2c1] | 1. 28% 2. ¹H-NMR (400 MHz, DMSO- d6): δ 12.80 (s, 1H), 7.60-7.59 (m, 1H), 7.50-7.49 (m, 1H), 7.22- 7.20 (m, 2H), 6.96 (d, J = 2.40 Hz, 1H), 6.71 (dd, J = 2.40, 8.80 Hz, 1H), 3.71-3.69 (m, 6H), 3.56-3.55 (m, 4H), 3.16-3.14 (m, 1H), 2.88-2.87 (m, 2H), 2.03- 2.01 (m, 4H). 3. 422.3 4. Procedure 5 |
| Example 62 4-(5-(4-(1H- indazol-3- yl)piperidin-1- yl)thiazolo[4,5- b]pyridin-2- yl)morpholine | 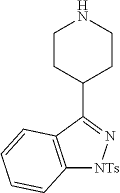 [SMILES: Cc1ccc(S(=O)(=O)n2nc(C3CCNCC3)c3ccccc32)cc1] | 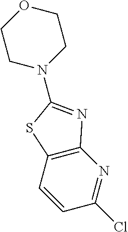 [SMILES: Clc1ccc2sc(N3CCOCC3)nc2n1] | 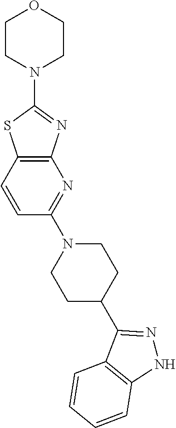 [SMILES: c1ccc2c(C3CCN(c4ccc5sc(N6CCOCC6)nc5n4)CC3)n[nH]c2c1] | 1. 62% 2. ¹H-NMR (400 MHz, DMSO- d6) : δ 12.66 (s, 1H), 7.91 (d, J = 8.40 Hz, 1H), 7.78 (d, J = 8.00 Hz, 1H), 7.47 (d, J = 8.00 Hz, 1H), 7.32-7.30 (m, 1H), 7.07 (t, J = 7.20 Hz, 1H), 6.66 (d, J = 8.80 Hz, 1H), 4.43-4.39 (m, 2H), 3.74-3.72 (m, 4H), 3.56-3.54 (m, 4H), 3.38-3.36 (m, 1H), 3.05 (t, J = 11.60 Hz, 2H), 2.06-2.02 (m, 2H), 1.85-1.84 (m, 2H). 3. 421.2 4. Procedure 5 |
| Example 63 4-(5-(4-(1H- indazol-3- yl)piperidin-1- yl)benzo[d]thia- zol-2- yl)morpholine | 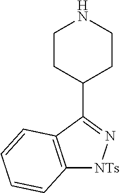 [SMILES: Cc1ccc(S(=O)(=O)n2nc(C3CCNCC3)c3ccccc32)cc1] | 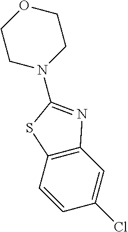 [SMILES: Clc1ccc2sc(N3CCOCC3)nc2c1] | 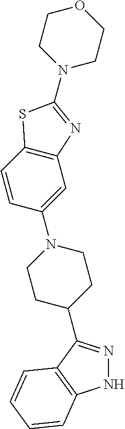 [SMILES: c1ccc2c(C3CCN(c4ccc5sc(N6CCOCC6)nc5c4)CC3)n[nH]c2c1] | 1. 62% 2. ¹H-NMR (400 MHz, DMSO- d6) : δ 12.66 (s, 1H), 7.81 (d, J = 8.00 Hz, 1H), 7.58 (d, J = 8.80 Hz, 1H), 7.48 (d, J = 8.40 Hz, 1H), 7.32 (t, J = 8.00 Hz, 1H), 7.07-7.05 (m, 2H), 6.86 (dd, J = 2.40, 8.60 Hz, 1H), 3.84-3.81 (m, 2H), 3.74-3.72 (m, 4H), 3.52- 3.51 (m, 4H), 3.21-3.18 (m, 1H), 2.89-2.88 (m, 2H), 2.00-1.99 (m, 4H). 3. 420.2 4. Procedure 5 |
| Example 64 5-(4-(1H- indazol-3- yl)piperidin-1- yl)-2- morpholinooxa- zolo[5,4- b]pyridine | 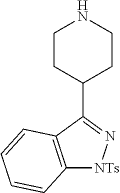 [SMILES: Cc1ccc(S(=O)(=O)n2nc(C3CCNCC3)c3ccccc32)cc1] | 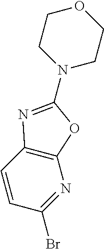 [SMILES: Brc1ccc2nc(N3CCOCC3)oc2n1] | 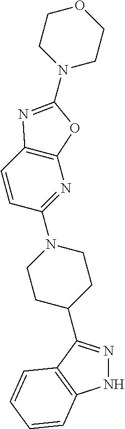 [SMILES: c1ccc2c(C3CCN(c4ccc5nc(N6CCOCC6)oc5n4)CC3)n[nH]c2c1] | 1. 51% 2. ¹H-NMR (400 MHz, DMSO- d6): δ 12.66 (s, 1H), 7.78 (d, J = 8.00 Hz, 1H), 7.55 (d, J = 8.80 Hz, 1H), 7.47 (d, J = 8.40 Hz, 1H), 7.31-7.30 (m, 1H), 7.05- 7.04 (m, 1H), 6.74 (d, J = 8.40 Hz, 1H), 4.29-4.26 (m, 2H), 3.72-3.71 (m, 4H), 3.53-3.52 (m, 4H), 3.01-3.00 (m, 3H), 2.04- 2.02 (m, 2H), 1.93-1.91 (m, 2H). 3. 405.2 4. Procedure 5 |
| Example 65 4-(6-(4-(1H- indol-3- yl)piperidin-1- yl)thiazolo[5,4- b]pyridin-2- yl)morpholine | 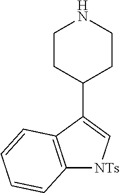 [SMILES: Cc1ccc(S(=O)(=O)n2cc(C3CCNCC3)c3ccccc32)cc1] | 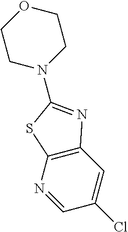 [SMILES: Clc1cnc2sc(N3CCOCC3)nc2c1] | 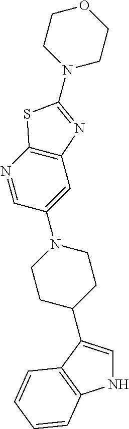 [SMILES: c1ccc2c(C3CCN(c4cnc5sc(N6CCOCC6)nc5c4)CC3)c[nH]c2c1] | 1. 53% 2. ¹H-NMR (400 MHz, DMSO- d6): δ 10.81 (s, 1H), 8.04 (d, J = 2.40 Hz, 1H), 7.59 (d, J = 8.00 Hz, 1H), 7.42 (d, J = 2.40 Hz, 1H), 7.35 (d, J = 8.00 Hz, 1H), 7.13 (d, J = 2.00 Hz, 1H), 7.07 (t, J = 7.60 Hz, 1H), 6.97 (t, J = 7.20 Hz, 1H), 3.85 (d, J = 12.40 Hz, 2H), 3.74-3.72 (m, 4H), 3.57- 3.56 (m, 4H), 2.92-2.89 (m, 3H), 2.07 (d, J = 12.40 Hz, 2H), 1.80- 1.78 (m, 2H). 3. 420.3 4. Procedure 5 |
| Example 66 4-(6-(4-(1H- indazol-3- yl)piperidin-1- yl)thiazolo[5,4- c]pyridin-2- yl)morpholine | 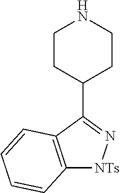 [SMILES: Cc1ccc(S(=O)(=O)n2nc(C3CCNCC3)c3ccccc32)cc1] | 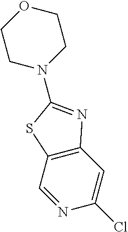 [SMILES: Clc1cc2nc(N3CCOCC3)sc2cn1] | 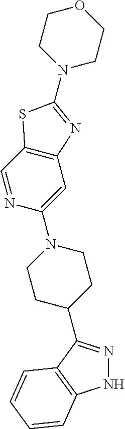 [SMILES: c1ccc2c(C3CCN(c4cc5nc(N6CCOCC6)sc5cn4)CC3)n[nH]c2c1] | 1. 27% 2. ¹H-NMR (400 MHz, DMSO- d6): δ 12.66 (s, 1H), 8.43 (s, 1H), 7.78 (d, J = 8.00 Hz, 1H), 7.46 (d, J = 8.00 Hz, 1H), 7.31 (t, J = 8.00 Hz, 1H), 7.06 (t, J = 7.20 Hz, 1H), 6.88 (s, 1H), 4.39 (d, J = 12.80 Hz, 2H), 3.73-3.72 (m, 4H), 3.61-3.60 (m, 4H), 3.02- 3.01 (m, 3H), 2.04-2.01 (m, 2H), 1.86-1.83 (m, 2H). 3. 421.0 4. Procedure 5 |
| Example 67 4-(6-(4-(1H- indol-3- yl)piperidin-1- yl)thiazolo[5,4- c]pyridin-2- yl)morpholine | 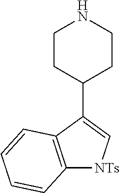 [SMILES: Cc1ccc(S(=O)(=O)n2cc(C3CCNCC3)c3ccccc32)cc1] | 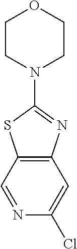 [SMILES: Clc1cc2nc(N3CCOCC3)sc2cn1] | 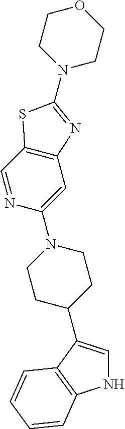 [SMILES: c1ccc2c(C3CCN(c4cc5nc(N6CCOCC6)sc5cn4)CC3)c[nH]c2c1] | 1. 55% 2. ¹H-NMR (400 MHz, DMSO- d6): δ 10.78 (s, 1H), 8.42 (s, 1H), 7.57 (d, J = 7.60 Hz, 1H), 7.34 (d, J = 8.00 Hz, 1H), 7.04-7.03 (m, 2H), 6.96-6.95 (m, 1H), 6.86 (s, 1H), 4.42-4.39 (m, 2H), 3.73- 3.72 (m, 4H), 3.60-3.58 (m, 4H), 2.98-2.95 (m, 3H), 2.01-2.00 (m, 2H), 1.66-1.64 (m, 2H). 3. 420.0 4. Procedure 2 |
| Example 68 5-(4-(1-methyl- 1H-indol-3- yl)piperidin-1- yl)-2- morpholinobenzo [d]oxazole | 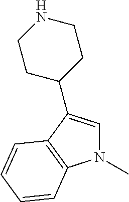 [SMILES: Cn1cc(C2CCNCC2)c2ccccc21] | 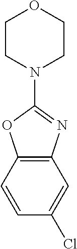 [SMILES: Clc1ccc2oc(N3CCOCC3)nc2c1] | 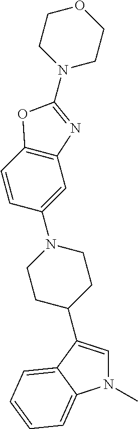 [SMILES: Cn1cc(C2CCN(c3ccc4oc(N5CCOCC5)nc4c3)CC2)c2ccccc21] | 1. 39% 2. ¹H-NMR (400 MHz, DMSO- d6) : δ 7.60 (d, J = 7.84 Hz, 1H), 7.38 (d, J = 8.24 Hz, 1H), 7.26 (d, J = 8.72 Hz, 1H), 7.14-7.13 (m, 2H), 7.01 (t, J = 7.60 Hz, 1H), 6.96 (d, J = 2.24 Hz, 1H), 6.70-6.69 (m, 1H), 3.71-3.67 (m, 9H), 3.56-3.55 (m, 4H), 2.82- 2.79 (m, 3H), 2.07-2.04 (m, 2H), 1.83-1.80 (m, 2H). 3. 417.1 4. Procedure 3 |
| Example 69 5-(4-(5-fluoro- 1H-indol-3- yl)piperidin-1- yl)-2- morpholinobenzo [d]oxazole | 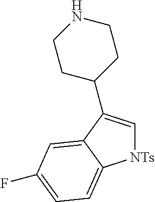 [SMILES: Cc1ccc(S(=O)(=O)n2cc(C3CCNCC3)c3cc(F)ccc32)cc1] | 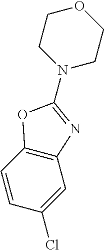 [SMILES: Clc1ccc2oc(N3CCOCC3)nc2c1] | 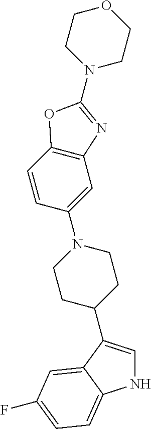 [SMILES: Fc1ccc2[nH]cc(C3CCN(c4ccc5oc(N6CCOCC6)nc5c4)CC3)c2c1] | 1. 28% 2. ¹H-NMR (400 MHz, DMSO- d6): δ 10.92 (s, 1H), 7.33-7.32 (m, 2H), 7.23-7.22 (m, 2H), 6.90- 6.89 (m, 2H), 6.71 (dd, J = 2.32, 8.68 Hz, 1H), 3.71-3.67 (m, 6H), 3.56-3.55 (m, 4H), 2.82-2.79 (m, 3H), 2.03-2.00 (m, 2H), 1.78- 1.76 (m, 2H). 3. 421.2 4. Procedure 3 |
| Example 70 6-(4-(5-fluoro-1- methyl-1H- pyrrolo[2,3- b]pyridin-3- yl)piperidin-1- yl)-2- morpholinobenzo [d]oxazole | 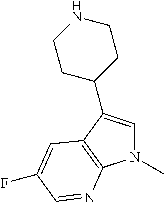 [SMILES: Cn1cc(C2CCNCC2)c2cc(F)cnc21] | 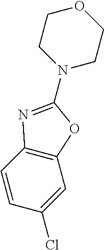 [SMILES: Clc1ccc2nc(N3CCOCC3)oc2c1] | 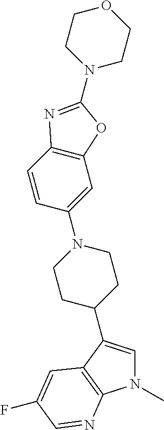 [SMILES: Cn1cc(C2CCN(c3ccc4nc(N5CCOCC5)oc4c3)CC2)c2cc(F)cnc21] | 1. 55% 2. ¹H-NMR (400 MHz, DMSO- d6): δ 8.23-8.22 (m, 1H), 7.97 (dd, J = 2.40, 9.80 Hz, 1H), 7.45 (s, 1H), 7.18 (d, J = 8.80 Hz, 1H), 7.12 (d, J = 2.40 Hz, 1H), 6.87 (dd, J = 2.00, 8.60 Hz, 1H), 3.78 (s, 3H), 3.71-3.68 (m, 6H), 3.53- 3.52 (m, 4H), 2.82-2.81 (m, 3H), 2.07-2.04 (m, 2H), 1.79-1.78 (m, 2H) 3. 436.2 4. Procedure 3 |
| Example 71 4-(5-(4-(1H- indol-3- yl)piperidin-1- yl)thiazolo[5,4- b]pyridin-2- yl)morpholine | 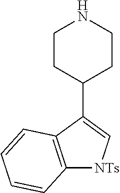 [SMILES: Cc1ccc(S(=O)(=O)n2cc(C3CCNCC3)c3ccccc32)cc1] | 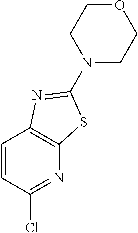 [SMILES: Clc1ccc2nc(N3CCOCC3)sc2n1] | 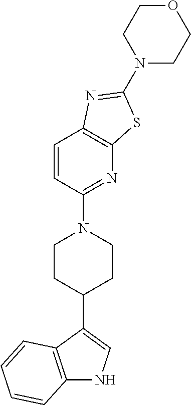 [SMILES: c1ccc2c(C3CCN(c4ccc5nc(N6CCOCC6)sc5n4)CC3)c[nH]c2c1] | 1. 17% 2. ¹H-NMR (400 MHz, DMSO- d6): δ 10.80 (s, 1H), 7.64 (d, J = 9.20 Hz, 1H), 7.58 (d, J = 8.00 Hz, 1H), 7.34 (d, J = 8.00 Hz, 1H), 7.11-7.10 (m, 1H), 7.06- 7.04 (m, 1H), 6.99-6.97 (m, 1H), 6.89 (d, J = 9.20 Hz, 1H), 4.36- 4.32 (m, 2H), 3.73-3.72 (m, 4H), 3.48-3.47 (m, 4H), 2.98-2.95 (m, 3H), 2.07-2.04 (m, 2H), 1.68- 1.67 (m, 2H). 3. 420.2 4. Procedure 1 |
| Example 72 6-(4-(1H- indazol-3- yl)piperidin-1- yl)-2- morpholinooxa- zolo[4,5- c]pyridine | 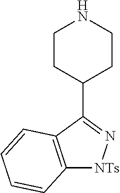 [SMILES: Cc1ccc(S(=O)(=O)n2nc(C3CCNCC3)c3ccccc32)cc1] | 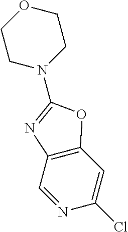 [SMILES: Clc1cc2oc(N3CCOCC3)nc2cn1] | 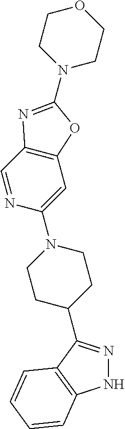 [SMILES: c1ccc2c(C3CCN(c4cc5oc(N6CCOCC6)nc5cn4)CC3)n[nH]c2c1] | 1. 4% 2. ¹H-NMR (400 MHz, DMSO- d6): δ 12.77 (s, 1H), 8.14 (s, 1H), 7.78 (d, J = 8.00 Hz, 1H), 7.47 (d, J = 8.40 Hz, 1H), 7.29-7.28 (m, 1H), 7.05-7.04 (m, 2H), 4.36- 4.33 (m, 2H), 3.73-3.72 (m, 4H), 3.55-3.54 (m, 4H), 2.98-2.97 (m, 3H), 1.90-1.88 (m, 4H). 3. 405.1 4. Procedure 5 |
| Example 73 5-(4-(4-fluoro- 1H-indazol-3- yl)piperidin-1- yl)-2- morpholinobenzo [d]oxazole | 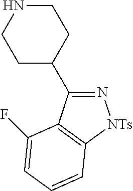 [SMILES: Cc1ccc(S(=O)(=O)n2nc(C3CCNCC3)c3c(F)cccc32)cc1] | 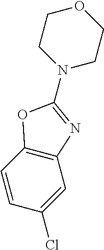 [SMILES: Clc1ccc2oc(N3CCOCC3)nc2c1] | 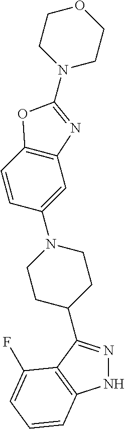 [SMILES: Fc1cccc2[nH]nc(C3CCN(c4ccc5oc(N6CCOCC6)nc5c4)CC3)c12] | 1. 44% 2. ¹H-NMR (400 MHz, DMSO- d6): δ 12.99 (s, 1H), 7.32-7.25 (m, 3H), 6.96 (d, J = 2.00 Hz, 1H), 6.86-6.81 (m, 1H), 6.71 (dd, J = 2.40, 8.80 Hz, 1H), 3.72 (t, J = 5.20 Hz, 6H), 3.57-3.55 (m, 4H), 3.25-3.20 (m,1H), 2.86- 2.83 (m, 2H), 2.05-2.01 (m, 4H). 3. 422.2 4. Procedure 3 |
| Example 74 5-(4-(6-fluoro- 1H-indazol-3- yl)piperidin-1- yl)-2- morpholinobenzo [d]oxazole | 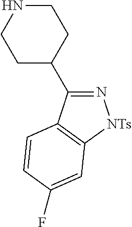 [SMILES: Cc1ccc(S(=O)(=O)n2nc(C3CCNCC3)c3ccc(F)cc32)cc1] | 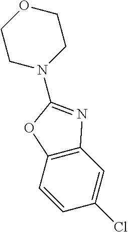 [SMILES: Clc1ccc2oc(N3CCOCC3)nc2c1] | 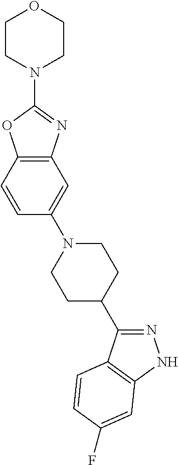 [SMILES: Fc1ccc2c(C3CCN(c4ccc5oc(N6CCOCC6)nc5c4)CC3)n[nH]c2c1] | 1. 31% 2. ¹H-NMR (400 MHz, DMSO- d6): δ 12.75 (s, 1H), 7.85 (dd, J = 5.20, 8.80 Hz, 1H), 7.27-7.23 (m, 2H), 6.98-6.93 (m, 2H), 6.71 (dd, J = 2.40, 8.80 Hz, 1H), 3.73- 3.69 (m, 6H), 3.57-3.55 (m, 4H), 3.22-3.16 (m, 1H), 2.88-2.83 (m, 2H), 2.08-1.91 (m, 4H). 3. 422.2 4. Procedure 3 |
| Example 75 5-(4-(7-fluoro- 1H-indazol-3- yl)piperidin-1- yl)-2- morpholinobenzo [d]oxazole | 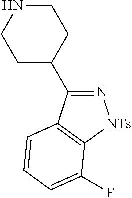 [SMILES: Cc1ccc(S(=O)(=O)n2nc(C3CCNCC3)c3cccc(F)c32)cc1] | 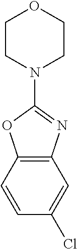 [SMILES: Clc1ccc2oc(N3CCOCC3)nc2c1] | 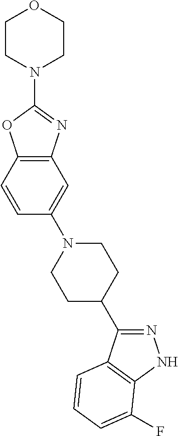 [SMILES: Fc1cccc2c(C3CCN(c4ccc5oc(N6CCOCC6)nc5c4)CC3)n[nH]c12] | 1. 17% 2. ¹H-NMR (400 MHz, DMSO- d6): δ 7.65 (d, J = 8.00 Hz, 1H), 7.26 (d, J = 8.80 Hz, 1H), 7.16- 7.14 (m, 1H), 7.06-7.05 (m, 1H), 6.97 (d, J = 2.40 Hz, 1H), 6.72 (dd, J = 2.40, 8.80 Hz, 1H), 3.71- 3.70 (m, 6H), 3.57-3.55 (m, 4H), 3.21-3.19 (m, 1H), 2.84-2.83 (m, 2H), 2.03-2.01 (m, 4H). 3. 422.2 4. Procedure 5 |
| Example 76 5-(4-(5-methyl- 1H-indazol-3- yl)piperidin-1- yl)-2- morpholinobenzo [d]oxazole | 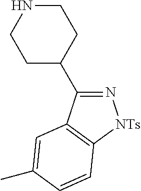 [SMILES: Cc1ccc(S(=O)(=O)n2nc(C3CCNCC3)c3cc(C)ccc32)cc1] | 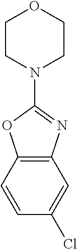 [SMILES: Clc1ccc2oc(N3CCOCC3)nc2c1] | 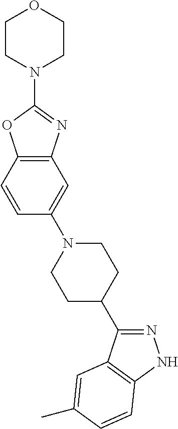 [SMILES: Cc1ccc2[nH]nc(C3CCN(c4ccc5oc(N6CCOCC6)nc5c4)CC3)c2c1] | 1. 30% 2. ¹H-NMR (400 MHz, DMSO- d6): δ 12.52 (s, 1H), 7.57 (s, 1H), 7.37 (d, J = 8.40 Hz, 1H), 7.26 (d, J = 8.80 Hz, 1H), 7.15 (dd, J = 0.80, 8.60 Hz, 1H), 6.96 (d, J = 2.00 Hz, 1H), 6.72 (dd, J = 2.00, 8.60 Hz, 1H), 3.71 (d, J = 6.80 Hz, 6H), 3.57-3.56 (m, 4H), 3.16-3.15 (m, 1H), 2.82-2.81 (m, 2H), 2.41 (s, 3H), 2.03-2.02 (m, 4H) 3. 418.3 4. Procedure 3 |
| Example 77 5-(4-(6-methyl- 1H-indazol-3- yl)piperidin-1- yl)-2- morpholinobenzo [d]oxazole | 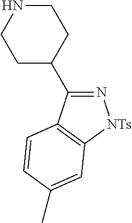 [SMILES: Cc1ccc(S(=O)(=O)n2nc(C3CCNCC3)c3ccc(C)cc32)cc1] | 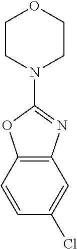 [SMILES: Clc1ccc2oc(N3CCOCC3)nc2c1] | 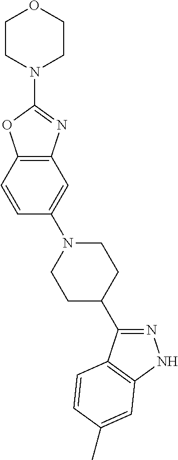 [SMILES: Cc1ccc2c(C3CCN(c4ccc5oc(N6CCOCC6)nc5c4)CC3)n[nH]c2c1] | 1. 64% 2. ¹H-NMR (400 MHz, DMSO- d6): δ 12.49 (s, 1H), 7.25-7.24 (m, 2H), 6.91-6.89 (m, 3H), 6.71 (dd, J = 2.00, 8.60 Hz, 1H), 3.72- 3.71 (m, 6H), 3.56-3.55 (m, 5H), 2.84-2.82 (m, 2H), 2.42 (s, 3H), 2.02-2.00 (m, 4H) 3. 418.3 4. Procedure 3 |
| Example 78 5-(4-(7-methyl- 1H-indazol-3- yl)piperidin-1- yl)-2- morpholinobenzo [d]oxazole | 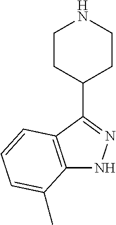 [SMILES: Cc1cccc2c(C3CCNCC3)n[nH]c12] | 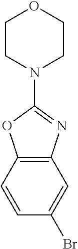 [SMILES: Brc1ccc2oc(N3CCOCC3)nc2c1] | 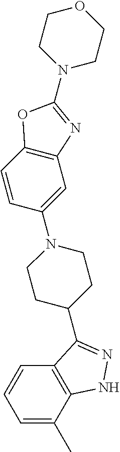 [SMILES: Cc1cccc2c(C3CCN(c4ccc5oc(N6CCOCC6)nc5c4)CC3)n[nH]c12] | 1. 9% 2. ¹H-NMR (400 MHz, DMSO- d6): δ 12.49 (s, 1H), 7.61 (d, J = 8.00 Hz, 1H), 7.26 (d, J = 8.40 Hz, 1H), 7.09 (d, J = 6.80 Hz, 1H), 6.97-6.96 (m, 2H), 6.72 (dd, J = 2.40, 8.80 Hz, 1H), 3.71-3.70 (m, 6H), 3.56-3.55 (m, 5H), 2.85- 2.83 (m, 2H), 2.51 (s, 3H), 2.05- 2.04 (m, 4H) 3. 418.2 4. Procedure 2 |
| Example 79 5-(4-(1H- indazol-3- yl)piperazin-1- yl)-2- morpholinobenzo [d]oxazole | 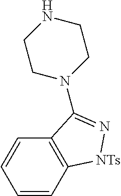 [SMILES: Cc1ccc(S(=O)(=O)n2nc(N3CCNCC3)c3ccccc32)cc1] | 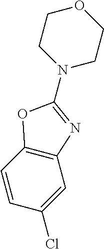 [SMILES: Clc1ccc2oc(N3CCOCC3)nc2c1] | 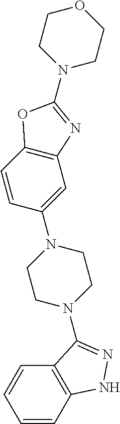 [SMILES: c1ccc2c(N3CCN(c4ccc5oc(N6CCOCC6)nc5c4)CC3)n[nH]c2c1] | 1. 59% 2. ¹H-NMR (400 MHz, DMSO- d6): δ 12.03 (s, 1H), 7.80 (d, J = 8.00 Hz, 1H), 7.39-7.37 (m, 1H), 7.28-7.26 (m, 2H), 7.00-6.99 (m, 2H), 6.73-6.72 (m, 1H), 3.72- 3.71 (m, 4H), 3.57-3.56 (m, 4H), 3.48-3.46 (m, 4H), 3.29-3.28 (m, 4H). 3. 405.2 4. Procedure 5 |
| Example 80 5-(4-(1H- indazol-3- yl)piperidin-1- yl)-2-(4- methoxypiperidin- 1- yl)benzo[d]oxa- zole | 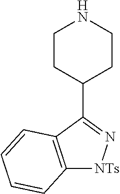 [SMILES: Cc1ccc(S(=O)(=O)n2nc(C3CCNCC3)c3ccccc32)cc1] | 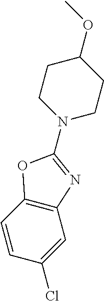 [SMILES: COC1CCN(c2nc3cc(Cl)ccc3o2)CC1] | 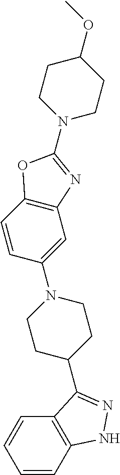 [SMILES: COC1CCN(c2nc3cc(N4CCC(c5n[nH]c6ccccc56)CC4)ccc3o2)CC1] | 1. 60% 2. ¹H-NMR (400 MHz, DMSO- d6): δ 12.66 (s, 1H), 7.81 (d, J = 8.00 Hz, 1H), 7.48 (d, J = 8.40 Hz, 1H), 7.30-7.31 (m, 1H), 7.23 (d, J = 8.80 Hz, 1H), 7.07 (t, J = 8.00 Hz, 1H), 6.93 (d, J = 2.00 Hz, 1H), 6.69 (dd, J = 2.40, 8.80 Hz, 1H), 3.81-3.80 (m, 2H), 3.72-3.69 (m, 2H), 3.39-3.38 (m, 3H), 3.30 (s, 3H), 3.20-3.19 (s, 1H), 2.86-2.83 (m, 2H), 1.94- 1.93 (m, 6H), 1.52-1.51 (m, 2H). 3. 432.3 4. Procedure 5 |
| Example 81 4-(5-(3-(5-fluoro- 1-methyl-1H- pyrrolo[2,3- b]pyridin-3- yl)pyrrolidin-1- yl)benzo[d]thia- zol-2- yl)morpholine | 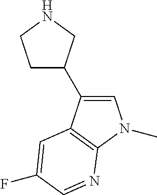 [SMILES: Cn1cc(C2CCNC2)c2cc(F)cnc21] | 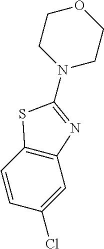 [SMILES: Clc1ccc2sc(N3CCOCC3)nc2c1] | 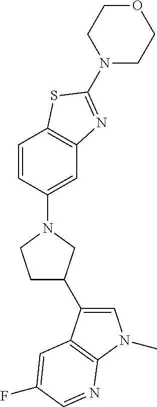 [SMILES: Cn1cc(C2CCN(c3ccc4sc(N5CCOCC5)nc4c3)C2)c2cc(F)cnc21] | 1. 43% 2. ¹H-NMR (400 MHz, DMSO- d6) : δ 8.25 (d, J = 2.40 Hz, 1H), 8.01 (dd, J = 2.80, 9.60 Hz, 1H), 7.54 (d, J = 6.00 Hz, 1H), 7.51 (s, 1H), 6.72 (d, J = 2.40 Hz, 1H), 6.45 (dd, J = 2.40, 8.80 Hz, 1H), 3.81-3.79 (m, 4H), 3.71-3.70 (m, 5H), 3.40-3.38 (m, 6H), 3.29 (t, J = 8.40 Hz, 1H), 2.46-2.45 (m, 1H), 2.14-2.13 (m, 1H),. 3. 438.1 4. Procedure 5 |
| Example 82 4-(6-(3-(5-fluoro- 1-methyl-1H- pyrrolo[2,3- b]pyridin-3- yl)pyrrolidin-1- yl)benzo[d]thia- zol-2- yl)morpholine Enantiopure 1 | 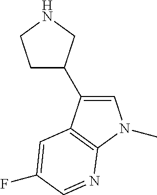 [SMILES: Cn1cc(C2CCNC2)c2cc(F)cnc21] | 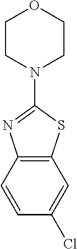 [SMILES: Clc1ccc2nc(N3CCOCC3)sc2c1] | 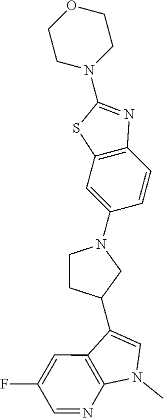 [SMILES: Cn1cc(C2CCN(c3ccc4nc(N5CCOCC5)sc4c3)C2)c2cc(F)cnc21] | 1. 28% 2. ¹H-NMR (400 MHz, DMSO- d6): δ 8.25 (q, J = 1.60 Hz, 1H), 8.01 (dd, J = 2.80, 9.80 Hz, 1H), 7.54 (s, 1H), 7.35 (d, J = 8.80 Hz, 1H), 7.01 (d, J = 2.40 Hz, 1H), 6.63 (dd, J = 2.40, 8.80 Hz, 1H), 3.80-3.79 (m, 5H), 3.71- 3.70 (m, 5H), 3.40-3.38 (m, 5H), 3.28 (t, J = 8.40 Hz, 1H), 2.44- 2.43 (m, 1H), 2.16-2.14 (m, 1H),. 3. 438.2 4. Procedure 5 |
| Example 83 4-(6-(3-(5-fluoro- 1-methyl-1H- pyrrolo[2,3- b]pyridin-3- yl)pyrrolidin-1- yl)benzo[d]thia- zol-2- yl)morpholine Enantiopure 2 | 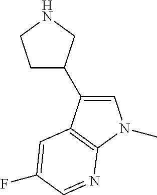 [SMILES: Cn1cc(C2CCNC2)c2cc(F)cnc21] | 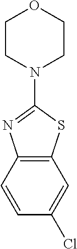 [SMILES: Clc1ccc2nc(N3CCOCC3)sc2c1] | 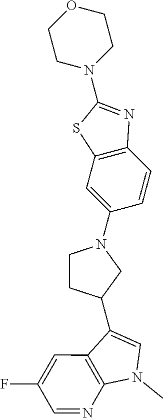 [SMILES: Cn1cc(C2CCN(c3ccc4nc(N5CCOCC5)sc4c3)C2)c2cc(F)cnc21] | 1. 40% 2. ¹H-NMR (400 MHz, DMSO- d6): δ 8.25 (t, J = 2.40 Hz, 1H), 8.01 (dd, J = 2.80, 9.80 Hz, 1H), 7.55 (s, 1H), 7.35 (d, J = 8.80 Hz, 1H), 7.01 (d, J = 2.40 Hz, 1H), 6.63 (dd, J = 2.00, 8.60 Hz, 1H), 3.81-3.79 (m, 4H), 3.73- 3.71 (m, 5H), 3.40-3.38 (m, 6H), 3.28-3.26 (m, 2H), 2.16 (q, J = Hz, 1H). 3. 438.2 4. Procedure |
| Example 84 6-(3-(5-fluoro-1- methyl-1H- pyrrolo[2,3- b]pyridin-3- yl)pyrrolidin-1- yl)-2- morpholinooxa- zolo[5,4- b]pyridine Enantiopure 1 | 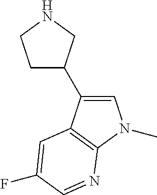 [SMILES: Cn1cc(C2CCNC2)c2cc(F)cnc21] | 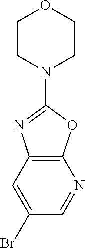 [SMILES: Brc1cnc2oc(N3CCOCC3)nc2c1] | 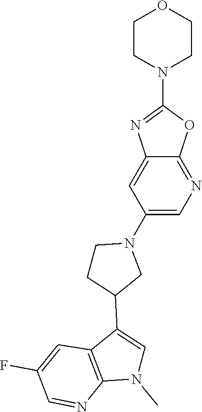 [SMILES: Cn1cc(C2CCN(c3cnc4oc(N5CCOCC5)nc4c3)C2)c2cc(F)cnc21] | 1. 30% 2. ¹H-NMR (400 MHz, DMSO- d6): δ 8.25 (q, J = 1.60 Hz, 1H), 8.02 (dd, J = 2.40, 9.80 Hz, 1H), 7.55 (s, 1H), 7.25 (d, J = 2.80 Hz, 1H), 6.96 (d, J = 2.80 Hz, 1H), 3.81 (t, J = 7.60 Hz, 4H), 3.72 (t, J = 5.20 Hz, 4H), 3.59 (t, J = 4.40 Hz, 4H), 3.40-3.42 (m, 2H), 3.30-3.29 (m, 2H), 2.50- 2.48 (m, 1H), 2.14-2.12 (m, 1H). 3. 423.2 4. Procedure 5 |
| Example 85 6-(3-(5-fluoro-1- methyl-1H- pyrrolo[2,3- b]pyridin-3- yl)pyrrolidin-1- yl)-2- morpholinooxa- zolo[5,4- b]pyridine Enantiopure 2 | 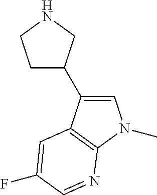 [SMILES: Cn1cc(C2CCNC2)c2cc(F)cnc21] | 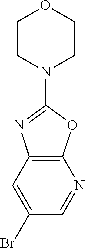 [SMILES: Brc1cnc2oc(N3CCOCC3)nc2c1] | 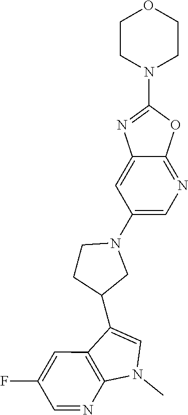 [SMILES: Cn1cc(C2CCN(c3cnc4oc(N5CCOCC5)nc4c3)C2)c2cc(F)cnc21] | 1. 32% 2. ¹H-NMR (400 MHz, DMSO- d6): δ 8.25 (q, J = 1.60 Hz, 1H), 8.02 (dd, J = 2.80, 9.60 Hz, 1H), 7.55 (s, 1H), 7.25 (d, J = 2.40 Hz, 1H), 6.97 (d, J = 2.80 Hz, 1H), 3.81 (t, J = 8.00 Hz, 4H), 3.72 (t, J = 5.20 Hz, 5H), 3.59 (t, J = 4.80 Hz, 5H), 3.40-3.42 (m, 2H), 3.28-3.26 (m, 1H), 2.17- 2.16 (m, 1H), 3. 423.2 4. Procedure 5 |
| Example 86 6-(3-(5-fluoro-1- methyl-1H- pyrrolo[2,3- b]pyridin-3- yl)pyrrolidin-1- yl)-2- morpholinooxa- zolo[5,4- c]pyridine Enantiopure 1 | 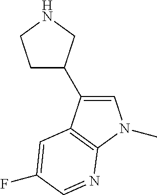 [SMILES: Cn1cc(C2CCNC2)c2cc(F)cnc21] | 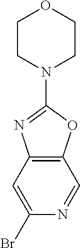 [SMILES: Brc1cc2nc(N3CCOCC3)oc2cn1] | 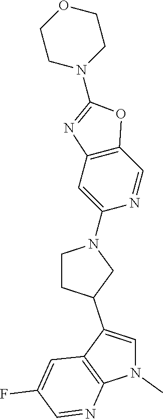 [SMILES: Cn1cc(C2CCN(c3cc4nc(N5CCOCC5)oc4cn3)C2)c2cc(F)cnc21] | 1. 12.8% 2. ¹H-NMR (400 MHz, DMSO- d6): δ 8.24 (t, J = 2.00 Hz, 1H), 8.11 (d, J = 0.80 Hz, 1H), 8.00 (dd, J = 2.80, 9.60 Hz, 1H), 7.53 (s, 1H), 6.34 (s, 1H), 3.94 (t, J = 7.60 Hz, 1H), 3.78 (s, 3H), 3.72 (t, J = 5.60 Hz, 4H), 3.62-3.61 (m, 5H), 3.46-3.44 (m, 1H), 3.39- 3.37 (m, 1H), 2.41-2.40 (m, 1H), 2.11-2.10 (m, 1H), 1.60 (s, 1H). 3. 423.2 4. Procedure 5 |
| Example 87 6-(3-(5-fluoro-1- methyl-1H- pyrrolo[2,3- b]pyridin-3- yl)pyrrolidin-1- yl)-2- morpholinooxa- zolo[5,4- c]pyridine Enantiopure 2 | 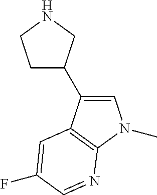 [SMILES: Cn1cc(C2CCNC2)c2cc(F)cnc21] | 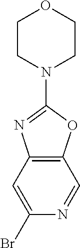 [SMILES: Brc1cc2nc(N3CCOCC3)oc2cn1] | 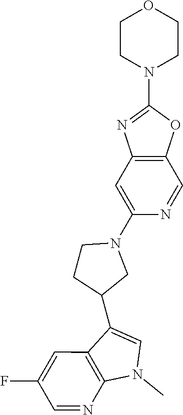 [SMILES: Cn1cc(C2CCN(c3cc4nc(N5CCOCC5)oc4cn3)C2)c2cc(F)cnc21] | 1. 12.8% 2. ¹H-NMR (400 MHz, DMSO- d6): δ 8.25 (t, J = 2.40 Hz, 1H), 8.11 (s, 1H), 8.00 (dd, J = 2.80, Hz, 1H), 7.54 (s, 1H), 6.35 (s, 1H), 3.96 (t, J = 2.40 Hz, 1H), 3.93 (s, 3H), 3.62-3.61 (m, 10H), 3.48-3.46 (m, 1H), 3.37-3.33 (m, 1H), 2.42-2.41 (m, 1H), 2.10- 2.08 (m, 1H). 3. 423.3 4. Procedure 5 |
| Example 88 6-(3-(5-fluoro-1- methyl-1H- pyrrolo[2,3- b]pyridin-3- yl)pyrrolidin-1- yl)-2- morpholinobenzo [d]oxazole Enantiopure 1 | 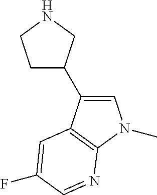 [SMILES: Cn1cc(C2CCNC2)c2cc(F)cnc21] | 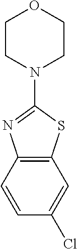 [SMILES: Clc1ccc2nc(N3CCOCC3)sc2c1] | 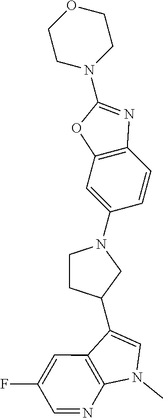 [SMILES: Cn1cc(C2CCN(c3ccc4nc(N5CCOCC5)oc4c3)C2)c2cc(F)cnc21] | 1. 39% 2. ¹H-NMR (400 MHz, DMSO- d6): δ 8.25 (s, 1H), 8.01 (dd, J = 2.80, 9.60 Hz, 1H), 7.54 (s, 1H), 7.15 (d, J = 8.40 Hz, 1H), 6.71 (d, J = 2.00 Hz, 1H), 6.45 (dd, J = 2.00, 8.80 Hz, 1H), 3.79-3.76 (m, 4H), 3.72 (t, J = 4.80 Hz, 5H), 3.50 (t, J = 4.40 Hz, 4H), 3.43-3.41 (m, 2H), 3.27 (t, J = 8.00 Hz, 1H), 2.46 (d, J = 8.00 Hz, 1H), 2.16 (q, J = 8.80 Hz, 1H). 3. 422.2 4. Procedure 5 |
| Example 89 6-(3-(5-fluoro-1- methyl-1H- pyrrolo[2,3- b]pyridin-3- yl)pyrrolidin-1- yl)-2- morpholinobenzo [d]oxazole Enantiopure 2 | 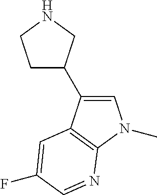 [SMILES: Cn1cc(C2CCNC2)c2cc(F)cnc21] | 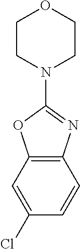 [SMILES: Clc1ccc2nc(N3CCOCC3)oc2c1] | 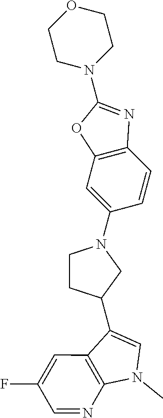 [SMILES: Cn1cc(C2CCN(c3ccc4nc(N5CCOCC5)oc4c3)C2)c2cc(F)cnc21] | 1. 42% 2. ¹H-NMR (400 MHz, DMSO- d6): δ 8.25 (d, J = 2.00 Hz, 1H), 8.01 (dd, J = 2.40, 9.80 Hz, 1H), 7.55 (s, 1H), 7.16 (d, J = 8.40 Hz, 1H), 6.71 (d, J = 2.00 Hz, 1H), 6.45 (dd, J = 2.00, 8.80 Hz, 1H), 3.72-3.71 (m, 9H), 3.45- 3.44 (m, 4H), 3.38-3.37 (m, 2H), 3.27 (t, J = 7.60 Hz, 2H), 2.46 (d, J = 6.80 Hz, 1H). 3. 422.2 4. Procedure 5 |
| Example 90 5-(3-(5-fluoro-1- methyl-1H- pyrrolo[2,3- b]pyridin-3- yl)pyrrolidin-1- yl)-2- morpholinooxa- zolo[4,5- b]pyridine Enantiopure 1 | 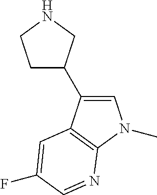 [SMILES: Cn1cc(C2CCNC2)c2cc(F)cnc21] | 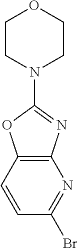 [SMILES: Brc1ccc2oc(N3CCOCC3)nc2n1] | 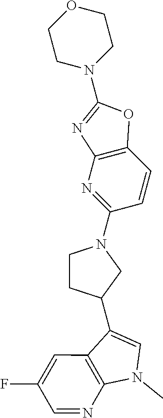 [SMILES: Cn1cc(C2CCN(c3ccc4oc(N5CCOCC5)nc4n3)C2)c2cc(F)cnc21] | 1. 29% 2. ¹H-NMR (400 MHz, DMSO- d6): δ 8.25 (t, J = 2.40 Hz, 1H), 8.00 (dd, J = 2.80, Hz, 1H), 7.57 (s, 1H), 7.54 (d, J = 2.80 Hz, 1H), 6.05 (d, J = 8.80 Hz, 1H), 3.94 (q, J = 7.20 Hz, 1H), 3.71 (s, 3H), 3.73-3.72 (m, 5H), 3.59-3.57 (m, 5H), 3.35-3.34 (m, 2H), 2.52- 2.51 (m, 1H), 2.33-2.32 (m, 1H). 3. 423.2 4. Procedure 5 |
| Example 91 5-(3-(5-fluoro-1- methyl-1H- pyrrolo[2,3- b]pyridin-3- yl)pyrrolidin-1- yl)-2- morpholinooxa- zolo[4,5- b]pyridine Enantiopure 2 | 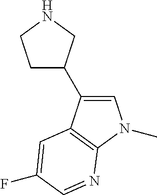 [SMILES: Cn1cc(C2CCNC2)c2cc(F)cnc21] | 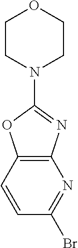 [SMILES: Brc1ccc2oc(N3CCOCC3)nc2n1] | 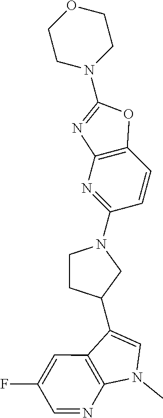 [SMILES: Cn1cc(C2CCN(c3ccc4oc(N5CCOCC5)nc4n3)C2)c2cc(F)cnc21] | 1. 20.4% 2. ¹H-NMR (400 MHz, DMSO- d6): δ 8.25 (t, J = 2.40 Hz, 1H), 8.00 (dd, J = 2.80, Hz, 1H), 7.57 (s, 1H), 7.54 (d, J = 2.00 Hz, 1H), 6.05 (d, J = 8.80 Hz, 1H), 3.94 (t, J = 2.00 Hz, 1H), 3.92 (s, 3H), 3.72-3.71 (m, 5H), 3.58-3.7 (m, 5H), 3.46-3.44 (m, 1H), 3.37- 3.34 (m, 1H), 2.42-2.41 (m, 1H), 2.12-2.11 (m, 1H). 3. 423.2 4. Procedure 5 |
| Example 92 5-(4-(7-methoxy- 1H-indazol-3- yl)piperidin-1- yl)-2- morpholinobenzo [d]oxazole | 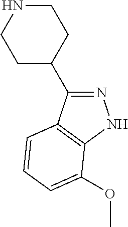 [SMILES: COc1cccc2c(C3CCNCC3)n[nH]c12] | 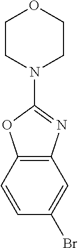 [SMILES: Brc1ccc2oc(N3CCOCC3)nc2c1] | 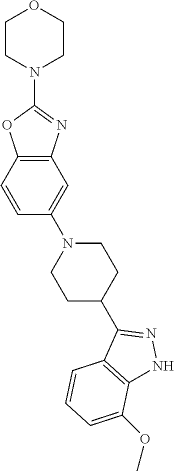 [SMILES: COc1cccc2c(C3CCN(c4ccc5oc(N6CCOCC6)nc5c4)CC3)n[nH]c12] | 1. 3% 2. ¹H-NMR (400 MHz, DMSO- d6): δ 12.91 (s, 1H), 7.34 (d, J = 8.00 Hz, 1H), 7.26 (d, J = 8.80 Hz, 1H), 6.97-6.96 (m, 2H), 6.81 (d, J = 7.20 Hz, 1H), 6.72 (dd, J = 2.40, 8.80 Hz, 1H), 3.93 (s, 3H), 3.72-3.71 (m, 6H), 3.57- 3.55 (m, 4H), 3.16-3.15 (m, 1H), 2.85-2.83 (m, 2H), 2.05-2.03 (m, 4H). 3. 434.2 4. Procedure 5 |
| Example 93 5-(4-(4-methyl- 1H-indazol-3- yl)piperidin-1- yl)-2- morpholinobenzo [d]oxazole | 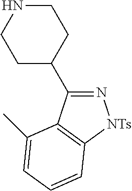 [SMILES: Cc1ccc(S(=O)(=O)n2nc(C3CCNCC3)c3c(C)cccc32)cc1] | 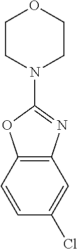 [SMILES: Clc1ccc2oc(N3CCOCC3)nc2c1] | 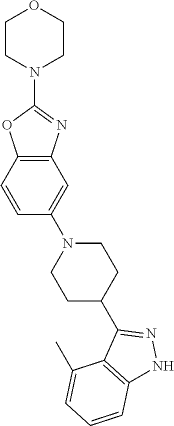 [SMILES: Cc1cccc2[nH]nc(C3CCN(c4ccc5oc(N6CCOCC6)nc5c4)CC3)c12] | 1. 62% 2. ¹H-NMR (400 MHz, DMSO- d6): δ 12.64 (s, 1H), 7.26-7.24 (m, 2H), 7.17-7.15 (m, 1H), 6.95 (d, J = 2.40 Hz, 1H), 6.82 (d, J = 6.80 Hz, 1H), 6.71 (dd, J = 2.40, 8.80 Hz, 1H), 3.71-3.70 (m, 6H), 3.56-3.55 (m, 4H), 3.34 (s, 1H), 2.82-2.81 (m, 2H), 2.67 (s, 3H), 2.01-1.98 (m, 4H). 3. 418.2 4. Procedure 3 |
| Example 94 5-(3-fluoro-4- (1H-indazol-3- yl)piperidin-1- yl)-2- morpholinobenzo [d]oxazole Enantiopure | 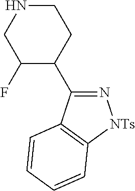 [SMILES: Cc1ccc(S(=O)(=O)n2nc(C3CCNCC3F)c3ccccc32)cc1] | 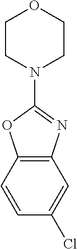 [SMILES: Clc1ccc2oc(N3CCOCC3)nc2c1] | 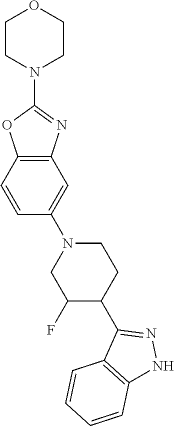 [SMILES: FC1CN(c2ccc3oc(N4CCOCC4)nc3c2)CCC1c1n[nH]c2ccccc12] | 1. 22% 2. ¹H-NMR (400 MHz, DMSO- d6): δ 12.88 (s, 1H), 7.81 (d, J = 8.00 Hz, 1H), 7.51 (d, J = 8.40 Hz, 1H), 7.33-7.32 (m, 1H), 7.29 (d, J = 8.40 Hz, 1H), 7.08-7.07 (m, 1H), 7.03 (d, J = 2.40 Hz, 1H), 6.77 (dd, J = 2.40, 8.80 Hz, 1H), 5.12-4.95 (m, 1H), 4.04- 3.99 (m, 1H), 3.73-3.71 (m, 4H), 3.59-3.58 (m, 5H), 3.42-3.41 (m, 1H), 2.91-2.90 (m, 2H), 2.10- 2.08 (m, 2H). 3. 422.2 4. Procedure 4 |
| Example 95 4-(5-(3-(5-fluoro- 1-methyl-1H- pyrrolo[2,3- b]pyridin-3- yl)pyrrolidin-1- yl)benzo[d]thia- zol-2- yl)morpholine Enantiopure 1 | 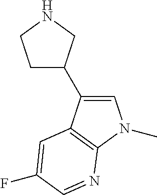 [SMILES: Cn1cc(C2CCNC2)c2cc(F)cnc21] | 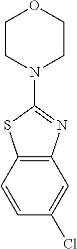 [SMILES: Clc1ccc2sc(N3CCOCC3)nc2c1] | 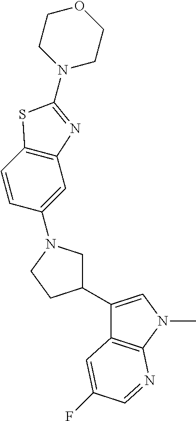 [SMILES: Cn1cc(C2CCN(c3ccc4sc(N5CCOCC5)nc4c3)C2)c2cc(F)cnc21] | 1. 45% 2. ¹H-NMR (400 MHz, DMSO- d6): δ 8.25 (t, J = 2.00 Hz, 1H), 8.01 (dd, J = 2.80, 9.80 Hz, 1H), 7.54 (d, J = 6.00 Hz, 1H), 7.51 (s, 1H), 6.72 (d, J = 2.00 Hz, 1H), 6.45 (dd, J = 2.00, 8.60 Hz, 1H), 3.81 (t, J = 11.20 Hz, 4H), 3.72 (q, J = 6.80 Hz, 5H), 3.38-3.40 (m, 6H), 3.30 (t, J = 8.40 Hz, 1H), 2.48-2.46 (m, 1H), 2.13- 2.11 (m, 1H). 3. 438.2 4. Procedure 5 |
| Example 96 5-(3-(5-fluoro-1- methyl-1H- pyrrolo[2,3- b]pyridin-3- yl)pyrrolidin-1- yl)-2-(4- methoxypiperidin- 1- yl)benzo[d]oxa- zole Enantiopure 1 | 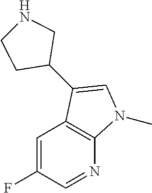 [SMILES: Cn1cc(C2CCNC2)c2cc(F)cnc21] | 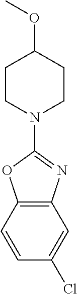 [SMILES: COC1CCN(c2nc3cc(Cl)ccc3o2)CC1] | 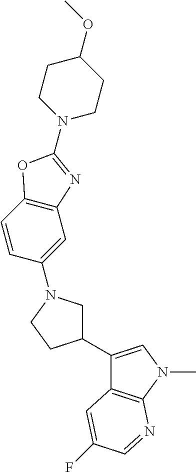 [SMILES: COC1CCN(c2nc3cc(N4CCC(c5cn(C)c6ncc(F)cc56)C4)ccc3o2)CC1] | 1. 47% 2. ¹H-NMR (400 MHz, DMSO- d6): δ 8.25 (q, J = 1.60 Hz, 1H), 8.00 (dd, J = 2.80, 9.60 Hz, 1H), 7.54 (s, 1H), 7.18 (d, J = 8.80 Hz, 1H), 6.51 (d, J = 2.40 Hz, 1H), 6.24 (dd, J = 2.40, 8.60 Hz, 1H), 3.73-3.71 (m, 6H), 3.43- 3.42 (m, 4H), 3.34-3.32 (m, 4H), 3.18-3.17 (m, 4H), 2.50-2.49 (m, 1H), 1.94-1.93 (m, 1H), 1.91- 1.90 (m, 1H), 1.51-1.50 (m, 1H). 3. 450.2 4. Procedure 5 |
| Example 97 5-(3-(5-fluoro-1- methyl-1H- pyrrolo[2,3- b]pyridin-3- yl)pyrrolidin-1- yl)-2-(4- methoxypiperidin- 1- yl)benzo[d]oxa- zole Enantiopure 2 | 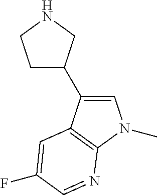 [SMILES: Cn1cc(C2CCNC2)c2cc(F)cnc21] | 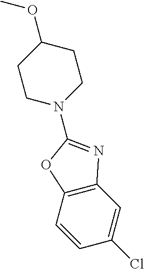 [SMILES: COC1CCN(c2nc3cc(Cl)ccc3o2)CC1] | 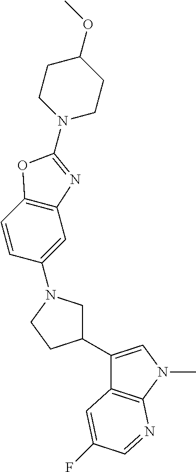 [SMILES: COC1CCN(c2nc3cc(N4CCC(c5cn(C)c6ncc(F)cc56)C4)ccc3o2)CC1] | 1. 33% 2. ¹H-NMR (400 MHz, DMSO- d6): δ 8.25 (t, J = 2.00 Hz, 1H), 8.00 (dd, J = 2.80, 9.60 Hz, 1H), 7.54 (s, 1H), 7.18 (d, J = 8.80 Hz, 1H), 6.51 (d, J = 2.00 Hz, 1H), 6.24 (dd, J = 2.40, 8.80 Hz, 1H), 3.81-3.80 (m, 2H), 3.76- 3.74 (m, 3H), 3.71-3.69 (m, 2H), 3.41-3.40 (m, 5H), 3.26-3.24 (m, 4H), 2.45-2.44 (m, 1H), 2.15- 2.14 (m, 1H), 1.91-1.90 (m, 2H), 1.50-1.49 (m, 2H). 3. 450.2 4. Procedure 5 |
| Example 98 3-(5-(3-(5-fluoro- 1-methyl-1H- pyrrolo[2,3- b]pyridin-3- yl)pyrrolidin-1- yl)benzo[d]oxa- zol-2-yl)-6-oxa-3- azabicyclo[3.1.1] heptane Enantiopure 1 | 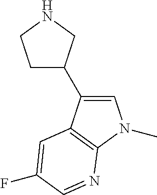 [SMILES: Cn1cc(C2CCNC2)c2cc(F)cnc21] | 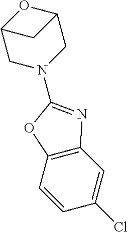 [SMILES: Clc1ccc2oc(N3CC4CC(C3)O4)nc2c1] | 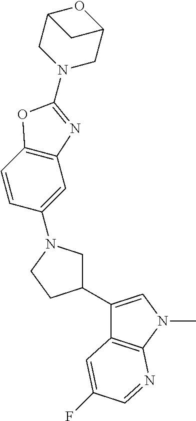 [SMILES: Cn1cc(C2CCN(c3ccc4oc(N5CC6CC(C5)O6)nc4c3)C2)c2cc(F)cnc21] | 1. 33% 2. ¹H-NMR (400 MHz, DMSO- d6): δ 8.25 (q, J = 1.60 Hz, 1H), 8.01 (dd, J = 2.80, 9.60 Hz, 1H), 7.55 (s, 1H), 7.24 (d, J = 8.40 Hz, 1H), 6.55 (d, J = 2.40 Hz, 1H), 6.25 (dd, J = 2.40, 8.80 Hz, 1H), 4.70-4.60 (m, 2H), 3.72- 3.70 (m, 10H), 3.44-3.43 (m, 2H), 3.27-3.25 (m, 1H), 3.16- 3.14 (m, 1H), 2.15-2.14 (m, 1H), 1.97-1.95 (m, 1H). 3. 434.2 4. Procedure 5 |
| Example 99 3-(5-(3-(5-fluoro- 1-methyl-1H- pyrrolo[2,3- b]pyridin-3- yl)pyrrolidin-1- yl)benzo[d]oxa- zol-2-yl)-6-oxa-3- azabicyclo[3.1.1] heptane Enantiopure 2 | 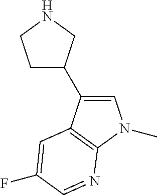 [SMILES: Cn1cc(C2CCNC2)c2cc(F)cnc21] | 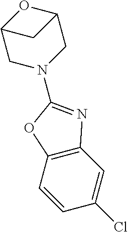 [SMILES: Clc1ccc2oc(N3CC4CC(C3)O4)nc2c1] | 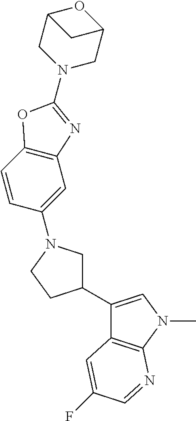 [SMILES: Cn1cc(C2CCN(c3ccc4oc(N5CC6CC(C5)O6)nc4c3)C2)c2cc(F)cnc21] | 1. 41% 2. ¹H-NMR (400 MHz, DMSO- d6): δ 8.25 (t, J = 2.00 Hz, 1H), 8.01 (dd, J = 2.40, 9.80 Hz, 1H), 7.55 (s, 1H), 7.24 (d, J = 8.40 Hz, 1H), 6.55 (d, J = 2.00 Hz, 1H), 6.25 (dd, J = 2.40, 8.60 Hz, 1H), 4.69 (d, J = 6.80 Hz, 2H), 3.72-3.70 (m, 10H), 3.41-3.39 (m, 2H), 3.27-3.25 (m, 1H), 3.16- 3.14 (m, 1H), 2.15-2.14 (m, 1H), 1.97-1.95 (m, 1H). 3. 434.2 4. Procedure 5 |
| Example 100 6-(4-(1H-indol-3- yl)piperidin-1- yl)-2- morpholinooxa- zolo[4,5- b]pyridine | 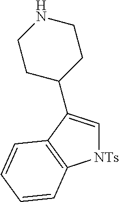 [SMILES: Cc1ccc(S(=O)(=O)n2cc(C3CCNCC3)c3ccccc32)cc1] | 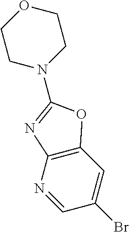 [SMILES: Brc1cnc2nc(N3CCOCC3)oc2c1] | 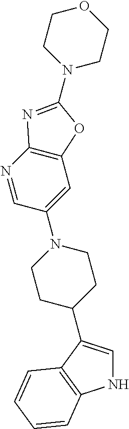 [SMILES: c1ccc2c(C3CCN(c4cnc5nc(N6CCOCC6)oc5c4)CC3)c[nH]c2c1] | 1. 18% 2. 1H NMR (400 MHz, DMSO- d6): δ : 10.82 (s, 1H), 7.95 (d, J = 2.40 Hz, 1H), 7.60-7.58 (m, 2H), 7.35 (d, J = 8.00 Hz, 1H), 7.14 (d, J = 2.00 Hz, 1H), 7.09-7.05 (m, 1H), 7.00-6.96 (m, 1H), 3.74- 3.70 (m, 6H), 3.62-3.59 (m, 4H), 2.92-2.85 (m, 3H), 2.08-2.06 (m, 2H), 1.90-1.84 (m, 2H). 3. 404.1 4. Procedure 5 |
| Example 101 5-(4-(1H-indol-3- yl)piperidin-1- yl)-2- morpholinooxa- zolo[5,4- b]pyridine | 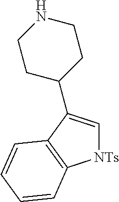 [SMILES: Cc1ccc(S(=O)(=O)n2cc(C3CCNCC3)c3ccccc32)cc1] | 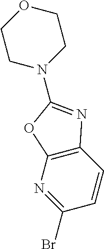 [SMILES: Brc1ccc2nc(N3CCOCC3)oc2n1] | 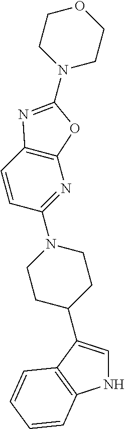 [SMILES: c1ccc2c(C3CCN(c4ccc5nc(N6CCOCC6)oc5n4)CC3)c[nH]c2c1] | 1. 27% 2. 1H NMR (400 MHz, DMSO- d6): δ 10.80 (s, 1H), 7.58-7.53 (m, 2H), 7.34 (d, J = 8.00 Hz, 1H), 7.11-7.04 (m, 2H), 6.97 (t, J = 7.20 Hz, 1H), 6.72 (d, J = 8.80 Hz, 1H), 4.28 (d, J = 12.80 Hz, 2H), 3.73-3.71 (m, 4H), 3.54- 3.51 (m, 4H), 3.03-2.93 (m, 3H), 2.06-2.00 (m, 2H), 1.71-1.67 (m, 2H). 3. 404.1 4. Procedure 1 |
| Example 102 5-(3-(5-fluoro-1- methyl-1H- pyrrolo[2,3- b]pyridin-3- yl)pyrrolidin-1- yl)-2- morpholinobenzo [d]oxazole | 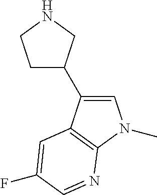 [SMILES: Cn1cc(C2CCNC2)c2cc(F)cnc21] | 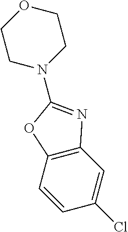 [SMILES: Clc1ccc2oc(N3CCOCC3)nc2c1] | 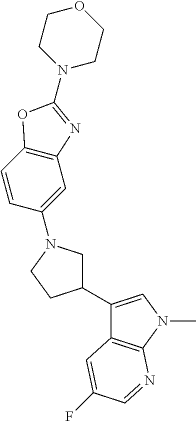 [SMILES: Cn1cc(C2CCN(c3ccc4oc(N5CCOCC5)nc4c3)C2)c2cc(F)cnc21] | 1. 33% 2. 1H NMR (400 MHz, DMSO- d6): δ 8.25-8.24 (m, 1H), 8.00 (dd, J = 2.80, 9.80 Hz, 1H), 7.54 (s, 1H), 7.21 (t, J = 4.40 Hz, 1H), 6.54 (d, J = 2.40 Hz, 1H), 6.27 (dd, J = 2.40, 8.80 Hz, 1H), 3.77- 3.70 (m, 8H), 3.56-3.54 (m, 4H), 3.43-3.41 (m, 2H), 3.35-3.25 (m, 2H), 2.50-2.47 (m, 1H), 2.18- 2.00 (m, 1H). 3. 422.2 4. Procedure 5 |
| Example 103 6-(4-(6-fluoro- 1H-pyrrolo[3,2- b]pyridin-3- yl)piperidin-1- yl)-2- morpholinobenzo [d]oxazole | 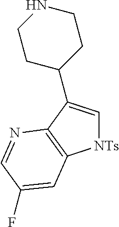 [SMILES: Cc1ccc(S(=O)(=O)n2cc(C3CCNCC3)c3ncc(F)cc32)cc1] | 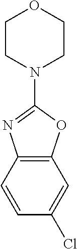 [SMILES: Clc1ccc2nc(N3CCOCC3)oc2c1] | 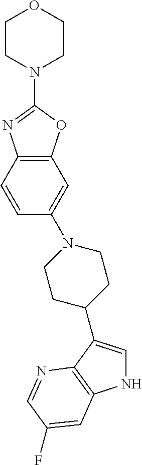 [SMILES: Fc1cnc2c(C3CCN(c4ccc5nc(N6CCOCC6)oc5c4)CC3)c[nH]c2c1] | 1. 22% 2. 1H-NMR (400 MHz, DMSO- d6): δ 11.13 (s, 1H), 8.30 (dd, J = 1.60, 2.40 Hz, 1H), 7.61 (dd, J = 2.40, 10.00 Hz, 1H), 7.45 (d, J = 2.00 Hz, 1H), 7.17 (d, J = 8.40 Hz, 1H), 7.12 (d, J = 2.00 Hz, 1H), 6.87 (dd, J = 2.00, 8.60 Hz, 1H), 3.73-3.66 (m, 6H), 3.53 (t, J = 4.40 Hz, 4H), 3.04-2.98 (m, 1H), 2.83-2.77 (m, 2H), 2.14 (d, J = 12.40 Hz, 2H), 1.96-1.87 (m, 2H). 3. 422.2 4. Procedure 5 |

Chiral Separation Conditions:

Conditions 1. The enantiopure Examples 33 and 34 above were obtained by chiral SFC separation starting from the corresponding racemic mixture (YMC Amylose-SA (flow rate: 5 ml/min); Supercritical carbon dioxide containing 40% of a co-solvent (0.5% isopropyl amine in methanol injected volume: 15 μL; Outlet Pressure: 100 bar at 35° C.)

Conditions 2 The enantiopure Examples 37 and 38 above were obtained by chiral SFC separation starting from the corresponding racemic mixture (YMC Cellulose-SC (flow rate: 5 ml/min; Supercritical carbon dioxide containing 40% of a co-solvent (0.5% isopropyl amine in methanol injected volume: 15 μL; Outlet Pressure: 100 bar at 35° C.)

Conditions 3. The enantiopure Examples 39 and 40 above were obtained by chiral SFC separation starting from the corresponding racemic mixture (YMC Cellulose-SC (flow rate: 3 ml/min; Supercritical carbon dioxide containing 40% of a co-solvent (0.5% isopropyl amine in methanol injected volume: 10 μL; Outlet Pressure: 100 bar at 35° C.)

Conditions 4: The enantiopure Example 94 above was obtained by chiral SFC separation starting from the corresponding racemic mixture (YMC Cellulose-SB (flow rate: 5 ml/min); Supercritical carbon dioxide containing 30% of a co-solvent (0.5% isopropyl amine in methanol injected volume: 15 μL; Outlet Pressure: 100 bar at 35° C.)

Conditions 5: The enantiopure Examples 82, 83, 86, 87, 88, 89, 90, 91, 95, 96, and 97 above were obtained by chiral SFC separation starting from the corresponding racemic mixture (Chiralcel OJ-H (flow rate: 3 ml/min); Supercritical carbon dioxide containing 15% of a co-solvent (0.5% isopropyl amine in methanol injected volume: 2 μL; Outlet Pressure: 100 bar at 35° C.)

### Example 104: 5-(4-(6-methoxy-1H-pyrrolo[2,3-b]pyridin-3-yl)piperidin-1-yl)-2-morpholinobenzo[d]oxazole

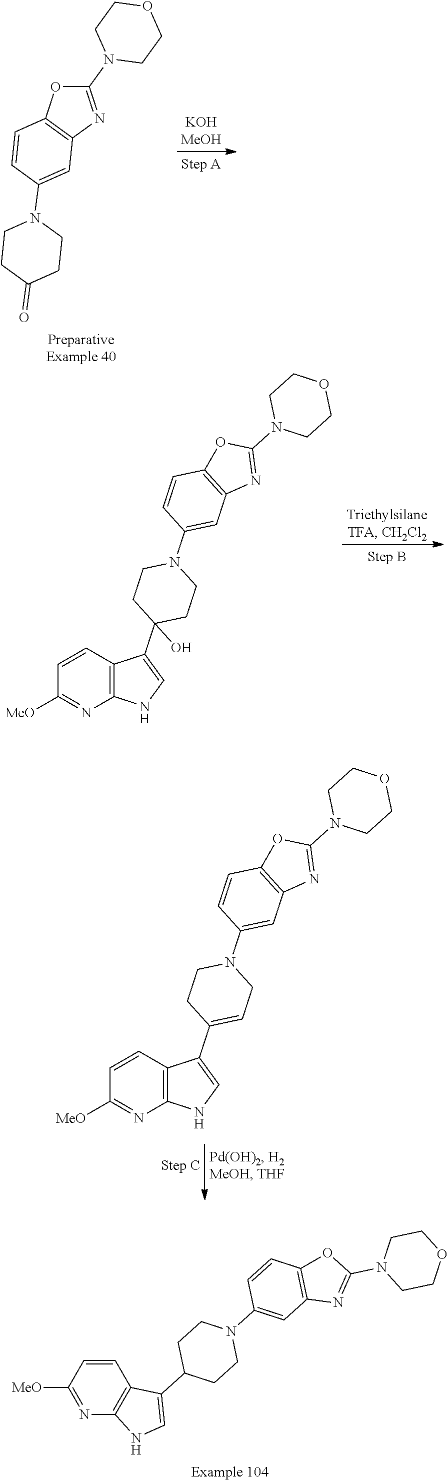 [SMILES: O=C1CCN(c2ccc3oc(N4CCOCC4)nc3c2)CC1 ; COc1ccc2c(C3(O)CCN(c4ccc5oc(N6CCOCC6)nc5c4)CC3)c[nH]c2n1 ; COc1ccc2c(C3=CCN(c4ccc5oc(N6CCOCC6)nc5c4)CC3)c[nH]c2n1 ; COc1ccc2c(C3CCN(c4ccc5oc(N6CCOCC6)nc5c4)CC3)c[nH]c2n1]

Step A

To a stirred solution of 6-methoxy-1H-pyrrolo[2,3-b]pyridine (0.250 g, 0.00169 mol) in methanol (10.00 ml), KOH (0.284 g, 0.00506 mol) and Preparative Example 40 (0.590 g, 0.00196 mol) were added. The reaction mixture was stirred at room temperature for 16 hours under nitrogen. The mixture was concentrated under reduced pressure then water (30 ml) followed by dichloromethane (30 ml) were added. The organic layer was separated and concentrated under reduced pressure. The crude was purified on HP-Silica column (Biotage), eluting with a gradient of petroleum ether/ethyl acetate (100/0 to 0/100) to afford 4-(6-methoxy-1H-pyrrolo[2,3-b]pyridin-3-yl)-1-(2-morpholinobenzo[d]oxazol-5-yl)piperidin-4-ol (100 mg, 9.19%) as a pale yellow solid. ¹H-NMR (400 MHz, DMSO-d6): δ 11.21 (s, 1H), 8.05 (d, J=8.40 Hz, 1H), 7.25-7.23 (m, 1H), 7.01-7.02 (m, 1H), 6.94 (s, 1H), 6.68-6.69 (m, 1H), 6.49 (d, J=8.40 Hz, 1H), 4.83 (s, 1H), 3.85 (s, 3H), 3.72-3.70 (m, 4H), 3.56-3.55 (m, 4H), 3.38-3.36 (m, 2H), 3.17 (t, J=9.60 Hz, 1H), 2.10-2.09 (m, 2H), 1.95-1.93 (m, 2H). MS: 450.1 (M+H)⁺.

Step B

To a stirred solution of compound from Step A above (0.140 g, 0.311 mmol) in dichloromethane (5.00 ml), triethylsilane (0.14 ml, 0.877 mmol) and TFA (0.14 ml, 1.88 mmol) were added at 0° C. and the mixture was stirred at room temperature for 12 hours under nitrogen. The reaction mixture was neutralized using a 10% aqueous NaHCO₃ solution and extracted using dichloromethane. The organic layer was separated and concentrated under reduced pressure. The crude was purified on HP-Sil column (Biotage), eluting with a gradient of petroleum ether/ethyl acetate (100/0 to 0/100) to afford 5-(4-(6-methoxy-1H-pyrrolo[2,3-b]pyridin-3-yl)-3,6-dihydropyridin-1(2H)-yl)-2-morpholino benzo[d]oxazole (70 mg, 46.8%) as a yellow brown solid. MS: 432.0 (M+H)⁺.

Step C

To a stirred solution of compound from Step B above (0.060 g, 0.125 mmol) in methanol (2.00 ml) and tetrahydrofuran (2.00 ml), Pd(OH)₂ (0.017 g, 0.125 mmol) was added and the mixture was stirred at room temperature for 2 hours under hydrogen pressure (1 bar) atmosphere. The mixture was filtered through celite and washed with methanol (3 ml). The filtrate was concentrated under reduced pressure. The crude was purified on HP-Sil column (Biotage), eluting with a gradient of petroleum ether/ethyl acetate (100/0 to 20/80) to afford the title compound (10 mg, 18.3%) as a pale yellow solid. ¹H-NMR (400 MHz, DMSO-d6): δ 11.19 (s, 1H), 7.92 (d, J=8.40 Hz, 1H), 7.25 (d, J=8.80 Hz, 1H), 6.95-6.94 (m, 2H), 6.69-6.68 (m, 1H), 6.49 (d, J=8.80 Hz, 1H), 3.85 (s, 3H), 3.66-3.69 (m, 6H), 3.56-3.55 (m, 4H), 2.79-2.76 (m, 3H), 2.02-2.00 (m, 2H), 1.82-1.79 (m, 2H). MS: 434.1 (M+H)⁺.

### Example 105: 5-(4-(6-fluoro-1-methyl-1H-pyrrolo[3,2-b]pyridin-3-yl)piperidin-1-yl)-2-morpholinobenzo[d]oxazole

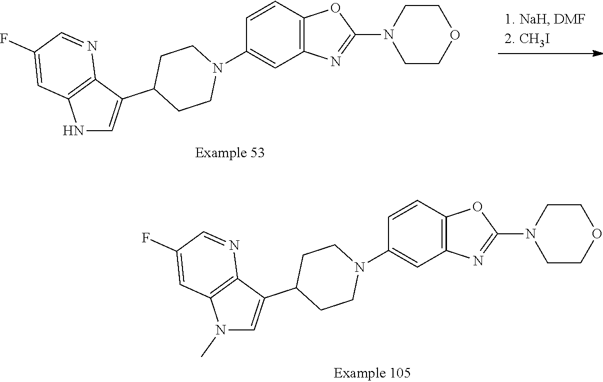 [SMILES: Fc1cnc2c(C3CCN(c4ccc5oc(N6CCOCC6)nc5c4)CC3)c[nH]c2c1 ; Cn1cc(C2CCN(c3ccc4oc(N5CCOCC5)nc4c3)CC2)c2ncc(F)cc21]

To a cooled (0° C.) solution of Example 53 (50 mg, 0.12 mmol) in DMF (10 ml), sodium hydride (60% in paraffin oil, 11 mg, 0.24 mmol) was added portion wise and the mixture was stirred at room temperature for 30 minutes. Then, methyl iodide (25 mg, 0.18 mmol) was added and the reaction was stirred at room temperature for 30 minutes. The latter was quenched with iced water and extracted with ethyl acetate (10 ml). The organic layer was separated, dried over sodium sulphate, filtered and then concentrated under vacuum. The crude was purified on HP-Silica column (Biotage), eluting with a gradient of petroleum ether/ethyl acetate (100/0 to 0/100) to afford the title compound (100 mg, 9.19%) as a pale-yellow solid. ¹H-NMR (400 MHz, DMSO-d6): δ 8.30-8.31 (m, 1H), 7.83 (dd, J=2.80, 10.20 Hz, 1H), 7.45 (s, 1H), 7.25 (d, J=8.80 Hz, 1H), 6.96 (s, 1H), 6.71 (dd, J=2.40, 8.80 Hz, 1H), 3.76 (s, 3H), 3.72-3.71 (m, 6H), 3.56-3.55 (m, 4H), 2.99-2.98 (m, 1H), 2.79-2.77 (m, 2H), 2.14-2.12 (m, 2H), 1.88-1.87 (m, 2H). MS: 436.2 (M+H)⁺.

### Example 106: 4-(5-(4-(1H-indazol-3-yl)piperidin-1-yl)thiazolo[5,4-b]pyridin-2-yl)morpholine

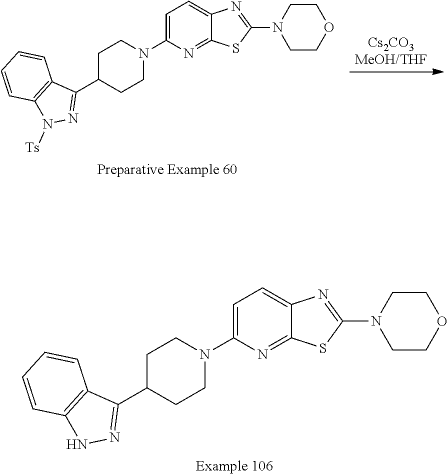 [SMILES: Cc1ccc(S(=O)(=O)n2nc(C3CCN(c4ccc5nc(N6CCOCC6)sc5n4)CC3)c3ccccc32)cc1 ; c1ccc2c(C3CCN(c4ccc5nc(N6CCOCC6)sc5n4)CC3)n[nH]c2c1]

The title compound from Preparative Example 60 (84 mg, 0.146 mmol) and cesium carbonate (95 mg, 0.292 mmol) were added into a microwave vial, followed by MeOH (1.5 ml) and THF (3 ml). The reaction mixture was heated at 110° C. for 30 minutes in the microwave and then allowed to cool down to room temperature. The solvents were removed under reduced pressure and the residue was purified on a KP-NH column using a Biotage Isolera One purification system with a gradient of dichloromethane/methanol (100/0 to 95/5). The fractions containing the product were combined and concentrated under reduced pressure. The residue was triturated in a mixture of heptane and ethyl acetate and the solid was collected by filtration to afford the title compound (6.1 mg, 10%) as a light-yellow powder. 1H-NMR (400 MHz, DMSO-d6): 5=12.65 (s, 1H), 7.78 (d, J=8.1 Hz, 1H), 7.64 (d, J=8.9 Hz, 1H), 7.46 (d, J=8.3 Hz, 1H), 7.37-7.26 (m, 1H), 7.11-7.01 (m, 1H), 6.90 (d, J=9.0 Hz, 1H), 4.32 (d, J=12.8 Hz, 2H), 3.72 (t, J=4.9 Hz, 4H), 3.47 (t, J=4.9 Hz, 4H), 3.03 (t, J=12.2 Hz, 2H), 2.54-2.52 (m, 1H), 2.05 (d, J=13.0 Hz, 2H), 1.96-1.79 (m, 2H). MS: 421.2 (M+H)⁺.

Following the deprotection procedure as described in Example 106, the following compounds were prepared.

| | | | 1. Yield; % |
|---|---|---|---|
| | Protected | | 2. ¹H-NMR |
| Example | Precursor | Product | 3. MH⁺ (ESI) |
| Example 107 5-(4-(1H-indol-3-yl)- 3,6-dihydropyridin- 1(2H)-yl)-2- morpholinobenzo [d]oxazole | 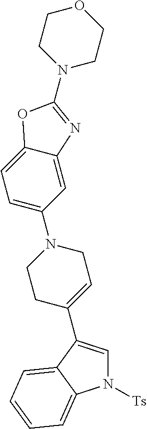 [SMILES: Cc1ccc(S(=O)(=O)n2cc(C3=CCN(c4ccc5oc(N6CCOCC6)nc5c4)CC3)c3ccccc32)cc1] | 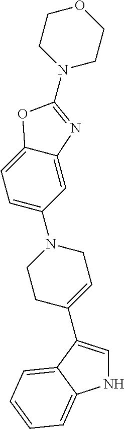 [SMILES: C1=C(c2c[nH]c3ccccc23)CCN(c2ccc3oc(N4CCOCC4)nc3c2)C1] | 1. 11% 2. ¹H-NMR (400 MHz, DMSO-d₆): δ = 11.14 (s, 1H), 7.86 (d, J = 7.9 Hz, 1H), 7.44 (s, 1H), 7.39 (d, J = 8.0 Hz, 1H), 7.27 (d, J = 8.8 Hz, 1H), 7.12 (t, J = 7.6 Hz, 1H), 7.05 (t, J = 7.6 Hz, 1H), 6.97 (d, 1H), 6.72 (d, J = 8.8 Hz, 1H), 6.30-6.24 (m, 1H), 3.85 (s, 2H), 3.71 (t, J = 5.5 Hz, 4H), 3.57 (t, J = 5.1 Hz, 4H), 3.44 (t, 2H), 2.67 (s, 2H). 3. 401.2 |

HCl Salt of Compounds of the Present Invention

General Procedure

To a solution of an Example compound (0.1 g) in dry DCM (10 ml), cooled to 0° C., was added 4 M HCl in diethyl ether (5 eq) or 4 M HCl in 1,4-dioxane (5 eq) and stirred for 15 minutes. The reaction mixture was concentrated under vacuum and triturated with diethyl ether to afford the desired product as indicated in Table 2.

### Examples 108-112

Following the hydrochloride salt procedure as described in the general procedure above, the following compounds were prepared:

TABLE 2

| | | | 1. Yield |
|---|---|---|---|
| | Free Base | | 2. ¹H-NMR |
| Example | Precursor | HCl Product | 3. MH⁺ (ESI) |
| Example 108 4-(5-(4-(1H-indol-3- yl)piperidin-1- yl)thiazolo[5,4- b]pyridin-2- yl)morpholine hydrochloride | 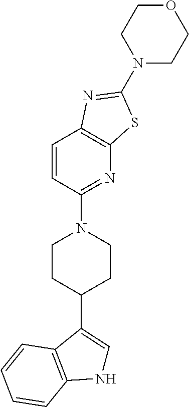 [SMILES: c1ccc2c(C3CCN(c4ccc5nc(N6CCOCC6)sc5n4)CC3)c[nH]c2c1] | 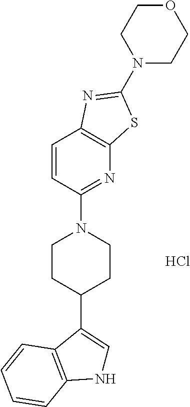 [SMILES: c1ccc2c(C3CCN(c4ccc5nc(N6CCOCC6)sc5n4)CC3)c[nH]c2c1 ; Cl] | 1. 83% 2. ¹H-NMR (400 MHz, DMSO-d6): δ 10.82 (s, 1H), 7.70 (d, J = 8.80 Hz, 1H), 7.59 (d, J = 8.00 Hz, 1H), 7.34 (d, J = 8.40 Hz, 1H), 7.12-7.11 (m, 1H), 7.04-7.05 (m, 1H), 6.95-6.94 (m, 2H), 4.31-4.28 (m, 2H), 3.74-3.72 (m, 4H), 3.51-3.50 (m, 4H), 3.06-3.03 (m, 3H), 2.09-2.06 (m, 2H), 1.77-1.74 (m, 2H), 3. 420.2 |
| Example 109 4-(6-(4-(5-fluoro-1- methyl-1H- pyrrolo[2,3-b]pyridin- 3-yl)piperidin-1- yl)thiazolo[4,5- b]pyridin-2- yl)morpholine hydrochloride | 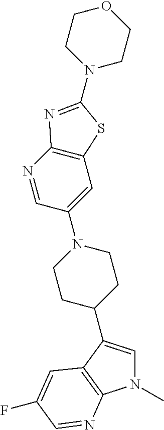 [SMILES: Cn1cc(C2CCN(c3cnc4nc(N5CCOCC5)sc4c3)CC2)c2cc(F)cnc21] | 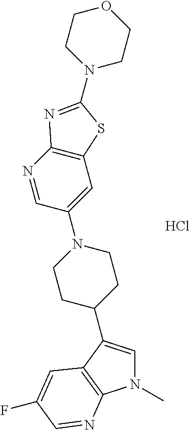 [SMILES: Cn1cc(C2CCN(c3cnc4nc(N5CCOCC5)sc4c3)CC2)c2cc(F)cnc21 ; Cl] | 1. 89% 2. ¹H-NMR (400 MHz, DMSO-d6): δ 8.75 (d, J = 2.40 Hz, 1H), 8.24-817 (m, 2H), 8.04-8.02 (m, 1H), 7.48 (s, 1H), 3.73-3.72 (m, 13H), 3.04-3.01 (m, 3H), 1.94-1.91 (m, 4H) 3. 453.2 |
| Example 110 4-(6-(4-(5-fluoro-1- methyl-1H- pyrrolo[2,3-b]pyridin- 3-yl)piperidin-1- yl)thiazolo[4,5- c]pyridin-2- yl)morpholine hydrochloride | 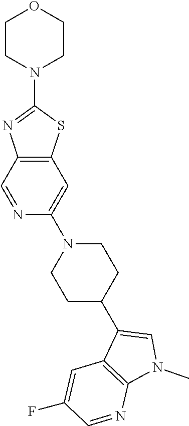 [SMILES: Cn1cc(C2CCN(c3cc4sc(N5CCOCC5)nc4cn3)CC2)c2cc(F)cnc21] | 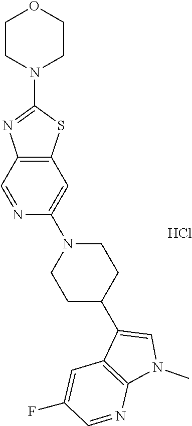 [SMILES: Cn1cc(C2CCN(c3cc4sc(N5CCOCC5)nc4cn3)CC2)c2cc(F)cnc21 ; Cl] | 1. 87% 2. ¹H-NMR (400 MHz, DMSO-d6): δ 8.25-8.23 (m, 1H), 8.18 (s, 1H), 8.06- 8.03 (m, 2H), 7.45 (s, 1H), 4.26 (d, J = 12.80 Hz, 2H), 3.77-3.74 (m, 7H), 3.64- 3.62 (m, 4H), 3.40-3.29 (m, 2H), 3.14- 3.11 (m, 1H), 2.12-2.10 (m, 2H), 1.81- 1.76 (m, 2H) 3. 453.0 |
| Example 111 6-(4-(5-fluoro-1- methyl-1H- pyrrolo[2,3-b]pyridin- 3-yl)piperidin-1-yl)-2- morpholinobenzo[d] oxazole hydrochloride | 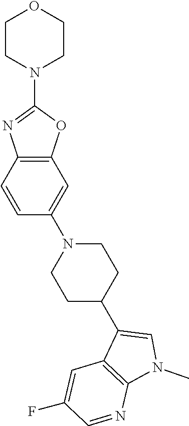 [SMILES: Cn1cc(C2CCN(c3ccc4nc(N5CCOCC5)oc4c3)CC2)c2cc(F)cnc21] | 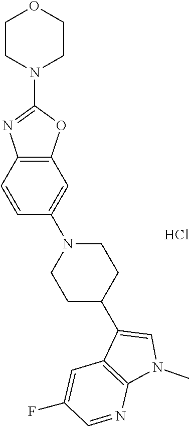 [SMILES: Cn1cc(C2CCN(c3ccc4nc(N5CCOCC5)oc4c3)CC2)c2cc(F)cnc21 ; Cl] | 1. 75% 2. ¹H-NMR (400 MHz, DMSO-d6): δ 13.01 (bs, 1H), 8.27 (d, J = 3.20 Hz, 2H), 8.15 (s, 1H), 7.79 (d, J = 6.40 Hz, 1H), 7.54 (s, 1H), 7.48 (d, J = 8.40 Hz, 1H), 3.81-3.82 (s, 4H), 3.76-3.70 (m, 5H), 3.66-3.63 (m, 6H), 3.24-3.17 (m, 1H), 2.51-2.52 (m, 2H), 2.18-2.15 (m, 2H) 3. 436.3 |
| Example 112 5-(5-(5-fluoro-1- methyl-1H- pyrrolo[2,3-b]pyridin- 3- yl)hexahydrocyclo- penta[c]pyrrol-2(1H)-yl)- 2- morpholinobenzo[d] oxazole hydrochloride | 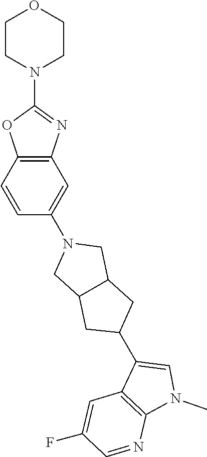 [SMILES: Cn1cc(C2CC3CN(c4ccc5oc(N6CCOCC6)nc5c4)CC3C2)c2cc(F)cnc21] | 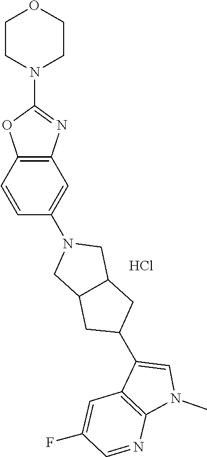 [SMILES: Cn1cc(C2CC3CN(c4ccc5oc(N6CCOCC6)nc5c4)CC3C2)c2cc(F)cnc21 ; Cl] | 1. 81% 2. ¹H-NMR (400 MHz, DMSO-d6): δ 8.22-8.23 (m, 1H), 7.98-8.00 (m, 1H), 7.42-7.43 (m, 2H), 6.98 (bs, 2H), 3.73- 3.74 (m, 9H), 3.60-3.62 (m, 5H), 3.29- 3.40 (m, 2H), 3.00-3.17 (m, 2H), 2.43- 2.45 (m, 2H), 1.73 (bs, 2H). 3. 462.2 |

Biological Assay Description

Full-Length Tau (flTau) Disaggregation Assay by Thioflavin T (ThT)

The longest isoform of human Tau (2N4R; 441 amino acids) was expressed in bacteria and purified (Biotechne). For the Tau disaggregation assay by ThT, 35 μM of recombinant full-length (fl) Tau in phosphate-buffered saline (PBS) were aggregated for 72 hours at 37° C. in presence of 35 μM of heparin (Sigma-Aldrich) and 10 mM of OTT (Sigma-Aldrich) under shaking at 1000 RPM. Compound 1 was dissolved in anhydrous dimethyl sulfoxide (DMSO, Sigma-Aldrich) to reach a concentration of 2.5 mM. flTau aggregates and serial dilutions of compound 1 were mixed together in PBS (volume 50 μL) to a final concentration of 60 nM of flTau aggregates and from 20 to 0.0012 μM of Compound 1. The mixture was incubated for 30 minutes at room temperature (RT), then 40 μL of this mixture were transferred into a black 384-well plate assay (Perkin-Elmer) and mixed with 10 μL of 20 μM ThT in 250 mM glycine (both from Sigma-Aldrich) in PBS. Fluorescence (relative fluorescence units; RFU) was measured in monoplicate or duplicate on a Tecan reader (excitation: 440 nm; emission: 485 nm). Percentage of flTau disaggregation was then calculated and the half maximal effective concentration (EC₅₀) was determined using GraphPad Prism version 8 (GraphPad Software) assuming a one-binding site fitting model, see Table 3 and Table 4.

Tau K18 Disaggregation Assay by ThT

The Tau K18 fragment, encompassing amino acids 244 to 372 of the longest isoform (2N4R) of human Tau441, was expressed in bacteria and purified (Biotechne). For the K18 disaggregation assay by ThT, 35 μM of recombinant K18 in PBS were aggregated for 24 hours at 37° C. in presence of 25 μM of heparin (Sigma-Aldrich) and 10 mM of 1,4-Dithiothreito (DTT from Sigma-Aldrich) under shaking at 750 RPM. Compound 1 was dissolved in anhydrous dimethyl sulfoxide (DMSO, Sigma-Aldrich) to reach a concentration of 10 mM. K18 aggregates and serial dilutions of compounds were mixed together in PBS (volume 50 μL) to a final concentration of 2 μM of K18 aggregates and from 400 to 0.1 μM of Compound 1. The mixture was incubated for 30 minutes at room temperature (RT), then 40 μL of this mixture were transferred into a black 384-well plate assay (Perkin-Elmer) and mixed with 10 μL of 100 μM ThT in 250 mM glycine (both from Sigma-Aldrich) in PBS. Fluorescence (relative fluorescence units; RFU) was measured in monoplicate or duplicate on a Tecan reader (excitation: 440 nm; emission: 485 nm). Percentage of K18 disaggregation was then calculated and half maximal effective concentration (EC₅₀) was determined using GraphPad Prism version 8 (GraphPad Software) assuming a one-binding site fitting model, see Table 3 and Table 4.

The following example compound was measured:

TABLE 3

| | Tau K18 | flTau |
|---|---|---|
| | disaggregation | disaggregation |
| | EC₅₀ | EC₅₀ |
| Compound | (μM) | (μM) |
| 1 | +++ | +++ |

TABLE 4

| | Tau K18 | flTau |
|---|---|---|
| | disaggregation | disaggregation |
| | EC₅₀ | EC₅₀ |
| Examples | (μM) | (μM) |
| 3 | − | ++ |
| 4 | − | +++ |
| 5 | − | +++ |
| 6 | − | ++ |
| 7 | +++ | +++ |
| 8 | − | +++ |
| 9 | +++ | +++ |
| 10 | − | +++ |
| 11 | − | +++ |
| 12 | − | +++ |
| 13 | − | +++ |
| 14 | + | − |
| 15 | + | − |
| 16 | + | − |
| 17 | ++ | +++ |
| 18 | − | ++ |
| 19 | ++ | ++ |
| 20 | ++ | +++ |
| 21 | − | ++ |
| 22 | − | ++ |
| 23 | + | − |
| 24 | +++ | +++ |
| 25 | − | ++ |
| 26 | − | ++ |
| 27 | − | ++ |
| 28 | − | +++ |
| 29 | + | ++ |
| 30 | − | +++ |
| 31 | − | ++ |
| 32 | ++ | +++ |
| 33 | ++ | ++ |
| 34 | ++ | ++ |
| 35 | ++ | ++ |
| 36 | + | ++ |
| 37 | +++ | ++ |
| 38 | ++ | ++ |
| 39 | − | ++ |
| 40 | − | +++ |
| 41 | ++ | ++ |
| 42 | + | − |
| 43 | ++ | ++ |
| 44 | ++ | ++ |
| 45 | − | ++ |
| 46 | ++ | ++ |
| 47 | + | ++ |
| 48 | − | ++ |
| 49 | − | + |
| 50 | +++ | +++ |
| 51 | + | ++ |
| 52 | + | − |
| 53 | ++ | − |
| 54 | + | − |
| 55 | ++ | − |
| 56 | ++ | − |
| 57 | + | − |
| 58 | + | − |
| 59 | ++ | − |
| 61 | − | ++ |
| 62 | ++ | − |
| 63 | + | − |
| 64 | ++ | − |
| 65 | ++ | − |
| 66 | +++ | − |
| 67 | +++ | − |
| 68 | − | ++ |
| 69 | ++ | − |
| 70 | ++ | − |
| 72 | − | + |
| 73 | − | ++ |
| 74 | − | ++ |
| 75 | − | +++ |
| 76 | − | ++ |
| 77 | − | ++ |
| 78 | − | ++ |
| 79 | − | +++ |
| 80 | − | ++ |
| 81 | − | +++ |
| 82 | − | +++ |
| 83 | − | +++ |
| 84 | − | ++ |
| 85 | − | +++ |
| 86 | − | ++ |
| 87 | − | ++ |
| 88 | − | +++ |
| 89 | − | +++ |
| 90 | − | +++ |
| 91 | − | +++ |
| 92 | − | ++ |
| 93 | − | ++ |
| 94 | − | ++ |
| 95 | − | ++ |
| 96 | − | ++ |
| 97 | − | ++ |
| 98 | − | ++ |
| 99 | − | ++ |
| 100 | − | ++ |
| 101 | + | − |
| 103 | ++ | − |
| 104 | ++ | ++ |
| 105 | − | + |
| 106 | − | ++ |
| 107 | − | +++ |
| 108 | + | − |
| 109 | + | ++ |
| 110 | + | ++ |
| 111 | ++ | ++ |
| 112 | ++ | − |

Legend: +++ EC₅₀ < 1 uM; ++ EC₅₀ 1< × <10 uM; + EC50 10< × <50 uM.

Reduction of Intracellular Misfolded Tau

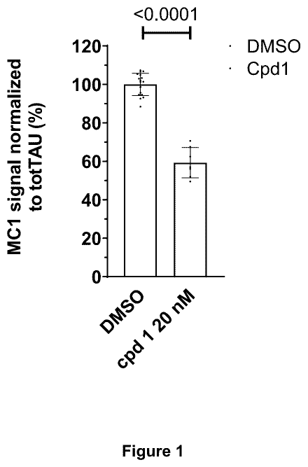

The SH-SY5Y cell line overexpressing the full-length form of human Tau carrying the P301L mutation were cultured in complete medium DMEM-F12 4.5 g/L Glutamax (Invitrogen), 15% FBS (Biochrom), 1% Peni/Strep (Invitrogen) supplemented with 2.5 μg/ml of G418 (Sigma-Aldrich) selection antibiotic. For the in vitro differentiation from neuroblastoma cells to neurons, cells were plated in 24-well plates (Costar 3337) on glass coverslips coated with Poly-D-Lysine at a seeding density of 2.5×103 cells. P301L SH-SY5Y cells were differentiated for 1 week at 37° C. in culture medium in presence of 10 μM retinoic acid (RA; Sigma, R2625). Every 48-72 h media was changed and fresh retinoic acid was added. For the treatment with compound 1, were dispensed on the cells at concentrations between 1-20 nM for 72 hours, with changing compounds every 24 h. After the incubation with Compound 1, cells were pre-fixed with 2% PFA for 5 min followed by a 15 min fixation with 4% PFA. Cell were then washed three times with PBS, blocked in 10% neat goat serum (NGS), 0.25% Triton X-100 in PBS for 1 h at room temperature. Permeabilized fixed cells were then incubated overnight in 10% NGS/0.25% Triton X-100 in PBS with monoclonal anti-mouse MC1 antibody detected misfolded Tau (provided by Prof. Peter Davies, Albert Einstein College of Medicine, New York, USA) and polyclonal anti-rabbit total Tau (Abcam; ab64193) both diluted 1:1000. Following the incubation with primary antibodies, cells were washed 3 times in PBS and then incubated 45 min with secondary Antibodies (Cy3-labeled goat-anti mouse (Jackson; 115-165-146) and Alexa Fluor 488-labeled goat anti-rabbit (Jackson; 111-545-144). Cells were then washed 3 times in PBS and mounted with Prolong Gold mounting media containing DAPI (Invitrogen, P36931). Images were then acquired with the Panoramic 250 slide scanner from 3DHISTECH and analysis was done with the VisioPharm software. Reduction of intracellular misfolded Tau observed with Compound 1 used at 20 nM was shown to be significantly different from DMSO control (FIG. 1, unpaired test).

In Vivo Efficacy of the Compounds of the Present Invention

Double transgenic rTg4510 mice express the full-length human Tau carrying the P301L mutation (Tau4RON-P301L) under the control of the tet-inducible (or tetracycline-inducible system) CaMKII promoter (Ramsden et al., J. Neurosci., 2005·25(46):10637-10647). Single transgenic mice expressing only the tetracycline-controlled transactivator (tTA) were used as genotype controls. The study comprised 4 treatment groups (n=15 female for tTa group and n=33 female mice/group for the treatment) with the following group distribution (see Table 5). Mice were distributed over 5 cohorts and cages were composed with an average of 3 mice per cage. Compounds or vehicle control were administered bi-daily by gavage for 4 weeks starting at the age of 5 months.

TABLE 5
In vivo study design for the testing of compound of Example 1

| Genotype | Number of mice | Treatment/dose |
|---|---|---|
| tTA | 15 | Vehicle^{(a)} |
| rTg4510 | 33 | Vehicle^{(a)} |
| rTg4510 | 33 | Example 1 (30 mg/kg bi-daily) |
| rTg4510 | 33 | Example 1 (100 mg/kg bi-daily) |

^{(a)}vehicle: 0.5% CMC(w/v) in water with 0.2% Tween 80

Total Cortical Homogenate Preparation and Analysis

To prepare cortical total brain homogenates (Cx-TBH), frozen brains were resuspended in 9 volumes/weight of ice-cold homogenization buffer [25 mM Tris-HCl pH 7.4, 150 mM NaCl, 1 mM EDTA, 1 mM EGTA containing phosphatase inhibitors (30 mM NaF, 0.2 mM Na₃VO₄, 1 nM Okadaic acid, 1 mM PMSF, 5 mM Na₄P₂O₇) and protease inhibitor cocktail (Complete™, Roche)] and homogenized in Eppendorf tubes with a VWR pellet mixer (47747-370). Samples were rapidly immersed in liquid nitrogen and stored at −80° C. until biochemical analysis was performed by AlphaLISA. Aggregated Tau in total cortical homogenate was quantified using the following antibody pairs: HT7-Acceptor beads+biotin (BT)-HT7-donor beads. Both HT7 antibodies, biotinylated or not were purchased (Thermofisher). For the final protocol, the following reagents were added in a 384-well white OptiPlate (PerkinElmer):
5 μL of test diluted sample
20 μL of the mixture biotin-mAb acceptor beads at the final concentrations: Tau13-BT at 0.6 nM in combination with HT7-Acc beads at 2.5 μg/ml

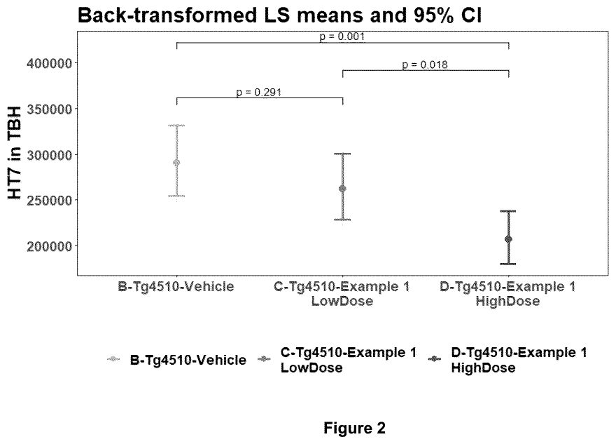

After incubation of this mixture at room temperature for 1 hour, 25 μL of Streptavidin Donor beads (Perkin Elmer) at 25 μg/mL were added in the dark. Plates were analyzed after 30 minutes incubation using the EnSpire Alpha instrument and EnSpire Workstation version 3.00. Results are presented as LS means (or Least Square mens) and statistical analysis is performed using Linear Mixed Model, Treatment Group and Cohort as fixed factors, Cage as random factor, uncorrected p-values As shown in FIG. 2, the compound of Example 1, when dosed at 100 mg/kg, significantly decreased the aggregated Tau in Cortical Total brain homogenate (Cx-TBH) of the rTg4510 mice. The significant decrease of aggregated Tau in Cortical Total brain homogenate (Cx-TBH) was accompanied by a significant decrease of the NeuroFibrillary Tangles (NFTs), as well as neuroinflammation markers Iba1 and CD68 analyzed by immunohistochemistry (data not shown).

1. A compound of formula (I): 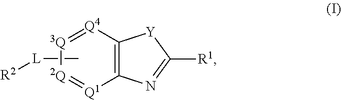
or a pharmaceutically acceptable salt thereof, wherein
Y is S or O;
R¹ is a mono or bicyclic heterocyclyl;
Q¹ and Q⁴ are different and independently selected from CH and N;
Q² and Q³ are different and independently selected from N, C, and C-L-R², wherein at least of Q² or Q³ is C-L-R²;
L is —NH(CO)—, C₂-C₄alkynyl, —NH—; or
L is a heteroaryl; or
L is a 5- to 8-membered saturated or unsaturated heterocyclyl optionally substituted with halo or C₁-C₄ alkyl; or
L is a bond
R² is selected from 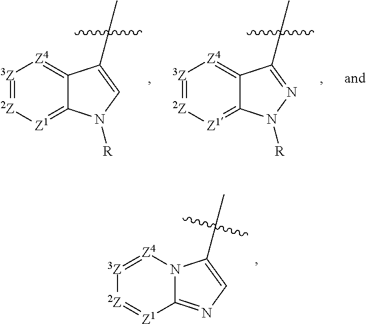
wherein
R is C₁-C₄ alkyl or H;
Z¹ is N, CH, C—F, and C—OCH₃;
Z^{1′} is N, CH, C—F, C—CH₃, and C—OCH₃;
Z² is N, CH, C—F, C—CH₃, and C—OCH₃;
Z³ or Z⁴ are independently selected from N, CH, C—F and C—CH₃; and
wherein when Z⁴ is N, at least one of Z¹, Z¹, Z², Z³ is C—F.

2. The compound according to claim 1, or a pharmaceutical acceptable salt thereof, having a formula (II): 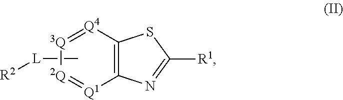
wherein R¹, R², L, Q¹, Q², Q³ and Q⁴ are as defined in claim 1.

3. The compound according to claim 1, or a pharmaceutical acceptable salt thereof, having a formula (III): 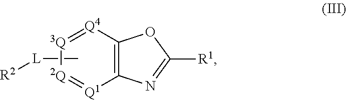
wherein R¹, R², L, Q¹, Q², Q³ and Q⁴ are as defined in claim 1.

4. The compound according to claim 1, wherein R² is selected from the following: 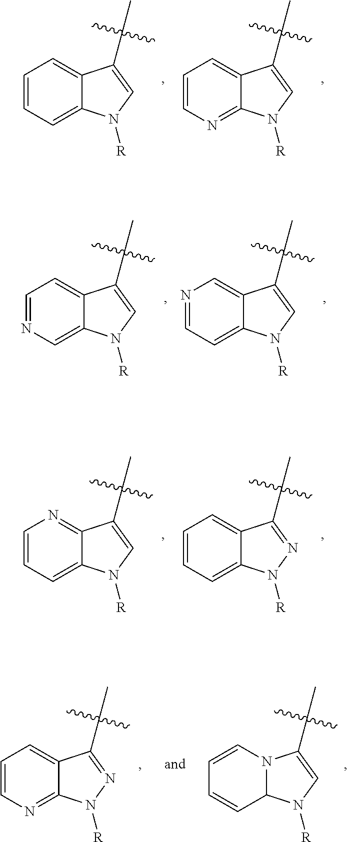
wherein R is C₁-C₄ alkyl or H; and R is optionally substituted with 1 to 2 substituents independently selected from F, CH₃ and OCH₃ as defined in claim 1.

5. The compound according to claim 1, wherein R¹ is selected from the following:

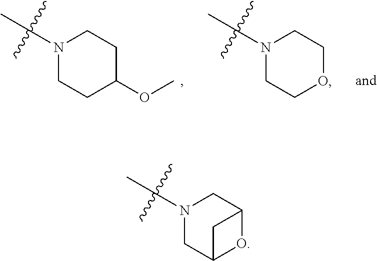

6. The compound according to claim 1, wherein L is selected from the following —NH(CO)—, C₂-C₄alkynyl, —NH—, heteroaryl, and 5- to 8-membered saturated or unsaturated heterocyclyl optionally substituted with halo or C₁-C₄ alkyl.

7. The compound according to claim 1, wherein Q¹, Q², Q³ and Q⁴ are all C, and wherein at least one of Q² or Q³ is C-L-R².

8. The compound according to claim 1, wherein only one of Q¹, Q², Q³ and Q⁴ is N.

9. The compound according to claim 1, wherein when Q¹ is N, R² comprises two nitrogen atoms.

10. The compound according to claim 1, wherein the compound is selected from:
5-(4-(1H-indazol-3-yl)piperidin-1-yl)-2-morpholinobenzo[d]oxazole;
5-(5-fluoro-1-methyl-1H-pyrrolo[2,3-b]pyridin-3-yl)-2-morpholinobenzo[d]oxazole;
5-(1H-indazol-3-yl)-2-morpholinobenzo[d]oxazole;
5-((1H-indazol-3-yl)ethynyl)-2-morpholinobenzo[d]oxazole;
5-((1H-indol-3-yl)ethynyl)-2-morpholinobenzo[d]oxazole;
N-(1H-indol-3-yl)-2-morpholinobenzo[d]oxazole-5-carboxamide;
5-(4-(1H-indazol-3-yl)-1H-pyrazol-1-yl)-2-morpholinobenzo[d]oxazole;
5-(4-(1H-indol-3-yl)-1H-pyrazol-1-yl)-2-morpholinobenzo[d]oxazole;
5-(3-(1H-indazol-3-yl)-1H-pyrazol-1-yl)-2-morpholinobenzo[d]oxazole;
5-(3-(1H-indol-3-yl)-1H-pyrazol-1-yl)-2-morpholinobenzo[d]oxazole;
N-(2-morpholinobenzo[d]oxazol-5-yl)-1H-indole-3-carboxamide;
N-(2-morpholinobenzo[d]oxazol-5-yl)-1H-pyrrolo[2,3-b]pyridine-3-carboxamide;
N-(2-morpholinobenzo[d]oxazol-6-yl)-1H-indole-3-carboxamide;
N-(2-morpholinobenzo[d]oxazol-6-yl)-1H-pyrrolo[2,3-b]pyridine-3-carboxamide;
N-(2-morpholinobenzo[d]oxazol-6-yl)-1H-indazole-3-carboxamide;
5-fluoro-N-(2-morpholinobenzo[d]thiazol-6-yl)-1H-indole-3-carboxamide;
N-(2-morpholinobenzo[d]oxazol-5-yl)-1H-indazole-3-carboxamide;
5-(4-(1H-indol-3-yl)piperidin-1-yl)-2-morpholinobenzo[d]oxazole;
5-(4-(1H-pyrrolo[2,3-b]pyridin-3-yl)piperidin-1-yl)-2-morpholinobenzo[d]oxazole;
5-(4-(1-methyl-1H-pyrrolo[2,3-b]pyridin-3-yl)piperidin-1-yl)-2-morpholinobenzo[d]oxazole;
5-(4-(5-fluoro-1H-pyrrolo[2,3-b]pyridin-3-yl)piperidin-1-yl)-2-morpholinobenzo[d]oxazole;
5-(4-(5-fluoro-1-methyl-1H-pyrrolo[2,3-b]pyridin-3-yl)piperidin-1-yl)-2-morpholinobenzo[d]oxazole;
4-(6-(4-(1H-indol-3-yl)piperidin-1-yl)thiazolo[4,5-c]pyridin-2-yl)morpholine;
5-(4-(imidazo[1,2-a]pyridin-3-yl)piperidin-1-yl)-2-morpholinobenzo[d]oxazole;
4-(6-(4-(5-fluoro-1-methyl-1H-pyrrolo[2,3-b]pyridin-3-yl)piperidin-1-yl)thiazolo[4,5-b]pyridin-2-yl)morpholine;
4-(6-(4-(5-fluoro-1-methyl-1H-pyrrolo[2,3-b]pyridin-3-yl)piperidin-1-yl)thiazolo[4,5-c]pyridin-2-yl)morpholine;
4-(6-(4-(1H-indol-3-yl)piperidin-1-yl)thiazolo[4,5-b]pyridin-2-yl)morpholine;
4-(6-(4-(imidazo[1,2-a]pyridin-3-yl)piperidin-1-yl)benzo[d]thiazol-2-yl)morpholine;
4-(6-(4-(6-fluoro-1H-pyrrolo[3,2-b]pyridin-3-yl)piperidin-1-yl)benzo[d]thiazol-2-yl)morpholine;
5-(4-(1-methyl-1H-pyrazolo[3,4-b]pyridin-3-yl)piperidin-1-yl)-2-morpholinobenzo[d]oxazole;
5-(4-(5-fluoro-1-methyl-1H-pyrrolo[2,3-b]pyridin-3-yl)-3,6-dihydropyridin-1(2H)-yl)-2-morpholinobenzo[d]oxazole;
5-(3-(5-fluoro-1-methyl-1H-pyrrolo[2,3-b]pyridin-3-yl)pyrrolidin-1-yl)-2-morpholinobenzo[d]oxazole;
5-(3-(5-fluoro-1-methyl-1H-pyrrolo[2,3-b]pyridin-3-yl)pyrrolidin-1-yl)-2-morpholinobenzo[d]oxazole;
4-(6-(4-(1H-indazol-3-yl)piperidin-1-yl)thiazolo[4,5-c]pyridin-2-yl)morpholine;
N-(1H-indazol-3-yl)-2-morpholinobenzo[d]oxazol-5-amine;
5-(3-(1H-indazol-3-yl)pyrrolidin-1-yl)-2-morpholinobenzo[d]oxazole;
5-(3-(1H-indazol-3-yl)pyrrolidin-1-yl)-2-morpholinobenzo[d]oxazole;
5-(3-(1H-indol-3-yl)pyrrolidin-1-yl)-2-morpholinobenzo[d]oxazole;
5-(3-(1H-indol-3-yl)pyrrolidin-1-yl)-2-morpholinobenzo[d]oxazole;
4-(6-(4-(1H-indazol-3-yl)piperidin-1-yl)thiazolo[4,5-b]pyridin-2-yl)morpholine;
4-(6-(4-(1H-indazol-3-yl)piperidin-1-yl)thiazolo[5,4-b]pyridin-2-yl)morpholine;
5-(4-(1H-indazol-3-yl)piperidin-1-yl)-2-morpholinooxazolo[4,5-b]pyridine;
4-(6-(4-(1H-indazol-3-yl)piperidin-1-yl)benzo[d]thiazol-2-yl)morpholine;
6-(4-(1H-indazol-3-yl)piperidin-1-yl)-2-morpholinooxazolo[5,4-c]pyridine;
5-((5-fluoro-1-methyl-1H-pyrrolo[2,3-b]pyridin-3-yl)ethynyl)-morpholinobenzo[d]oxazole;
6-(4-(1H-indazol-3-yl)piperidin-1-yl)-2-morpholinooxazolo[4,5-b]pyridine;
6-(4-(1H-indol-3-yl)piperidin-1-yl)-2-morpholinooxazolo[5,4-c]pyridine;
6-(4-(1H-indazol-3-yl)piperidin-1-yl)-2-morpholinooxazolo[5,4-b]pyridine;
5-(4-(1H-indazol-3-yl)-3,6-dihydropyridin-1(2H)-yl)-2-morpholinobenzo[d]oxazole;
5-(4-(5-fluoro-1-methyl-1H-indol-3-yl)piperidin-1-yl)-2-morpholinobenzo[d]oxazole;
6-(4-(1H-indol-3-yl)piperidin-1-yl)-2-morpholinobenzo[d]oxazole;
5-(4-(6-fluoro-1H-pyrrolo[3,2-b]pyridin-3-yl)piperidin-1-yl)-2-morpholinobenzo[d]oxazole;
5-(4-(1H-pyrrolo[2,3-c]pyridin-3-yl)piperidin-1-yl)-2-morpholinobenzo[d]oxazole;
5-(4-(1H-pyrrolo[3,2-c]pyridin-3-yl)piperidin-1-yl)-2-morpholinobenzo[d]oxazole;
5-(4-(1H-indol-3-yl)piperazin-1-yl)-2-morpholinobenzo[d]oxazole;
4-(5-(4-(5-fluoro-1-methyl-1H-pyrrolo[2,3-b]pyridin-3-yl)piperidin-1-yl)thiazolo[5,4-b]pyridin-2-yl)morpholine;
6-(4-(1H-indol-3-yl)piperidin-1-yl)-2-morpholinooxazolo[4,5-c]pyridine;
6-(4-(imidazo[1,2-a]pyridin-3-yl)piperidin-1-yl)-2-morpholinobenzo[d]oxazole;
5-(5-(5-fluoro-1-methyl-1H-pyrrolo[2,3-b]pyridin-3-yl)hexahydrocyclopenta[c]pyrrol-2(1H)-yl)-2-morpholinobenzo[d]oxazole;
5-(4-(5-fluoro-1H-indazol-3-yl)piperidin-1-yl)-2-morpholinobenzo[d]oxazole;
4-(5-(4-(1H-indazol-3-yl)piperidin-1-yl)thiazolo[4,5-b]pyridin-2-yl)morpholine;
4-(5-(4-(1H-indazol-3-yl)piperidin-1-yl)benzo[d]thiazol-2-yl)morpholine;
5-(4-(1H-indazol-3-yl)piperidin-1-yl)-2-morpholinooxazolo[5,4-b]pyridine;
4-(6-(4-(1H-indol-3-yl)piperidin-1-yl)thiazolo[5,4-b]pyridin-2-yl)morpholine;
4-(6-(4-(1H-indazol-3-yl)piperidin-1-yl)thiazolo[5,4-c]pyridin-2-yl)morpholine;
4-(6-(4-(1H-indol-3-yl)piperidin-1-yl)thiazolo[5,4-c]pyridin-2-yl)morpholine;
5-(4-(1-methyl-1H-indol-3-yl)piperidin-1-yl)-2-morpholinobenzo[d]oxazole;
5-(4-(5-fluoro-1H-indol-3-yl)piperidin-1-yl)-2-morpholinobenzo[d]oxazole;
6-(4-(5-fluoro-1-methyl-1H-pyrrolo[2,3-b]pyridin-3-yl)piperidin-1-yl)-2-morpholinobenzo[d]oxazole;
4-(5-(4-(1H-indol-3-yl)piperidin-1-yl)thiazolo[5,4-b]pyridin-2-yl)morpholine; 6-(4-(1H-indazol-3-yl)piperidin-1-yl)-2-morpholinooxazolo[4,5-c]pyridine;
5-(4-(4-fluoro-1H-indazol-3-yl)piperidin-1-yl)-2-morpholinobenzo[d]oxazole;
5-(4-(6-fluoro-1H-indazol-3-yl)piperidin-1-yl)-2-morpholinobenzo[d]oxazole;
5-(4-(7-fluoro-1H-indazol-3-yl)piperidin-1-yl)-2-morpholinobenzo[d]oxazole;
5-(4-(5-methyl-1H-indazol-3-yl)piperidin-1-yl)-2-morpholinobenzo[d]oxazole;
5-(4-(6-methyl-1H-indazol-3-yl)piperidin-1-yl)-2-morpholinobenzo[d]oxazole;
5-(4-(7-methyl-1H-indazol-3-yl)piperidin-1-yl)-2-morpholinobenzo[d]oxazole;
5-(4-(1H-indazol-3-yl)piperazin-1-yl)-2-morpholinobenzo[d]oxazole;
5-(4-(1H-indazol-3-yl)piperidin-1-yl)-2-(4-methoxypiperidin-1-yl)benzo[d]oxazole;
4-(5-(3-(5-fluoro-1-methyl-1H-pyrrolo[2,3-b]pyridin-3-yl)pyrrolidin-1-yl)benzo[d]thiazol-2-yl)morpholine;
4-(6-(3-(5-fluoro-1-methyl-1H-pyrrolo[2,3-b]pyridin-3-yl)pyrrolidin-1-yl)benzo[d]thiazol-2-yl)morpholine;
4-(6-(3-(5-fluoro-1-methyl-1H-pyrrolo[2,3-b]pyridin-3-yl)pyrrolidin-1-yl)benzo[d]thiazol-2-yl)morpholine;
6-(3-(5-fluoro-1-methyl-1H-pyrrolo[2,3-b]pyridin-3-yl)pyrrolidin-1-yl)-2-morpholinooxazolo[5,4-b]pyridine;
6-(3-(5-fluoro-1-methyl-1H-pyrrolo[2,3-b]pyridin-3-yl)pyrrolidin-1-yl)-2-morpholinooxazolo[5,4-b]pyridine;
6-(3-(5-fluoro-1-methyl-1H-pyrrolo[2,3-b]pyridin-3-yl)pyrrolidin-1-yl)-2-morpholinooxazolo[5,4-c]pyridine;
6-(3-(5-fluoro-1-methyl-1H-pyrrolo[2,3-b]pyridin-3-yl)pyrrolidin-1-yl)-2-morpholinooxazolo[5,4-c]pyridine;
6-(3-(5-fluoro-1-methyl-1H-pyrrolo[2,3-b]pyridin-3-yl)pyrrolidin-1-yl)-2-morpholinobenzo[d]oxazole;
6-(3-(5-fluoro-1-methyl-1H-pyrrolo[2,3-b]pyridin-3-yl)pyrrolidin-1-yl)-2-morpholinobenzo[d]oxazole;
5-(3-(5-fluoro-1-methyl-1H-pyrrolo[2,3-b]pyridin-3-yl)pyrrolidin-1-yl)-2-morpholinooxazolo[4,5-b]pyridine;
5-(3-(5-fluoro-1-methyl-1H-pyrrolo[2,3-b]pyridin-3-yl)pyrrolidin-1-yl)-2-morpholinooxazolo[4,5-b]pyridine;
5-(4-(7-methoxy-1H-indazol-3-yl)piperidin-1-yl)-2-morpholinobenzo[d]oxazole;
5-(4-(4-methyl-1H-indazol-3-yl)piperidin-1-yl)-2-morpholinobenzo[d]oxazole;
5-(3-fluoro-4-(1H-indazol-3-yl)piperidin-1-yl)-2-morpholinobenzo[d]oxazole;
4-(5-(3-(5-fluoro-1-methyl-1H-pyrrolo[2,3-b]pyridin-3-yl)pyrrolidin-1-yl)benzo[d]thiazol-2-yl)morpholine;
5-(3-(5-fluoro-1-methyl-1H-pyrrolo[2,3-b]pyridin-3-yl)pyrrolidin-1-yl)-2-(4-methoxypiperidin-1-yl)benzo[d]oxazole;
5-(3-(5-fluoro-1-methyl-1H-pyrrolo[2,3-b]pyridin-3-yl)pyrrolidin-1-yl)-2-(4-methoxypiperidin-1-yl)benzo[d]oxazole;
3-(5-(3-(5-fluoro-1-methyl-1H-pyrrolo[2,3-b]pyridin-3-yl)pyrrolidin-1-yl)benzo[d]oxazol-2-yl)-6-oxa-3-azabicyclo[3.1.1]heptane;
3-(5-(3-(5-fluoro-1-methyl-1H-pyrrolo[2,3-b]pyridin-3-yl)pyrrolidin-1-yl)benzo[d]oxazol-2-yl)-6-oxa-3-azabicyclo[3.1.1]heptane;
6-(4-(1H-indol-3-yl)piperidin-1-yl)-2-morpholinooxazolo[4,5-b]pyridine;
5-(4-(1H-indol-3-yl)piperidin-1-yl)-2-morpholinooxazolo[5,4-b]pyridine;
5-(3-(5-fluoro-1-methyl-1H-pyrrolo[2,3-b]pyridin-3-yl)pyrrolidin-1-yl)-2-morpholinobenzo[d]oxazole;
6-(4-(6-fluoro-1H-pyrrolo[3,2-b]pyridin-3-yl)piperidin-1-yl)-2-morpholinobenzo[d]oxazole;
5-(4-(6-methoxy-1H-pyrrolo[2,3-b]pyridin-3-yl)piperidin-1-yl)-2-morpholinobenzo[d]oxazole;
5-(4-(6-fluoro-1-methyl-1H-pyrrolo[3,2-b]pyridin-3-yl)piperidin-1-yl)-2-morpholinobenzo[d]oxazole;
4-(5-(4-(1H-indazol-3-yl)piperidin-1-yl)thiazolo[5,4-b]pyridin-2-yl)morpholine;
5-(4-(1H-indol-3-yl)-3,6-dihydropyridin-1(2H)-yl)-2-morpholinobenzo[d]oxazole;
4-(5-(4-(1H-indol-3-yl)piperidin-1-yl)thiazolo[5,4-b]pyridin-2-yl)morpholine hydrochloride;
4-(6-(4-(5-fluoro-1-methyl-1H-pyrrolo[2,3-b]pyridin-3-yl)piperidin-1-yl)thiazolo[4,5-b]pyridin-2-yl)morpholine hydrochloride;
4-(6-(4-(5-fluoro-1-methyl-1H-pyrrolo[2,3-b]pyridin-3-yl)piperidin-1-yl)thiazolo[4,5-c]pyridin-2-yl)morpholine hydrochloride;
6-(4-(5-fluoro-1-methyl-1H-pyrrolo[2,3-b]pyridin-3-yl)piperidin-1-yl)-2-morpholinobenzo[d]oxazole hydrochloride;
5-(5-(5-fluoro-1-methyl-1H-pyrrolo[2,3-b]pyridin-3-yl)hexahydrocyclopenta[c]pyrrol-2(1H)-yl)-2-morpholinobenzo[d]oxazole hydrochloride;
or a pharmaceutically acceptable salts thereof.

11. A pharmaceutical composition comprising a compound according to claim 1, and optionally a pharmaceutically acceptable carrier, diluent, adjuvant and/or excipient.

12. (canceled)

13. (canceled)

14. A method of treating, alleviating or preventing a disease, disorder or abnormality associated with misfolding of Tau protein and/or pathological aggregation of Tau protein comprising administering a compound according to claim 1.

15. The method according to claim 14, wherein the compound is administered with one or more additional therapeutic agent.

16. A method of treating, alleviating, or preventing a disease, disorder, or abnormality associated with misfolding of tau protein and/or pathological aggregation of Tau protein comprising administering a pharmaceutical composition as defined in claim 11.

17. A method of decreasing Tau aggregation, preventing the formation of Tau aggregates, inhibiting Tau aggregation, and/or interfering intracellularly with Tau aggregates, the method comprising administering a compound of claim 1, to a subject in need thereof.

18. (canceled)

19. (canceled)

20. The method of claim 14, wherein the disease, disorder or abnormality associated with misfolding of Tau protein and/or pathological aggregation of Tau protein is selected from Alzheimer's disease (AD), familial Alzheimer's disease (AD), Primary Age-Related Tauopathy (PART), Creutzfeldt-Jacob disease, dementia pugilistica, Down's Syndrome, Gerstmann-Straussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17) also known familiar FTLD-tau (MAPT), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, subacute sclerosing panencephalitis (SSPE), Senile dementia of the neurofibrillary tangle type, Parkinson's disease, HIV-related dementia, adult onset diabetes, senile cardiac amyloidosis, glaucoma, ischemic stroke, psychosis in Alzheimer's disease (AD), Lafora disease and Huntington's disease.

21. The method of claim 14, wherein the disease, disorder or abnormality associated with misfolding of Tau protein and/or pathological aggregation of Tau protein is Alzheimer's disease (AD).

22. The method of claim 14, wherein the disease, disorder or abnormality associated with misfolding of Tau protein and/or pathological aggregation of Tau protein is progressive supranuclear palsy (PSP).

23. The method of claim 14, wherein the disease, disorder or abnormality associated with misfolding of Tau protein and/or pathological aggregation of Tau protein is frontotemporal dementia with Parkinsonism linked to chromosome 17 (FTDP-17) also known familiar FTLD-Tau (MAPT).

24. A combination comprising a therapeutically effective amount of a compound according to claim 1, and one or more additional therapeutic agents.

25. A mixture comprising a compound according to claim 1, and one or more therapeutic agent different from the compound as defined in claim 1, and optionally a pharmaceutically acceptable carrier, diluent, adjuvant and/or excipient.

26. The combination according to claim 24, wherein the one or more therapeutic agents are selected from the group consisting of compounds against oxidative stress; anti-amyloid drug; anti-apoptotic compounds; metal chelators; inhibitors of DNA repair such as pirenzepine and metabolites; 3-amino-1-propanesulfonic acid (3APS); 1,3-propanedisulfonate (1,3PDS); alpha-secretase activators; beta- and gamma-secretase inhibitors including BACE1; Tau proteins; neurotransmitters; beta-sheet breakers; attractants for amyloid beta clearing/depleting cellular components; inhibitors of N-terminal truncated amyloid beta including pyroglutamated amyloid beta 3-42; anti-inflammatory molecules; cholinesterase inhibitors (ChEIs) such as tacrine, rivastigmine, donepezil, and/or galantamine; M1 agonists; amyloid-beta or Tau modifying drugs; nutritive supplements; neurological drugs; corticosteroids, antibiotics, or antiviral agents.

27. A method of using a compound of claim 1, as an analytical reference or an in vitro screening tool.
